(19) United States
(12) Patent Application Publication
(10) Pub. No.: US 2026/0240817 A1
(43) Pub. Date: Aug. 20, 2026

(54) COMPOSITIONS AND METHODS FOR TREATING CHARCOT-MARIE-TOOTH DISEASE

(71) Applicant: NMD PHARMA A/S, Aarhus N (DK)

(72) Inventors: John Blundell HUTCHISON, Reading (GB); Jorge Armando Quiroz BASTIAS, Aarhus N (DK); Thomas Holm PEDERSEN, Aarhus N (DK); Thomas Skjærlund GRØNNEBÆK, Aarhus N (DK); Ole Baekgaard NIELSEN, Aarhus N (DK); Martin Brandhøj SKOV, Aarhus N (DK)

(73) Assignee: NMD PHARMA A/S, Aarhus N (DK)

(21) Appl. No.: 19/161,100

(22) PCT Filed: Mar. 1, 2024
(86) PCT No.: PCT/EP2024/055306

§ 371 Date: Aug. 29, 2025

Related U.S. Application Data
(60) Provisional application No. 63/488,058, filed on Mar. 2, 2023.

(30) Foreign Application Priority Data
Mar. 27, 2023 (EP) 23164242.2
Nov. 8, 2023 (EP) 23208561.3

(51) Int. Cl. A61K 31/416; A61K 9/00; A61K 9/20; A61K 31/717

(52) U.S. Cl. CPC A61K 31/416; A61K 9/0053; A61K 9/2013; A61K 9/2054; A61K 9/2059; A61K 31/717

(57) ABSTRACT

Disclosed herein are methods of treatment for Charcot-Marie-Tooth disease (CMTD) that involve administering (2S)-2-[4-bromo-2-(1,2-oxazol-3-yl)phenoxy]propanoic acid, or a pharmaceutical composition thereof, to a patient suffering from symptoms of CMTD. Pharmaceutical compositions and kits-to-parts including (2S)-2-[4-bromo-2-(1,2-oxazol-3-yl)phenoxy]propanoic acid are also disclosed.

### TECHNICAL FIELD

The present disclosure relates to (2S)-2-[4-bromo-2-(1,2-oxazol-3-yl)phenoxy]propanoic acid for use in the treatment of Charcot-Marie-Tooth disease (CMTD), its pharmaceutical composition for use in the treatment of CMTD and methods of treatment thereof.

### BACKGROUND

Charcot-Marie-Tooth disease (CMTD), also referred to as Hereditary Motor and Sensory Neuropathy, is a hereditary motor and sensory neuropathy of the peripheral nervous system characterized by progressive loss of skeletal muscle tissue and function, and touch sensation across various parts of the body. This disease is the most commonly inherited neurological disorder involving the peripheral nerves, affecting about one in 2,500 people (Krajewski et al, 2000). CMTD is a heterogeneous disease with more than 100 different genes causally linked to CMT. It can be classified into 4 major disease types (Fridman et al, 2015): CMT1, CMT2, CMT4 and CMTX (table 1).

TABLE 1
CMT classification of major disease types

| CMT | | Most Common | |
|---|---|---|---|
| type | Disease Characteristics | Subtypes | Disease onset |
| CMT1 | Autosomal dominant | CMT1A | Infancy to later decades of |
| | demyelinating form | CMT1B | life |
| CMT2 | Axonal form - typically | CMT2A | Infancy to later decades of |
| | autosomal dominant | CMT2E | life. CMT2A typically |
| | inheritance | | childhood |
| CMT4 | Autosomal recessive | CMT4A | Typically, infancy |
| | demyelinating form | CMT4C | |
| CMTX | X-linked | CMTX1 | Typically, adolescence or |
| | | | early adulthood |

Progressive skeletal muscle weakness is hallmark of CMT, and distal muscles (feet, hands, lower legs) are generally more severely affected (Saporta et al 2011). Most patients have a “classical” CMT phenotype characterized by onset in the first two decades of life, distal weakness, sensory loss, foot deformities (pes cavus and hammer toes), loss of hand grip, and absent ankle reflexes (Saporta et al 2011). Quality of life (QoL) can be severely affected in patients with CMT and impaired QoL is closely related to symptoms of weakness and fatigue as well as to loss of physical capabilities (Boentert et al 2010; Vinci et al 2005).

There are no approved treatments for CMT. Treatments that could address muscle weakness, loss of hand grip strength and generally improve the QoL of CMT patients are therefore required.

Skeletal muscle specific CIC-1 chloride ion channels carry the inhibitory currents that counteract neuromuscular transmission. Inhibition of CIC-1 reduces the inhibitory current and thereby increases muscle membrane excitability and enhances neuromuscular transmission. This was shown to lead to recovery of muscle function under conditions mimicking neuromuscular disorders (Pedersen et al 2021).

(2S)-2-[4-bromo-2-(1,2-oxazol-3-yl)phenoxy]propanoic acid, hereinafter NMD670, is an inhibitor of the skeletal muscle-specific CIC-1 channel. The chemical structure of NMD670 is provided below.

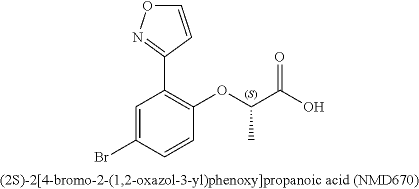

NMD670 alters the voltage sensitivity of the CIC-1 channel resulting in decreased membrane conductance for Cl⁻ in muscle fibres and increased muscle fibre excitability. CIC-1 inhibition with NMD670 restores muscle activation under conditions of failing neuromuscular transmission or compromised muscle fibre excitability and under these conditions. NMD670 can restore force production of skeletal muscle.

Accordingly, the CIC-1 channel is emerging as a target for potential drugs, although its potential has been largely unrealized. U.S. Pat. No. 10,385,028 discloses the synthesis of compounds which have been designed to inhibit the action of the CIC-1 channel to treat neuromuscular disorders. One of the compounds discussed in U.S. Pat. No. 10,385,028 is NMD670.

Though U.S. Pat. No. 10,385,028 discloses a set of compounds which can inhibit the CIC-1 channel to treat neuromuscular disorders, there is no discussion into how to design treatment methods to where these compounds can effectively alleviate the wide range of symptoms associated with Charcot-Marie-Tooth disease. WO2020/254554, herein incorporated by reference, discloses methods for manufacturing NMD670.

Accordingly, there is a need for safe and efficacious therapies to improve muscle function in patients with CMT.

### SUMMARY

The present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein the composition is for administration at a therapeutic dose of 100 to 1500 mg of (2S)-2-[4-bromo-2-(1,2-oxazol-3-yl)phenoxy]propanoic acid. The present disclosure further relates to a kit-of-parts comprising (2S)-2-[4-bromo-2-(1,2-oxazol-3-yl)phenoxy]propanoic acid, or a pharmaceutically acceptable salt, hydrate, polymorph, tautomer, or solvate thereof, and a peripheral myelin protein 22 (PMP22) down regulator.

### DESCRIPTION OF DRAWINGS

FIG. 1

FIG. 1 depicts the 30 first endplate potential (EPP) amplitude recordings from single muscle fibres from mouse soleus muscle. Intracellular recordings of EPPs were obtained from single Conotoxin-treated muscle fibres during stimulation of the motor nerve at 80 Hz for 1 second in nerve-muscle preparation isolated from wild type (WT) (34 fibres) and CMT2D mice (60 fibres). Only fibres that were able to sustain generation of EPPs for the entire stimulus train were included in the above analysis. The average EPP amplitude with SEM is shown in FIG. 1, for WT mice (black circle), CMT2D mice pre-treatment (open triangles) and CMT2D mice after treatment with NMD670 (grey squares). The average EPP amplitude in CMT2D mice was lower than in WT mice but after addition of 10 μM NMD670 to the CMT2D fibres, a significant increase in EPP amplitude was observed (p-val<0.001) in a paired t-test.

FIG. 2

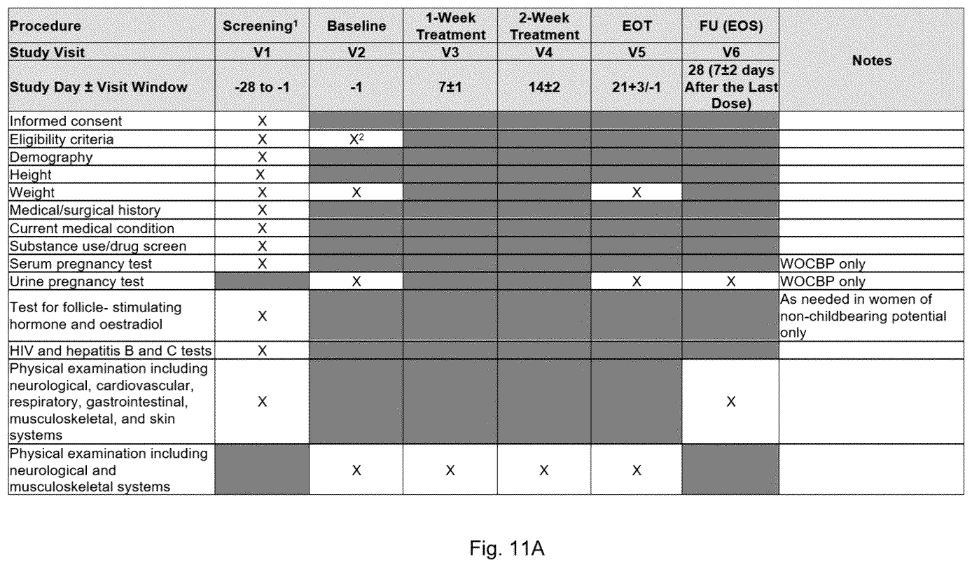

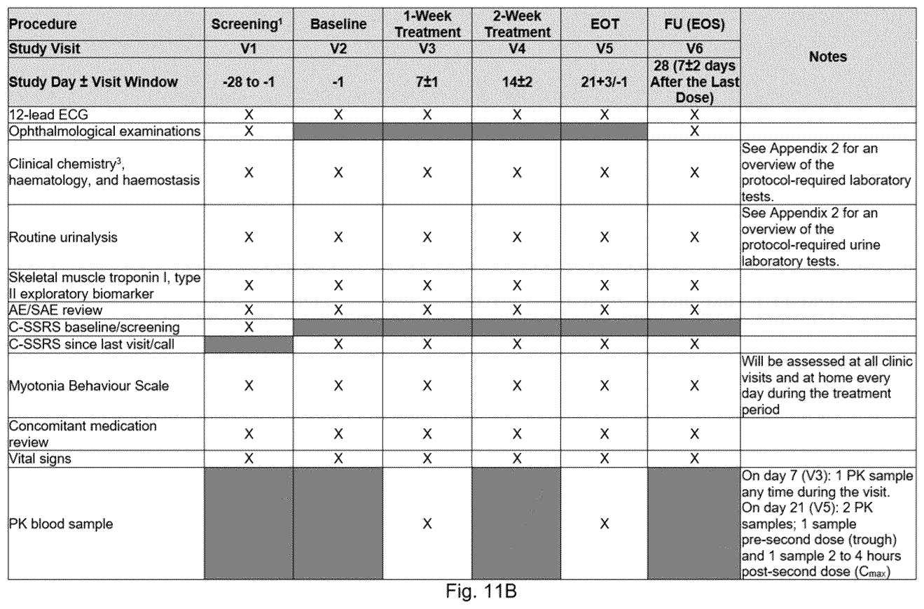

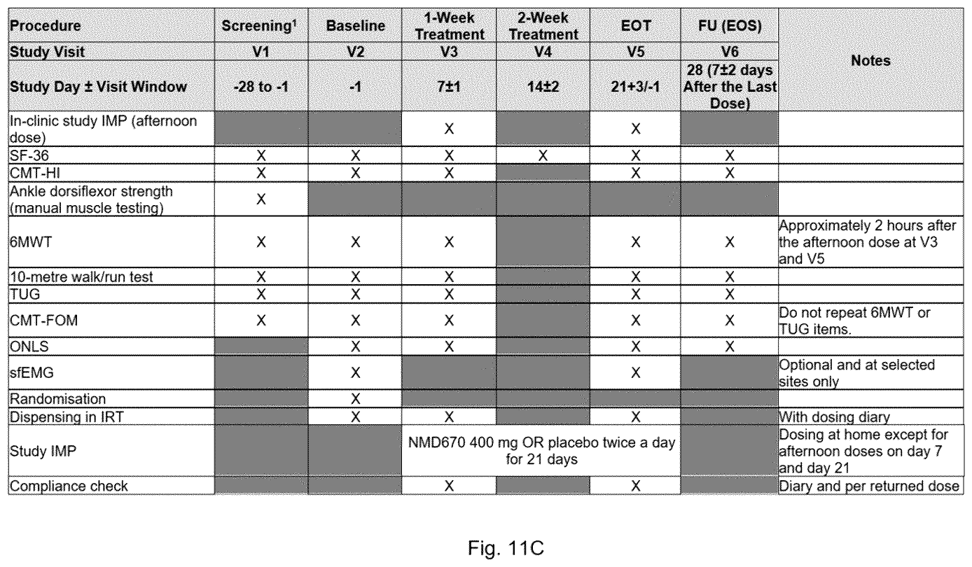

Action potentials were recorded from mouse soleus muscle fibres with intracellular electrodes during repeated stimulations of the motor nerve of isolated nerve-muscle preparations. FIG. 2 depicts action potential excitation failures during repeated stimulations of the motor nerve at different frequencies. To determine this, the action potentials that failed to be generated by nerve-stimulation were determined for each fibre for the respective stimulus pulse number in the stimulation trains. The observations from all fibres were combined to get a percentage of action potential excitation failures across all fibres per group and plotted against stimulation number in the trains of nerve-stimulation pulses. This was done for two groups of fibres: before addition of 10 μM NMD670 (black triangles) and after (grey squares). The average failure was 3.6, 4.9 and 29.6% before addition, and 1.9, 0.8 and 20.4% after addition of NMD670 at 12 Hz (FIG. 2A), 30 Hz (FIG. 2B), and 80 Hz (FIG. 2C), respectively.

FIG. 3

FIG. 3 depicts force and compound muscle action potentials (CMAP) from the triceps surae muscle group in the hind limb of CMT2 mouse model, Gars^{P278KY} mouse. Animals were placed in anaesthesia and mechanically ventilated. The achilleas tendon was cut and attached to force transducer by a string. The sciatic nerve was stimulated with electrodes that had been inserted near the nerve. Separate electrodes were used to record electromyographic signals of CMAP. The setup enabled combined recordings of CMAP and force from the living animal.
A) upper panel left trace shows force trace resulting from 12 Hz stimulation, 10 pulses, in a representative mouse, showing a decrease in force with consecutive stimulations, indicated by the dotted line form the first peak. Lower panel left trace shows simultaneously recorded CMAP from the same mouse during the same stimulation, also displaying reduced amplitude with consecutive stimulations. After administration of 8 mg/kg NMD670 i.v. both force and CMAP signal display less decrease in amplitudes with consecutive stimulation.
B) upper panel left, force trace from 120 Hz stimulation for 1 s, in black, for a CMT animal showing an initial peak in force generation followed by reduced force for the remainder of stimulation duration and in grey a force trace from a healthy mouse of similar age/gender from the same strain. Left lower panel shows CMAP recorded simultaneously with the force trace. After administration of 8 mg/kg NMD670 i.v. to the CMT mouse (right traces), force and CMAP were improved.
C) shows the average improvement in force for 12, 30 and 120 Hz stimulation, after administration of 8 mg/kg NMD670 i.v. in experiments conducted as explained above.
D) shows the relative CMAP amplitude of the 5th peak compared to the 1st, during 120 Hz stimulation before and after administration of 8 mg/kg NMD670 i.v.

FIG. 4

FIG. 4 depicts the study design of a clinical observational study that enrolled patients with CMT types 1 and 2 and healthy age-matched controls at two study sites. Clinical tests are listed in the order of testing. CMTES2: CMT Examination Score2. RNS: Repetitive Nerve Stimulation. SFEMG: Single Fibre EMG. *CMTES2 only performed at visit 1.

FIG. 5

FIG. 5 depicts jitter and blocking from visit 1 of a clinical observational study of healthy subjects and CMT patients displayed as individual data with medians for healthy controls and CMT patients (A and B) and for CMT1 and CMT2 patients (C and D). Mean Consecutive Difference (MCD).

FIG. 6

FIG. 6 depicts the study design of a mouse CMT1A study. Abbreviations: CMAP, compound muscle action potentials.

FIG. 7

FIG. 7 depicts the difference in decrement for each individual animal with averages±SEM, at each stimulation frequency. Circle=NMD670; triangle=Vehicle, n=8 in both groups.

FIG. 8

FIG. 8 depicts the difference in maximal stimulated torque for each individual animal with averages±SEM, at each stimulation frequency. Circle=NMD670; triangle=Vehicle, n=8 in both groups.

FIG. 9

FIG. 9 depicts the difference in fade for each individual animal with averages±SEM, at each stimulation frequency. Circle=NMD670; triangle=Vehicle, n=8 in both groups.

FIG. 10

FIG. 10 depicts the study design of a randomised, double-blind, placebo-controlled study to evaluate the efficacy, safety, and tolerability of NMD670 over 21 days in ambulatory adult patients with Type 1 and Type 2 Charcot-Marie-Tooth Disease.

FIG. 11

FIGS. 11A, 11B and 11C depict the schedule of activities during the clinical trial.

NOTES: If possible, assessments should be conducted at the same time at the different visits. If possible, efficacy assessments should be conducted in the order listed in the SoA. Unscheduled visits may be performed at any time. At unscheduled visits, all examinations are optional and should be reported in the CRF if completed.

Screening procedures may be conducted over several days within the 28-day screening period.

The eligibility check conducted at the baseline Visit involves a check that all eligibility criteria during the screening period are fulfilled for randomisation.

Blood samples will be collected for testing for glucose concentration after at least 4-hour fasting as part of the safety laboratory assessments.

Abbreviations: 6MWT=6-minute walk test; AE=adverse event; C_{maX}=maximal concentration; CMT-FOM=Charcot-Marie-Tooth Functional Outcome Measure; CMT-HI=Charcot-Marie-Tooth Health Index; CRF=case report form; C-SSRS=Columbia-Suicide Severity Rating Scale; ECG=electrocardiogram; EOS=end-of-study; EOT=end-of-treatment; FU=follow-up; HIV=human immunodeficiency virus; IMP=investigational medicinal product; IRT=interactive response technology; ONLS=Overall Neuropathy Limitations Scale; PK=pharmacokinetic; SAE=serious adverse event; SF-36=Short Form 36; sfEMG=single fibre electromyography; SoA=schedule of activities; TUG=Timed Up and Go test; V=Visit; WOCBP=women of childbearing potential.

### Definitions

All publications, patent applications, patents and other references mentioned herein are incorporated by reference in their entirety.

The nomenclature used in the present application is based on IUPAC systematic nomenclature, unless indicated otherwise.

The term “patient” or “subject” refers to a human (such as a male or female human) who has been diagnosed with Charcot-Marie-Tooth disease (CMT). CMT can be diagnosed through three different forms of tests: measurement of the speed of nerve impulses (nerve conduction studies), a biopsy of the nerve, and DNA testing.

The term “improvement” refers to a lessening of a patient's Charcot-Marie-Tooth disease (CMT) symptoms when the patient is administered a composition described herein. The improvement may be lessening of a patient's CMT symptoms after the patient has been administered a composition as described herein as compared to before the administration of said composition. The term “improvement” may also refer to lessening of a group of patients' CMT symptoms after the group of patients has been administered a composition as described herein, e.g. as evaluated based on comparative test scores between the patient group being administered the composition as described herein with a control group receiving e.g. placebo. An improvement in CMT symptoms can be determined for example as a decrease in the Overall Neuropathy Limitations Score (ONLS); increase in the total distance walked when determined using a 6-minute walk test; reduction in fatigue when determined using a fatigue index calculated from the distance walked in the 1st minute to the distance walked in the 6th minute of a 6-minute walk test.; a decrease in the CMT Neuropathy Score 2 (CMTNS2) score; decrease in the CMT examination score (second version) (CMTES2) score; an increase in the motor function measure 32-item score; a decrease in the CMT Functional Outcome Measure (CMT-FOM) score; an increase in the Berg Balance Scale score; a decrease in the Individualised Neuromuscular Quality of Life score; a decrease in the Fatigue Severity Scale score; a reduction in jitter; a reduction in blocking; an improvement in finger dexterity when determined using a nine-hole peg test (9-HPT); an improvement in walking ability when determined using a Six Spot Step Test; an improvement in walking ability when determined using a 10-meter walk/run test; an improvement in balance and mobility when determined using the Timed “Up and Go” test; an increase in muscle strength as assessed with a hand held dynamometer, a fixed dynamometer or manually e.g. using manual muscle testing.

The term “jitter” refers to the variability in the arrival time of muscle fibre action potentials to the recording electrode between consecutive electrical discharges when measuring neuromuscular function using single fiber electromyography (sfEMG).

The term “blocking” refers to complete NMJ transmission failure of muscle fibre action potentials to the recording electrode between consecutive electrical discharges when measuring neuromuscular function using sfEMG.

The term “placebo” refers to a dosage form possessing no therapeutic activity.

The term “active pharmaceutical ingredient” (or “API”) denotes the compound or molecule in a pharmaceutical composition that has a particular biological activity.

The terms “pharmaceutically acceptable excipient”, “pharmaceutically acceptable carrier” and “therapeutically inert excipient” can be used interchangeably and denote any pharmaceutically acceptable ingredient in a pharmaceutical composition having no therapeutic activity and being non-toxic to the subject administered, such as disintegrators, binders, fillers, solvents, buffers, tonicity agents, stabilizers, antioxidants, surfactants, carriers, diluents or lubricants used in formulating pharmaceutical products.

The term “pharmaceutical composition” refers to a preparation which is in such form as to permit the biological activity of an active ingredient contained therein to be effective, and which contains no additional components which are unacceptably toxic to a subject to which the composition would be administered.

The term “pharmaceutically acceptable” denotes an attribute of a material which is useful in preparing a pharmaceutical composition that is generally safe, non-toxic, and neither biologically nor otherwise undesirable and is acceptable for veterinary as well as human pharmaceutical use.

A “pharmaceutically acceptable carrier” refers to an ingredient in a pharmaceutical composition, other than an active ingredient, which is nontoxic to a subject. A pharmaceutically acceptable carrier includes, but is not limited to, a buffer or acidifier, excipient, stabilizer, or preservative.

The term “solid dosage form releases” means the amount of compound that is released or dissolved into solution after a specified period of time when using a United States Pharmacopeia (USP) type 2 dissolution apparatus, paddle speed of 75 rpm, at a temperature of 37° C.±0.5° C. in 900 mL of pH 6.8 phosphate/citric acid buffer as described in Example 10.

The term “Cₘₐₓ” (expressed in units of ng/mL) means maximum observed plasma concentration of NMD670. The term “mean Cₘₐₓ” means the arithmetic mean of the individual Cₘₐₓ values.

The term “Tₘₐₓ” (expressed in units of hours, or as a median number of hours for Tₘₐₓ in the study population) means the observed time to reach Cₘₐₓ following drug administration; if it occurs at more than one time point Tₘₐₓ is defined as the first time point with this value.

The term “dose” means the dose of NMD670 as free acid that was given to the subject In addition, the term “dose’ may be inclusive of NMD670 in combination with a pharmaceutically acceptable salt.

The term “therapeutically effective dose” as used herein refers to the amount of NMD670 required to cause a therapeutic response in a subject. The terms “therapeutically effective dose” and “therapeutic dose” are used interchangeably herein.

The composition comprising the (therapeutic) dose may be administered in one or more unit dosage forms. As used herein, “unit dosage forms” refers to physically discrete units suitable for human and animal subjects. Each unit dosage includes a predetermined quantity of the therapeutically active compound, in association with, when required, a pharmaceutical carrier, vehicle or diluent. Examples of unit dosage forms include tablets, capsules, pills, powders, granules, sterile parenteral solutions or suspensions, ampoules and syringes, and oral solutions or suspensions, and oil-water emulsions. Unit dosage forms can be individually packaged as is known in the art, such as in blister packs. Unit dosage forms can be administered in fractions or multiples thereof.

The term “T_{1/2}” (expressed in units of hours) means the terminal elimination half-life of NMD 670 in plasma.

The term “AUC_{0-infinity}” (expressed in units of h·ng/mL) means the cumulative area under the plasma time concentration curve (AUC) calculated using the trapezoidal method from time 0 to infinity after a single dose of NMD670. The term “mean AUC_{0-infinity}” means the arithmetic mean of the individual AUC_{0-infinity} values.

The term “AUC_{0-24 hours}” (expressed in units of h·ng/mL) means the cumulative area under the plasma time concentration curve (AUC) calculated using the trapezoidal method from time 0 to 24 hours after a single dose of NMD670. The term “mean AUC_{0-24 hours}” means the arithmetic mean of the individual AUC_{0-24 hours} values.

As used within the following disclosure, the term “NMD670” refers to (2S)-2-[4-bromo-2-(1,2-oxazol-3-yl)phenoxy]propanoic acid, in addition to any pharmaceutically acceptable salt, hydrate, polymorph, tautomer, or solvate thereof. (2S)-2-[4-bromo-2-(1,2-oxazol-3-yl)phenoxy]propanoic acid, according to the present disclosure, refers to a compound of formula (1) below, CAS Number 2354321-33-6.

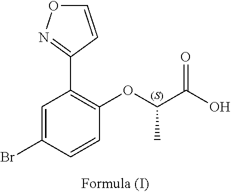

All publications, patent applications, patents and other references mentioned herein are incorporated by reference in their entirety.

### DETAILED DESCRIPTION

CMT2D mouse models show NMJ maturation defects, reduced endplate function, accelerated endplate function run-down during repeated stimulation and treatment efficacy with physostigmine, but not with 3,4-DAP (Sleigh et al., 2014; Spaulding et al., 2016).

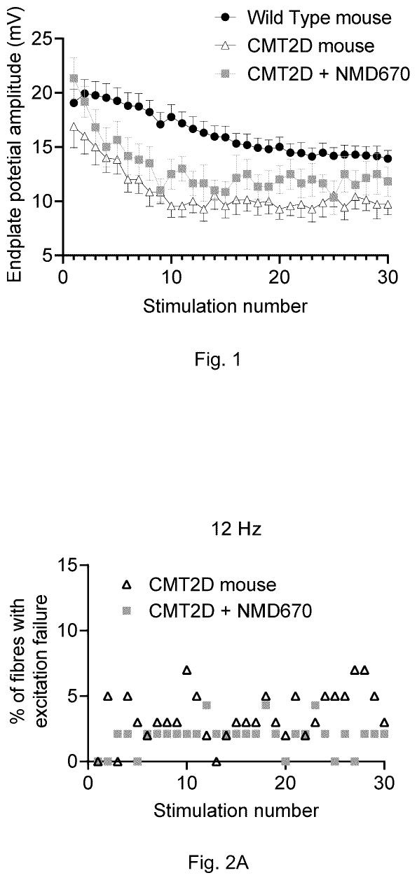

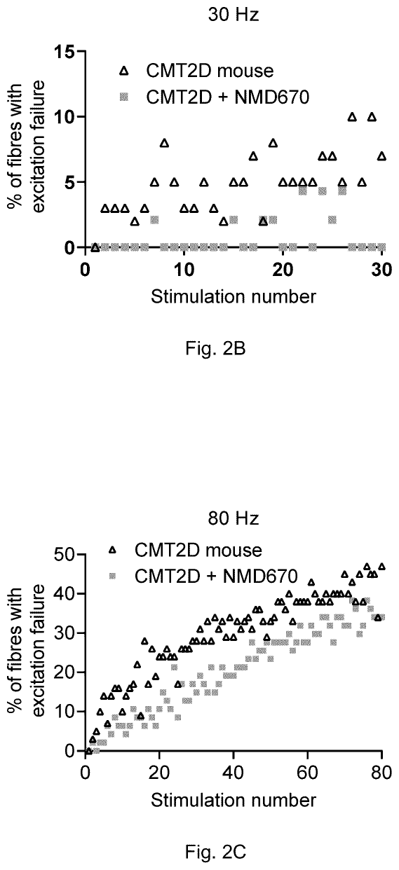

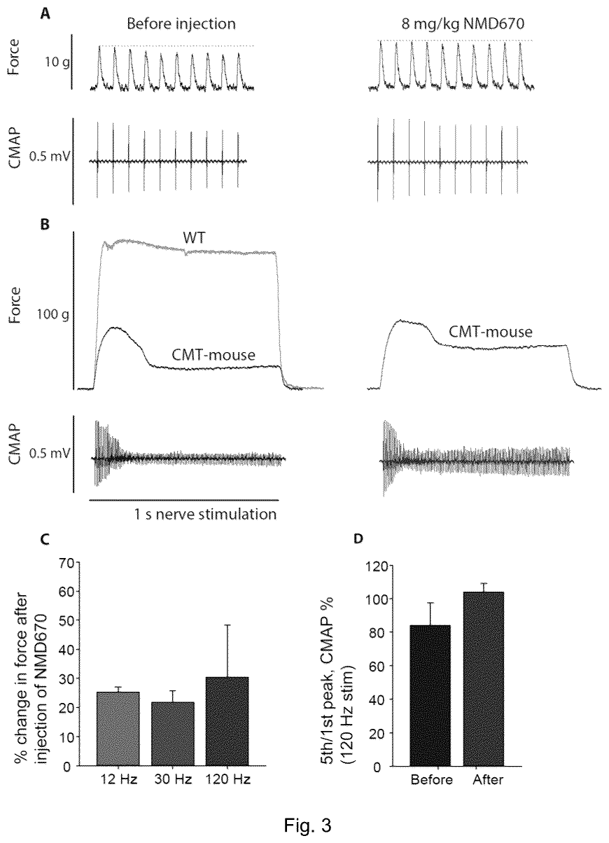

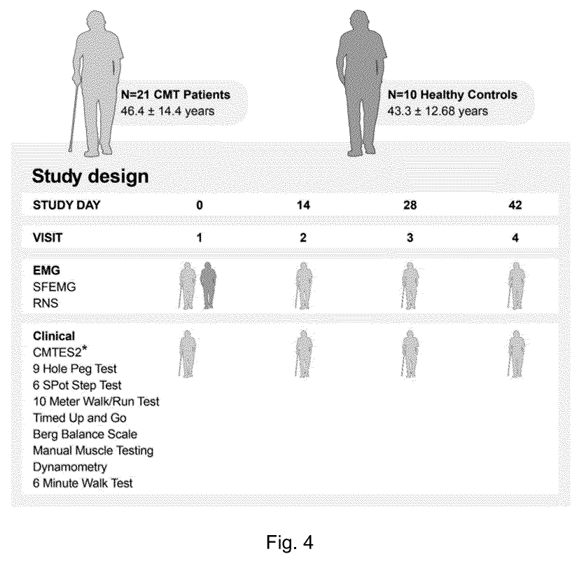

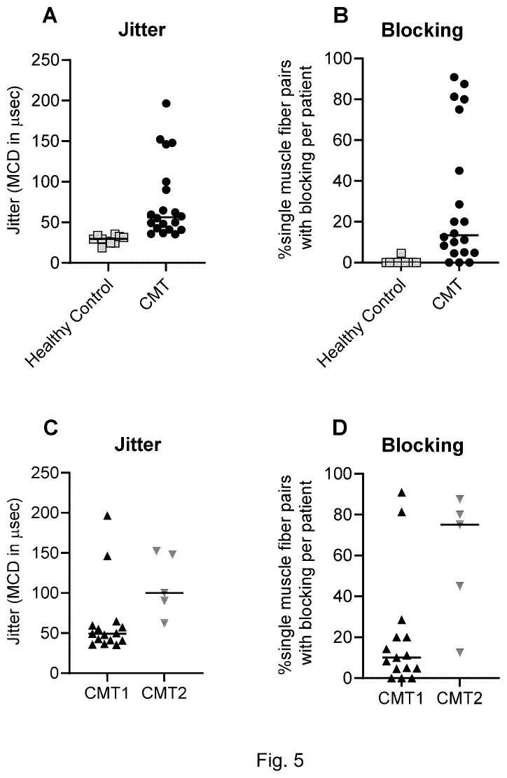

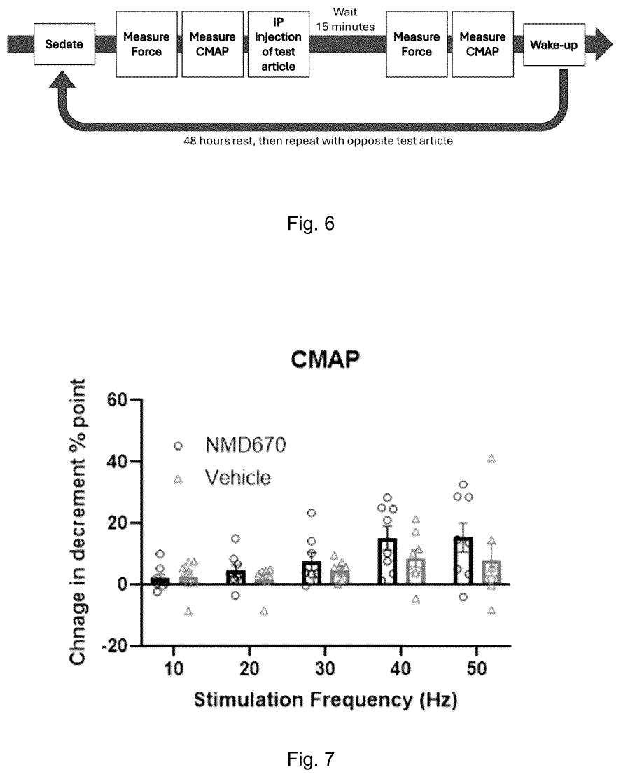

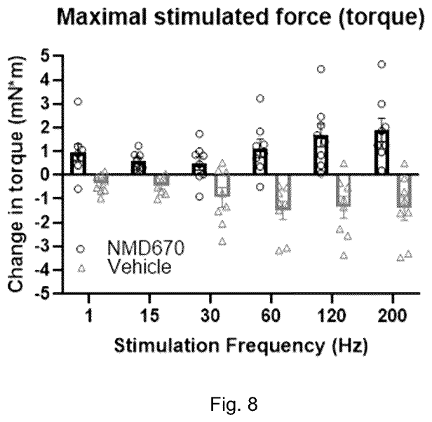

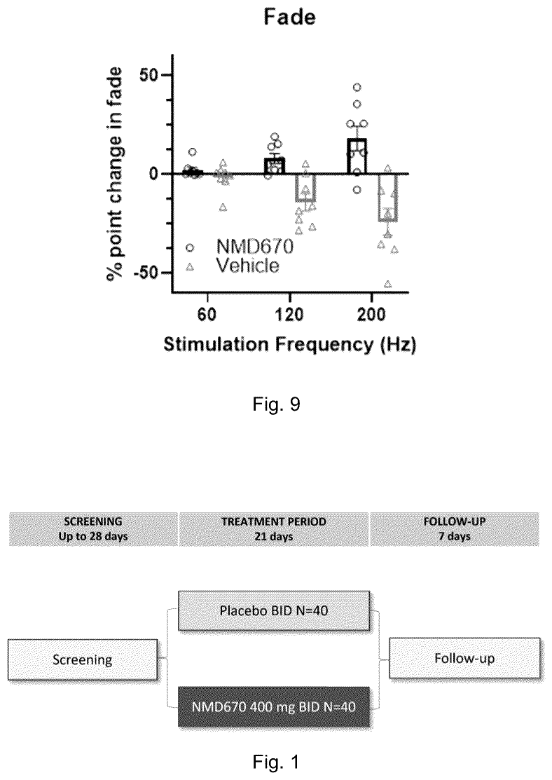

Herein we demonstrate that administration of the CIC-1 inhibitor NMD670 to isolated nerve-muscle preparations from CMT2D mice resulted in a higher endplate potential (EPP) and a reduction in excessive run-down of action potential firing during repeated stimulation of the motor nerve compared to prior to compound administration (FIG. 1). Further, the percentage of action potentials that failed to be generated in response to stimulation of all measured fibres in each group per stimulation was reduced after addition of NMD670. This demonstrates that addition of a CIC-1 inhibitor, specifically NMD670, to ex vivo muscle preparations can enhance neuromuscular transmission in CMT2D mice. Further, administration of a CIC-1 inhibitor, specifically NMD670, to CMT2D mice resulted in an increase in force, compound muscle action potential (CMAP) and relative force peaks (T4 or T10/T1). This demonstrates that NMD670 can enhance neuromuscular transmission and thereby restore muscle function in vivo.

Herein we further demonstrate that patients with CMT1 or CMT2 have significant NMJ transmission deficits. Specifically, by measuring single fibre electromyography (sfEMG) it was discovered that blocking and jitter were higher in CMT patients compared with healthy aged-matched controls (FIG. 5). It was also discovered that dysfunction in clinical parameters related to strength, balance and mobility were correlated with jitter and blocking.

From the discoveries provided by the above studies, the inventors were able to develop the compositions for use in CMT treatment methods and CMT treatment methods described herein. Exemplified embodiments of these compositions and methods are provided below. The compositions and methods described herein are not intended to be limited to the following exemplary embodiments.

### Compositions for Use

One aspect of the present disclosure relates to compositions for use in a method of treatment of Charcot-Marie-Tooth disease in a subject. These compositions for use comprise administering a therapeutically effective dose of (2S)-2-[4-bromo-2-(1,2-oxazol-3-yl)phenoxy]propanoic acid (NMD670) to the patient, wherein the therapeutic dose is within the range of 100 mgs to 1500 mgs.

Thus one aspect of the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein the composition is for administration at a therapeutic dose of 100 to 1500 mg of (2S)-2-[4-bromo-2-(1,2-oxazol-3-yl)phenoxy]propanoic acid.

In exemplary embodiments, the therapeutically effective dose of NMD670 that is administered to the patient is less than 1500 mg, less than 1450 mg, less than 1300 mg, less than 1250 mg, less than 1200 mg, less than 1150 mg, less than 1100 mg, less than 1050 mg, less than 1000 mg, less than 950 mg, less than 900 mg, less than 850 mg, less than 800 mg, less than 750 mg, less than 700 mg, less than 650 mg, less than 600 mg, less than 550 mg, less than 500 mg, less than 450 mg, less than 400 mg, less than 350 mg, less than 300 mg, or less than 250 mg.

In other embodiments, the therapeutically effective dose of NMD670 that is administered to the patient is at least 100 mg, at least 150 mg, at least 200 mg, at least 250 mg, at least 300 mg, at least 350 mg, at least 400 mg, at least 450 mg, at least 500 mg, at least 550 mg, at least 600 mg, at least 650 mg, at least 700 mg, at least 750 mg, at least 800 mg, at least 850 mg, at least 900 mg, at least 950 mg, at least 1000 mg, at least 1050 mg, at least 1100 mg, at least 1150 mg, at least 1200 mg, at least 1250 mg, at least 1300 mg, at least 1350 mg, at least 1400 mg, or at least 1450 mg.

In some exemplary embodiments, the therapeutically effective dose of NMD670 that is administered to the patient is from 100 to 600 mg, from 200 to 600 mg, from 250 to 550 mg, from 300 to 500 mg, from 350 to 450 mg, from 375 to 425 mg, or 400 mg. In other exemplary embodiments, the therapeutically effective dose of NMD670 that is administered to the patient is from 700 to 1400 mg, from 800 to 1350 mg, from 900 to 1300 mg, from 1000 to 1250 mg, from 1100 to 1250 mg, or about 1200 mg. In exemplary embodiments, the therapeutically effective dose of NMD670 that is administered to the patient is about 100 mg. In exemplary embodiments, the therapeutically effective dose of NMD670 that is administered to the patient is about 150 mg. In exemplary embodiments, the therapeutically effective dose of NMD670 that is administered to the patient is about 200 mg. In exemplary embodiments, the therapeutically effective dose of NMD670 that is administered to the patient is about 250 mg. In exemplary embodiments, the therapeutically effective dose of NMD670 that is administered to the patient is about 300 mg. In exemplary embodiments, the therapeutically effective dose of NMD670 that is administered to the patient is about 350 mg. In exemplary embodiments, the therapeutically effective dose of NMD670 that is administered to the patient is about 400 mg. In exemplary embodiments, the therapeutically effective dose of NMD670 that is administered to the patient is about 500 mg. In exemplary embodiments, the therapeutically effective dose of NMD670 that is administered to the patient is about 600 mg.

In exemplary embodiments, the therapeutic dose is to be administered at least one time daily. In exemplary embodiments, the therapeutic dose is to be administered one time daily. In exemplary embodiments, the therapeutic dose is to be administered two times daily. In exemplary embodiments, the therapeutic dose is to be administered three times daily. In exemplary embodiments, the therapeutic dose is to be administered four times daily.

In exemplary embodiments, the therapeutic dose is administered one time daily, i.e. the therapeutic dose is the total daily dosage. In exemplary embodiments, the therapeutic dose is 100 to 600 mg and is administered one time daily. In exemplary embodiments, the therapeutic dose is 200 to 600 mg and is administered one time daily. In exemplary embodiments, the therapeutic dose is 300 to 500 mg and is administered one time daily. In exemplary embodiments, the therapeutic dose is about 100 mg and is administered one time daily. In exemplary embodiments, the therapeutic dose is about 150 mg and is administered one time daily. In exemplary embodiments, the therapeutic dose is about 200 mg and is administered one time daily. In exemplary embodiments, the therapeutic dose is about 250 mg and is administered one time daily. In exemplary embodiments, the therapeutic dose is about 300 mg and is administered one time daily. In exemplary embodiments, the therapeutic dose is about 350 mg and is administered one time daily. In exemplary embodiments, the therapeutic dose is about 400 mg and is administered one time daily. In exemplary embodiments, the therapeutic dose is about 500 mg and is administered one time daily. In exemplary embodiments, the therapeutic dose is about 600 mg and is administered one time daily.

In exemplary embodiments, the therapeutic dose is administered two times daily, i.e. the total daily dosage is twice the therapeutic dose. In exemplary embodiments, the therapeutic dose is 100 to 600 mg and is administered two times daily. In exemplary embodiments, the therapeutic dose is 200 to 600 mg and is administered two times daily. In exemplary embodiments, the therapeutic dose is 300 to 500 mg and is administered two times daily. In exemplary embodiments, the therapeutic dose is about 100 mg and is administered two times daily. In exemplary embodiments, the therapeutic dose is about 150 mg and is administered two times daily. In exemplary embodiments, the therapeutic dose is about 200 mg and is administered two times daily. In exemplary embodiments, the therapeutic dose is about 250 mg and is administered two times daily. In exemplary embodiments, the therapeutic dose is about 300 mg and is administered two times daily. In exemplary embodiments, the therapeutic dose is about 350 mg and is administered two times daily. In exemplary embodiments, the therapeutic dose is about 400 mg and is administered two times daily. In exemplary embodiments, the therapeutic dose is about 500 mg and is administered two times daily. In exemplary embodiments, the therapeutic dose is about 600 mg and is administered two times daily.

In exemplary embodiments, the therapeutic dose is administered three times daily, i.e. the total daily dosage is three times the therapeutic dose. In exemplary embodiments, the therapeutic dose is 100 to 600 mg and is administered three times daily. In exemplary embodiments, the therapeutic dose is 200 to 600 mg and is administered three times daily. In exemplary embodiments, the therapeutic dose is 300 to 500 mg and is administered three times daily. In exemplary embodiments, the therapeutic dose is about 100 mg and is administered three times daily. In exemplary embodiments, the therapeutic dose is about 150 mg and is administered three times daily. In exemplary embodiments, the therapeutic dose is about 200 mg and is administered three times daily. In exemplary embodiments, the therapeutic dose is about 250 mg and is administered three times daily. In exemplary embodiments, the therapeutic dose is about 300 mg and is administered three times daily. In exemplary embodiments, the therapeutic dose is about 350 mg and is administered three times daily. In exemplary embodiments, the therapeutic dose is about 400 mg and is administered three times daily. In exemplary embodiments, the therapeutic dose is about 500 mg and is administered three times daily. In exemplary embodiments, the therapeutic dose is about 600 mg and is administered three times daily.

The composition comprising the therapeutic dose may be administered in one or more unit dosage forms. A therapeutic dose of 400 mg may for example be administered as one unit dosage form comprising 400 mg, or two unit dosage forms comprising 200 mg, or four unit dosage forms comprising 100 mg.

In exemplary embodiments, the therapeutic dose is the total daily dosage.

In exemplary embodiments, the composition for use is dosed orally, i.e. the composition is for oral administration. In exemplary embodiments, the composition for use is a solid dosage form. In exemplary embodiments, the solid dosage form is dosed orally. In exemplary embodiments, the solid dosage form is selected from the group consisting of capsule (such as sprinkle capsule and gelatine capsule), tablet (such as uncoated tablet, coated tablet, slow-release tablet) and sprinkle. In exemplary embodiments, the composition is in the form of a liquid, liquid suspension, oil, emulsion, or syrup. In exemplary embodiments, the solid dosage form releases not less than 80% of the compound after 30 minutes, when measured in a United States Pharmacopeia (USP) type 2 dissolution apparatus, paddle at 75 rpm, at a temperature of 37° C.±0.5° C. in 900 mL of pH 6.8 phosphate/citric acid buffer.

In exemplary embodiments, the composition for use is to be dosed orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where the mean Cₘₐₓ is 12,760 to 27,440 ng/mL, such as 15,000 to 25,000 ng/mL, such as 16,000 to 24,000 ng/mL, such as 16,080 to 25,125 ng/mL, such as 17,000 to 23,000 ng/mL, such as 18,000 to 22,000 ng/mL, such as 19,000 to 21,000 ng/mL, such as about 20,100 ng/mL after administration with a single dose of 400 mg (2S)-2-[4-bromo-2-(1,2-oxazol-3-yl)phenoxy]propanoic acid, or a pharmaceutically acceptable salt, hydrate, polymorph, tautomer, or solvate thereof. In exemplary embodiments, mean Cₘₐₓ is 20,100 ng/mL and the standard deviation is 7,340 ng/mL.

In exemplary embodiments, the composition for use is to be dosed orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where the mean Cₘₐₓ is about 80% to about 125%, such as 80.00% to 125.00%, of 20,100 ng/mL after administration with a single dose of 400 mg (2S)-2-[4-bromo-2-(1,2-oxazol-3-yl)phenoxy]propanoic acid, or a pharmaceutically acceptable salt, hydrate, polymorph, tautomer, or solvate thereof.

In exemplary embodiments, the composition for use is to be dosed orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where the mean AUC_{0-infinity} is 66,300 to 109,100 h·ng/mL, such as 70,000 to 105,000 h·ng/mL, such as 70,160 to 109,625 h·ng/mL, such as 75,000 to 100,000 h·ng/mL, such as 80,000 to 95,000 h·ng/mL, such as 85,000 to 90,000 h·ng/mL, such as about 87,700 h·ng/mL after administration with a single dose of 400 mg (2S)-2-[4-bromo-2-(1,2-oxazol-3-yl)phenoxy]propanoic acid, or a pharmaceutically acceptable salt, hydrate, polymorph, tautomer, or solvate thereof. In exemplary embodiments, mean AUC_{0-infinity} is 87,700 ng/mL and the standard deviation is 21,400 ng/mL.

In exemplary embodiments, the composition for use is to be dosed orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where the mean AUC_{0-infinity} is about 80% to about 125%, such as 80.00% to 125.00%, of 87,700 h·ng/mL after administration with a single dose of 400 mg (2S)-2-[4-bromo-2-(1,2-oxazol-3-yl)phenoxy]propanoic acid, or a pharmaceutically acceptable salt, hydrate, polymorph, tautomer, or solvate thereof.

In exemplary embodiments, the composition or use is to be administered orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where Tₘₐₓ is reached within 1 to 6 hours after administration.

In exemplary embodiments, the composition or use is to be administered orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where Tₘₐₓ is reached within 3 to 7 hours after administration.

In exemplary embodiments, the AUC_{0-24 infinity}, Cₘₐₓ or Tₘₐₓ is measured after administration of a single dose to a human subject suffering from Charcot-Marie-Tooth disease.

The compositions for use described herein can be formulated for administrating either orally, parenterally, intravenously, inhaled, topically, enterally, rectally, buccally or as an aerosol.

In exemplary embodiments, the composition for use further comprises at least one pharmaceutically acceptable adjuvant and/or excipient. In exemplary embodiments, the composition for use comprises at least one pharmaceutically acceptable adjuvant and/or excipient selected from the group consisting of filler, binder, lubricant and disintegrant. In exemplary embodiments, the composition for use comprises at least one pharmaceutically acceptable adjuvant and/or excipient selected from the group consisting of silicified microcrystalline cellulose, microcrystalline cellulose, maltodextrin, magnesium stearate and croscarmellose sodium.

In exemplary embodiments, the composition for use comprises 10 to 80 wt %, such as to 65 wt %, such as 50 to 55 wt %, such as about 53 wt % (2S)-2-[4-bromo-2-(1,2-oxazol-3-yl)phenoxy]propanoic acid, or a pharmaceutically acceptable salt, hydrate, polymorph, tautomer, or solvate thereof.

In exemplary embodiments, the composition for use comprises 10 to 80 wt %, such as to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof.

In exemplary embodiments, the composition for use is in the form of one or more solid dosage forms comprising:
a. 10 to 80 wt %, such as 40 to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof;
b. 20 to 80 wt %, such as 25 to 50 wt % filler;
c. 2 to 20 wt %, such as 3 to 16 wt % binder;
d. 0.25 to 3 wt %, such as 0.4 to 2.0 wt % lubricant; and
e. 0.25 to 5 wt %, such as 0.3 to 2.5 wt % disintegrant;
   with the proviso that the sum of the wt % of the components does not exceed 100 wt %.

In exemplary embodiments, the composition for use is in the form of one or more solid dosage forms comprising:
a. 10 to 80 wt %, such as 40 to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof;
b. 20 to 80 wt %, such as 25 to 50 wt % filler;
c. 2 to 20 wt %, such as 3 to 16 wt % binder;
d. 0.25 to 3 wt %, such as 0.4 to 2.0 wt % lubricant;
e. 0.25 to 5 wt %, such as 0.3 to 2.5 wt % disintegrant; and f. 1 to 10 wt % film coating;
   with the proviso that the sum of the wt % of the components does not exceed 100 wt %.

In exemplary embodiments, the composition for use is in the form of one or more solid dosage forms comprising:
a. 10 to 80 wt %, such as 40 to 65 wt %, such as 50 to 60 wt %, such as 50 to 55 wt %, such as 55 to 60 wt % such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof;
b. 5 to 60 wt %, such as 20 to 40 wt %, such as 21 to 37 wt % silicified microcrystalline cellulose;
c. 2 to 60 wt %, such as 5 to 16 wt % microcrystalline cellulose;
d. 1 to 15 wt %, such as 1.5 to 7 wt %, such as 1.8 to 6.0 wt % maltodextrin;
e. 0.25 to 3 wt %, such as 0.4 to 2.0 wt % magnesium stearate; and
f. 0.25 to 5 wt %, such as 0.3 to 2.5 wt % Croscarmellose sodium;
   with the proviso that the sum of the wt % of the components does not exceed 100 wt %.

In exemplary embodiments, the composition for use is in the form of a solid dosage form and comprises:
a. 10 to 80 wt %, such as 40 to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof;
b. 5 to 60 wt %, such as 20 to 40 wt %, such as 21 to 37 wt % silicified microcrystalline cellulose;
c. 2 to 60 wt %, such as 5 to 16 wt % microcrystalline cellulose;
d. 1 to 15 wt %, such as 1.5 to 7 wt %, such as 1.8 to 6.0 wt % maltodextrin;
e. 0.25 to 3 wt %, such as 0.4 to 2.0 wt % magnesium stearate; and
f. 0.25 to 5 wt %, such as 0.3 to 2.5 wt % Croscarmellose sodium;
   with the proviso that the sum of the wt % of the components does not exceed 100 wt %.

In exemplary embodiments, the composition for use is in the form of one or more solid dosage forms comprising or consisting of:
a. 10 to 80 wt %, such as 40 to 65 wt %, such as 50 to 55 wt %, such as about 53 wt % (2S)-2-[4-bromo-2-(1,2-oxazol-3-yl)phenoxy]propanoic acid, or a pharmaceutically acceptable salt, hydrate, polymorph, tautomer, or solvate thereof;
b. 5 to 60 wt %, such as 20 to 40 wt %, such as 21 to 37 wt % silicified microcrystalline cellulose;
c. 2 to 60 wt %, such as 5 to 16 wt % microcrystalline cellulose;
d. 1 to 15 wt %, such as 1.5 to 7 wt %, such as 1.8 to 6.0 wt % maltodextrin;
e. 0.25 to 3 wt %, such as 0.4 to 2.0 wt % magnesium stearate; and
f. 0.25 to 5 wt %, such as 0.3 to 2.5 wt % Croscarmellose sodium;
g. 1 to 10 wt % film coating composition such as Opadry white,
   with the proviso that the sum of the wt % of the components does not exceed 100 wt %.

In exemplary embodiments, the composition for use is in the form of a solid dosage form and comprises or consists of:
a. 10 to 80 wt %, such as 40 to 65 wt %, such as 50 to 55 wt %, such as about 53 wt % (2S)-2-[4-bromo-2-(1,2-oxazol-3-yl)phenoxy]propanoic acid, or a pharmaceutically acceptable salt, hydrate, polymorph, tautomer, or solvate thereof;
b. 5 to 60 wt %, such as 20 to 40 wt %, such as 21 to 37 wt % silicified microcrystalline cellulose;
c. 2 to 60 wt %, such as 5 to 16 wt % microcrystalline cellulose;
d. 1 to 15 wt %, such as 1.5 to 7 wt %, such as 1.8 to 6.0 wt % maltodextrin;
e. 0.25 to 3 wt %, such as 0.4 to 2.0 wt % magnesium stearate;
f. 0.25 to 5 wt %, such as 0.3 to 2.5 wt % Croscarmellose sodium; and
g. 1 to 10 wt % film coating composition such as Opadry white,
   with the proviso that the sum of the wt % of the components does not exceed 100 wt %.

In exemplary embodiments, the subject has a level of serum uric acid below 6.5 mg/dL.

In exemplary embodiments, the subject has been diagnosed with CMTD. In exemplary embodiments, the subject has been diagnosed with CMT1. In exemplary embodiments the subject has been diagnosed with CMT1A. In exemplary embodiments, the subject has been diagnosed with CMT1B. In exemplary embodiments, the subject has been diagnosed with CMT1C. In exemplary embodiments, the subject has been diagnosed with CMT1D. In exemplary embodiments, the subject has been diagnosed with CMT1E. In exemplary embodiments, the subject has been diagnosed with CMT1F. In exemplary embodiments, the subject has been diagnosed with CMT1G.

In exemplary embodiments, the subject has been diagnosed with CMT2. In exemplary embodiments, the subject has been diagnosed with CMT2A. In exemplary embodiments, the subject has been diagnosed with CMT2A1. In exemplary embodiments, the subject has been diagnosed with CMT2A2A. In exemplary embodiments, the subject has been diagnosed with CMT2A2B. In exemplary embodiments, the subject has been diagnosed with CMT2B. In exemplary embodiments, the subject has been diagnosed with CMT2B1. In exemplary embodiments, the subject has been diagnosed with CMT2B2. In exemplary embodiments, the subject has been diagnosed with CMT2C. In exemplary embodiments, the subject has been diagnosed with CMT2D. In exemplary embodiments, the subject has been diagnosed with CMT2E. In exemplary embodiments, the subject has been diagnosed with CMT2F. In exemplary embodiments, the subject has been diagnosed with CMT2H. In exemplary embodiments, the subject has been diagnosed with CMT2I. In exemplary embodiments, the subject has been diagnosed with CMT2J. In exemplary embodiments, the subject has been diagnosed with CMT2K. In exemplary embodiments, the subject has been diagnosed with CMT2L. In exemplary embodiments, the subject has been diagnosed with CMT2M. In exemplary embodiments, the subject has been diagnosed with CMT2N. In exemplary embodiments, the subject has been diagnosed with CMT20. In exemplary embodiments, the subject has been diagnosed with CMT2P. In exemplary embodiments, the subject has been diagnosed with CMT2Q. In exemplary embodiments, the subject has been diagnosed with CMT2R. In exemplary embodiments, the subject has been diagnosed with CMT2S. In exemplary embodiments, the subject has been diagnosed with CMT2T. In exemplary embodiments, the subject has been diagnosed with CMT2U. In exemplary embodiments, the subject has been diagnosed with CMT2V. In exemplary embodiments, the subject has been diagnosed with CMT2Z. In exemplary embodiments, the subject has been diagnosed with CMT2X. In exemplary embodiments, the subject has been diagnosed with CMT2Y. In exemplary embodiments, the subject has been diagnosed with CMT2CC. In exemplary embodiments, the subject has been diagnosed with CMT2DD. In exemplary embodiments, the subject has been diagnosed with CMT2EE.

In exemplary embodiments, the subject has been diagnosed with CMT3.

In exemplary embodiments, the subject has been diagnosed with CMT4. In exemplary embodiments, the subject has been diagnosed with CMT4A. In exemplary embodiments, the subject has been diagnosed with CMT4B1. In exemplary embodiments, the subject has been diagnosed with CMT4B2. In exemplary embodiments, the subject has been diagnosed with CMT4B3. In exemplary embodiments, the subject has been diagnosed with CMT4C. In exemplary embodiments, the subject has been diagnosed with CMT4D. In exemplary embodiments, the subject has been diagnosed with CMT4E. In exemplary embodiments, the subject has been diagnosed with CMT4F. In exemplary embodiments, the subject has been diagnosed with CMT4G. In exemplary embodiments, the subject has been diagnosed with CMT4H. In exemplary embodiments, the subject has been diagnosed with CMT4J.

In exemplary embodiments, the subject has been diagnosed with CMTDI. In exemplary embodiments, the subject has been diagnosed with CMTDIA. In exemplary embodiments, the subject has been diagnosed with CMTDIB. In exemplary embodiments, the subject has been diagnosed with CMTDIC. In exemplary embodiments, the subject has been diagnosed with CMTDID. In exemplary embodiments, the subject has been diagnosed with CMTDIE. In exemplary embodiments, the subject has been diagnosed with CMTDIF.

In exemplary embodiments, the subject has been diagnosed with CMTRI. In exemplary embodiments, the subject has been diagnosed with CMTRIA. In exemplary embodiments, the subject has been diagnosed with CMTRIB.

In exemplary embodiments, the subject has been diagnosed with CMTX1 (also called CMT1X). In exemplary embodiments, the subject has been diagnosed with CMTX2. In exemplary embodiments, the subject has been diagnosed with CMTX3. In exemplary embodiments, the subject has been diagnosed with CMTX4. In exemplary embodiments, the subject has been diagnosed with CMTX5. In exemplary embodiments, the subject has been diagnosed with CMTX6.

In exemplary embodiments, the subject has been diagnosed with CMT1, CMT2 or CMTX. In exemplary embodiments, the subject has been diagnosed with CMT1 or CMT2. In exemplary embodiments, the subject has been diagnosed with CMT1A, CMT1B, CMT2A or CMTX1.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 100 to 600 mg of (2S)-2-[4-bromo-2-(1,2-oxazol-3-yl)phenoxy]propanoic acid; and
the composition is to be administered one time daily.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 200 to 600 mg of (2S)-2-[4-bromo-2-(1,2-oxazol-3-yl)phenoxy]propanoic acid; and
the composition is to be administered one time daily.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 100 to 600 mg of (2S)-2-[4-bromo-2-(1,2-oxazol-3-yl)phenoxy]propanoic acid; and
the composition is to be administered two times daily.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 200 to 600 mg of (2S)-2-[4-bromo-2-(1,2-oxazol-3-yl)phenoxy]propanoic acid; and
the composition is to be administered two times daily.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 100 to 600 mg of (2S)-2-[4-bromo-2-(1,2-oxazol-3-yl)phenoxy]propanoic acid; and
the composition is to be administered three times daily.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 200 to 600 mg of (2S)-2-[4-bromo-2-(1,2-oxazol-3-yl)phenoxy]propanoic acid; and
the composition is to be administered three times daily.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 100 to 600 mg of (2S)-2-[4-bromo-2-(1,2-oxazol-3-yl)phenoxy]propanoic acid; and
the composition is to be administered four times daily.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 200 to 600 mg of (2S)-2-[4-bromo-2-(1,2-oxazol-3-yl)phenoxy]propanoic acid; and
the composition is to be administered four times daily.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 100 to 600 mg of (2S)-2-[4-bromo-2-(1,2-oxazol-3-yl)phenoxy]propanoic acid;
the composition is to be administered one time daily; and
the composition is in the form of a solid dosage form and is to be dosed orally.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily; and
the composition is in the form of a solid dosage form and is to be dosed orally.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 100 to 600 mg of (2S)-2-[4-bromo-2-(1,2-oxazol-3-yl)phenoxy]propanoic acid;
the composition is to be administered three times daily; and
the composition is in the form of a solid dosage form and is to be dosed orally.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 100 to 600 mg of (2S)-2-[4-bromo-2-(1,2-oxazol-3-yl)phenoxy]propanoic acid;
the composition is to be administered four times daily; and
the composition is in the form of a solid dosage form and is to be dosed orally.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 200 to 600 mg of (2S)-2-[4-bromo-2-(1,2-oxazol-3-yl)phenoxy]propanoic acid;
the composition is to be administered one time daily; and
the composition is in the form of a solid dosage form and is to be dosed orally.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily; and
the composition is in the form of a solid dosage form and is to be dosed orally.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 200 to 600 mg of (2S)-2-[4-bromo-2-(1,2-oxazol-3-yl)phenoxy]propanoic acid;
the composition is to be administered three times daily; and
the composition is in the form of a solid dosage form and is to be dosed orally.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 200 to 600 mg of (2S)-2-[4-bromo-2-(1,2-oxazol-3-yl)phenoxy]propanoic acid;
the composition is to be administered four times daily; and
the composition is in the form of a solid dosage form and is to be dosed orally.

In exemplary embodiments, the subject experiences a lessening of Charcot-Marie-Tooth disease symptoms.

In exemplary embodiments, the subject experiences a decrease in the Overall Neuropathy Limitations Score (ONLS) after treatment with NMD670 (Graham et al, 2006). In exemplary embodiments, a decrease in the Overall Neuropathy Limitations Score can be determined by comparing the change from baseline in the Overall Neuropathy Limitations Score after a defined period of time (e.g., 21 days) of NMD670 treatment with the change from baseline in the Overall Neuropathy Limitations Score after a defined period of time (e.g., 21 days) of placebo treatment. In exemplary embodiments, the subject experiences a decrease in the Overall Neuropathy Limitations Score after treatment with NMD670, wherein the score has decreased by at least 0.3 points, such as at least 0.5 points, such as at least 0.75 points, such as at least 1 point, such as at least 2 points, such as at least 3 points, such as at least 4 points, such as at least 5 points, such as at least 6 points, such as at least 8 points, such as at least 10 points, such as between 0.3 and 10 points, such as between 0.5 and 6 points, such as between 0.3 and 5 points.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and
the subject experiences a decrease in the Overall Neuropathy Limitations Score.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and
the subject experiences a decrease in the Overall Neuropathy Limitations Score.

In exemplary embodiments, the subject experiences an increase in the total distance walked after treatment with NMD670. An increase in the total distance walked can be determined using a 6-minute walk test (ATS, 2002). In exemplary embodiments, an improvement in the total distance walked can be determined by comparing the change from baseline in the total distance walked after a defined period of time (e.g., 21 days) of NMD670 treatment with the change from baseline in the total distance walked after a defined period of time (e.g., 21 days) of placebo treatment. In exemplary embodiments, the subject experiences an increase in the total distance walked when determined using the 6-minute walk test after treatment with NMD670. In exemplary embodiments, the subject experiences an increase in the total distance walked when determined using the 6-minute walk test after treatment with NMD670, wherein the total distance walked has increased by at least 5%, such as at least 10%, such as at least 15%, such as at least 20%, such as at least 25%, such as at least 30%, such as at least 50%, such as at least 75%, such as at least 100%, such as at least 150%, such as at least 200%, such as between 5% and 400%, such as between 5% and 200%, such as between 10% and 200%. In exemplary embodiments, the subject experiences an increase in the total distance walked when determined using the 6-minute walk test after treatment with NMD670, wherein the total distance walked has increased by at least 20 metres, such as at least 30 metres, such as at least 40 metres, such as at least 50 metres, such as at least 60 metres, such as at least 80 metres, such as at least 100 metres, such as at least 150 metres, such as at least 200 metres, such as at least 250 metres, such as at least 300 metres, such as between 20 and 400 metres, such as between 30 and 300 metres, such as between 40 and 200 metres.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and
the subject experiences an increase in the total distance walked when determined using the 6-minute walk test.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and
the subject experiences an increase in the total distance walked when determined using the 6-minute walk test.

In exemplary embodiments, the subject experiences a reduction in fatigue after treatment with NMD670 when determined using a fatigue index. The fatigue index is calculated from the distance walked in the 1st minute to the distance walked in the 6th minute of a 6-minute walk test. In exemplary embodiments, a reduction in fatigue can be determined by comparing the change from baseline in the fatigue index after a defined period of time (e.g., 21 days) of NMD670 treatment with the change from baseline in the fatigue index after a defined period of time (e.g., 21 days) of placebo treatment. In exemplary embodiments the subject experiences a reduction in fatigue after treatment with NMD670 when determined using a fatigue index, wherein fatigue has decreased by at least 5%, such as at least 10%, such as at least 15%, such as at least 20%, such as at least 25%, such as at least 30%, such as at least 40%, such as at least 50%, such as at least 60%, such as at least 70%, such as at least 80%, such as between 5% and 95%, such as between 5% and 80%, such as between 10% and 50%.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and
the subject experiences a reduction in fatigue when determined using a fatigue index.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and
the subject experiences a reduction in fatigue when determined using a fatigue index.

In exemplary embodiments, the subject experiences a decrease in the CMT Neuropathy Score 2 (CMTNS2) score after treatment with NMD670 (Murphy et al, 2011). In exemplary embodiments, a decrease in the CMTNS2 score can be determined by comparing the change from baseline in the CMTNS2 score after a defined period of time (e.g., 21 days) of NMD670 treatment with the change from baseline in the CMTNS2 score after a defined period of time (e.g., 21 days) of placebo treatment. In exemplary embodiments, the subject experiences a decrease in the CMTNS2 score after treatment with NMD670, wherein the score has decreased by at least 0.3 points, such as at least 0.5 points, such as at least 0.75 points, such as at least 1 point, such as at least 2 points, such as at least 3 points, such as at least 4 points, such as at least 5 points, such as at least 6 points, such as at least 8 points, such as at least 10 points, such as between 0.3 and 20 points, such as between 0.5 and 6 points, such as between 0.3 and 5 points.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and
the subject experiences a decrease in the CMT Neuropathy Score 2 score.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and
the subject experiences a decrease in the CMT Neuropathy Score 2 score.

In exemplary embodiments, the subject experiences a decrease in the CMT examination score (second version) (CMTES2) score after treatment with NMD670 (Murphy et al, 2011). In exemplary embodiments, a decrease in the CMTES2 score can be determined by comparing the change from baseline in the CMTES2 score after a defined period of time (e.g., 21 days) of NMD670 treatment with the change from baseline in the CMTES2 score after a defined period of time (e.g., 21 days) of placebo treatment. In exemplary embodiments, the subject experiences a decrease in the CMTES2 score after treatment with NMD670, wherein the score has decreased by at least 0.3 points, such as at least 0.5 points, such as at least 0.75 points, such as at least 1 point, such as at least 2 points, such as at least 3 points, such as at least 4 points, such as at least 5 points, such as at least 6 points, such as at least 8 points, such as at least 10 points, such as between 0.3 and 20 points, such as between 0.5 and 6 points, such as between 0.3 and 5 points.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and
the subject experiences a decrease in the CMT examination score (second version) score.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and
the subject experiences a decrease in the CMT examination score (second version) score.

In exemplary embodiments, the subject experiences an increase in the motor function measure 32-item score after treatment with NMD670 (Allard et al 2014). In exemplary embodiments, an increase in the motor function measure 32-item score can be determined by comparing the change from baseline in the motor function measure 32-item score after a defined period of time (e.g., 21 days) of NMD670 treatment with the change from baseline in the motor function measure 32-item score after a defined period of time (e.g., 21 days) of placebo treatment. In exemplary embodiments the subject experiences an increase in the motor function measure 32-item score after treatment with NMD670, wherein the score has increased by at least 5%, such as at least 10%, such as at least 15%, such as at least 20%, such as at least 25%, such as at least 30%, such as at least 40%, such as at least 50%, such as at least 60%, such as at least 70%, such as at least 80%, such as between 5% and 95%, such as between 5% and 80%, such as between 10% and 50%.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and
the subject experiences an increase in the motor function measure 32-item score.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and
the subject experiences an increase in the motor function measure 32-item score.

In exemplary embodiments, the subject experiences a decrease in the CMT Functional Outcome Measure (CMT-FOM) score after treatment with NMD670 (Eichinger et al, 2018). In exemplary embodiments, a decrease in the CMT-FOM score can be determined by comparing the change from baseline in the CMT-FOM score after a defined period of time (e.g., 21 days) of NMD670 treatment with the change from baseline in the CMT-FOM score after a defined period of time (e.g., 21 days) of placebo treatment. In exemplary embodiments, the subject experiences a decrease in the CMT-FOM score after treatment with NMD670, wherein the score has decreased by at least 0.3 points, such as at least 0.5 points, such as at least 0.75 points, such as at least 1 point, such as at least 2 points, such as at least 3 points, such as at least 4 points, such as at least 5 points, such as at least 6 points, such as at least 8 points, such as at least 10 points, such as at least 15 points, such as at least 20 points, such as between 0.3 and 30 points, such as between 0.5 and 20 points, such as between 0.3 and 10 points.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and
the subject experiences a decrease in the CMT Functional Outcome Measure score.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and
the subject experiences a decrease in the CMT Functional Outcome Measure score.

In exemplary embodiments, the subject experiences an increase in the Berg Balance Scale score after treatment with NMD670 (Berg et al, 1989). In exemplary embodiments, an increase in the Berg Balance Scale score can be determined by comparing the change from baseline in the Berg Balance Scale score after a defined period of time (e.g., 21 days) of NMD670 treatment with the change from baseline in the Berg Balance Scale score after a defined period of time (e.g., 21 days) of placebo treatment. In exemplary embodiments, the subject experiences an increase in the Berg Balance Scale score after treatment with NMD670, wherein the score has increased by at least 0.5 points, such as at least 1 point, such as at least 2 points, such as at least 3 points, such as at least 4 points, such as at least 5 points, such as at least 6 points, such as at least 8 points, such as at least 10 points, such as at least 15 points, such as at least 20 points, such as between 0.5 and 30 points, such as between 1 and 20 points, such as between 0.5 and 10 points.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and
the subject experiences an increase in the Berg Balance Scale score.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and
the subject experiences an increase in the Berg Balance Scale score.

In exemplary embodiments, the subject a decrease in the Individualised Neuromuscular Quality of Life score after treatment with NMD670 (Vincent et al 2007). In exemplary embodiments, a decrease in the Individualised Neuromuscular Quality of Life score can be determined by comparing the change from baseline in the Individualised Neuromuscular Quality of Life score after a defined period of time (e.g., 21 days) of NMD670 treatment with the change from baseline the Individualised Neuromuscular Quality of Life score after a defined period of time (e.g., 21 days) of placebo treatment. In exemplary embodiments, the subject experiences a decrease in the Individualised Neuromuscular Quality of Life score after treatment with NMD670, wherein the score has decreased 0.5 points, such as at least 1 point, such as at least 2 points, such as at least 3 points, such as at least 4 points, such as at least 5 points, such as at least 6 points, such as at least 8 points, such as at least 10 points, such as at least 15 points, such as at least 20 points, such as between 0.5 and 30 points, such as between 1 and 20 points, such as between 0.5 and 10 points.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and
the subject experiences a decrease in the Individualised Neuromuscular Quality of Life score.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and
the subject experiences a decrease in the Individualised Neuromuscular Quality of Life score.

In exemplary embodiments, the subject a decrease in the Fatigue Severity Scale score after treatment with NMD670 (Krupp et al, 1989). In exemplary embodiments, a decrease in the Fatigue Severity Scale score can be determined by comparing the change from baseline in the Fatigue Severity Scale score after a defined period of time (e.g., 21 days) of NMD670 treatment with the change from baseline the Fatigue Severity Scale score after a defined period of time (e.g., 21 days) of placebo treatment. In exemplary embodiments, the subject experiences a decrease in the Fatigue Severity Scale score after treatment with NMD670, wherein the score has decreased 0.5 points, such as at least 1 point, such as at least 2 points, such as at least 3 points, such as at least 4 points, such as at least 5 points, such as at least 6 points, such as at least 8 points, such as at least 10 points, such as at least 15 points, such as at least 20 points, such as between 0.5 and 30 points, such as between 1 and 20 points, such as between 0.5 and 10 points.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and
the subject experiences a decrease in the Fatigue Severity Scale score.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and
the subject experiences a decrease in the Fatigue Severity Scale score.

In exemplary embodiments, the subject experiences a reduction in jitter after treatment with NMD670. In exemplary embodiments, the subject experiences a reduction in jitter after treatment with NMD670 when determined using single fibre electromyography (Sanders et al, 2019). In exemplary embodiments, a reduction in jitter can be determined by comparing the change from baseline in jitter after a defined period of time (e.g., 21 days) of NMD670 treatment with the change from baseline in jitter after a defined period of time (e.g., 21 days) of placebo treatment. In exemplary embodiments the subject experiences a reduction in jitter after treatment with NMD670 when determined using single fibre electromyography, wherein jitter has been reduced by at least 10%, such as at least 15%, such as at least 20%, such as at least 25%, such as at least 30%, such as at least 50%, such as at least 75%, such as at least 100%, such as at least 150%, such as at least 200%, such as between 5% and 95%, such as between 5% and 80%, such as between 10% and 50%. In exemplary embodiments the subject experiences a reduction in jitter after treatment with NMD670 when determined using single fibre electromyography, wherein jitter has been reduced by at least 5 μs, such as at least 10 μs, such as at least 15 μs, such as at least 20 μs, such as at least 25 μs, such as at least 30 μs, such as at least 40 μs, such as at least 50 μs, such as at least 75 μs, such as at least 100 μs, such as between 5 μs and 200 μs, such as between 5 μs and 100 μs, such as between 10 μs and 50 μs.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and
the subject experiences a reduction in jitter when determined using single fibre electromyography.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and
the subject experiences a reduction in jitter when determined using single fibre electromyography.

In exemplary embodiments, the subject experiences a reduction in blocking after treatment with NMD670. In exemplary embodiments, the subject experiences a reduction in blocking after treatment with NMD670 when determined using single fibre electromyography (Sanders et al, 2019). In exemplary embodiments, a reduction in blocking can be determined by comparing the change from baseline in blocking after a defined period of time (e.g., 21 days) of NMD670 treatment with the change from baseline in blocking after a defined period of time (e.g., 21 days) of placebo treatment. In exemplary embodiments the subject experiences a reduction in blocking after treatment with NMD670 when determined using single fibre electromyography, wherein blocking has been reduced by at least 5%, such as at least 10%, such as at least 15%, such as at least 20%, such as at least 25%, such as at least 30%, such as at least 50%, such as at least 75%, such as at least 100%, such as at least 150%, such as at least 200%, such as between 5% and 95%, such as between 5% and 80%, such as between 10% and 50%.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and
the subject experiences a reduction in blocking when determined using single fibre electromyography.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and
the subject experiences a reduction in blocking when determined using single fibre electromyography.

In exemplary embodiments, the subject experiences an improvement in finger dexterity after treatment with NMD670. In exemplary embodiments, the subject experiences an improvement in finger dexterity after treatment with NMD670 when determined using a nine-hole peg test (9-HPT) (Svensson et al, 2006; Mathiowetz et al, 1985). In exemplary embodiments, an improvement in finger dexterity can be determined by comparing the change from baseline in the time to complete the 9-HPT after a defined period of time (e.g., 21 days) of NMD670 treatment with the change from baseline in the time to complete the 9-HPT after a defined period of time (e.g., 21 days) of placebo treatment. In exemplary embodiments the subject experiences an improvement in finger dexterity after treatment with NMD670 when determined using a 9-HPT, wherein
the time to complete the 9-HPT has been reduced by at least 5%, such as at least 10%, such as at least 15%, such as at least 20%, such as at least 25%, such as at least 30%, such as at least 50%, such as at least 75%, such as at least 100%, such as between 5% and 95%, such as between 5% and 80%, such as between 10% and 50%.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and
the subject experiences an improvement in finger dexterity when determined using a nine-hole peg test.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and
the subject experiences an improvement in finger dexterity when determined using a nine-hole peg test.

In exemplary embodiments, the subject experiences an improvement in walking ability after treatment with NMD670. In exemplary embodiments, the subject experiences an improvement in walking ability after treatment with NMD670 when determined using the Six Spot Step Test (SSST) (Nieuwenhuis et al, 2006). In exemplary embodiments, an improvement in walking ability can be determined by comparing the time to complete the SSST after a defined period of time (e.g., 21 days) of NMD670 treatment with the time to complete the SSST after a defined period of time (e.g., 21 days) of placebo treatment. In exemplary embodiments the subject experiences an improvement in walking ability after treatment with NMD670 when determined using the SSST, wherein the time to complete the SSST has been reduced by at least 10%, such as at least 15%, such as at least 20%, such as at least 25%, such as at least 30%, such as at least 50%, such as at least 75%, such as at least 100%, such as at least 150%, such as at least 200%, such as between 5% and 95%, such as between 5% and 80%, such as between 10% and 50%. In exemplary embodiments the subject experiences an improvement in walking ability after treatment with NMD670 when determined using the SSST, wherein the time to complete the SSST has been reduced by at least 0.3 seconds, such as at least 0.5 s, such as at least 0.7 s, such as at least 1.0 s, such as at least 1.5 s, such as at least 2 s, such as at least 2.5 s, such as at least 3 s, such as at least 4 s, such as at least 5 s, such as between 0.3 s and 10 s, such as between 0.3 s and 5 s, such as between 0.5 s and 5 s.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and
the subject experiences an improvement in walking ability when determined using the Six Spot Step Test.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and
the subject experiences an improvement in walking ability when determined using the Six Spot Step Test.

In exemplary embodiments, the subject experiences an improvement in walking ability after treatment with NMD670. In exemplary embodiments, the subject experiences an improvement in walking ability after treatment with NMD670 when determined using the 10-meter walk/run test (10MWRT) (Hiu et al, 2017; Krosschell et al, 2022). In exemplary embodiments, an improvement in walking ability can be determined by comparing the time to complete the 10MWRT after a defined period of time (e.g., 21 days) of NMD670 treatment with the time to complete the 10MWRT after a defined period of time (e.g., 21 days) of placebo treatment. In exemplary embodiments the subject experiences an improvement in walking ability after treatment with NMD670 when determined using the 10MWRT, wherein the time to complete the 10MWRT has been reduced by at least 10%, such as at least 15%, such as at least 20%, such as at least 25%, such as at least 30%, such as at least 50%, such as at least 75%, such as at least 100%, such as at least 150%, such as at least 200%, such as between 5% and 95%, such as between 5% and 80%, such as between 10% and 50%. In exemplary embodiments the subject experiences an improvement in walking ability after treatment with NMD670 when determined using the 10MWRT, wherein the time to complete the 10MWRT has been reduced by at least 0.3 seconds, such as at least 0.5 s, such as at least 0.7 s, such as at least 1.0 s, such as at least 1.5 s, such as at least 2 s, such as at least 2.5 s, such as at least 3 s, such as at least 4 s, such as at least 5 s, such as between 0.3 s and 10 s, such as between 0.3 s and 5 s, such as between 0.5 s and 5 S.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and
the subject experiences an improvement in walking ability when determined using the 10-meter walk/run test.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and
the subject experiences an improvement in walking ability when determined using the 10-meter walk/run test.

In exemplary embodiments, the subject experiences an improvement in balance and mobility after treatment with NMD670. In exemplary embodiments, the subject experiences an improvement in balance and mobility after treatment with NMD670 when determined using the Timed “Up and Go” (TUG) test (Podsiadlo et al, 1991). In exemplary embodiments, an improvement in balance and mobility can be determined by comparing the time to complete the TUG test after a defined period of time (e.g., 21 days) of NMD670 treatment with the time to complete the TUG test after a defined period of time (e.g., 21 days) of placebo treatment. In exemplary embodiments the subject experiences an improvement in balance and mobility after treatment with NMD670 when determined using the TUG test, wherein the time to complete the TUG test has been reduced by at least 10%, such as at least 15%, such as at least 20%, such as at least 25%, such as at least 30%, such as at least 50%, such as at least 75%, such as at least 100%, such as at least 150%, such as at least 200%, such as between 5% and 95%, such as between 5% and 80%, such as between 10% and 50%. In exemplary embodiments the subject experiences an improvement in balance and mobility after treatment with NMD670 when determined using the TUG test, wherein the time to complete the TUG test has been reduced by at least 0.3 seconds, such as at least 0.5 s, such as at least 0.7 s, such as at least 1.0 s, such as at least 1.5 s, such as at least 2 s, such as at least 2.5 s, such as at least 3 s, such as at least 4 s, such as at least 5 s, such as between 0.3 s and 10 s, such as between 0.3 s and 5 s, such as between 0.5 s and 5 s.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and
the subject experiences an improvement in balance and mobility when determined using the Timed “Up and Go” test.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and
the subject experiences an improvement in balance and mobility when determined using the Timed “Up and Go” test.

In exemplary embodiments, the subject or group of subjects experience an increase in muscle strength after treatment with NMD670. Grip strength is one measure of muscular strength and can be used to determine the maximum force/tension generated by one's forearm muscles. Grip strength can be used as a screening tool for the measurement of upper body strength and overall strength. In exemplary embodiments, increase in muscle strength can also be determined by measuring the strength of the thigh (knee flexors), the upper arm (elbow flexor and extension) and/or the shoulder (shoulder abduction). In exemplary embodiments, the subject or group of subjects experience an increase in muscle strength after treatment with NMD670 when determined by measuring grip strength using a handheld dynamometer (Febrer et al, 2010; Merlini et al, 2002). In exemplary embodiments, an increase in muscle strength can be determined by comparing the change from baseline in muscle strength after a defined period of time (e.g., 21 days) of NMD670 treatment with the change from baseline in muscle strength after a defined period of time (e.g., 21 days) of placebo treatment. In exemplary embodiments the subject or group of subjects experience an increase in muscle strength after treatment with NMD670 when determined by measuring muscle strength using a handheld dynamometer, wherein muscle strength has increased by at least 5%, such as at least 10%, such as at least 15%, such as at least 20%, such as at least 25%, such as at least 30%, such as at least 50%, such as at least 75%, such as at least 100%, such as at least 150%, such as at least 200% such as between 10% and 400%, such as between 15% and 200%, such as between 20% and 100%.

In exemplary embodiments the subject or group of subjects experience an increase in muscle strength after treatment with NMD670 when determined by measuring hand grip strength using a handheld dynamometer, wherein hand grip strength has increased by at least 0.25 kg, such as at least 0.50 kg, such as at least 0.75 kg, such as at least 1.0 kg, such as at least 1.25 kg, such as at least 1.5 kg, such as at least 1.75 kg, such as at least 2.0 kg, such as at least 2.5 kg, such as at least 3.0 kg, such as between 0.25 and 5.0 kg, such as between 0.25 and 4.0 kg, such as between 0.5 and 4.0 kg.

In exemplary embodiments the subject or group of subjects experience an increase in muscle strength after treatment with NMD670 when determined by measuring knee flexor strength using a handheld dynamometer, wherein knee flexor strength has increased by at least 0.25 kg, such as at least 0.50 kg, such as at least 0.75 kg, such as at least 1.0 kg, such as at least 1.25 kg, such as at least 1.5 kg, such as at least 1.75 kg, such as at least 2.0 kg, such as at least 2.5 kg, such as at least 3.0 kg, such as between 0.25 and 5.0 kg, such as between 0.25 and 4.0 kg, such as between 0.5 and 4.0 kg.

In exemplary embodiments the subject or group of subjects experience an increase in muscle strength after treatment with NMD670 when determined by measuring elbow flexor strength using a handheld dynamometer, wherein elbow flexor strength has increased by at least 0.25 kg, such as at least 0.50 kg, such as at least 0.75 kg, such as at least 1.0 kg, such as at least 1.25 kg, such as at least 1.5 kg, such as at least 1.75 kg, such as at least 2.0 kg, such as at least 2.5 kg, such as at least 3.0 kg, such as between 0.25 and 5.0 kg, such as between 0.25 and 4.0 kg, such as between 0.5 and 4.0 kg.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences an increase in muscle strength when determined by measuring muscle strength using a handheld dynamometer.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences an increase in muscle strength when determined by measuring muscle strength using a handheld dynamometer.

In exemplary embodiments, the subject or group of subjects experience an increase in muscle strength after treatment with NMD670. In exemplary embodiments, the subject or group of subjects experience an increase in muscle strength after treatment with NMD670 when determined by measuring muscle strength using a fixed dynamometer (e.g. an isokinetic dynamometer) (Anders et al, 2012; Harbo et al, 2012). In exemplary embodiments, an increase in muscle strength can be determined by comparing the change from baseline in muscle strength after a defined period of time (e.g., 21 days) of NMD670 treatment with the change from baseline in muscle strength after a defined period of time (e.g., 21 days) of placebo treatment. In exemplary embodiments the subject or group of subjects experience an increase in muscle strength after treatment with NMD670 when determined by measuring muscle strength using a fixed dynamometer, wherein muscle strength has increased by at least 5%, such as at least 10%, such as at least 15%, such as at least 20%, such as at least 25%, such as at least 30%, such as at least 50%, such as at least 75%, such as at least 100%, such as at least 150%, such as at least 200% such as between 10% and 400%, such as between 15% and 200%, such as between 20% and 100%.

In exemplary embodiments the subject or group of subjects experience an increase in muscle strength after treatment with NMD670 when determined by measuring muscle strength of the ankle dorsiflexion using a fixed dynamometer, wherein ankle dorsiflexion strength has increased by at least 0.25 kg, such as at least 0.50 kg, such as at least 0.75 kg, such as at least 1.0 kg, such as at least 1.25 kg, such as at least 1.5 kg, such as at least 1.75 kg, such as at least 2.0 kg, such as at least 2.5 kg, such as at least 3.0 kg, such as between 0.25 and 5.0 kg, such as between 0.25 and 4.0 kg, such as between 0.5 and 4.0 kg.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences an increase in isometric strength when determined by measuring muscle strength using a fixed dynamometer.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences an increase in isometric strength when determined by measuring muscle strength using a fixed dynamometer.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein the subject has a level of serum uric acid below 6.5 mg/dL. In exemplary embodiments, the composition for use is for administration at a therapeutic dose of 100 to 1500 mg of (2S)-2-[4-bromo-2-(1,2-oxazol-3-yl)phenoxy]propanoic acid. In exemplary embodiments, the composition for use is for administration at a therapeutic dose as defined herein.

In one aspect, the present invention relates to use of a composition comprising (2S)-2-[4-bromo-2-(1,2-oxazol-3-yl)phenoxy]propanoic acid, or a pharmaceutically acceptable salt, hydrate, polymorph, tautomer, or solvate thereof, in the manufacture of a medicament for treatment of Charcot-Marie-Tooth disease in a subject, wherein the composition is for administration at a therapeutic dose of 100 to 1500 mg of (2S)-2-[4-bromo-2-(1,2-oxazol-3-yl)phenoxy]propanoic acid.

### Pharmaceutical Compositions

Another aspect of the present disclosure relates to compositions comprising a therapeutically effective dose of NMD670 for use in treating or ameliorating symptoms of Charcot-Marie-Tooth disease in a patient suffering from Charcot-Marie-Tooth disease. A further aspect of the present disclosure relates to compositions comprising a therapeutically effective dose of NMD670 in treating or ameliorating symptoms of Charcot-Marie-Tooth disease in a patient suffering from Charcot-Marie-Tooth disease. The following exemplified embodiments of the composition can be used in the treatment methods described herein.

In one aspect, the present invention relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof. In one embodiment, the composition comprises 50 to 400 mg (2S)-2-[4-bromo-2-(1,2-oxazol-3-yl)phenoxy]propanoic acid, or a pharmaceutically acceptable salt, hydrate, polymorph, tautomer, or solvate thereof. In one aspect, the present invention relates to a composition, formulated as a solid dosage form, comprising (2S)-2-[4-bromo-2-(1,2-oxazol-3-yl)phenoxy]propanoic acid, or a pharmaceutically acceptable salt, hydrate, polymorph, tautomer, or solvate thereof, wherein the composition comprises 50 to 400 mg (2S)-2-[4-bromo-2-(1,2-oxazol-3-yl)phenoxy]propanoic acid. In exemplary embodiments, the composition further comprises at least one pharmaceutically acceptable adjuvant and/or excipient. In exemplary embodiments, the composition is for oral administration. In exemplary embodiments, the composition further comprises a pharmaceutically acceptable adjuvant and/or excipient selected from the group consisting of filler, binder, lubricant and disintegrant. In exemplary embodiments, the composition comprises a pharmaceutically acceptable adjuvant and/or excipient selected from the group consisting of silicified microcrystalline cellulose, microcrystalline cellulose, maltodextrin, magnesium stearate and croscarmellose sodium.

In exemplary embodiments, the composition comprises 50 mg (2S)-2-[4-bromo-2-(1,2-oxazol-3-yl)phenoxy]propanoic acid, or a pharmaceutically acceptable salt, hydrate, polymorph, tautomer, or solvate thereof. In exemplary embodiments, the composition comprises 100 mg (2S)-2-[4-bromo-2-(1,2-oxazol-3-yl)phenoxy]propanoic acid, or a pharmaceutically acceptable salt, hydrate, polymorph, tautomer, or solvate thereof. In exemplary embodiments, the composition comprises 150 mg (2S)-2-[4-bromo-2-(1,2-oxazol-3-yl)phenoxy]propanoic acid, or a pharmaceutically acceptable salt, hydrate, polymorph, tautomer, or solvate thereof. In exemplary embodiments, the composition comprises 200 mg (2S)-2-[4-bromo-2-(1,2-oxazol-3-yl)phenoxy]propanoic acid, or a pharmaceutically acceptable salt, hydrate, polymorph, tautomer, or solvate thereof. In exemplary embodiments, the composition comprises 250 mg (2S)-2-[4-bromo-2-(1.2-oxazol-3-yl)phenoxy]propanoic acid, or a pharmaceutically acceptable salt, hydrate, polymorph, tautomer, or solvate thereof. In exemplary embodiments, the composition comprises 300 mg (2S)-2-[4-bromo-2-(1,2-oxazol-3-yl)phenoxy]propanoic acid, or a pharmaceutically acceptable salt, hydrate, polymorph, tautomer, or solvate thereof. In exemplary embodiments, the composition comprises 350 mg (2S)-2-[4-bromo-2-(1,2-oxazol-3-yl)phenoxy]propanoic acid, or a pharmaceutically acceptable salt, hydrate, polymorph, tautomer, or solvate thereof. In exemplary embodiments, the composition comprises 400 mg (2S)-2-[4-bromo-2-(1,2-oxazol-3-yl)phenoxy]propanoic acid, or a pharmaceutically acceptable salt, hydrate, polymorph, tautomer, or solvate thereof.

In exemplary embodiments, the composition comprises 10% to 80 wt %, such as 40 to 65 wt %, such as 50 to 55 wt %, such as about 53 wt % (2S)-2-[4-bromo-2-(1,2-oxazol-3-yl)phenoxy]propanoic acid, or a pharmaceutically acceptable salt, hydrate, polymorph, tautomer, or solvate thereof. In exemplary embodiments, the composition comprises 10% to 80 wt %, such as 40 to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof.

In exemplary embodiments, the composition comprises:
a. 10 to 80 wt %, such as 40 to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof;
b. 20 to 80 wt %, such as 25 to 50 wt % filler;
c. 2 to 20 wt %, such as 3 to 16 wt % binder;
d. 0.25 to 3 wt %, such as 0.4 to 2.0 wt % lubricant; and
e. 0.25 to 5 wt %, such as 0.3 to 2.5 wt % disintegrant;
   with the proviso that the sum of the wt % of the components does not exceed 100 wt %.

In exemplary embodiments, the composition comprises:
a. 10 to 80 wt %, such as 40 to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof;
b. 20 to 80 wt %, such as 25 to 50 wt % filler;
c. 2 to 20 wt %, such as 3 to 16 wt % binder;
d. 0.25 to 3 wt %, such as 0.4 to 2.0 wt % lubricant;
e. 0.25 to 5 wt %, such as 0.3 to 2.5 wt % disintegrant; and
f. 1 to 10 wt % film coating;
   with the proviso that the sum of the wt % of the components does not exceed 100 wt %.

In exemplary embodiments, the composition comprises:
a. 10 to 80 wt %, such as 40 to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof;
b. 5 to 60 wt %, such as 20 to 40 wt %, such as 21 to 37 wt % silicified microcrystalline cellulose;
c. 2 to 60 wt %, such as 5 to 16 wt % microcrystalline cellulose;
d. 1 to 15 wt %, such as 1.5 to 7 wt %, such as 1.8 to 6.0 wt % maltodextrin; and
e. 0.25 to 3 wt %, such as 0.4 to 2.0 wt % magnesium stearate; and
f. 0.25 to 5 wt %, such as 0.3 to 2.5 wt % Croscarmellose sodium;
   with the proviso that the sum of the wt % of the components does not exceed 100 wt %.

In exemplary embodiments, the composition comprises or consists of:
a. 10 to 80 wt %, such as 40 to 65 wt %, such as 50 to 55 wt %, such as about 53 wt % (2S)-2-[4-bromo-2-(1,2-oxazol-3-yl)phenoxy]propanoic acid, or a pharmaceutically acceptable salt, hydrate, polymorph, tautomer, or solvate thereof;
b. 5 to 60 wt %, such as 20 to 40 wt %, such as 21 to 37 wt % silicified microcrystalline cellulose;
c. 2 to 60 wt %, such as 5 to 16 wt % microcrystalline cellulose;
d. 1 to 15 wt %, such as 1.5 to 7 wt %, such as 1.8 to 6.0 wt % maltodextrin; and
e. 0.25 to 3 wt %, such as 0.4 to 2.0 wt % magnesium stearate;
f. 0.25 to 5 wt %, such as 0.3 to 2.5 wt % Croscarmellose sodium; and
g. 1 to 10 wt % film coating composition such as Opadry white,
   with the proviso that the sum of the wt % of the components does not exceed 100 wt %.

In exemplary embodiments, the present disclosure relates to a composition comprising a therapeutically effective dose of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mgs to 1500 mgs.

In exemplary embodiments, the present disclosure relates to a composition, formulated as a solid dosage form, comprising a therapeutically effective dose of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 50 mg to 400 mg. In exemplary embodiments, the therapeutically effective dose is 100 mgs. In exemplary embodiments, the therapeutically effective dose is 150 mgs. In exemplary embodiments, the therapeutically effective dose is 200 mgs. In exemplary embodiments, the therapeutically effective dose is 250 mgs. In exemplary embodiments, the therapeutically effective dose is 300 mgs. In exemplary embodiments, the therapeutically effective dose is 350 mgs. In exemplary embodiments, the therapeutically effective dose is 400 mgs. In exemplary embodiments, the therapeutically effective dose is given once daily. In exemplary embodiments, the therapeutically effective dose is given twice daily. In exemplary embodiments, the therapeutically effective dose is given three times daily. In exemplary embodiments, the therapeutically effective dose is given four times daily.

In exemplary embodiments, the present disclosure relates to a composition, formulated as a solid dosage form, comprising 50 mg to 400 mg of (2S)-2-[4-bromo-2-(1,2-oxazol-3-yl)phenoxy]propanoic acid or a pharmaceutically acceptable salt, hydrate, polymorph, tautomer, or solvate thereof. In exemplary embodiments, the solid dosage form comprises 100 mg of NMD670. In exemplary embodiments, the solid dosage form comprises 150 mg of NMD670. In exemplary embodiments, the solid dosage form comprises 200 mg of NMD670. In exemplary embodiments, the solid dosage form comprises 250 mg of NMD670. In exemplary embodiments, the solid dosage form comprises 300 mg of NMD670. In exemplary embodiments, the solid dosage form comprises 350 mg of NMD670. In exemplary embodiments, the solid dosage form comprises 400 mg of NMD670.

In exemplary embodiment, the composition is a solid dosage form. In exemplary embodiments, the solid dosage form is selected from the group consisting of capsule (such as sprinkle capsule and gelatine capsule), tablet (such as uncoated tablet, coated tablet, slow-release tablet) and sprinkle. In exemplary embodiments, the composition is in the form of a liquid, liquid suspension, oil, emulsion, or syrup. In exemplary embodiments, the solid dosage form releases not less than 80% of the compound after 30 minutes, when measured in a United States Pharmacopeia (USP) type 2 dissolution apparatus, paddle at 75 rpm, at a temperature of 37° C.±0.5° C. in 900 mL of pH 6.8 phosphate/citric acid buffer.

### Methods

In one aspect, the present disclosure relates to a method of treatment of Charcot-Marie-Tooth disease in a subject, said method comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.

In exemplary embodiments, the methods for treating a patient suffering from symptoms of Charcot-Marie-Tooth disease may result in a decrease in the Overall Neuropathy Limitations Score; an increase in the total distance walked during a 6-minute walk test; a reduction in fatigue when determined using a fatigue index calculated from the 6-minute walk test; a decrease in the CMT Neuropathy Score 2 score; a decrease in the CMT examination score (second version) score; an increase in the motor function measure 32-item score; a decrease in the CMT Functional Outcome Measure score; an increase in the Berg Balance Scale score; a decrease in the Individualised Neuromuscular Quality of Life score; a decrease in the Fatigue Severity Scale score; and/or an improvement in neuromuscular junction transmission (wherein an improvement is a reduction of jitter and/or blocking when measured using sfEMG); an improvement in finger dexterity when determined using a nine-hole peg test (9-HPT); an improvement in walking ability when determined using a Six Spot Step Test; an improvement in walking ability when determined using a 10-meter walk/run test; an improvement in balance and mobility when determined using the Timed “Up and Go” test; an increase in muscle strength as assessed with a hand held dynamometer; and/or an increase in muscle strength as assessed with a fixed dynamometer.

Accordingly, one aspect of the present disclosure relates to methods for treating Charcot-Marie-Tooth disease that result in a decrease in the Overall Neuropathy Limitations Score (ONLS) in a patient, these methods comprising administering a therapeutically effective dose of NMD670 to the patient, wherein the therapeutic dose is within the range of 100 mg to 1500 mg. An improvement in the Overall Neuropathy Limitations Score (ONLS) is a decrease in score (Graham et al, 2006).

One aspect of the present disclosure relates to methods for treating Charcot-Marie-Tooth disease that result in an improvement in the total distance walked in a patient, these methods comprising administering a therapeutically effective dose of NMD670 to the patient, wherein the therapeutic dose is within the range of 100 mg to 1500 mg. An improvement in the total distance walked can be determined using a 6-minute walk test (ATS, 2002).

One aspect of the present disclosure relates to methods for treating Charcot-Marie-Tooth disease that result in a reduction in fatigue, these methods comprising administering a therapeutically effective dose of NMD670 to the patient, wherein the therapeutic dose is within the range of 100 mg to 1500 mg. A reduction in fatigue can be calculated from the distance walked in the 1st minute to the distance walked in the 6th minute of a 6-minute walk test (ATS, 2002)

One aspect of the present disclosure relates to methods for treating Charcot-Marie-Tooth disease that result in a decrease in the CMT Neuropathy Score 2 (CMTNS2) score in a patient, these methods comprising administering a therapeutically effective dose of NMD670 to the patient, wherein the therapeutic dose is within the range of 100 mg to 1500 mg. An improvement in the CMT Neuropathy Score 2 score is a decrease in score (Murphy et al, 2011).

One aspect of the present disclosure relates to methods for treating Charcot-Marie-Tooth disease that result in a decrease in the CMT examination score (second version) (CMTES2) score in a patient, these methods comprising administering a therapeutically effective dose of NMD670 to the patient, wherein the therapeutic dose is within the range of 100 mg to 1500 mg. An improvement in the CMT examination score (second version) score is a decrease in score (Murphy et al, 2011).

One aspect of the present disclosure relates to methods for treating Charcot-Marie-Tooth disease that result in an increase in the motor function measure 32-item score in a patient, these methods comprising administering a therapeutically effective dose of NMD670 to the patient, wherein the therapeutic dose is within the range of 100 mg to 1500 mg. An improvement in the motor function measure 32-item score is an increase in score (Allard et al 2014).

One aspect of the present disclosure relates to methods for treating Charcot-Marie-Tooth disease that result in a decrease in the CMT Functional Outcome Measure (CMT-FOM) score in a patient, these methods comprising administering a therapeutically effective dose of NMD670 to the patient, wherein the therapeutic dose is within the range of 100 mg to 1500 mg. An improvement in the CMT Functional Outcome Measure score is a decrease in score (Eichinger et al, 2018).

One aspect of the present disclosure relates to methods for treating Charcot-Marie-Tooth disease that result in an increase in the Berg Balance Scale score in a patient, these methods comprising administering a therapeutically effective dose of NMD670 to the patient, wherein the therapeutic dose is within the range of 100 mg to 1500 mg. An improvement in the Berg Balance Scale score is an increase in score (Berg et al, 1989).

One aspect of the present disclosure relates to methods for treating Charcot-Marie-Tooth disease that result in a decrease in the Individualised Neuromuscular Quality of Life score, these methods comprising administering a therapeutically effective dose of NMD670 to the patient, wherein the therapeutic dose is within the range of 100 mg to 1500 mg. An improvement in the Individualised Neuromuscular Quality of Life score is a decrease in score (Vincent et al 2007).

One aspect of the present disclosure relates to methods for treating Charcot-Marie-Tooth disease that result in a decrease in the Fatigue Severity Scale score in a patient, these methods comprising administering a therapeutically effective dose of NMD670 to the patient, wherein the therapeutic dose is within the range of 100 mg to 1500 mg. An improvement in the Fatigue Severity Scale score is a decrease in score (Krupp et al, 1989).

One aspect of the present disclosure relates to methods for Charcot-Marie-Tooth disease that result in an improvement in neuromuscular junction transmission, these methods comprising administering a therapeutically effective dose of NMD670 to the patient, wherein the therapeutic dose is within the range of 100 mg to 1500 mg. An improvement in neuromuscular junction transmission can be a reduction in jitter and/or blocking and can be determined using single fibre electromyography (Sanders et al, 2019).

One aspect of the present disclosure relates to methods for Charcot-Marie-Tooth disease that result in an improvement in finger dexterity, these methods comprising administering a therapeutically effective dose of NMD670 to the patient, wherein the therapeutic dose is within the range of 100 mg to 1500 mg. An improvement in finger dexterity can be determined using a nine-hole peg test (Svensson et al, 2006; Mathiowetz et al, 1985).

One aspect of the present disclosure relates to methods for Charcot-Marie-Tooth disease that result in an improvement in walking ability, these methods comprising administering a therapeutically effective dose of NMD670 to the patient, wherein the therapeutic dose is within the range of 100 mg to 1500 mg. An improvement in walking ability can be determined using the Six Spot Step Test (Nieuwenhuis et al, 2006).

One aspect of the present disclosure relates to methods for Charcot-Marie-Tooth disease that result in an improvement in walking ability, these methods comprising administering a therapeutically effective dose of NMD670 to the patient, wherein the therapeutic dose is within the range of 100 mg to 1500 mg. An improvement in walking ability can be determined using the 10-meter walk/run test (Hiu et al, 2017; Krosschell et al, 2022).

One aspect of the present disclosure relates to methods for Charcot-Marie-Tooth disease that result in an improvement in balance and mobility, these methods comprising administering a therapeutically effective dose of NMD670 to the patient, wherein the therapeutic dose is within the range of 100 mg to 1500 mg. An improvement in balance and mobility can be determined using the Timed “Up and Go” test (Podsiadlo et al, 1991).

One aspect of the present disclosure relates to methods for Charcot-Marie-Tooth disease that result in an improvement in muscle strength, these methods comprising administering a therapeutically effective dose of NMD670 to the patient, wherein the therapeutic dose is within the range of 100 mg to 1500 mg. An improvement in muscle strength can be determined by measuring grip strength using a handheld dynamometer (Febrer et al, 2010; Merlini et al, 2002).

One aspect of the present disclosure relates to methods for Charcot-Marie-Tooth disease that result in an improvement in muscle strength, these methods comprising administering a therapeutically effective dose of NMD670 to the patient, wherein the therapeutic dose is within the range of 100 mg to 1500 mg. An improvement in muscle strength can be determined by measuring muscle strength using a fixed dynamometer (e.g. an isokinetic dynamometer) (Anders et al, 2012; Harbo et al, 2012).

In one aspect, the present disclosure relates to a method for treatment of Charcot-Marie-Tooth disease in a subject in need thereof, comprising administering a composition comprising (2S)-2-[4-bromo-2-(1,2-oxazol-3-yl)phenoxy]propanoic acid, or a pharmaceutically acceptable salt, hydrate, polymorph, tautomer, or solvate thereof, at a therapeutic dose of 100 to 1500 mg of (2S)-2-[4-bromo-2-(1,2-oxazol-3-yl)phenoxy]propanoic acid to said subject.

In one aspect, the present disclosure relates to a method for enhancing neuromuscular transmission and/or restoration of skeletal muscle function, comprising administering a composition comprising (2S)-2-[4-bromo-2-(1,2-oxazol-3-yl)phenoxy]propanoic acid, or a pharmaceutically acceptable salt, hydrate, polymorph, tautomer, or solvate thereof, at a therapeutic dose of 100 to 1500 mg of (2S)-2-[4-bromo-2-(1,2-oxazol-3-yl)phenoxy]propanoic acid to said subject.

In one aspect, the present disclosure relates to a method for treatment of Charcot-Marie-Tooth disease in a subject with serum uric acid levels above 6.5 mg/dL, said method comprising administering a low dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject until the patient's serum uric acid level falls below 6.5 mg/dL, and then administering a therapeutic dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the low dose is within the range of 20 mg to 150 mg, such as 25 mg to 100 mg, such as 25 mg to 50 mg, and the therapeutic dose is within the range of 200 mg to 1500 mg.

In exemplary embodiments, the composition is administered at a therapeutic dose of 100 to 600 mg of (2S)-2-[4-bromo-2-(1,2-oxazol-3-yl)phenoxy]propanoic acid and the composition is to be administered one time daily.

In exemplary embodiments, the composition is administered at a therapeutic dose of 100 to 600 mg of (2S)-2-[4-bromo-2-(1,2-oxazol-3-yl)phenoxy]propanoic acid and the composition is to be administered two times daily.

In exemplary embodiments, the composition is administered at a therapeutic dose of 100 to 600 mg of (2S)-2-[4-bromo-2-(1,2-oxazol-3-yl)phenoxy]propanoic acid and the composition is to be administered three times daily.

In exemplary embodiments, the composition is administered at a therapeutic dose of 100 to 600 mg of (2S)-2-[4-bromo-2-(1,2-oxazol-3-yl)phenoxy]propanoic acid and the composition is to be administered four times daily.

In exemplary embodiments, the composition is administered at a therapeutic dose of 200 to 600 mg of (2S)-2-[4-bromo-2-(1,2-oxazol-3-yl)phenoxy]propanoic acid and the composition is to be administered one time daily.

In exemplary embodiments, the composition is administered at a therapeutic dose of 200 to 600 mg of (2S)-2-[4-bromo-2-(1,2-oxazol-3-yl)phenoxy]propanoic acid and the composition is to be administered two times daily.

In exemplary embodiments, the composition is administered at a therapeutic dose of 200 to 600 mg of (2S)-2-[4-bromo-2-(1,2-oxazol-3-yl)phenoxy]propanoic acid and the composition is to be administered three times daily.

In exemplary embodiments, the composition is administered at a therapeutic dose of 200 to 600 mg of (2S)-2-[4-bromo-2-(1,2-oxazol-3-yl)phenoxy]propanoic acid and the composition is to be administered four times daily.

In exemplary embodiments, the composition is administered at a therapeutic dose of about 100 mg of (2S)-2-[4-bromo-2-(1,2-oxazol-3-yl)phenoxy]propanoic acid and the composition is to be administered two times daily.

In exemplary embodiments, the composition is administered at a therapeutic dose of about 100 mg of (2S)-2-[4-bromo-2-(1,2-oxazol-3-yl)phenoxy]propanoic acid and the composition is to be administered three times daily.

In exemplary embodiments, the composition is administered at a therapeutic dose of about 100 mg of (2S)-2-[4-bromo-2-(1,2-oxazol-3-yl)phenoxy]propanoic acid and the composition is to be administered four times daily.

In exemplary embodiments, the composition is administered at a therapeutic dose of about 150 mg of (2S)-2-[4-bromo-2-(1,2-oxazol-3-yl)phenoxy]propanoic acid and the composition is to be administered two times daily.

In exemplary embodiments, the composition is administered at a therapeutic dose of about 150 mg of (2S)-2-[4-bromo-2-(1,2-oxazol-3-yl)phenoxy]propanoic acid and the composition is to be administered three times daily.

In exemplary embodiments, the composition is administered at a therapeutic dose of about 150 mg of (2S)-2-[4-bromo-2-(1,2-oxazol-3-yl)phenoxy]propanoic acid and the composition is to be administered four times daily.

In exemplary embodiments, the composition is administered at a therapeutic dose of about 200 mg of (2S)-2-[4-bromo-2-(1,2-oxazol-3-yl)phenoxy]propanoic acid and the composition is to be administered one time daily.

In exemplary embodiments, the composition is administered at a therapeutic dose of about 200 mg of (2S)-2-[4-bromo-2-(1,2-oxazol-3-yl)phenoxy]propanoic acid and the composition is to be administered two times daily.

In exemplary embodiments, the composition is administered at a therapeutic dose of about 200 mg of (2S)-2-[4-bromo-2-(1,2-oxazol-3-yl)phenoxy]propanoic acid and the composition is to be administered three times daily.

In exemplary embodiments, the composition is administered at a therapeutic dose of about 200 mg of (2S)-2-[4-bromo-2-(1,2-oxazol-3-yl)phenoxy]propanoic acid and the composition is to be administered four times daily.

In exemplary embodiments, the composition is administered at a therapeutic dose of about 250 mg of (2S)-2-[4-bromo-2-(1,2-oxazol-3-yl)phenoxy]propanoic acid and the composition is to be administered one time daily.

In exemplary embodiments, the composition is administered at a therapeutic dose of about 250 mg of (2S)-2-[4-bromo-2-(1,2-oxazol-3-yl)phenoxy]propanoic acid and the composition is to be administered two times daily.

In exemplary embodiments, the composition is administered at a therapeutic dose of about 250 mg of (2S)-2-[4-bromo-2-(1,2-oxazol-3-yl)phenoxy]propanoic acid and the composition is to be administered three times daily.

In exemplary embodiments, the composition is administered at a therapeutic dose of about 250 mg of (2S)-2-[4-bromo-2-(1,2-oxazol-3-yl)phenoxy]propanoic acid and the composition is to be administered four times daily.

In exemplary embodiments, the composition is administered at a therapeutic dose of about 300 mg of (2S)-2-[4-bromo-2-(1,2-oxazol-3-yl)phenoxy]propanoic acid and the composition is to be administered one time daily.

In exemplary embodiments, the composition is administered at a therapeutic dose of about 300 mg of (2S)-2-[4-bromo-2-(1,2-oxazol-3-yl)phenoxy]propanoic acid and the composition is to be administered two times daily.

In exemplary embodiments, the composition is administered at a therapeutic dose of about 300 mg of (2S)-2-[4-bromo-2-(1,2-oxazol-3-yl)phenoxy]propanoic acid and the composition is to be administered three times daily.

In exemplary embodiments, the composition is administered at a therapeutic dose of about 300 mg of (2S)-2-[4-bromo-2-(1,2-oxazol-3-yl)phenoxy]propanoic acid and the composition is to be administered four times daily.

In exemplary embodiments, the composition is administered at a therapeutic dose of about 350 mg of (2S)-2-[4-bromo-2-(1,2-oxazol-3-yl)phenoxy]propanoic acid and the composition is to be administered one time daily.

In exemplary embodiments, the composition is administered at a therapeutic dose of about 350 mg of (2S)-2-[4-bromo-2-(1,2-oxazol-3-yl)phenoxy]propanoic acid and the composition is to be administered two times daily.

In exemplary embodiments, the composition is administered at a therapeutic dose of about 350 mg of (2S)-2-[4-bromo-2-(1,2-oxazol-3-yl)phenoxy]propanoic acid and the composition is to be administered three times daily.

In exemplary embodiments, the composition is administered at a therapeutic dose of about 350 mg of (2S)-2-[4-bromo-2-(1,2-oxazol-3-yl)phenoxy]propanoic acid and the composition is to be administered four times daily.

In exemplary embodiments, the composition is administered at a therapeutic dose of about 400 mg of (2S)-2-[4-bromo-2-(1,2-oxazol-3-yl)phenoxy]propanoic acid and the composition is to be administered one time daily.

In exemplary embodiments, the composition is administered at a therapeutic dose of about 400 mg of (2S)-2-[4-bromo-2-(1,2-oxazol-3-yl)phenoxy]propanoic acid and the composition is to be administered two times daily.

In exemplary embodiments, the composition is administered at a therapeutic dose of about 400 mg of (2S)-2-[4-bromo-2-(1,2-oxazol-3-yl)phenoxy]propanoic acid and the composition is to be administered three times daily.

In exemplary embodiments, the composition is administered at a therapeutic dose of about 400 mg of (2S)-2-[4-bromo-2-(1,2-oxazol-3-yl)phenoxy]propanoic acid and the composition is to be administered four times daily.

In exemplary embodiments, the composition is administered at a therapeutic dose of about 500 mg of (2S)-2-[4-bromo-2-(1,2-oxazol-3-yl)phenoxy]propanoic acid and the composition is to be administered one time daily.

In exemplary embodiments, the composition is administered at a therapeutic dose of about 500 mg of (2S)-2-[4-bromo-2-(1,2-oxazol-3-yl)phenoxy]propanoic acid and the composition is to be administered two times daily.

In exemplary embodiments, the composition is administered at a therapeutic dose of about 500 mg of (2S)-2-[4-bromo-2-(1,2-oxazol-3-yl)phenoxy]propanoic acid and the composition is to be administered three times daily.

In exemplary embodiments, the composition is administered at a therapeutic dose of about 600 mg of (2S)-2-[4-bromo-2-(1,2-oxazol-3-yl)phenoxy]propanoic acid and the composition is to be administered one time daily.

In exemplary embodiments, the composition is administered at a therapeutic dose of about 600 mg of (2S)-2-[4-bromo-2-(1,2-oxazol-3-yl)phenoxy]propanoic acid and the composition is to be administered two times daily.

In exemplary embodiments, the composition is administered at a therapeutic dose of about 600 mg of (2S)-2-[4-bromo-2-(1,2-oxazol-3-yl)phenoxy]propanoic acid and the composition is to be administered three times daily.

In one aspect, the present disclosure relates to a method of treating Charcot-Marie-Tooth disease that results in a decrease in the Overall Neuropathy Limitations Score in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences an improvement in the Overall Neuropathy Limitations Score.

In exemplary embodiments, the patient experiences a decrease in the Overall Neuropathy Limitations Score of at least 0.3 points, such as at least 0.5 points, such as at least 0.75 points, such as at least 1 point, such as at least 2 points, such as at least 3 points, such as at least 4 points, such as at least 5 points, such as at least 6 points, such as at least 8 points, such as at least 10 points, such as between 0.3 and 10 points, such as between 0.5 and 6 points, such as between 0.3 and 5 points.

In one aspect, the present disclosure relates to a method of treating Charcot-Marie-Tooth disease that results in an increase in total distance walked in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences an increase in the total distance walked when determined using a 6-minute walk test.

In exemplary embodiments, the improvement in the total distance walked is determined by comparing the change from baseline in the total distance walked after a defined period of time (e.g., 21 days) of NMD670 treatment with the change from baseline in the total distance walked after a defined period of time (e.g., 21 days) of placebo treatment. In exemplary embodiments, the patient experiences an increase in total distance walked of at least 5%, such as at least 10%, such as at least 15%, such as at least 20%, such as at least 25%, such as at least 30%, such as at least 50%, such as at least 75%, such as at least 100%, such as at least 150%, such as at least 200%, such as between 5% and 400%, such as between 5% and 200%, such as between 10% and 200%. In exemplary embodiments, the patient experiences an increase in total distance walked of at least 20 metres, such as at least 30 metres, such as at least metres, such as at least 50 metres, such as at least 60 metres, such as at least 80 metres, such as at least 100 metres, such as at least 150 metres, such as at least 200 metres, such as at least 250 metres, such as at least 300 metres, such as between 20 and 400 metres, such as between 30 and 300 metres, such as between 40 and 200 metres.

In one aspect, the present disclosure relates to a method of treating Charcot-Marie-Tooth disease that results in a reduction in fatigue in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences a reduction in fatigue when determined using a fatigue index calculated from the distance walked in the 1st minute to the distance walked in the 6th minute of a 6-minute walk test.

In exemplary embodiments, the patient experiences a reduction in fatigue of at least 5%, such as at least 10%, such as at least 15%, such as at least 20%, such as at least 25%, such as at least 30%, such as at least 40%, such as at least 50%, such as at least 60%, such as at least 70%, such as at least 80%, such as between 5% and 95%, such as between 5% and 80%, such as between 10% and 50%.

In one aspect, the present disclosure relates to a method of treating Charcot-Marie-Tooth disease that results in a decrease in the CMT Neuropathy Score 2 (CMTNS2) score in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences a decrease in the CMTNS2 score.

In exemplary embodiments, the patient experiences a decrease in the CMTNS2 score of at least 0.3 points, such as at least 0.5 points, such as at least 0.75 points, such as at least 1 point, such as at least 2 points, such as at least 3 points, such as at least 4 points, such as at least 5 points, such as at least 6 points, such as at least 8 points, such as at least 10 points, such as between 0.3 and 20 points, such as between 0.5 and 6 points, such as between 0.3 and 5 points.

In one aspect, the present disclosure relates to a method of treating Charcot-Marie-Tooth disease that results in a decrease in the CMT examination score (second version) (CMTES2) score in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences a decrease in the CMTES2 score.

In exemplary embodiments, the patient experiences a decrease in the CMTES2 score of at least 0.3 points, such as at least 0.5 points, such as at least 0.75 points, such as at least 1 point, such as at least 2 points, such as at least 3 points, such as at least 4 points, such as at least 5 points, such as at least 6 points, such as at least 8 points, such as at least 10 points, such as between 0.3 and 20 points, such as between 0.5 and 6 points, such as between 0.3 and 5 points.

In one aspect, the present disclosure relates to a method of treating Charcot-Marie-Tooth disease that results in an increase in the motor function measure 32-item score in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences an increase in the motor function measure 32-item score.

In exemplary embodiments, the patient experiences an increase in the motor function measure 32-item score of at least 5%, such as at least 10%, such as at least 15%, such as at least 20%, such as at least 25%, such as at least 30%, such as at least 40%, such as at least 50%, such as at least 60%, such as at least 70%, such as at least 80%, such as between 5% and 95%, such as between 5% and 80%, such as between 10% and 50%.

In one aspect, the present disclosure relates to a method of treating Charcot-Marie-Tooth disease that results in a decrease in the CMT Functional Outcome Measure (CMT-FOM) score in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences a decrease in the CMT-FOM score.

In exemplary embodiments, the patient experiences a decrease in the CMT-FOM score of at least 0.3 points, such as at least 0.5 points, such as at least 0.75 points, such as at least 1 point, such as at least 2 points, such as at least 3 points, such as at least 4 points, such as at least 5 points, such as at least 6 points, such as at least 8 points, such as at least 10 points, such as at least 15 points, such as at least 20 points, such as between 0.3 and 30 points, such as between 0.5 and 20 points, such as between 0.3 and 10 points.

In one aspect, the present disclosure relates to a method of treating Charcot-Marie-Tooth disease that results in an increase in the Berg Balance Scale score in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences increase in the Berg Balance Scale score.

In exemplary embodiments, the patient experiences an increase in the Berg Balance Scale score of at least 0.3 points, such as at least 0.5 points, such as at least 0.75 points, such as at least 1 point, such as at least 2 points, such as at least 3 points, such as at least 4 points, such as at least 5 points, such as at least 6 points, such as at least 8 points, such as at least 10 points, such as at least 15 points, such as at least 20 points, such as between 0.3 and 30 points, such as between 0.5 and 20 points, such as between 0.3 and 10 points.

In one aspect, the present disclosure relates to a method of treating Charcot-Marie-Tooth disease that results in a decrease in the Individualised Neuromuscular Quality of Life score in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences a decrease in the Individualised Neuromuscular Quality of Life score.

In exemplary embodiments, the patient experiences a decrease in the Individualised Neuromuscular Quality of Life score of at least 0.3 points, such as at least 0.5 points, such as at least 0.75 points, such as at least 1 point, such as at least 2 points, such as at least 3 points, such as at least 4 points, such as at least 5 points, such as at least 6 points, such as at least 8 points, such as at least 10 points, such as at least 15 points, such as at least 20 points, such as between 0.3 and 30 points, such as between 0.5 and 20 points, such as between 0.3 and 10 points.

In one aspect, the present disclosure relates to a method of treating Charcot-Marie-Tooth disease that results in a decrease in the Fatigue Severity Scale score in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences a decrease in the Fatigue Severity Scale score.

In exemplary embodiments, the patient experiences a decrease in the Fatigue Severity Scale score of at least 0.3 points, such as at least 0.5 points, such as at least 0.75 points, such as at least 1 point, such as at least 2 points, such as at least 3 points, such as at least 4 points, such as at least 5 points, such as at least 6 points, such as at least 8 points, such as at least 10 points, such as at least 15 points, such as at least 20 points, such as between 0.3 and 30 points, such as between 0.5 and 20 points, such as between 0.3 and 10 points.

In one aspect, the present disclosure relates to a method of treating Charcot-Marie-Tooth disease that results in a reduction in jitter in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences a reduction in jitter when determined using single fibre electromyography.

In exemplary embodiments, the patient experiences a reduction in jitter of at least 10%, such as at least 15%, such as at least 20%, such as at least 25%, such as at least 30%, such as at least 50%, such as at least 75%, such as at least 100%, such as at least 150%, such as at least 200%, such as between 5% and 95%, such as between 5% and 80%, such as between 10% and 50%. In exemplary embodiments the subject experiences a reduction in jitter of at least 5 μs, such as at least 10 μs, such as at least 15 μs, such as at least 20 μs, such as at least 25 μs, such as at least 30 μs, such as at least 40 μs, such as at least 50 μs, such as at least 75 μs, such as at least 100 μs, such as between 5 μs and 200 μs, such as between 5 μs and 100 μs, such as between 10 μs and 50 μs.

In one aspect, the present disclosure relates to a method of treating Charcot-Marie-Tooth disease that results in a reduction in blocking in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences a reduction in blocking when determined using single fibre electromyography.

In exemplary embodiments, the patient experiences a reduction in blocking of at least 10%, such as at least 15%, such as at least 20%, such as at least 25%, such as at least 30%, such as at least 50%, such as at least 75%, such as at least 100%, such as at least 150%, such as at least 200%, such as between 5% and 95%, such as between 5% and 80%, such as between 10% and 50%.

In one aspect, the present disclosure relates to a method of treating Charcot-Marie-Tooth disease that results in an improvement in finger dexterity in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences an improvement in finger dexterity when determined using a nine-hole peg test.

In exemplary embodiments, the patient experiences an improvement in finger dexterity of at least 10%, such as at least 15%, such as at least 20%, such as at least 25%, such as at least 30%, such as at least 50%, such as at least 75%, such as at least 100%, such as at least 150%, such as at least 200%, such as between 5% and 95%, such as between 5% and 80%, such as between 10% and 50%.

In one aspect, the present disclosure relates to a method of treating Charcot-Marie-Tooth disease that results in an improvement in walking ability in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences an improvement in walking ability.

In exemplary embodiments, the patient experiences an improvement in walking ability of at least 10%, such as at least 15%, such as at least 20%, such as at least 25%, such as at least 30%, such as at least 50%, such as at least 75%, such as at least 100%, such as at least 150%, such as at least 200%, such as between 5% and 95%, such as between 5% and 80%, such as between 10% and 50%.

In exemplary embodiments, the patient experiences an improvement in walking ability wherein the time to complete the Six Spot Step Test has been reduced by at least 0.3 seconds, such as at least 0.5 s, such as at least 0.7 s, such as at least 1.0 s, such as at least 1.5 s, such as at least 2 s, such as at least 2.5 s, such as at least 3 s, such as at least 4 s, such as at least 5 s, such as between 0.3 s and 10 s, such as between 0.3 s and 5 s, such as between 0.5 s and 5 s.

In exemplary embodiments, the patient experiences an improvement in walking ability wherein the time to complete the 10-meter walk/run test has been reduced by at least 0.3 seconds, such as at least 0.5 s, such as at least 0.7 s, such as at least 1.0 s, such as at least 1.5 s, such as at least 2 s, such as at least 2.5 s, such as at least 3 s, such as at least 4 s, such as at least 5 s, such as between 0.3 s and 10 s, such as between 0.3 s and 5 s, such as between 0.5 s and 5 s.

In one aspect, the present disclosure relates to a method of treating Charcot-Marie-Tooth disease that results in an improvement in balance and mobility in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences an improvement in balance and mobility.

In exemplary embodiments, the patient experiences an improvement in balance and mobility of at least 10%, such as at least 15%, such as at least 20%, such as at least 25%, such as at least 30%, such as at least 50%, such as at least 75%, such as at least 100%, such as at least 150%, such as at least 200%, such as between 5% and 95%, such as between 5% and 80%, such as between 10% and 50%.

In exemplary embodiments, the patient experiences an improvement in walking ability wherein the time to complete the Timed “Up and Go” test has been reduced by at least 0.3 seconds, such as at least 0.5 s, such as at least 0.7 s, such as at least 1.0 s, such as at least 1.5 s, such as at least 2 s, such as at least 2.5 s, such as at least 3 s, such as at least 4 s, such as at least 5 s, such as between 0.3 s and 10 s, such as between 0.3 s and 5 s, such as between 0.5 s and 5 s.

In one aspect, the present disclosure relates to a method of treating Charcot-Marie-Tooth disease that results in an increase in muscle strength in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences an increase in muscle strength.

In exemplary embodiments, the patient experiences an increase in muscle strength of at least 5%, such as at least 10%, such as at least 15%, such as at least 20%, such as at least 25%, such as at least 30%, such as at least 50%, such as at least 75%, such as at least 100%, such as at least 150%, such as at least 200% such as between 10% and 400%, such as between 15% and 200%, such as between 20% and 100%.

In exemplary embodiments, the patient experiences an increase in muscle strength when determined by measuring hand grip strength using a handheld dynamometer, wherein hand grip strength has increased by at least 0.25 kg, such as at least 0.50 kg, such as at least 0.75 kg, such as at least 1.0 kg, such as at least 1.25 kg, such as at least 1.5 kg, such as at least 1.75 kg, such as at least 2.0 kg, such as at least 2.5 kg, such as at least 3.0 kg, such as between 0.25 and 5.0 kg, such as between 0.25 and 4.0 kg, such as between 0.5 and 4.0 kg.

In exemplary embodiments, the patient experiences an increase in muscle strength when determined by measuring muscle strength of the ankle dorsiflexion using a fixed dynamometer, wherein ankle dorsiflexion strength has increased by at least 0.25 kg, such as at least 0.50 kg, such as at least 0.75 kg, such as at least 1.0 kg, such as at least 1.25 kg, such as at least 1.5 kg, such as at least 1.75 kg, such as at least 2.0 kg, such as at least 2.5 kg, such as at least 3.0 kg, such as between 0.25 and 5.0 kg, such as between 0.25 and 4.0 kg, such as between 0.5 and 4.0 kg.

In exemplary embodiments, the therapeutically effective dose of the compound is administered orally to the patient. In exemplary embodiments, the therapeutically effective dose is within the range of 100 mg to 600 mg. In exemplary embodiments, the therapeutically effective dose is within the range of 200 mgs to 600 mgs. In exemplary embodiments, the therapeutically effective dose is 100 mg. In exemplary embodiments the therapeutically effective dose is 150 mg. In exemplary embodiments, the therapeutically effective dose is 200 mg. In exemplary embodiments, the therapeutically effective dose is 250 mg. In exemplary embodiments, the therapeutically effective dose is 300 mg. In exemplary embodiments, the therapeutically effective dose is 350 mg. In exemplary embodiments, the therapeutically effective dose is 400 mg. In exemplary embodiments, the therapeutically effective dose is 500 mg. In exemplary embodiments, the therapeutically effective dose is 600 mg. In exemplary embodiments, the therapeutically effective dose is administered once, twice, three times or four times daily.

The methods of the present disclosure may further comprise administering a second therapeutically effective dose of NMD670 to the patient either one day, two days, three days, four days, five days, six days or at least seven days after a first therapeutically effective dose is administered. The second therapeutically effective dose of NMD670 can range from 100 mg to about 1500 mg. In exemplary embodiments, the second therapeutically effective dose of NMD670 is any dosage disclosed herein. In other embodiments, the second therapeutically effective dose of NMD670 is the same as the first therapeutically effective dose administered to the patient.

In other exemplary embodiments, the methods of the present disclosure further comprise administering a third therapeutically effective dose of NMD670 to the patient either one day, two days, three days, four days, five days, six days or at least seven days after the second therapeutically effective dose has been administered. The third therapeutically effective dose of NMD670 can range from 100 mg to about 1500 mg. In exemplary embodiments, the third therapeutically effective dose of NMD670 is any dosage disclosed herein. In other embodiments, the third therapeutically effective dose of NMD670 is the same as the first therapeutically effective dose and/or the second therapeutically effective dose administered to the patient.

In some exemplary embodiments, the administration of the therapeutically effective doses of NMD670 are repeated at least 1, 2, 3, 4, 5 or 6 times weekly. In other exemplary embodiments, the administration is repeated at least 1 to 3 times weekly, 2 to 5 times weekly or 3 to 6 times weekly.

In some exemplary embodiments, the administration of the therapeutically effective doses of NMD670 are repeated daily. The administration of the therapeutically effective doses of NMD670 may for example be repeated 1, 2, 3, 4, 5, 6, 7 or 8 times daily. In other embodiments, the administration is repeated 1 to 8 times daily or 2 to 5 times daily.

In some embodiments, the therapeutically effective dose of NMD670 is administered at least one time daily. In exemplary embodiments, the therapeutically effective dose of NMD670 is administered one time daily.

In other embodiments, the therapeutically effective dose of NMD670 is administered either two times daily, three times daily, or four times daily.

In exemplary embodiments, the therapeutically effective dose of NMD670 is 100 to 600 mg, 300 to 500 mg, or about 400 mg and is administered one time daily. In exemplary embodiments, the therapeutically effective dose of NMD670 is 200 to 600 mg, 300 to 500 mg, or about 400 mg and is administered one time daily.

In other exemplary embodiments, the therapeutically effective dose of NMD670 is 100 to 600 mg, 300 to 500 mg, or about 400 mg and is administered two times daily. In other exemplary embodiments, the therapeutically effective dose of NMD670 is 200 to 600 mg, 300 to 500 mg, or about 400 mg and is administered two times daily.

In other exemplary embodiments, the therapeutically effective dose of NMD670 is 100 to 600 mg, 300 to 500 mg, or about 400 mg and is administered three times daily. In other exemplary embodiments, the therapeutically effective dose of NMD670 is 200 to 600 mg, 300 to 500 mg, or about 400 mg and is administered three times daily.

In other exemplary embodiments, the therapeutically effective dose of NMD670 is 100 to 600 mg, 300 to 500 mg, or about 400 mg and is administered four times daily. In other exemplary embodiments, the therapeutically effective dose of NMD670 is 200 to 600 mg, 300 to 500 mg, or about 400 mg and is administered four times daily.

In some exemplary embodiments, the therapeutically effective dose of NMD670 is the daily dosage amount of NMD670. In these embodiments, the daily dosage amount of NMD670 can either be administered as a single dosage or can be administered in smaller dosages throughout the day. That is, in some embodiments the daily dosage of NMD670 is administered either once a day or at least one time daily, administered twice a day or at least at two different time points throughout the day, or administered three times a day or at least at three different time points throughout the day.

In other exemplary embodiments, the patient being administered the therapeutically effective dose of NMD670 does not have hyperuricemia. For example, the patient being administered the therapeutically effective dose of NMD670 has a level of serum uric acid below 6.5 mg/dL. Patients with serum uric acid levels above 6.5 mg/dL may not be suitable to receive the therapeutically effective dose. In exemplary embodiments wherein the patient possesses a serum uric acid level above 6.5 mg/dL, the treatment method may further comprise a step of administering a low dose of NMD670 until the patient's serum uric acid level falls below 6.5 mg/dL. A low dose of NMD670 can be from 20 mg to 150 mg, such as 25 mg to 100 mg, such as 25 mg to 50 mg. Once the patient's serum uric acid level falls below 6.5 mg/dL, they can begin receiving the therapeutically effective dose of NMD670.

### Kit of Parts

In one aspect, the present invention relates to a kit-of-parts comprising:
(2S)-2-[4-bromo-2-(1,2-oxazol-3-yl)phenoxy]propanoic acid, or a pharmaceutically acceptable salt, hydrate, polymorph, tautomer, or solvate thereof, and
a peripheral myelin protein 22 (PMP22) down regulator.

In exemplary embodiments, the kit-of-parts comprises 100 to 1500 mg of (2S)-2-[4-bromo-2-(1,2-oxazol-3-yl)phenoxy]propanoic acid, or a pharmaceutically acceptable salt, hydrate, polymorph, tautomer, or solvate thereof. In exemplary embodiments, the PMP22 down regulator is PXT3003 (a combination of (RS)-baclofen, naltrexone hydrochloride and D-sorbitol). In exemplary embodiments, the PMP22 down regulator is a non-viral DNA plasmid such as Engensis (VM202).

In one aspect, the present invention relates to (2S)-2-[4-bromo-2-(1,2-oxazol-3-yl)phenoxy]propanoic acid, or a pharmaceutically acceptable salt, hydrate, polymorph, tautomer, or solvate thereof, and a peripheral myelin protein 22 (PMP22) down regulator for use in the treatment of Charcot-Marie-Tooth disease.

In one aspect, the present invention relates to a method for treatment of Charcot-Marie-Tooth disease comprising administering (2S)-2-[4-bromo-2-(1,2-oxazol-3-yl)phenoxy]propanoic acid, or a pharmaceutically acceptable salt, hydrate, polymorph, tautomer, or solvate thereof, and a peripheral myelin protein 22 (PMP22) down regulator to a subject in need thereof.

In one aspect, the present invention relates to use of a kit-of-parts or a composition comprising (2S)-2-[4-bromo-2-(1,2-oxazol-3-yl)phenoxy]propanoic acid, or a pharmaceutically acceptable salt, hydrate, polymorph, tautomer, or solvate thereof, and a peripheral myelin protein 22 (PMP22) down regulator for the manufacture of a medicament for the treatment of Charcot-Marie-Tooth disease.

In one aspect, the present invention relates to a kit-of-parts comprising:
(2S)-2-[4-bromo-2-(1,2-oxazol-3-yl)phenoxy]propanoic acid, or a pharmaceutically acceptable salt, hydrate, polymorph, tautomer, or solvate thereof, and
biotin, or a pharmaceutically acceptable salt, hydrate, polymorph, tautomer, or solvate thereof.

In exemplary embodiments, the kit-of-parts comprises 100 to 1500 mg of (2S)-2-[4-bromo-2-(1,2-oxazol-3-yl)phenoxy]propanoic acid, or a pharmaceutically acceptable salt, hydrate, polymorph, tautomer, or solvate thereof. In exemplary embodiments, the kit-of-parts comprises 100 to 300 mg biotin (MD1003).

In other exemplary embodiments, kit-of-parts further comprises one or more additional compounds, such as IFB-088, donaperminogene seltosplasmid, icerguastat, CKD-510, AGT-100216, MiM-111, EN-001, DTx-1252, Ricolinstat, AT-007 (govorestat), Reldesemtiv and/or pyridostigmine.

In one aspect, the kit-of-parts is for use in a method of treatment of Charcot-Marie-Tooth disease in a subject.

### Items

1. A composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein the composition is for administration at a therapeutic dose of 100 to 1500 mg of (2S)-2-[4-bromo-2-(1,2-oxazol-3-yl)phenoxy]propanoic acid.
2. The composition for use according to item 1, wherein the therapeutic dose is less than 1500 mg, such as less than 1450 mg, such as less than 1300 mg, such as less than 1250 mg, such as less than 1200 mg, such as less than 1150 mg, such as less than 1100 mg, such as less than 1050 mg, such as less than 1000 mg, such as less than 950 mg, such as less than 900 mg, such as less than 850 mg, such as less than 800 mg, such as less than 750 mg, such as less than 700 mg, such as less than 650 mg, such as less than 600 mg, such as less than 550 mg, such as less than 500 mg, such as less than 450 mg, such as less than 400 mg, such as less than 350 mg, such as less than 300 mg, such as less than 250 mg.
3. The composition for use according to any one of the preceding items, wherein the therapeutic dose is at least 100 mg, such as at least 150 mg, such as at least 200 mg, such as at least 250 mg, such as at least 300 mg, such as at least 350 mg, such as at least 400 mg, such as at least 450 mg, such as at least 500 mg, such as at least 550 mg, such as at least 600 mg, such as at least 650 mg, such as at least 700 mg, such as at least 750 mg, such as at least 800 mg, such as at least 850 mg, such as at least 900 mg, such as at least 950 mg, such as at least 1000 mg, such as at least 1050 mg, such as at least 1100 mg, such as at least 1150 mg, such as at least 1200 mg, such as at least 1250 mg, such as at least 1300 mg, such as at least 1350 mg, such as at least 1400 mg, such as at least 1450 mg.
4. The composition for use according to item 1, wherein the therapeutic dose is 200 to 600 mg, such as 250 to 550 mg, such as 300 to 500 mg, such as 350 to 450 mg, such as 375 to 425 mg, such as 400 mg.
5. The composition for use according to item 1, wherein the therapeutic dose is 700 to 1400 mg, such as 800 to 1350 mg, such as 900 to 1300 mg, such as 1000 to 1250 mg, such as 1100 to 1250 mg, such as about 1200 mg.
6. The composition for use according to item 1, wherein the therapeutic dose is about 100 mg.
7. The composition for use according to item 1, wherein the therapeutic dose is about 150 mg.
8. The composition for use according to item 1, wherein the therapeutic dose is about 200 mg.
9. The composition for use according to item 1, wherein the therapeutic dose is about 250 mg.
10. The composition for use according to item 1, wherein the therapeutic dose is about 300 mg.
11. The composition for use according to item 1, wherein the therapeutic dose is about 350 mg.
12. The composition for use according to item 1, wherein the therapeutic dose is about 400 mg.
13. The composition for use according to item 1, wherein the therapeutic dose is about 500 mg.
14. The composition for use according to item 1, wherein the therapeutic dose is about 600 mg.
15. The composition for use according to any one of the preceding items, wherein the therapeutic dose is to be administered at least one time daily.
16. The composition for use according to any one of the preceding items, wherein the therapeutic dose is to be administered one time daily.
17. The composition for use according to any one of items 1 to 15, wherein the therapeutic dose is to be administered two times daily.
18. The composition for use according to any one of items 1 to 15, wherein the therapeutic dose is to be administered three times daily.
19. The composition for use according to any one of items 1 to 15, wherein the therapeutic dose is to be administered four times daily.
20. The composition for use according to item 1, wherein the therapeutic dose is 100 to 600 mg and the composition is to be administered one time daily.
21. The composition for use according to item 1, wherein the therapeutic dose is 200 to 600 mg and the composition is to be administered one time daily.
22. The composition for use according to item 1, wherein the therapeutic dose is 300 to 500 mg and the composition is to be administered one time daily.
23. The composition for use according to item 1, wherein the therapeutic dose is about 100 mg and the composition is to be administered one time daily.
24. The composition for use according to item 1, wherein the therapeutic dose is about 150 mg and the composition is to be administered one time daily.
25. The composition for use according to item 1, wherein the therapeutic dose is about 200 mg and the composition is to be administered one time daily.
26. The composition for use according to item 1, wherein the therapeutic dose is about 250 mg and the composition is to be administered one time daily.
27. The composition for use according to item 1, wherein the therapeutic dose is about 300 mg and the composition is to be administered one time daily.
28. The composition for use according to item 1, wherein the therapeutic dose is about 350 mg and the composition is to be administered one time daily.
29. The composition for use according to item 1, wherein the therapeutic dose is about 400 mg and the composition is to be administered one time daily.
30. The composition for use according to item 1, wherein the therapeutic dose is about 500 mg and the composition is to be administered one time daily.
31. The composition for use according to item 1, wherein the therapeutic dose is about 600 mg and the composition is to be administered one time daily.
32. The composition for use according to item 1, wherein the therapeutic dose is 100 to 600 mg and the composition is to be administered two times daily.
33. The composition for use according to item 1, wherein the therapeutic dose is 200 to 600 mg and the composition is to be administered two times daily.
34. The composition for use according to any one of items 1 to 5, wherein the therapeutic dose is 300 to 500 mg and the composition is to be administered two times daily.
35. The composition for use according to item 1, wherein the therapeutic dose is about 100 mg and the composition is to be administered two times daily.
36. The composition for use according to item 1, wherein the therapeutic dose is about 150 mg and the composition is to be administered two times daily.
37. The composition for use according to item 1, wherein the therapeutic dose is about 200 mg and the composition is to be administered two times daily.
38. The composition for use according to item 1, wherein the therapeutic dose is about 250 mg and the composition is to be administered two times daily.
39. The composition for use according to item 1, wherein the therapeutic dose is about 300 mg and the composition is to be administered two times daily.
40. The composition for use according to item 1, wherein the therapeutic dose is about 350 mg and the composition is to be administered two times daily.
41. The composition for use according to item 1, wherein the therapeutic dose is about 400 mg and the composition is to be administered two times daily.
42. The composition for use according to item 1, wherein the therapeutic dose is about 500 mg and the composition is to be administered two times daily.
43. The composition for use according to item 1, wherein the therapeutic dose is about 600 mg and the composition is to be administered two times daily.
44. The composition for use according to item 1, wherein the therapeutic dose is 100 to 600 mg and the composition is to be administered three times daily.
45. The composition for use according to item 1, wherein the therapeutic dose is 200 to 600 mg and the composition is to be administered three times daily.
46. The composition for use according to item 1, wherein the therapeutic dose is 300 to 500 mg and the composition is to be administered three times daily.
47. The composition for use according to item 1, wherein the therapeutic dose is about 100 mg and the composition is to be administered three times daily.
48. The composition for use according to item 1, wherein the therapeutic dose is about 150 mg and the composition is to be administered three times daily.
49. The composition for use according to item 1, wherein the therapeutic dose is about 200 mg and the composition is to be administered three times daily.
50. The composition for use according to item 1, wherein the therapeutic dose is about 250 mg and the composition is to be administered three times daily.
51. The composition for use according to item 1, wherein the therapeutic dose is about 300 mg and the composition is to be administered three times daily.
52. The composition for use according to item 1, wherein the therapeutic dose is about 350 mg and the composition is to be administered three times daily.
53. The composition for use according to item 1, wherein the therapeutic dose is about 400 mg and the composition is to be administered three times daily.
54. The composition for use according to item 1, wherein the therapeutic dose is about 500 mg and the composition is to be administered three times daily.
55. The composition for use according to item 1, wherein the therapeutic dose is about 600 mg and the composition is to be administered three times daily.
56. The composition for use according to item 1, wherein the therapeutic dose is 100 to 600 mg and the composition is to be administered four times daily.
57. The composition for use according to item 1, wherein the therapeutic dose is 200 to 600 mg and the composition is to be administered four times daily.
58. The composition for use according to any one of items 1 to 5, wherein the therapeutic dose is the total daily dosage.
59. The composition for use according to any one of the preceding items, wherein the composition is administered orally, parenterally, intravenously, inhaled, topically, enterally, rectally, buccally or as an aerosol.
60. The composition for use according to any one of items 1 to 59, wherein the composition is in the form of a liquid, liquid suspension, oil, emulsion, or syrup.
61. The composition for use according to any one of items 1 to 59, wherein the composition is a solid dosage form.
62. The composition for use according to item 61, wherein the solid dosage form is administered orally.
63. The composition for use according to any one of items 1 to 62, wherein the composition is in the form of a solid dosage form and is to be dosed orally, the therapeutic dose is 100 to 600 mg and the composition is to be administered one time daily.
64. The composition for use according to any one of items 1 to 62, wherein the composition is in the form of a solid dosage form and is to be dosed orally, the therapeutic dose is 200 to 600 mg and the composition is to be administered one time daily.
65. The composition for use according to any one of items 1 to 62, wherein the composition is in the form of a solid dosage form and is to be dosed orally, the therapeutic dose is 100 to 600 mg and the composition is to be administered two times daily.
66. The composition for use according to any one of items 1 to 62, wherein the composition is in the form of a solid dosage form and is to be dosed orally, the therapeutic dose is 200 to 600 mg and the composition is to be administered two times daily.
67. The composition for use according to any one of items 1 to 62, wherein the composition is in the form of a solid dosage form and is to be dosed orally, the therapeutic dose is 100 to 600 mg and the composition is to be administered three times daily.
68. The composition for use according to any one of items 1 to 62, wherein the composition is in the form of a solid dosage form and is to be dosed orally, the therapeutic dose is 200 to 600 mg and the composition is to be administered three times daily.
69. The composition for use according to any one of items 1 to 62, wherein the composition is in the form of a solid dosage form and is to be dosed orally, the therapeutic dose is 100 to 600 mg and the composition is to be administered four times daily.
70. The composition for use according to any one of items 1 to 62, wherein the composition is in the form of a solid dosage form and is to be dosed orally, the therapeutic dose is 200 to 600 mg and the composition is to be administered four times daily.
71. The composition for use according to any of items 61 to 70, wherein the solid dosage form is selected from the group consisting of capsule (such as sprinkle capsule and gelatine capsule), tablet (such as uncoated tablet, coated tablet, slow-release tablet) and sprinkle.
72. The composition for use according to any of items 61 to 71, wherein the solid dosage form releases not less than 80% of the compound after 30 minutes, when measured in a United States Pharmacopeia (USP) type 2 dissolution apparatus, paddle at 75 rpm, at a temperature of 37° C.±0.5° C. in 900 mL of pH 6.8 phosphate/citric acid buffer.
73. The composition for use according to any one of the preceding items, wherein the composition is to be administered orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where Tₘₐₓ is reached within 1 to 6 hours after administration.
74. The composition for use according to any one of the preceding items, wherein the composition is to be dosed orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where Tₘₐₓ is reached within 1 to 5 hours, such 1.5 to 4 hours, such as about 2 hours or about 3 hours, after administration.
75. The composition for use according to any one of the preceding items, wherein the composition is to be administered orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where Tₘₐₓ is reached within 3 to 7 hours after administration.
76. The composition for use according to any one of the preceding items, wherein the composition is to be administered orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where the mean Cₘₐₓ is 12,760 to 27,440 ng/mL, such as 15,000 to 25,000 ng/mL, such as 16,000 to 24,000 ng/mL, such as 16,080 to 25,125 ng/mL, such as 17,000 to 23,000 ng/mL, such as 18,000 to 22,000 ng/mL, such as 19,000 to 21,000 ng/mL, such as about 20,100 ng/mL after administration with a single dose of 400 mg (2S)-2-[4-bromo-2-(1,2-oxazol-3-yl)phenoxy]propanoic acid, or a pharmaceutically acceptable salt, hydrate, polymorph, tautomer, or solvate thereof.
77. The composition for use according to item 76, wherein the composition is to be administered orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where the mean Cₘₐₓ is 20,100 ng/mL and the standard deviation is 7,340 ng/mL.
78. The composition for use according to any one of the preceding items, wherein the composition is to be administered orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where the mean Cₘₐₓ is about 80% to about 125%, such as 80.00% to 125.00%, of 20,100 ng/mL after administration with a single dose of 400 mg (2S)-2-[4-bromo-2-(1,2-oxazol-3-yl)phenoxy]propanoic acid, or a pharmaceutically acceptable salt, hydrate, polymorph, tautomer, or solvate thereof.
79. The composition for use according to any one of the preceding items, wherein the composition is to be administered orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where the mean AUC_{0-nfn}ty is 66,300 to 109,100 h·ng/mL, such as 70,000 to 105,000 h·ng/mL, such as 70,160 to 109,625 h·ng/mL, such as 75,000 to 100,000 h·ng/mL, such as 80,000 to 95,000 h·ng/mL, such as 85,000 to 90,000 h·ng/mL, such as about 87,700 h·ng/mL after administration with a single dose of 400 mg (2S)-2-[4-bromo-2-(1,2-oxazol-3-yl)phenoxy]propanoic acid, or a pharmaceutically acceptable salt, hydrate, polymorph, tautomer, or solvate thereof.
80. The composition for use according to any one of the preceding items, wherein the composition is to be administered orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where the mean AUC_{0-infinity} is 87,700 ng/mL and the standard deviation is 21,400 ng/mL.
81. The composition for use according to any one of the preceding items, wherein the composition is to be administered orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where the mean AUC_{0-infinity} is about 80% to about 125%, such as 80.00% to 125.00%, of 87,700 h·ng/mL after administration with a single dose of 400 mg (2S)-2-[4-bromo-2-(1,2-oxazol-3-yl)phenoxy]propanoic acid, or a pharmaceutically acceptable salt, hydrate, polymorph, tautomer, or solvate thereof.
82. The composition for use according to any of the preceding items, wherein said AUC₀₋₂₄ AUC_{infinity}, Cₘₐₓ or Tₘₐₓ is measured after administration of a single dose to a human subject suffering from Charcot-Marie-Tooth disease.
83. The composition for use according to any one of the preceding items, wherein the composition further comprises at least one pharmaceutically acceptable adjuvant and/or excipient.
84. The composition for use according to item 83, wherein the pharmaceutically acceptable adjuvant and/or excipient is selected from the group consisting of filler, binder, lubricant and disintegrant.
85. The composition for use according to item 83, wherein the pharmaceutically acceptable adjuvant and/or excipient is selected from the group consisting of silicified microcrystalline cellulose, microcrystalline cellulose, maltodextrin, magnesium stearate and croscarmellose sodium.
86. The composition for use according to any of items 61 to 85, wherein the composition comprises 10 to 80 wt %, such as 40 to 65 wt %, such as 50 to 55 wt %, such as about 53 wt % (2S)-2-[4-bromo-2-(1,2-oxazol-3-yl)phenoxy]propanoic acid, or a pharmaceutically acceptable salt, hydrate, polymorph, tautomer, or solvate thereof.
87. The composition for use according to any of items 61 to 85, wherein the composition comprises 10 to 80 wt %, such as 40 to 65 wt %, such as 50 to 60 wt %, such as 50 to 55 wt %, such as 55 to 60 wt %, such as about 53 wt %, such as 56 wt % (2S)-2-[4-bromo-2-(1,2-oxazol-3-yl)phenoxy]propanoic acid, or a pharmaceutically acceptable salt, hydrate, polymorph, tautomer, or solvate thereof.
88. The composition for use according to any of items 61 to 85, wherein the composition comprises:
   a. 10 to 80 wt %, such as 40 to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof;
   b. 20 to 80 wt %, such as 25 to 50 wt % filler;
   c. 2 to 20 wt %, such as 3 to 16 wt % binder;
   d. 0.25 to 3 wt %, such as 0.4 to 2.0 wt % lubricant; and
   e. 0.25 to 5 wt %, such as 0.3 to 2.5 wt % disintegrant;
with the proviso that the sum of the wt % of the components does not exceed 100 wt %.
89. The composition for use according to any of items 61 to 85, wherein the composition comprises:
   a. 10 to 80 wt %, such as 40 to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof;
   b. 5 to 60 wt %, such as 20 to 40 wt %, such as 21 to 37 wt % silicified microcrystalline cellulose;
   c. 2 to 60 wt %, such as 5 to 16 wt % microcrystalline cellulose;
   d. 1 to 15 wt %, such as 1.5 to 7 wt %, such as 1.8 to 6.0 wt % maltodextrin;
   e. 0.25 to 3 wt %, such as 0.4 to 2.0 wt % magnesium stearate; and
   f. 0.25 to 5 wt %, such as 0.3 to 2.5 wt % Croscarmellose sodium;
with the proviso that the sum of the wt % of the components does not exceed 100 wt %.
90. The composition for use according to any one of items 61 to 85, wherein the composition comprises:
   a. 10 to 80 wt %, such as 40 to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof;
   b. 20 to 80 wt %, such as 25 to 50 wt % filler;
   c. 2 to 20 wt %, such as 3 to 16 wt % binder;
   d. 0.25 to 3 wt %, such as 0.4 to 2.0 wt % lubricant;
   e. 0.25 to 5 wt %, such as 0.3 to 2.5 wt % disintegrant; and
   f. 1 to 10 wt % film coating:
with the proviso that the sum of the wt % of the components does not exceed 100 wt %.
91. The composition for use according to any of items 61 to 85, wherein the composition comprises or consists of:
   a. 10 to 80 wt %, such as 40 to 65 wt %, such as 50 to 55 wt %, such as about 53 wt % (2S)-2-[4-bromo-2-(1,2-oxazol-3-yl)phenoxy]propanoic acid, or a pharmaceutically acceptable salt, hydrate, polymorph, tautomer, or solvate thereof;
   b. 5 to 60 wt %, such as 20 to 40 wt %, such as 21 to 37 wt % silicified microcrystalline cellulose;
   c. 2 to 60 wt %, such as 5 to 16 wt % microcrystalline cellulose;
   d. 1 to 15 wt %, such as 1.5 to 7 wt %, such as 1.8 to 6.0 wt % maltodextrin; and
   e. 0.25 to 3 wt %, such as 0.4 to 2.0 wt % magnesium stearate;
   f. 0.25 to 5 wt %, such as 0.3 to 2.5 wt % Croscarmellose sodium; and
   g. 1 to 10 wt % film coating composition such as Opadry white,
with the proviso that the sum of the wt % of the components does not exceed 100 wt %.
92. The composition for use according to any one of the preceding items, wherein the therapeutic dose is 100 to 600 mg, the composition is in the form of a solid dosage form and is to be administered orally one time daily.
93. The composition for use according to any one of the preceding items, wherein the therapeutic dose is 200 to 600 mg, the composition is in the form of a solid dosage form and is to be administered orally one time daily.
94. The composition for use according to any one of the preceding items, wherein the therapeutic dose is 100 to 600 mg, the composition is in the form of a solid dosage form and is to be administered orally two times daily.
95. The composition for use according to any one of the preceding items, wherein the therapeutic dose is 200 to 600 mg, the composition is in the form of a solid dosage form and is to be administered orally two times daily.
96. The composition for use according to any one of the preceding items, wherein the therapeutic dose is 100 to 600 mg, the composition is in the form of a solid dosage form and is to be administered orally three times daily.
97. The composition for use according to any one of the preceding items, wherein the therapeutic dose is 200 to 600 mg, the composition is in the form of a solid dosage form and is to be administered orally three times daily.
98. The composition for use according to any one of the preceding items, wherein the therapeutic dose is 100 to 600 mg, the composition is in the form of a solid dosage form and is to be administered orally four times daily.
99. The composition for use according to any one of the preceding items, wherein the therapeutic dose is 200 to 600 mg, the composition is in the form of a solid dosage form and is to be administered orally four times daily.
100. The composition for use according to any one of the preceding items, wherein the composition is administered as one or more unit dosage forms.
101. The composition for use according to any one of the preceding items, wherein the subject has a level of serum uric acid below 6.5 mg/dL.
102. The composition for use according to any one of the preceding items, wherein the subject experiences a lessening of Charcot-Marie-Tooth disease symptoms.
103. The composition for use according to any one of the preceding items, wherein the subject or a group of subjects experience an increase in the total distance walked determined using a 6-minute walk test after treatment with the composition.
104. The composition for use according to item 103, wherein the increase in the total distance walked is determined by comparing the change from baseline in the total distance walked after a defined period of time of treatment with the composition comprising (2S)-2-[4-bromo-2-(1,2-oxazol-3-yl)phenoxy]propanoic acid, or a pharmaceutically acceptable salt, hydrate, polymorph, tautomer, or solvate thereof, with the change from baseline in the total distance walked after a defined period of time of placebo treatment.
105. The composition for use according to item 104, wherein the period of time is 21 days.
106. The composition for use according to any one of items 103 to 105, wherein the total distance walked has increased by at least 5%, such as at least 10%, such as at least 15%, such as at least 20%, such as at least 25%, such as at least 30%, such as at least 50%, such as at least 75%, such as at least 100%, such as at least 150%, such as at least 200%.
107. The composition for use according to any one of items 103 to 105, wherein the total distance walked has increased by between 5% and 400%, such as between 5% and 200%, such as between 10% and 200%.
108. The composition for use according to any one of items 103 to 105, wherein the total distance walked has increased by at least 20 metres, such as at least 30 metres, such as at least 40 metres, such as at least 50 metres, such as at least 60 metres, such as at least 80 metres, such as at least 100 metres, such as at least 150 metres, such as at least 200 metres, such as at least 250 metres, such as at least 300 metres.
109. The composition for use according to any one of items 103 to 105, wherein the total distance walked has increased by between 20 and 400 metres, such as between 30 and 300 metres, such as between 40 and 200 metres.
110. The composition for use according to any one of the preceding items, wherein the composition is for administration at a therapeutic dose of 100 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an increase in the total distance walked when determined using the 6-minute walk test.
111. The composition for use according to any one of the preceding items, wherein the composition is for administration at a therapeutic dose of 200 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an increase in the total distance walked when determined using the 6-minute walk test.
112. The composition for use according to any one of the preceding items, wherein the subject or a group of subjects experience an increase in muscle strength after treatment with the composition.
113. The composition for use according to item 112, wherein muscle strength is measured using a handheld dynamometer.
114. The composition for use according to item 112, wherein muscle strength is measured using a fixed dynamometer.
115. The composition for use according to any one of items 112 or 113, wherein muscle strength is measured as grip strength.
116. The composition for use according to any one of items 112 or 113, wherein muscle strength is measured as the strength of the thigh (knee flexors), the upper arm (elbow flexor and extension) and/or the shoulder (shoulder abduction).
117. The composition for use according to any one of items 112 or 114, wherein muscle strength is measured as the strength of the ankle dorsiflexion.
118. The composition for use according to any one of items 112 to 117, wherein the increase muscle strength is determined by comparing the change from baseline in muscle strength after a defined period of time treatment with the composition comprising (2S)-2-[4-bromo-2-(1,2-oxazol-3-yl)phenoxy]propanoic acid, or a pharmaceutically acceptable salt, hydrate, polymorph, tautomer, or solvate thereof, with the change from baseline in muscle strength after a defined period of time of placebo treatment.
119. The composition for use according to item 118, wherein the period of time is 21 days.
120. The composition for use according to any one of the preceding items, wherein muscle strength has increased by at least 5%, such as at least 10%, such as at least 15%, such as at least 20%, such as at least 25%, such as at least 30%, such as at least 50%, such as at least 75%, such as at least 100%, such as at least 150%, such as at least 200%.
121. The composition for use according to any one of the preceding items, wherein muscle strength has increased by between 10% and 400%, such as between 15% and 200%, such as between 20% and 100%.
122. The composition for use according to any one of the preceding items, wherein muscle strength determined by measuring grip strength using a handheld dynamometer has increased by at least 5%, such as at least 10%, such as at least 15%, such as at least 20%, such as at least 25%, such as at least 30%, such as at least 50%, such as at least 75%, such as at least 100%, such as at least 150%, such as at least 200%.
123. The composition for use according to any one of the preceding items, wherein muscle strength determined by measuring grip strength using a handheld dynamometer has increased by between 10% and 400%, such as between 15% and 200%, such as between 20% and 100%.
124. The composition for use according to any one of the preceding items, wherein muscle strength determined by measuring grip strength using a handheld dynamometer has increased by at least 0.25 kg, such as at least 0.50 kg, such as at least 0.75 kg, such as at least 1.0 kg, such as at least 1.25 kg, such as at least 1.5 kg, such as at least 1.75 kg, such as at least 2.0 kg, such as at least 2.5 kg, such as at least 3.0 kg.
125. The composition for use according to any one of the preceding items, wherein muscle strength determined by measuring grip strength using a handheld dynamometer has increased by between 0.25 and 5.0 kg, such as between 0.25 and 4.0 kg, such as between 0.5 and 4.0 kg.
126. The composition for use according to any one of the preceding items, wherein muscle strength determined by measuring knee flexor strength using a handheld dynamometer has increased by at least 0.25 kg, such as at least 0.50 kg, such as at least 0.75 kg, such as at least 1.0 kg, such as at least 1.25 kg, such as at least 1.5 kg, such as at least 1.75 kg, such as at least 2.0 kg, such as at least 2.5 kg, such as at least 3.0 kg.
127. The composition for use according to any one of the preceding items, wherein muscle strength determined by measuring knee flexor strength using a handheld dynamometer has increased by between 0.25 and 5.0 kg, such as between 0.25 and 4.0 kg, such as between 0.5 and 4.0 kg.
128. The composition for use according to any one of the preceding items, wherein muscle strength determined by measuring elbow flexor strength using a handheld dynamometer has increased by at least 0.25 kg, such as at least 0.50 kg, such as at least 0.75 kg, such as at least 1.0 kg, such as at least 1.25 kg, such as at least 1.5 kg, such as at least 1.75 kg, such as at least 2.0 kg, such as at least 2.5 kg, such as at least 3.0 kg.
129. The composition for use according to any one of the preceding items, wherein muscle strength determined by measuring elbow flexor strength using a handheld dynamometer has increased by between 0.25 and 5.0 kg, such as between 0.25 and 4.0 kg, such as between 0.5 and 4.0 kg.
130. The composition for use according to any one of the preceding items, wherein the composition is for administration at a therapeutic dose of 100 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an increase in in muscle strength when determined by measuring grip strength using a handheld dynamometer.
131. The composition for use according to any one of the preceding items, wherein the composition is for administration at a therapeutic dose of 200 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an increase in in muscle strength when determined by measuring grip strength using a handheld dynamometer.
132. The composition for use according to any one of the preceding items, wherein muscle strength determined by measuring muscle strength of the ankle dorsiflexion using a fixed dynamometer has increased by at least 0.25 kg, such as at least 0.50 kg, such as at least 0.75 kg, such as at least 1.0 kg, such as at least 1.25 kg, such as at least 1.5 kg, such as at least 1.75 kg, such as at least 2.0 kg, such as at least 2.5 kg, such as at least 3.0 kg.
133. The composition for use according to any one of the preceding items, wherein muscle strength determined by measuring muscle strength of the ankle dorsiflexion using a fixed dynamometer has increased by between 0.25 and 5.0 kg, such as between 0.25 and 4.0 kg, such as between 0.5 and 4.0 kg.
134. The composition for use according to any one of the preceding items, wherein the composition is for administration at a therapeutic dose of 100 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an increase in isometric strength when determined by measuring isometric strength using a fixed dynamometer.
135. The composition for use according to any one of the preceding items, wherein the composition is for administration at a therapeutic dose of 200 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an increase in isometric strength when determined by measuring isometric strength using a fixed dynamometer.
136. The composition for use according to any one of the preceding items, wherein the subject or a group of subjects experience a decrease in the Overall Neuropathy Limitations Score (ONLS) after treatment with the composition.
137. The composition for use according to item 136, wherein the Overall Neuropathy Limitations Score (ONLS) is determined by comparing the change from baseline in the a decrease in the Overall Neuropathy Limitations Score (ONLS) after a defined period of time treatment with the composition comprising (2S)-2-[4-bromo-2-(1,2-oxazol-3-yl)phenoxy]propanoic acid, or a pharmaceutically acceptable salt, hydrate, polymorph, tautomer, or solvate thereof, with the change from baseline in a decrease in the Overall Neuropathy Limitations Score (ONLS) after a defined period of time of placebo treatment.
138. The composition for use according to item 137, wherein the period of time is 21 days.
139. The composition for use according to any one of the preceding items, wherein the Overall Neuropathy Limitations Score (ONLS) has decreased by at least 0.3 points, such as at least 0.5 points, such as at least 0.75 points, such as at least 1 point, such as at least 2 points, such as at least 3 points, such as at least 4 points, such as at least 5 points, such as at least 6 points, such as at least 8 points, such as at least 10 points.
140. The composition for use according to any one of the preceding items, wherein the Overall Neuropathy Limitations Score (ONLS) has decreased by between has increased by between 0.3 and 10 points, such as between 0.5 and 6 points, such as between 0.3 and 5 points.
141. The composition for use according to any one of the preceding items, wherein the composition is for administration at a therapeutic dose of 100 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences a decrease in the Overall Neuropathy Limitations Score (ONLS).
142. The composition for use according to any one of the preceding items, wherein the composition is for administration at a therapeutic dose of 200 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences a decrease in the Overall Neuropathy Limitations Score (ONLS).
143. The composition for use according to any one of the preceding items, wherein the subject or a group of subjects experience an improvement in finger dexterity after treatment with the composition.
144. The composition for use according to item 143, wherein improvement in improvement in finger dexterity is determined using a nine-hole peg test (9-HPT).
145. The composition for use according to item 144, wherein the improvement in finger dexterity is determined by comparing the change from baseline in the nine-hole peg test after a defined period of time treatment with the composition comprising (2S)-2-[4-bromo-2-(1,2-oxazol-3-yl)phenoxy]propanoic acid, or a pharmaceutically acceptable salt, hydrate, polymorph, tautomer, or solvate thereof, with the change from baseline in the nine-hole peg test after a defined period of time of placebo treatment.
146. The composition for use according to item 145, wherein the period of time is 21 days.
147. The composition for use according to any one of the preceding items, wherein finger dexterity determined using a nine-hole peg test has increased by at least 5%, such as at least 10%, such as at least 15%, such as at least 20%, such as at least 25%, such as at least 30%, such as at least 50%, such as at least 75%, such as at least 100%, such as at least 150%, such as at least 200%.
148. The composition for use according to any one of the preceding items, wherein finger dexterity determined using a nine-hole peg test has increased by between 5% and 400%, such as between 10% and 300%, such as between 10% and 200%.
149. The composition for use according to any one of the preceding items, wherein the composition is for administration at a therapeutic dose of 100 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an improvement in finger dexterity when determined using a nine-hole peg test.
150. The composition for use according to any one of the preceding items, wherein the composition is for administration at a therapeutic dose of 200 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an improvement in finger dexterity when determined using a nine-hole peg test.
151. The composition for use according to any one of the preceding items, wherein the subject or a group of subjects experience a decrease in the CMT Neuropathy Score 2 (CMTNS2) score after treatment with the composition.
152. The composition for use according to item 151, wherein the decrease in the CMTNS2 score is determined by comparing the change from baseline in the decrease in the CMTNS2 score after a defined period of time treatment with the composition comprising (2S)-2-[4-bromo-2-(1,2-oxazol-3-yl)phenoxy]propanoic acid, or a pharmaceutically acceptable salt, hydrate, polymorph, tautomer, or solvate thereof, with the change from baseline in the decrease in the CMTNS2 score after a defined period of time of placebo treatment.
153. The composition for use according to item 152, wherein the period of time is 21 days.
154. The composition for use according to any one of the preceding items, wherein the CMTNS2 score has decreased by at least 0.3 points, such as at least 0.5 points, such as at least 0.75 points, such as at least 1 point, such as at least 2 points, such as at least 3 points, such as at least 4 points, such as at least 5 points, such as at least 6 points, such as at least 8 points, such as at least 10 points.
155. The composition for use according to any one of the preceding items, wherein the CMTNS2 score has decreased by between 0.3 and 20 points, such as between 0.5 and 6 points, such as between 0.3 and 5 points.
156. The composition for use according to any one of the preceding items, wherein the composition is for administration at a therapeutic dose of 100 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences a decrease in the CMT Neuropathy Score 2 score.
157. The composition for use according to any one of the preceding items, wherein the composition is for administration at a therapeutic dose of 200 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences a decrease in the CMT Neuropathy Score 2 score.
158. The composition for use according to any one of the preceding items, wherein the subject or a group of subjects experience a decrease in the CMT examination score (second version) (CMTES2) score after treatment with the composition.
159. The composition for use according to item 158, wherein the decrease in the CMTES2 score is determined by comparing the change from baseline in the decrease in the CMTES2 score after a defined period of time treatment with the composition comprising (2S)-2-[4-bromo-2-(1,2-oxazol-3-yl)phenoxy]propanoic acid, or a pharmaceutically acceptable salt, hydrate, polymorph, tautomer, or solvate thereof, with the change from baseline in the decrease in the CMTES2 score after a defined period of time of placebo treatment.
160. The composition for use according to item 159, wherein the period of time is 21 days.
161. The composition for use according to any one of the preceding items, wherein the CMTES2 score has decreased by at least 0.3 points, such as at least 0.5 points, such as at least 0.75 points, such as at least 1 point, such as at least 2 points, such as at least 3 points, such as at least 4 points, such as at least 5 points, such as at least 6 points, such as at least 8 points, such as at least 10 points.
162. The composition for use according to any one of the preceding items, wherein the CMTES2 score has decreased by between 0.3 and 20 points, such as between 0.5 and 6 points, such as between 0.3 and 5 points.
163. The composition for use according to any one of the preceding items, wherein the composition is for administration at a therapeutic dose of 100 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences a decrease in the CMT examination score (second version) score.
164. The composition for use according to any one of the preceding items, wherein the composition is for administration at a therapeutic dose of 200 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences a decrease in the CMT examination score (second version) score.
165. The composition for use according to any one of the preceding items, wherein the subject or a group of subjects experience a reduction in fatigue after treatment with the composition.
166. The composition for use according to item 165, wherein the reduction in fatigue is determined using a fatigue index which is calculated from the distance walked in the 1st minute to the distance walked in the 6th minute of a 6-minute walk test.
167. The composition for use according to item 166, wherein the reduction in fatigue is determined by comparing the change from baseline in the fatigue index after a defined period of time treatment with the composition comprising (2S)-2-[4-bromo-2-(1,2-oxazol-3-yl)phenoxy]propanoic acid, or a pharmaceutically acceptable salt, hydrate, polymorph, tautomer, or solvate thereof, with the change from baseline in the drop-out rate in the fatigue index after a defined period of time of placebo treatment.
168. The composition for use according to item 167, wherein the period of time is 21 days.
169. The composition for use according to any one of the preceding items, wherein the reduction in fatigue determined using a fatigue index has decreased by at least 5%, such as at least 10%, such as at least 15%, such as at least 20%, such as at least 25%, such as at least 30%, such as at least 40%, such as at least 50%, such as at least 60%, such as at least 70%, such as at least 80%.
170. The composition for use according to any one of the preceding items, wherein the reduction in fatigue determined using a fatigue index has decreased by between 5% and 95%, such as between 5% and 80%, such as between 10% and 50%.
171. The composition for use according to any one of the preceding items, wherein the composition is for administration at a therapeutic dose of 100 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences a reduction in fatigue when determined using a fatigue index.
172. The composition for use according to any one of the preceding items, wherein the composition is for administration at a therapeutic dose of 200 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences a reduction in fatigue when determined using a fatigue index.
173. The composition for use according to any one of the preceding items, wherein the subject or a group of subjects experience an increase in the motor function measure 32-item score after treatment with the composition.
174. The composition for use according to item 173, wherein the increase in the motor function measure 32-item score is determined by comparing the change from baseline in the motor function measure 32-item score after a defined period of time treatment with the composition comprising (2S)-2-[4-bromo-2-(1,2-oxazol-3-yl)phenoxy]propanoic acid, or a pharmaceutically acceptable salt, hydrate, polymorph, tautomer, or solvate thereof, with the change from baseline in the motor function measure 32-item score after a defined period of time of placebo treatment.
175. The composition for use according to item 174, wherein the period of time is 21 days.
176. The composition for use according to any one of the preceding items, wherein the motor function measure 32-item score has increased by at least 5%, such as at least 10%, such as at least 15%, such as at least 20%, such as at least 25%, such as at least 30%, such as at least 40%, such as at least 50%, such as at least 60%, such as at least 70%, such as at least 80%.
177. The composition for use according to any one of the preceding items, wherein the motor function measure 32-item score has increased by between 5% and 95%, such as between 5% and 80%, such as between 10% and 50%.
178. The composition for use according to any one of the preceding items, wherein the composition is for administration at a therapeutic dose of 100 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an increase in the motor function measure 32-item score.
179. The composition for use according to any one of the preceding items, wherein the composition is for administration at a therapeutic dose of 200 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an increase in the motor function measure 32-item score.
180. The composition for use according to any one of the preceding items, wherein the subject or a group of subjects experience a decrease in the CMT Functional Outcome Measure (CMT-FOM) score after treatment with the composition.
181. The composition for use according to item 180, wherein the decrease in the CMT-FOM score is determined by comparing the change from baseline in the decrease in the CMT-FOM score after a defined period of time treatment with the composition comprising (2S)-2-[4-bromo-2-(1,2-oxazol-3-yl)phenoxy]propanoic acid, or a pharmaceutically acceptable salt, hydrate, polymorph, tautomer, or solvate thereof, with the change from baseline in the decrease in the CMT-FOM score after a defined period of time of placebo treatment.
182. The composition for use according to item 181, wherein the period of time is 21 days.
183. The composition for use according to any one of the preceding items, wherein the CMT-FOM score has decreased by at least 0.3 points, such as at least 0.5 points, such as at least 0.75 points, such as at least 1 point, such as at least 2 points, such as at least 3 points, such as at least 4 points, such as at least 5 points, such as at least 6 points, such as at least 8 points, such as at least 10 points, such as at least 15 points, such as at least 20 points.
184. The composition for use according to any one of the preceding items, wherein the CMT-FOM score has decreased by between 0.3 and 30 points, such as between 0.5 and 20 points, such as between 0.3 and 10 points.
185. The composition for use according to any one of the preceding items, wherein the composition is for administration at a therapeutic dose of 100 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences a decrease in the CMT Functional Outcome Measure score.
186. The composition for use according to any one of the preceding items, wherein the composition is for administration at a therapeutic dose of 200 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences a decrease in the CMT Functional Outcome Measure score.
187. The composition for use according to any one of the preceding items, wherein the subject or a group of subjects experience a decrease in the Fatigue Severity Scale score after treatment with the composition.
188. The composition for use according to item 187, wherein the decrease in the Fatigue Severity Scale score is determined by comparing the change from baseline in the Fatigue Severity Scale score after a defined period of time treatment with the composition comprising (2S)-2-[4-bromo-2-(1,2-oxazol-3-yl)phenoxy]propanoic acid, or a pharmaceutically acceptable salt, hydrate, polymorph, tautomer, or solvate thereof, with the change from baseline in the Fatigue Severity Scale score after a defined period of time of placebo treatment.
189. The composition for use according to item 188, wherein the period of time is 21 days.
190. The composition for use according to any one of items 187 to 189, wherein the Fatigue Severity Scale score has decreased by 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points.
191. The composition for use according to any one of items 187 to 190, wherein the Fatigue Severity Scale score has decreased by between 1 and 20 points, such as between 0.5 and 10 points.
192. The composition for use according to any one of the preceding items, wherein the composition is for administration at a therapeutic dose of 100 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences a decrease in the Fatigue Severity Scale score.
193. The composition for use according to any one of the preceding items, wherein the composition is for administration at a therapeutic dose of 200 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences a decrease in the Fatigue Severity Scale score.
194. The composition for use according to any one of the preceding items, wherein the subject or a group of subjects experience a decrease in the Individualised Neuromuscular Quality of Life score after treatment with the composition.
195. The composition for use according to item 194, wherein the decrease in the Individualised Neuromuscular Quality of Life score is determined by comparing the change from baseline in the Individualised Neuromuscular Quality of Life score after a defined period of time treatment with the composition comprising (2S)-2-[4-bromo-2-(1,2-oxazol-3-yl)phenoxy]propanoic acid, or a pharmaceutically acceptable salt, hydrate, polymorph, tautomer, or solvate thereof, with the change from baseline in the Individualised Neuromuscular Quality of Life score after a defined period of time of placebo treatment.
196. The composition for use according to item 195, wherein the period of time is 21 days.
197. The composition for use according to any one of the preceding items, wherein the Individualised Neuromuscular Quality of Life score has decreased by 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points.
198. The composition for use according to any one of the preceding items, wherein the Individualised Neuromuscular Quality of Life score has decreased by between 0.5 and 30 points, such as between 1 and 20 points, such as between 0.5 and 10 points.
199. The composition for use according to any one of the preceding items, wherein the composition is for administration at a therapeutic dose of 100 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences a decrease in the Individualised Neuromuscular Quality of Life score.
200. The composition for use according to any one of the preceding items, wherein the composition is for administration at a therapeutic dose of 200 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences a decrease in the Individualised Neuromuscular Quality of Life score.
201. The composition for use according to any one of the preceding items, wherein the subject or a group of subjects experience a reduction in jitter after treatment with the composition.
202. The composition for use according to item 201, wherein jitter is determined using single fibre electromyography.
203. The composition for use according to any one of items 201 or 202, wherein the reduction in jitter is determined by comparing the change from baseline in jitter after a defined period of time treatment with the composition comprising (2S)-2-[4-bromo-2-(1,2-oxazol-3-yl)phenoxy]propanoic acid, or a pharmaceutically acceptable salt, hydrate, polymorph, tautomer, or solvate thereof, with the change from baseline in jitter after a defined period of time of placebo treatment.
204. The composition for use according to item 203, wherein the period of time is 21 days.
205. The composition for use according to any one items 201 to 204, wherein jitter has been reduced by at least 10%, such as at least 15%, such as at least 20%, such as at least 25%, such as at least 30%, such as at least 50%, such as at least 75%, such as at least 100%, such as at least 150%, such as at least 200%,
206. The composition for use according to any one of items 201 to 204, wherein jitter has been reduced by between 5% and 95%, such as between 5% and 80%, such as between 10% and 50%,
207. The composition for use according to any one items 201 to 204, wherein jitter determined using single fibre electromyography has been reduced by at least 5 μs, such as at least 10 μs, such as at least 15 μs, such as at least 20 μs, such as at least 25 μs, such as at least 30 μs, such as at least 40 μs, such as at least 50 μs, such as at least 75 μs, such as at least 100 μs.
208. The composition for use according to any one items 201 to 204, wherein jitter determined using single fibre electromyography has been reduced by between 5 μs and 200 μs, such as between 5 μs and 100 μs, such as between 10 μs and 50 μs.
209. The composition for use according to any one of the preceding items, wherein the composition is for administration at a therapeutic dose of 100 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences a reduction in jitter when determined using single fibre electromyography.
210. The composition for use according to any one of the preceding items, wherein the composition is for administration at a therapeutic dose of 200 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences a reduction in jitter when determined using single fibre electromyography.
211. The composition for use according to any one of the preceding items, wherein the subject or a group of subjects experience a reduction in blocking after treatment with the composition.
212. The composition for use according to item 211, wherein blocking is determined using single fibre electromyography.
213. The composition for use according to any one of items 211 or 212, wherein the reduction in blocking is determined by comparing the change from baseline in blocking after a defined period of time treatment with the composition comprising
(2S)-2-[4-bromo-2-(1,2-oxazol-3-yl)phenoxy]propanoic acid, or a pharmaceutically acceptable salt, hydrate, polymorph, tautomer, or solvate thereof, with the change from baseline in blocking after a defined period of time of placebo treatment.
214. The composition for use according to item 213, wherein the period of time is 21 days.
215. The composition for use according to any one of items 211 to 214, wherein blocking has been reduced by at least 5%, such as at least 10%, such as at least 15%, such as at least 20%, such as at least 25%, such as at least 30%, such as at least 50%, such as at least 75%, such as at least 100%, such as at least 150%, such as at least 200%.
216. The composition for use according to any one of items 211 to 215, wherein blocking has been reduced by between 5% and 95%, such as between 5% and 80%, such as between 10% and 50%,
217. The composition for use according to any one of the preceding items, wherein the composition is for administration at a therapeutic dose of 100 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences a reduction in blocking when determined using single fibre electromyography.
218. The composition for use according to any one of the preceding items, wherein the composition is for administration at a therapeutic dose of 200 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences a reduction in blocking when determined using single fibre electromyography.
219. The composition for use according to any one of the preceding items, wherein the subject or a group of subjects experience an increase in the Berg Balance Scale score after treatment with the composition.
220. The composition for use according to item 219, wherein the increase in the Berg Balance Scale score is determined by comparing the change from baseline in the Berg Balance Scale score after a defined period of time treatment with the composition comprising (2S)-2-[4-bromo-2-(1,2-oxazol-3-yl)phenoxy]propanoic acid, or a pharmaceutically acceptable salt, hydrate, polymorph, tautomer, or solvate thereof, with the change from baseline in Berg Balance Scale score after a defined period of time of placebo treatment.
221. The composition for use according to item 220, wherein the period of time is 21 days.
222. The composition for use according to any one items 220 to 221, wherein the Berg Balance Scale score has increased by at 0.5 points, such as at least 1 point, such as at least 2 points, such as at least 3 points, such as at least 4 points, such as at least 5 points, such as at least 6 points, such as at least 8 points, such as at least 10 points, such as at least 15 points, such as at least 20 points.
223. The composition for use according to any one items 220 to 222, wherein the Berg Balance Scale score has increased by between 0.5 and 30 points, such as between 1 and 20 points, such as between 0.5 and 10 points.
224. The composition for use according to any one of the preceding items, wherein the composition is for administration at a therapeutic dose of 100 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an increase in the Berg Balance Scale score.
225. The composition for use according to any one of the preceding items, wherein the composition is for administration at a therapeutic dose of 200 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an increase in the Berg Balance Scale score.
226. The composition for use according to any one of the preceding items, wherein the subject or a group of subjects experience an improvement in walking ability after treatment with the composition.
227. The composition for use according to item 226, wherein walking ability is determined using the Six Spot Step Test (SSST).
228. The composition for use according to any one of items 226 or 227, wherein the improvement in walking ability is determined by comparing the time to complete the SSST after a defined period of time treatment with the composition comprising (2S)-2-[4-bromo-2-(1,2-oxazol-3-yl)phenoxy]propanoic acid, or a pharmaceutically acceptable salt, hydrate, polymorph, tautomer, or solvate thereof, with the change from baseline in time to complete the SSST after a defined period of time of placebo treatment.
229. The composition for use according to item 228, wherein the period of time is 21 days.
230. The composition for use according to any one of items 226 to 229, wherein the time to complete the SSST has been reduced by at least 10%, such as at least 15%, such as at least 20%, such as at least 25%, such as at least 30%, such as at least 50%, such as at least 75%, such as at least 100%, such as at least 150%, such as at least 200%.
231. The composition for use according to any one of items 226 to 229, wherein the time to complete the SSST has been reduced by between 5% and 95%, such as between 5% and 80%, such as between 10% and 50%.
232. The composition for use according to any one of items 226 to 229, wherein the time to complete the SSST has been reduced by at least 0.3 seconds, such as at least 0.5 s, such as at least 0.7 s, such as at least 1.0 s, such as at least 1.5 s, such as at least 2 s, such as at least 2.5 s, such as at least 3 s, such as at least 4 s, such as at least 5 s.
233. The composition for use according to any one of items 226 to 229, wherein the time to complete the SSST has been reduced by between 0.3 s and 10 s, such as between 0.3 s and 5 s, such as between 0.5 s and 5 s.
234. The composition for use according to any one of the preceding items, wherein the composition is for administration at a therapeutic dose of 100 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an improvement in walking ability when determined using the Six Spot Step Test.
235. The composition for use according to any one of the preceding items, wherein the composition is for administration at a therapeutic dose of 200 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an improvement in walking ability when determined using the Six Spot Step Test.
236. The composition for use according to item 169, wherein walking ability is determined using the 10-meter walk/run test (10MWRT).
237. The composition for use according to item 236, wherein the improvement in walking ability is determined by comparing the change from baseline in the 10MWRT after a defined period of time treatment with the composition comprising (2S)-2-[4-bromo-2-(1,2-oxazol-3-yl)phenoxy]propanoic acid, or a pharmaceutically acceptable salt, hydrate, polymorph, tautomer, or solvate thereof, with the change from baseline in 10MWRT after a defined period of time of placebo treatment.
238. The composition for use according to item 237, wherein the period of time is 21 days.
239. The composition for use according to any one of items 237 or 238, wherein the time to complete the 10MWRT has been reduced by at least 10%, such as at least 15%, such as at least 20%, such as at least 25%, such as at least 30%, such as at least 50%, such as at least 75%, such as at least 100%, such as at least 150%, such as at least 200%.
240. The composition for use according to any one of items 237 or 238, wherein the time to complete the 10MWRT has been reduced by between 5% and 95%, such as between 5% and 80%, such as between 10% and 50%.
241. The composition for use according to any one of items 237 or 238, wherein the time to complete the 10MWRT has been reduced by at least 0.3 seconds, such as at least 0.5 s, such as at least 0.7 s, such as at least 1.0 s, such as at least 1.5 s, such as at least 2 s, such as at least 2.5 s, such as at least 3 s, such as at least 4 s, such as at least 5 s.
242. The composition for use according to any one of items 237 or 238, wherein the time to complete the 10MWRT has been reduced by between 0.3 s and 10 s, such as between 0.3 s and 5 s, such as between 0.5 s and 5 s.
243. The composition for use according to any one of the preceding items, wherein the composition is for administration at a therapeutic dose of 100 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an improvement in walking ability when determined using the 10-meter walk/run test.
244. The composition for use according to any one of the preceding items, wherein the composition is for administration at a therapeutic dose of 200 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an improvement in walking ability when determined using the 10-meter walk/run test.
245. The composition for use according to any one of the preceding items, wherein the subject or a group of subjects experience an improvement in balance and mobility after treatment with the composition.
246. The composition for use according to item 245, wherein the improvement in balance and mobility is determined by comparing the change from baseline in the Timed “Up and Go” (TUG) test after a defined period of time treatment with the composition comprising (2S)-2-[4-bromo-2-(1,2-oxazol-3-yl)phenoxy]propanoic acid, or a pharmaceutically acceptable salt, hydrate, polymorph, tautomer, or solvate thereof, with the change from baseline in TUG test after a defined period of time of placebo treatment.
247. The composition for use according to item 246, wherein the period of time is 21 days.
248. The composition for use according to any one of items 245 to 247, wherein the time to complete the TUG test has been reduced by at least 10%, such as at least 15%, such as at least 20%, such as at least 25%, such as at least 30%, such as at least 50%, such as at least 75%, such as at least 100%, such as at least 150%, such as at least 200%.
249. The composition for use according to any one of items 245 to 247, wherein the time to complete the TUG test has been reduced by between 5% and 95%, such as between 5% and 80%, such as between 10% and 50%, 250. The composition for use according to any one of items 245 to 247, wherein the time to complete the TUG test has been reduced by at least 0.3 seconds, such as at least 0.5 s, such as at least 0.7 s, such as at least 1.0 s, such as at least 1.5 s, such as at least 2 s, such as at least 2.5 s, such as at least 3 s, such as at least 4 s, such as at least 5 s.
251. The composition for use according to any one of items 245 to 247, wherein the time to complete the TUG test has been reduced by as between 0.3 s and 10 s, such as between 0.3 s and 5 s, such as between 0.5 s and 5 s.
252. The composition for use according to any one of the preceding items, wherein the composition is for administration at a therapeutic dose of 100 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an improvement in balance and mobility when determined using the Timed “Up and Go” test.
253. The composition for use according to any one of the preceding items, wherein the composition is for administration at a therapeutic dose of 200 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an improvement in balance and mobility when determined using the Timed “Up and Go” test.
254. A composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of Charcot-Marie-Tooth disease in a subject, wherein the subject has a level of serum uric acid below 6.5 mg/dL.
255. The composition for use according to item 254, wherein the composition is for administration at a therapeutic dose of 100 to 1500 mg of (2S)-2-[4-bromo-2-(1,2-oxazol-3-yl)phenoxy]propanoic acid.
256. A method for treatment of Charcot-Marie-Tooth disease in a subject, said method comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.
257. The method according to item 256, wherein the method results in:
   a. a decrease in the Overall Neuropathy Limitations Score;
   b. an increase in the total distance walked during a 6-minute walk test;
   c. a reduction in fatigue when determined using a fatigue index calculated from the 6-minute walk test;
   d. a decrease in the CMT Neuropathy Score 2 score;
   e. a decrease in the CMT examination score (second version) score;
   f. an increase in the motor function measure 32-item score;
   g. a decrease in the CMT Functional Outcome Measure score;
   h. an increase in the Berg Balance Scale score;
   i. a decrease in the Individualised Neuromuscular Quality of Life score;
   j. a decrease in the Fatigue Severity Scale score;
   k. an improvement in neuromuscular junction transmission;
   l. an improvement in finger dexterity when determined using a nine-hole peg test (9-HPT);
   m. an improvement in walking ability when determined using a Six Spot Step Test;
   n. an improvement in walking ability when determined using a 10-meter walk/run test;
   o. an improvement in balance and mobility when determined using the Timed “Up and Go” test;
   p. an increase in muscle strength as assessed with a hand held dynamometer; and/or
   q. an increase in muscle strength as assessed with a fixed dynamometer.
258. The method according to item 257, wherein the improvement in neuromuscular junction transmission is an improvement is a reduction of jitter and/or blocking when measured using sfEMG.
259. A method for treating Charcot-Marie-Tooth disease in a subject that result in an increase in the total distance walked, said method comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.
260. The method according to item 259, wherein the increase in the total distance walked is determined using a 6-minute walk test.
261. The method according to one of items 259 or 260, wherein the therapeutically effective dose provides a Cₘₐₓ in the range of 2,790 ng/mL to 76,700 ng/mL in the subject.
   The method according to one of items 259 to 261, wherein the improvement in the total distance walked is determined by comparing the change from baseline in the total distance walked after a defined period of time of treatment with the composition with the change from baseline in the total distance walked after the defined period of time of placebo treatment.
262. The method according to one of items 259 to 261, wherein the subject experiences an increase in total distance walked of at least 5%, such as at least 10%, such as at least 15%, such as at least 20%, such as at least 25%, such as at least 30%, such as at least 50%, such as at least 75%, such as at least 100%, such as at least 150%, such as at least 200%.
263. The method according to one of items 260 or 262, wherein the subject experiences an increase in total distance walked of between 5% and 400%, such as between 5% and 200%, such as between 10% and 200%.
264. The method according to one of items 260 or 262, wherein the subject experiences an increase in total distance walked of least 20 metres, such as at least 30 metres, such as at least 40 metres, such as at least 50 metres, such as at least 60 metres, such as at least 80 metres, such as at least 100 metres, such as at least 150 metres, such as at least 200 metres, such as at least 250 metres, such as at least 300 metres.
265. The method according to one of items 260 or 262, wherein the subject experiences an increase in total distance walked of between 20 and 400 metres, such as between 30 and 300 metres, such as between 40 and 200 metres.
266. A method for treating Charcot-Marie-Tooth disease in a subject that result in an increase in muscle strength, said method comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.
267. The method according to item 266, wherein the increase in muscle strength is determined by measuring the strength of the thigh (knee flexors), the upper arm (elbow flexor and extension) and/or the shoulder (shoulder abduction) or by measuring grip strength.
268. The method according to one of items 266 to 267, wherein the strength is determined by measuring grip strength using a handheld dynamometer.
269. The method according to one of items 266 to 268, wherein the strength is determined by measuring muscle strength using a fixed dynamometer.
270. The method according to one of items 266 to 269, wherein the subject experiences an increase in muscle strength of at least 5%, such as at least 10%, such as at least 15%, such as at least 20%, such as at least 25%, such as at least 30%, such as at least 50%, such as at least 75%, such as at least 100%, such as at least 150%, such as at least 200% such as between 10% and 400%, such as between 15% and 200%, such as between 20% and 100%.
271. The method according to one of items 266 to 270, wherein the subject experiences an increase in muscle strength when determined by measuring hand grip strength using a handheld dynamometer, wherein hand grip strength has increased by at least 0.25 kg, such as at least 0.50 kg, such as at least 0.75 kg, such as at least 1.0 kg, such as at least 1.25 kg, such as at least 1.5 kg, such as at least 1.75 kg, such as at least 2.0 kg, such as at least 2.5 kg, such as at least 3.0 kg, such as between 0.25 and 5.0 kg, such as between 0.25 and 4.0 kg, such as between 0.5 and 4.0 kg.
272. The method according to one of items 266 to 271, wherein the subject experiences an increase in muscle strength when determined by measuring hand grip strength using a handheld dynamometer, wherein hand grip strength has increased by at least 0.25 kg, such as at least 0.50 kg, such as at least 0.75 kg, such as at least 1.0 kg, such as at least 1.25 kg, such as at least 1.5 kg, such as at least 1.75 kg, such as at least 2.0 kg, such as at least 2.5 kg, such as at least 3.0 kg, such as between 0.25 and 5.0 kg, such as between 0.25 and 4.0 kg, such as between 0.5 and 4.0 kg.
273. The method according to one of items 266 to 272, wherein the therapeutically effective dose provides a Cₘₐₓ in the range of 2,790 ng/mL to 76,700 ng/mL in the subject.
274. A method for treating Charcot-Marie-Tooth disease in a subject that result in an improvement in finger dexterity, said method comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.
275. The method according to item 274, wherein the improvement in finger dexterity can be determined using a nine-hole peg test.
276. The method according to item 275, wherein the therapeutically effective dose provides a Cₘₐₓ in the range of 2,790 ng/mL to 76,700 ng/mL in the subject.
277. The method according to one of items 274 to 276, wherein the subject experiences an improvement in finger dexterity of at least 10%, such as at least 15%, such as at least 20%, such as at least 25%, such as at least 30%, such as at least 50%, such as at least 75%, such as at least 100%, such as at least 150%, such as at least 200%, such as between 5% and 95%, such as between 5% and 80%, such as between 10% and 50%.
278. A method for treating Charcot-Marie-Tooth disease in a subject that result in an improvement in walking ability, said method comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.
279. The method according to item 278, wherein the improvement in walking ability is determined using the Six Spot Step Test.
280. The method according to item 278, wherein the improvement in walking ability is determined using the 10-meter walk/run test.
281. The method according to one of items 278 to 280, wherein the therapeutically effective dose provides a Cₘₐₓ in the range of 2,790 ng/mL to 76,700 ng/mL in the subject.
282. The method according to one of items 278 to 281, wherein the subject experiences an improvement in walking ability of at least 10%, such as at least 15%, such as at least 20%, such as at least 25%, such as at least 30%, such as at least 50%, such as at least 75%, such as at least 100%, such as at least 150%, such as at least 200%, such as between 5% and 95%, such as between 5% and 80%, such as between 10% and 50%.
283. The method according to one of items 278 to 282, wherein the subject experiences an improvement in walking ability wherein the time to complete the Six Spot Step Test has been reduced by at least 0.3 seconds, such as at least 0.5 s, such as at least 0.7 s, such as at least 1.0 s, such as at least 1.5 s, such as at least 2 s, such as at least 2.5 s, such as at least 3 s, such as at least 4 s, such as at least 5 s, such as between 0.3 s and 10 s, such as between 0.3 s and 5 s, such as between 0.5 s and 5 s.
284. The method according to one of items 278 to 283, wherein the subject experiences an improvement in walking ability wherein the time to complete the 10-meter walk/run test has been reduced by at least 0.3 seconds, such as at least 0.5 s, such as at least 0.7 s, such as at least 1.0 s, such as at least 1.5 s, such as at least 2 s, such as at least 2.5 s, such as at least 3 s, such as at least 4 s, such as at least 5 s, such as between 0.3 s and 10 s, such as between 0.3 s and 5 s, such as between 0.5 s and 5 s.
285. A method for treating Charcot-Marie-Tooth disease in a subject that result in a reduction in fatigue, said method comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.
286. The method according to item 285, wherein the reduction in fatigue is calculated from the distance walked in the 1st minute to the distance walked in the 6th minute of a 6-minute walk test.
287. The method according to one of items 285 to 286, wherein the subject experiences a reduction in fatigue of at least 5%, such as at least 10%, such as at least 15%, such as at least 20%, such as at least 25%, such as at least 30%, such as at least 40%, such as at least 50%, such as at least 60%, such as at least 70%, such as at least 80%, such as between 5% and 95%, such as between 5% and 80%, such as between 10% and 50%.
288. The method according to one of items 285 to 287, wherein the therapeutically effective dose provides a Cₘₐₓ in the range of 2,790 ng/mL to 76,700 ng/mL in the subject.
289. A method for treating Charcot-Marie-Tooth disease in a subject that result in an increase in the motor function measure 32-item score, said method comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.
290. The method according to item 289, wherein the subject experiences an increase in the motor function measure 32-item score of at least 5%, such as at least 10%, such as at least 15%, such as at least 20%, such as at least 25%, such as at least 30%, such as at least 40%, such as at least 50%, such as at least 60%, such as at least 70%, such as at least 80%, such as between 5% and 95%, such as between 5% and 80%, such as between 10% and 50%.
291. The method according to one of items 289 or 290, wherein the therapeutically effective dose provides a Cₘₐₓ in the range of 2,790 ng/mL to 76,700 ng/mL in the subject.
292. A method for treating Charcot-Marie-Tooth disease in a subject that result in a decrease in the Fatigue Severity Scale score, said method comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.
293. The method according to item 292, wherein the subject experiences a decrease in the Fatigue Severity Scale score of at least 0.3 points, such as at least 0.5 points, such as at least 0.75 points, such as at least 1 point, such as at least 2 points, such as at least 3 points, such as at least 4 points, such as at least 5 points, such as at least 6 points, such as at least 8 points, such as at least 10 points, such as at least 15 points, such as at least 20 points, such as between 0.3 and 30 points, such as between 0.5 and 20 points, such as between 0.3 and points.
294. The method according to one of items 292 or 293, wherein the therapeutically effective dose provides a Cₘₐₓ in the range of 2,790 ng/mL to 76,700 ng/mL in the subject.
295. A method for treating Charcot-Marie-Tooth disease in a that result in an improvement in balance and mobility, said method comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.
296. The method according to item 295, wherein the improvement in balance and mobility can be determined using the Timed “Up and Go” test.
297. The method according to one of items 295 to 296, wherein the subject an improvement in balance and mobility of at least 10%, such as at least 15%, such as at least 20%, such as at least 25%, such as at least 30%, such as at least 50%, such as at least 75%, such as at least 100%, such as at least 150%, such as at least 200%, such as between 5% and 95%, such as between 5% and 80%, such as between 10% and 50%.
298. The method according to one of items 295 to 296, wherein the subject an improvement in walking ability wherein the time to complete the Timed “Up and Go” test has been reduced by at least 0.3 seconds, such as at least 0.5 s, such as at least 0.7 s, such as at least 1.0 s, such as at least 1.5 s, such as at least 2 s, such as at least 2.5 s, such as at least 3 s, such as at least 4 s, such as at least 5 s, such as between 0.3 s and 10 s, such as between 0.3 s and 5 s, such as between 0.5 s and 5 s.
299. The method according to one of items 295 to 298, wherein the therapeutically effective dose provides a Cₘₐₓ in the range of 2,790 ng/mL to 76,700 ng/mL in the subject.
300. A method for treating Charcot-Marie-Tooth disease in a subject that result in an improvement in neuromuscular junction transmission, said method comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.
301. The method according to item 300, wherein the improvement in neuromuscular junction is a reduction in jitter and/or blocking and is determined using single fibre electromyography.
302. The method according to one of items 300 or 301, wherein the subject experiences a reduction in jitter of at least 10%, such as at least 15%, such as at least 20%, such as at least 25%, such as at least 30%, such as at least 50%, such as at least 75%, such as at least 100%, such as at least 150%, such as at least 200%, such as between 5% and 95%, such as between 5% and 80%, such as between 10% and 50%,
303. The method according to one of items 300 or 301, wherein the subject experiences a reduction in jitter of at least 5 μs, such as at least 10 μs, such as at least 15 μs, such as at least 20 μs, such as at least 25 μs, such as at least 30 μs, such as at least 40 μs, such as at least 50 μs, such as at least 75 μs, such as at least 100 μs, such as between 5 μs and 200 μs, such as between 5 μs and 100 μs, such as between 10 μs and 50 μs.
304. The method according to one of items 300 or 301, wherein the subject experiences a reduction in blocking of at least 10%, such as at least 15%, such as at least 20%, such as at least 25%, such as at least 30%, such as at least 50%, such as at least 75%, such as at least 100%, such as at least 150%, such as at least 200%, such as between 5% and 95%, such as between 5% and 80%, such as between 10% and 50%.
305. The method according to one of items 300 to 304, wherein the therapeutically effective dose provides a Cₘₐₓ in the range of 2,790 ng/mL to 76,700 ng/mL in the subject.
306. A method for treating Charcot-Marie-Tooth disease in a subject that result in a decrease in the Overall Neuropathy Limitations Score (ONLS), said method comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.
307. The method according to item 306, wherein the subject experiences subject experiences a decrease in the Overall Neuropathy Limitations Score after treatment with NMD670, wherein the score has decreased by at least 0.3 points, such as at least 0.5 points, such as at least 0.75 points, such as at least 1 point, such as at least 2 points, such as at least 3 points, such as at least 4 points, such as at least 5 points, such as at least 6 points, such as at least 8 points, such as at least 10 points, such as between 0.3 and 10 points, such as between 0.5 and 6 points, such as between 0.3 and 5 points.
308. The method according to one of items 306 or 307, wherein the therapeutically effective dose provides a Cₘₐₓ in the range of 2,790 ng/mL to 76,700 ng/mL in the subject.
309. A method for treating Charcot-Marie-Tooth disease in a subject that result in a decrease in the CMT Neuropathy Score 2 (CMTNS2) score, said method comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.
310. The method according to item 309, wherein the subject a decreased in the CMTNS2 score of at least 0.3 points, such as at least 0.5 points, such as at least 0.75 points, such as at least 1 point, such as at least 2 points, such as at least 3 points, such as at least 4 points, such as at least 5 points, such as at least 6 points, such as at least 8 points, such as at least 10 points, such as between 0.3 and 20 points, such as between 0.5 and 6 points, such as between 0.3 and 5 points.
311. The method according to one of items 309 or 310, wherein the therapeutically effective dose provides a Cₘₐₓ in the range of 2,790 ng/mL to 76,700 ng/mL in the subject.
312. A method for treating Charcot-Marie-Tooth disease in a subject that result in a decrease in the CMT examination score (second version) (CMTES2) score, said method comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.
313. The method according to item 312, wherein the subject experiences a decrease in the CMTES2 score of at least 0.3 points, such as at least 0.5 points, such as at least 0.75 points, such as at least 1 point, such as at least 2 points, such as at least 3 points, such as at least 4 points, such as at least 5 points, such as at least 6 points, such as at least 8 points, such as at least 10 points, such as between 0.3 and 20 points, such as between 0.5 and 6 points, such as between 0.3 and 5 points.
314. The method according to one of items 312 or 313, wherein the therapeutically effective dose provides a Cₘₐₓ in the range of 2,790 ng/mL to 76,700 ng/mL in the subject.
315. A method for treating Charcot-Marie-Tooth disease in a subject that result in a decrease in the CMT Functional Outcome Measure (CMT-FOM) score, said method comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.
316. The method according to item 315, wherein the subject experiences a decrease in the CMT-FOM score of at least 0.3 points, such as at least 0.5 points, such as at least 0.75 points, such as at least 1 point, such as at least 2 points, such as at least 3 points, such as at least 4 points, such as at least 5 points, such as at least 6 points, such as at least 8 points, such as at least 10 points, such as at least 15 points, such as at least 20 points, such as between 0.3 and 30 points, such as between 0.5 and 20 points, such as between 0.3 and 10 points.
317. The method according to one of items 315 or 316, wherein the therapeutically effective dose provides a Cₘₐₓ in the range of 2,790 ng/mL to 76,700 ng/mL in the subject.
318. A method for treating Charcot-Marie-Tooth disease in a that result in an increase in the Berg Balance Scale score, said method comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.
319. The method according to item 318, wherein the subject experiences an increase in the Berg Balance Scale score of at least 0.3 points, such as at least 0.5 points, such as at least 0.75 points, such as at least 1 point, such as at least 2 points, such as at least 3 points, such as at least 4 points, such as at least 5 points, such as at least 6 points, such as at least 8 points, such as at least 10 points, such as at least 15 points, such as at least 20 points, such as between 0.3 and 30 points, such as between 0.5 and 20 points, such as between 0.3 and 10 points.
320. The method according to one of items 318 or 319, wherein the therapeutically effective dose provides a Cₘₐₓ in the range of 2,790 ng/mL to 76,700 ng/mL in the subject.
321. A method for treating Charcot-Marie-Tooth disease in a subject that result in a decrease in the Individualised Neuromuscular Quality of Life score, said method comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.
322. The method according to item 321, wherein the subject experiences a decrease in the Individualised Neuromuscular Quality of Life score of at least 0.3 points, such as at least 0.5 points, such as at least 0.75 points, such as at least 1 point, such as at least 2 points, such as at least 3 points, such as at least 4 points, such as at least 5 points, such as at least 6 points, such as at least 8 points, such as at least 10 points, such as at least 15 points, such as at least 20 points, such as between 0.3 and 30 points, such as between 0.5 and 20 points, such as between 0.3 and 10 points.
323. The method according to one of items 321 or 322, wherein the therapeutically effective dose provides a Cₘₐₓ in the range of 2,790 ng/mL to 76,700 ng/mL in the subject.
324. A method for treating a subject suffering from symptoms of Charcot-Marie-Tooth disease, the method comprising administering a therapeutically effective dose of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg.
325. A method for treatment of Charcot-Marie-Tooth disease in a subject with serum uric acid levels above 6.5 mg/dL, said method comprising administering a low dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject until the patient's serum uric acid level falls below 6.5 mg/dL, and then administering a therapeutic dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the low dose is within the range of 20 mg to 150 mg, such as 25 mg to 100 mg, such as 25 mg to 50 mg, and the therapeutic dose is within the range of 200 mg to 1500 mg.
326. A method for enhancing neuromuscular transmission and/or restoration of skeletal muscle function, comprising administering a composition comprising (2S)-2-[4-bromo-2-(1,2-oxazol-3-yl)phenoxy]propanoic acid, or a pharmaceutically acceptable salt, hydrate, polymorph, tautomer, or solvate thereof, at a therapeutic dose of 100 to 1500 mg of (2S)-2-[4-bromo-2-(1,2-oxazol-3-yl)phenoxy]propanoic acid to said subject.
327. The method according to one of items 256 to 326, wherein the defined period of time is 21 days.
328. The method according to one of items 256 to 327, wherein the therapeutically effective dose provides a Cₘₐₓ in the range of 2,790 ng/mL to 76,700 ng/mL in the subject.
329. The method according to one of items 256 to 328, wherein the therapeutically effective dose provides an AUC_{inf} in the range of 16,700 ng/mL to 534,000 ng/mL in the subject.
330. The method according to one of items 256 to 329, wherein the therapeutically effective dose of the compound has a Tₘₐₓ in the subject ranging from 1 to 6 hours.
331. The method according to one of items 256 to 329, wherein the therapeutically effective dose of the compound has a Tₘₐₓ in the subject ranging from 3 hours to 7 hours.
332. The method according to one of items 256 to 331, wherein the therapeutically effective dose is within the range of 100 mg to 600 mg.
333. The method according to one of items 256 to 331, wherein the therapeutically effective dose is within the range of 200 mg to 600 mg.
334. The method according to one of items 256 to 331, wherein the therapeutically effective dose is 100 mg.
335. The method according to one of items 256 to 331, wherein the therapeutically effective dose is 150 mg.
336. The method according to one of items 256 to 331, wherein the therapeutically effective dose is 200 mg.
337. The method according to one of items 256 to 331, wherein the therapeutically effective dose is 250 mg.
338. The method according to one of items 256 to 331, wherein the therapeutically effective dose is 300 mg.
339. The method according to one of items 256 to 331, wherein the therapeutically effective dose is 350 mg.
340. The method according to one of items 256 to 331, wherein the therapeutically effective dose is 400 mg.
341. The method according to one of items 256 to 331, wherein the therapeutically effective dose is 500 mg.
342. The method according to one of items 256 to 331, wherein the therapeutically effective dose is 600 mg.
343. The method according to one of items 256 to 342, wherein the therapeutically effective dose is administered once, twice, three times or four times daily.
344. The method according to one of items 256 to 326, wherein the therapeutically effective dose is 100 to 600 mg of (2S)-2-[4-bromo-2-(1,2-oxazol-3-yl)phenoxy]propanoic acid and the composition is to be administered one time daily.
345. The method according to one of items 256 to 326, wherein the therapeutically effective dose is 100 to 600 mg of (2S)-2-[4-bromo-2-(1,2-oxazol-3-yl)phenoxy]propanoic acid and the composition is to be administered two times daily.
346. The method according to one of items 256 to 326, wherein the therapeutically effective dose is 100 to 600 mg of (2S)-2-[4-bromo-2-(1,2-oxazol-3-yl)phenoxy]propanoic acid and the composition is to be administered three times daily.
347. The method according to one of items 256 to 326, wherein the therapeutically effective dose is 100 to 600 mg of (2S)-2-[4-bromo-2-(1,2-oxazol-3-yl)phenoxy]propanoic acid and the composition is to be administered four times daily.
348. The method according to one of items 256 to 326, wherein the therapeutically effective dose is 200 to 600 mg of (2S)-2-[4-bromo-2-(1,2-oxazol-3-yl)phenoxy]propanoic acid and the composition is to be administered one time daily.
349. The method according to one of items 256 to 326, wherein the therapeutically effective dose is 200 to 600 mg of (2S)-2-[4-bromo-2-(1,2-oxazol-3-yl)phenoxy]propanoic acid and the composition is to be administered two times daily.
350. The method according to one of items 256 to 326, wherein the therapeutically effective dose is 200 to 600 mg of (2S)-2-[4-bromo-2-(1,2-oxazol-3-yl)phenoxy]propanoic acid and the composition is to be administered three times daily.
351. The method according to one of items 256 to 326, wherein the therapeutically effective dose is 200 to 600 mg of (2S)-2-[4-bromo-2-(1,2-oxazol-3-yl)phenoxy]propanoic acid and the composition is to be administered four times daily.
352. The method according to one of items 256 to 326, wherein the therapeutically effective dose is about 100 mg of (2S)-2-[4-bromo-2-(1,2-oxazol-3-yl)phenoxy]propanoic acid and the composition is to be administered one time daily.
353. The method according to one of items 256 to 326, wherein the therapeutically effective dose is about 100 mg of (2S)-2-[4-bromo-2-(1,2-oxazol-3-yl)phenoxy]propanoic acid and the composition is to be administered two times daily.
354. The method according to one of items 256 to 326, wherein the therapeutically effective dose is about 100 mg of (2S)-2-[4-bromo-2-(1,2-oxazol-3-yl)phenoxy]propanoic acid and the composition is to be administered three times daily.
355. The method according to one of items 256 to 326, wherein the therapeutically effective dose is about 100 mg of (2S)-2-[4-bromo-2-(1,2-oxazol-3-yl)phenoxy]propanoic acid and the composition is to be administered four times daily.
356. The method according to one of items 256 to 326, wherein the therapeutically effective dose is about 150 mg of (2S)-2-[4-bromo-2-(1,2-oxazol-3-yl)phenoxy]propanoic acid and the composition is to be administered one time daily.
357. The method according to one of items 256 to 326, wherein the therapeutically effective dose is about 150 mg of (2S)-2-[4-bromo-2-(1,2-oxazol-3-yl)phenoxy]propanoic acid and the composition is to be administered two times daily.
358. The method according to one of items 256 to 326, wherein the therapeutically effective dose is about 150 mg of (2S)-2-[4-bromo-2-(1,2-oxazol-3-yl)phenoxy]propanoic acid and the composition is to be administered three times daily.
359. The method according to one of items 256 to 326, wherein the therapeutically effective dose is about 150 mg of (2S)-2-[4-bromo-2-(1,2-oxazol-3-yl)phenoxy]propanoic acid and the composition is to be administered four times daily.
360. The method according to one of items 256 to 326, wherein the therapeutically effective dose is about 200 mg of (2S)-2-[4-bromo-2-(1,2-oxazol-3-yl)phenoxy]propanoic acid and the composition is to be administered one time daily.
361. The method according to one of items 256 to 326, wherein the therapeutically effective dose is about 200 mg of (2S)-2-[4-bromo-2-(1,2-oxazol-3-yl)phenoxy]propanoic acid and the composition is to be administered two times daily.
362. The method according to one of items 256 to 326, wherein the therapeutically effective dose is about 200 mg of (2S)-2-[4-bromo-2-(1,2-oxazol-3-yl)phenoxy]propanoic acid and the composition is to be administered three times daily.
363. The method according to one of items 256 to 326, wherein the therapeutically effective dose is about 200 mg of (2S)-2-[4-bromo-2-(1,2-oxazol-3-yl)phenoxy]propanoic acid and the composition is to be administered four times daily.
364. The method according to one of items 256 to 326, wherein the therapeutically effective dose is about 250 mg of (2S)-2-[4-bromo-2-(1,2-oxazol-3-yl)phenoxy]propanoic acid and the composition is to be administered one time daily.
365. The method according to one of items 256 to 326, wherein the therapeutically effective dose is about 250 mg of (2S)-2-[4-bromo-2-(1,2-oxazol-3-yl)phenoxy]propanoic acid and the composition is to be administered two times daily.
366. The method according to one of items 256 to 326, wherein the therapeutically effective dose is about 250 mg of (2S)-2-[4-bromo-2-(1,2-oxazol-3-yl)phenoxy]propanoic acid and the composition is to be administered three times daily.
367. The method according to one of items 256 to 326, wherein the therapeutically effective dose is about 250 mg of (2S)-2-[4-bromo-2-(1,2-oxazol-3-yl)phenoxy]propanoic acid and the composition is to be administered four times daily.
368. The method according to one of items 256 to 326, wherein the therapeutically effective dose is about 300 mg of (2S)-2-[4-bromo-2-(1,2-oxazol-3-yl)phenoxy]propanoic acid and the composition is to be administered one time daily.
369. The method according to one of items 256 to 326, wherein the therapeutically effective dose is about 300 mg of (2S)-2-[4-bromo-2-(1,2-oxazol-3-yl)phenoxy]propanoic acid and the composition is to be administered two times daily.
370. The method according to one of items 256 to 326, wherein the therapeutically effective dose is about 300 mg of (2S)-2-[4-bromo-2-(1,2-oxazol-3-yl)phenoxy]propanoic acid and the composition is to be administered three times daily.
371. The method according to one of items 256 to 326, wherein the therapeutically effective dose is about 300 mg of (2S)-2-[4-bromo-2-(1,2-oxazol-3-yl)phenoxy]propanoic acid and the composition is to be administered four times daily.
372. The method according to one of items 256 to 326, wherein the therapeutically effective dose is about 350 mg of (2S)-2-[4-bromo-2-(1,2-oxazol-3-yl)phenoxy]propanoic acid and the composition is to be administered one time daily.
373. The method according to one of items 256 to 326, wherein the therapeutically effective dose is about 350 mg of (2S)-2-[4-bromo-2-(1,2-oxazol-3-yl)phenoxy]propanoic acid and the composition is to be administered two times daily.
374. The method according to one of items 256 to 326, wherein the therapeutically effective dose is about 350 mg of (2S)-2-[4-bromo-2-(1,2-oxazol-3-yl)phenoxy]propanoic acid and the composition is to be administered three times daily.
375. The method according to one of items 256 to 326, wherein the therapeutically effective dose is about 350 mg of (2S)-2-[4-bromo-2-(1,2-oxazol-3-yl)phenoxy]propanoic acid and the composition is to be administered four times daily.
376. The method according to one of items 256 to 326, wherein the therapeutically effective dose is about 400 mg of (2S)-2-[4-bromo-2-(1,2-oxazol-3-yl)phenoxy]propanoic acid and the composition is to be administered one time daily.
377. The method according to one of items 256 to 326, wherein the therapeutically effective dose is about 400 mg of (2S)-2-[4-bromo-2-(1,2-oxazol-3-yl)phenoxy]propanoic acid and the composition is to be administered two times daily.
378. The method according to one of items 256 to 326, wherein the therapeutically effective dose is about 400 mg of (2S)-2-[4-bromo-2-(1,2-oxazol-3-yl)phenoxy]propanoic acid and the composition is to be administered three times daily.
379. The method according to one of items 256 to 326, wherein the therapeutically effective dose is about 400 mg of (2S)-2-[4-bromo-2-(1,2-oxazol-3-yl)phenoxy]propanoic acid and the composition is to be administered four times daily.
380. The method according to one of items 256 to 326, wherein the therapeutically effective dose is about 500 mg of (2S)-2-[4-bromo-2-(1,2-oxazol-3-yl)phenoxy]propanoic acid and the composition is to be administered two times daily.
381. The method according to one of items 256 to 326, wherein the therapeutically effective dose is about 500 mg of (2S)-2-[4-bromo-2-(1,2-oxazol-3-yl)phenoxy]propanoic acid and the composition is to be administered three times daily.
382. The method according to one of items 256 to 326, wherein the therapeutically effective dose is about 600 mg of (2S)-2-[4-bromo-2-(1,2-oxazol-3-yl)phenoxy]propanoic acid and the composition is to be administered two times daily.
383. The method according to one of items 256 to 326, wherein the therapeutically effective dose is about 600 mg of (2S)-2-[4-bromo-2-(1,2-oxazol-3-yl)phenoxy]propanoic acid and the composition is to be administered three times daily.
384. The method according to one of items 256 to 383, wherein the therapeutically effective dose is administered orally to the subject.
385. The composition for use or the method according to any one of the preceding items, wherein the method further comprise administering a second therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject either one day, two days, three days, four days, five days, six days or at least seven days after a first therapeutically effective dose is administered.
386. The composition for use or the method according to item 385, wherein the second therapeutically effective dose is from 100 mg to about 1500 mg.
387. The composition for use or the method according to item 385, wherein the second therapeutically effective dose is the same as the first therapeutically effective dose administered to the subject.
388. The composition for use or the method according to any one of items 385 to 387, wherein the method further comprise administering a third therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject either one day, two days, three days, four days, five days, six days or at least seven days after the therapeutically effective dose is administered.
389. The composition for use or the method according to item 388, wherein the third therapeutically effective dose is from 100 mg to about 1500 mg.
390. The composition for use or the method according to item 388, wherein the third therapeutically effective dose is the same as the first therapeutically effective dose and/or the second therapeutically effective dose administered to the subject.
391. The composition for use or the method according to any one of the preceding items, wherein the administration of the therapeutically effective doses of (2S)-2-[4-bromo-2-(1,2-oxazol-3-yl)phenoxy]propanoic acid, or a pharmaceutically acceptable salt, hydrate, polymorph, tautomer, or solvate thereof, are repeated at least 1, 2, 3, 4, 5 or 6 times weekly.
392. The composition for use or the method according to any one of the preceding items, wherein the administration of the therapeutically effective doses of (2S)-2-[4-bromo-2-(1,2-oxazol-3-yl)phenoxy]propanoic acid, or a pharmaceutically acceptable salt, hydrate, polymorph, tautomer, or solvate thereof, are repeated at least 1 to 3 times weekly, 2 to 5 times weekly or 3 to 6 times weekly.
393. The composition for use or the method according to any one of the preceding items, wherein the administration of the therapeutically effective doses of (2S)-2-[4-bromo-2-(1,2-oxazol-3-yl)phenoxy]propanoic acid, or a pharmaceutically acceptable salt, hydrate, polymorph, tautomer, or solvate thereof, are repeated daily.
394. The composition for use or the method according to any one of the preceding items, wherein the administration of the therapeutically effective doses of (2S)-2-[4-bromo-2-(1,2-oxazol-3-yl)phenoxy]propanoic acid, or a pharmaceutically acceptable salt, hydrate, polymorph, tautomer, or solvate thereof, is repeated 1, 2, 3, 4, 5, 6, 7 or 8 times daily.
395. The composition for use or the method according to any one of the preceding items, wherein the administration of the therapeutically effective doses of (2S)-2-[4-bromo-2-(1,2-oxazol-3-yl)phenoxy]propanoic acid, or a pharmaceutically acceptable salt, hydrate, polymorph, tautomer, or solvate thereof, is repeated 1 to 8 times daily or 2 to 5 times daily.
396. The composition for use or the method according to any one of the preceding items, wherein the therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, is administered at least one time daily.
397. The composition for use or the method according to any one of the preceding items, wherein the therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, is administered one time daily.
398. The composition for use or the method according to any one of the preceding items, wherein the therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, is administered two times daily, three times daily, or four times daily.
399. The composition for use or the method according to any one of the preceding items, wherein the therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, is the daily dosage amount of (2S)-2-[4-bromo-2-(1,2-oxazol-3-yl)phenoxy]propanoic acid, or a pharmaceutically acceptable salt, hydrate, polymorph, tautomer, or solvate thereof.
400. The composition for use or the method according to item 399, wherein the daily dosage amount of (2S)-2-[4-bromo-2-(1,2-oxazol-3-yl)phenoxy]propanoic acid, or a pharmaceutically acceptable salt, hydrate, polymorph, tautomer, or solvate thereof is administered as a single dosage.
401. The composition for use or the method according to item 399, wherein the daily dosage amount of (2S)-2-[4-bromo-2-(1,2-oxazol-3-yl)phenoxy]propanoic acid, or a pharmaceutically acceptable salt, hydrate, polymorph, tautomer, or solvate thereof is administered in smaller dosages throughout the day.
402. The composition for use or the method according to item 399, wherein the daily dosage amount of (2S)-2-[4-bromo-2-(1,2-oxazol-3-yl)phenoxy]propanoic acid, or a pharmaceutically acceptable salt, hydrate, polymorph, tautomer, or solvate thereof is administered once a day or at least one time daily, such as twice a day, at least at two different time points throughout the day, three times a day or at least at three different time points throughout the day.
403. Use of a composition comprising (2S)-2-[4-bromo-2-(1,2-oxazol-3-yl)phenoxy]propanoic acid, or a pharmaceutically acceptable salt, hydrate, polymorph, tautomer, or solvate thereof, in the manufacture of a medicament for treatment of Charcot-Marie-Tooth disease in a subject, wherein the composition is for administration at a therapeutic dose of 100 to 1500 mg of (2S)-2-[4-bromo-2-(1,2-oxazol-3-yl)phenoxy]propanoic acid.
404. A composition comprising a therapeutically effective dose of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg.
405. The composition according to item 404, wherein the composition comprises 50 to 400 mg (2S)-2-[4-bromo-2-(1,2-oxazol-3-yl)phenoxy]propanoic acid, or a pharmaceutically acceptable salt, hydrate, polymorph, tautomer, or solvate thereof.
406. The composition according to item 404, wherein the composition comprises 50 mg (2S)-2-[4-bromo-2-(1,2-oxazol-3-yl)phenoxy]propanoic acid, or a pharmaceutically acceptable salt, hydrate, polymorph, tautomer, or solvate thereof.
407. The composition according to item 404, wherein the composition comprises 100 mg (2S)-2-[4-bromo-2-(1,2-oxazol-3-yl)phenoxy]propanoic acid, or a pharmaceutically acceptable salt, hydrate, polymorph, tautomer, or solvate thereof.
408. The composition according to item 404, wherein the composition comprises 150 mg (2S)-2-[4-bromo-2-(1,2-oxazol-3-yl)phenoxy]propanoic acid, or a pharmaceutically acceptable salt, hydrate, polymorph, tautomer, or solvate thereof.
409. The composition according to item 404, wherein the composition comprises 200 mg (2S)-2-[4-bromo-2-(1,2-oxazol-3-yl)phenoxy]propanoic acid, or a pharmaceutically acceptable salt, hydrate, polymorph, tautomer, or solvate thereof.
410. The composition according to item 404, wherein the composition comprises 250 mg (2S)-2-[4-bromo-2-(1,2-oxazol-3-yl)phenoxy]propanoic acid, or a pharmaceutically acceptable salt, hydrate, polymorph, tautomer, or solvate thereof.
411. The composition according to item 404, wherein the composition comprises 300 mg (2S)-2-[4-bromo-2-(1,2-oxazol-3-yl)phenoxy]propanoic acid, or a pharmaceutically acceptable salt, hydrate, polymorph, tautomer, or solvate thereof.
412. The composition according to item 404, wherein the composition comprises 350 mg (2S)-2-[4-bromo-2-(1,2-oxazol-3-yl)phenoxy]propanoic acid, or a pharmaceutically acceptable salt, hydrate, polymorph, tautomer, or solvate thereof.
413. The composition according to item 404, wherein the composition comprises 400 mg (2S)-2-[4-bromo-2-(1,2-oxazol-3-yl)phenoxy]propanoic acid, or a pharmaceutically acceptable salt, hydrate, polymorph, tautomer, or solvate thereof.
414. The composition according to any one of items 404 to 413, wherein the composition further comprises at least one pharmaceutically acceptable adjuvant and/or excipient.
415. The composition according to any of items 404 to 414, wherein the composition is for oral administration.
416. The composition according to any one of items 404 to 415, wherein the composition is a solid dosage form.
417. The composition according to item 416, wherein the solid dosage form is selected from the group consisting of capsule, such as sprinkle capsule and gelatine capsule; tablet, such as uncoated tablet, coated tablet and slow-release tablet; and sprinkle.
418. The composition according to any one of items 416 to 417, wherein the solid dosage form comprises 50 mg to 400 mg of (2S)-2-[4-bromo-2-(1,2-oxazol-3-yl)phenoxy]propanoic acid or a pharmaceutically acceptable salt, hydrate, polymorph, tautomer, or solvate thereof.
419. The composition according to any one of items 416 to 417, wherein the solid dosage form comprises 100 mg of (2S)-2-[4-bromo-2-(1,2-oxazol-3-yl)phenoxy]propanoic acid or a pharmaceutically acceptable salt, hydrate, polymorph, tautomer, or solvate thereof.
420. The composition according to any one of items 416 to 417, wherein the solid dosage form comprises 100 mg of (2S)-2-[4-bromo-2-(1,2-oxazol-3-yl)phenoxy]propanoic acid or a pharmaceutically acceptable salt, hydrate, polymorph, tautomer, or solvate thereof.
421. The composition according to any one of items 416 to 417, wherein the solid dosage form comprises 200 mg of (2S)-2-[4-bromo-2-(1,2-oxazol-3-yl)phenoxy]propanoic acid or a pharmaceutically acceptable salt, hydrate, polymorph, tautomer, or solvate thereof.
422. The composition according to any one of items 416 to 417, wherein the solid dosage form comprises 250 mg of (2S)-2-[4-bromo-2-(1,2-oxazol-3-yl)phenoxy]propanoic acid or a pharmaceutically acceptable salt, hydrate, polymorph, tautomer, or solvate thereof.
423. The composition according to any one of items 416 to 417, wherein the solid dosage form comprises 300 mg of (2S)-2-[4-bromo-2-(1,2-oxazol-3-yl)phenoxy]propanoic acid or a pharmaceutically acceptable salt, hydrate, polymorph, tautomer, or solvate thereof.
424. The composition according to any one of items 416 to 417, wherein the solid dosage form comprises 350 mg of (2S)-2-[4-bromo-2-(1,2-oxazol-3-yl)phenoxy]propanoic acid or a pharmaceutically acceptable salt, hydrate, polymorph, tautomer, or solvate thereof.
425. The composition according to any one of items 416 to 417, wherein the solid dosage form comprises 400 mg of (2S)-2-[4-bromo-2-(1,2-oxazol-3-yl)phenoxy]propanoic acid or a pharmaceutically acceptable salt, hydrate, polymorph, tautomer, or solvate thereof.
426. The composition according to any of items 406 to 417, wherein the pharmaceutically acceptable adjuvant and/or excipient is selected from the group consisting of filler, binder, lubricant and disintegrant.
427. The composition according to any of items 406 to 417, wherein the pharmaceutically acceptable adjuvant and/or excipient is selected from the group consisting of silicified microcrystalline cellulose, microcrystalline cellulose, maltodextrin, magnesium stearate and croscarmellose sodium.
428. The composition according to any one of items 416 to 427, wherein the solid dosage form releases not less than 80% of the compound after 30 minutes, when measured in a United States Pharmacopeia (USP) type 2 dissolution apparatus, paddle at 75 rpm, at a temperature of 37° C.±0.5° C. in 900 mL of pH 6.8 phosphate/citric acid buffer.
429. The composition according to any one of items 404 to 415, wherein the composition is in the form of a liquid, liquid suspension, oil, emulsion, or syrup.
430. The composition according to any of items 404 to 429, wherein the composition comprises 10 to 80 wt %, such as 40 to 65 wt %, such as 50 to 55 wt %, such as about 53 wt % (2S)-2-[4-bromo-2-(1,2-oxazol-3-yl)phenoxy]propanoic acid, or a pharmaceutically acceptable salt, hydrate, polymorph, tautomer, or solvate thereof.
431. The composition according to any of items 404 to 429, wherein the composition comprises 10 to 80 wt %, such as 40 to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof.
432. The composition according to any of items 404 to 429, wherein the composition comprises:
   a. 10 to 80 wt %, such as 40 to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof;
   b. 20 to 80 wt %, such as 25 to 50 wt % filler;
   c. 2 to 20 wt %, such as 3 to 16 wt % binder;
   d. 0.25 to 3 wt %, such as 0.4 to 2.0 wt % lubricant; and
   e. 0.25 to 5 wt %, such as 0.3 to 2.5 wt % disintegrant;
      with the proviso that the sum of the wt % of the components does not exceed 100 wt %.
433. The composition according to any of items 404 to 429, wherein the composition comprises:
   a. 10 to 80 wt %, such as 40 to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof;
   b. 5 to 60 wt %, such as 20 to 40 wt %, such as 21 to 37 wt % silicified microcrystalline cellulose;
   c. 2 to 60 wt %, such as 5 to 16 wt % microcrystalline cellulose;
   d. 1 to 15 wt %, such as 1.5 to 7 wt %, such as 1.8 to 6.0 wt % maltodextrin;
   e. 0.25 to 3 wt %, such as 0.4 to 2.0 wt % magnesium stearate; and
   f. 0.25 to 5 wt %, such as 0.3 to 2.5 wt % Croscarmellose sodium;
with the proviso that the sum of the wt % of the components does not exceed 100 wt %.
434. The composition according to any of items 404 to 429, wherein the composition comprises:
   a. 10 to 80 wt %, such as 40 to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof;
   b. 20 to 80 wt %, such as 25 to 50 wt % filler;
   c. 2 to 20 wt %, such as 3 to 16 wt % binder;
   d. 0.25 to 3 wt %, such as 0.4 to 2.0 wt % lubricant;
   e. 0.25 to 5 wt %, such as 0.3 to 2.5 wt % disintegrant; and
   f. 1 to 10 wt % film coating;
with the proviso that the sum of the wt % of the components does not exceed 100 wt %.
435. The composition according to any of items 404 to 429, wherein the composition comprises or consists of:
   a. 10 to 80 wt %, such as 40 to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof;
   b. 5 to 60 wt %, such as 20 to 40 wt %, such as 21 to 37 wt % silicified microcrystalline cellulose;
   c. 2 to 60 wt %, such as 5 to 16 wt % microcrystalline cellulose;
   d. 1 to 15 wt %, such as 1.5 to 7 wt %, such as 1.8 to 6.0 wt % maltodextrin;
   e. 0.25 to 3 wt %, such as 0.4 to 2.0 wt % magnesium stearate;
   f. 0.25 to 5 wt %, such as 0.3 to 2.5 wt % Croscarmellose sodium; and
   g. 1 to 10 wt % film coating composition such as Opadry white;
with the proviso that the sum of the wt % of the components does not exceed 100 wt %.
436. The composition according to any of items 404 to 429, wherein the composition comprises or consists of:
   a. 10 to 80 wt %, such as 40 to 65 wt %, such as 50 to 55 wt %, such as about 53 wt % (2S)-2-[4-bromo-2-(1,2-oxazol-3-yl)phenoxy]propanoic acid, or a pharmaceutically acceptable salt, hydrate, polymorph, tautomer, or solvate thereof;
   b. 5 to 60 wt %, such as 20 to 40 wt %, such as 21 to 37 wt % silicified microcrystalline cellulose;
   c. 2 to 60 wt %, such as 5 to 16 wt % microcrystalline cellulose;
   d. 1 to 15 wt %, such as 1.5 to 7 wt %, such as 1.8 to 6.0 wt % maltodextrin; and
   e. 0.25 to 3 wt %, such as 0.4 to 2.0 wt % magnesium stearate;
   f. 0.25 to 5 wt %, such as 0.3 to 2.5 wt % Croscarmellose sodium; and
   g. 1 to 10 wt % film coating composition such as Opadry white,
with the proviso that the sum of the wt % of the components does not exceed 100 wt %.
437. The composition according to any one of items 404 to 436 for use in the treatment of Charcot-Marie-Tooth disease.
438. The composition according to any one of items 404 to 436 for use in a method of treatment of Charcot-Marie-Tooth disease in a subject, wherein the composition is for administration at a therapeutic dose of 100 to 1500 mg of (2S)-2-[4-bromo-2-(1,2-oxazol-3-yl)phenoxy]propanoic acid.
439. A method for treatment of Charcot-Marie-Tooth disease comprising administering kit-of-parts according to any one of items 404 to 436 to a subject in need thereof.
440. Use of a kit-of-parts according to any one of items 404 to 436 for the manufacture of a medicament for the treatment of Charcot-Marie-Tooth disease.
441. A kit-of-parts comprising:
   a. (2S)-2-[4-bromo-2-(1,2-oxazol-3-yl)phenoxy]propanoic acid, or a pharmaceutically acceptable salt, hydrate, polymorph, tautomer, or solvate thereof; and
   b. a peripheral myelin protein 22 (PMP22) down regulator.
442. The kit-of-parts according to item 441, wherein the kit-of-parts comprises 100 to 1500 mg of (2S)-2-[4-bromo-2-(1,2-oxazol-3-yl)phenoxy]propanoic acid, or a pharmaceutically acceptable salt, hydrate, polymorph, tautomer, or solvate thereof.
443. The kit-of-parts according to any one of items 441 or 442, wherein the PMP22 down regulator is a combination of (RS)-baclofen, naltrexone hydrochloride and D-sorbitol.
444. The kit-of-parts according to any one of items 441 to 443, wherein the PMP22 down regulator is PXT3003.
445. The kit-of-parts according to any one of items 441 or 442, wherein the PMP22 down regulator is a non-viral DNA plasmid such as Engensis.
446. The kit-of-parts according to any one of items 441, 442 or 445, wherein the PMP22 down regulator is VM202.
447. A kit-of-parts comprising:
   a. (2S)-2-[4-bromo-2-(1,2-oxazol-3-yl)phenoxy]propanoic acid, or a pharmaceutically acceptable salt, hydrate, polymorph, tautomer, or solvate thereof; and
   b. biotin, or a pharmaceutically acceptable salt, hydrate, polymorph, tautomer, or solvate thereof.
448. The kit-of-parts according to item 447, wherein the kit-of-parts comprises 100 to 1500 mg of (2S)-2-[4-bromo-2-(1,2-oxazol-3-yl)phenoxy]propanoic acid, or a pharmaceutically acceptable salt, hydrate, polymorph, tautomer, or solvate thereof.
449. The kit-of-parts according to any one of items 447 or 448, wherein kit-of-parts comprises 100 to 300 mg biotin.
450. The kit-of-part according to any one of items 441 to 449, wherein the kit-of-parts further comprises one or more additional compounds.
451. The kit-of-parts according to item 450, wherein the additional compound is IFB-088.
452. The kit-of-parts according to item 450, wherein the additional compound is CKD-510.
453. The kit-of-parts according to item 450, wherein the additional compound is donaperminogene seltosplasmid.
454. The kit-of-parts according to item 450, wherein the additional compound is icerguastat
455. The kit-of-parts according to item 450, wherein the additional compound is CKD-510.
456. The kit-of-parts according to item 450, wherein the additional compound is AGT-100216.
457. The kit-of-parts according to item 450, wherein the additional compound is MiM-111.
458. The kit-of-parts according to item 450, wherein the additional compound is EN-001.
459. The kit-of-parts according to item 450, wherein the additional compound is DTx-1252
460. The kit-of-parts according to item 450, wherein the additional compound is Ricolinstat
461. The kit-of-parts according to item 450, wherein the additional compound is AT-007 (govorestat),
462. The kit-of-parts according to any one of items 441 to 461 for use in the treatment of Charcot-Marie-Tooth disease.
463. The kit-of-parts according to any one of items 441 to 461 use in a method of treatment of Charcot-Marie-Tooth disease in a subject, wherein the composition is for administration at a therapeutic dose of 100 to 1500 mg of (2S)-2-[4-bromo-2-(1,2-oxazol-3-yl)phenoxy]propanoic acid.
464. A method for treatment of Charcot-Marie-Tooth disease comprising administering kit-of-parts according to one of items 441 to 461 to a subject in need thereof.
465. Use of a kit-of-parts according to any one of items 441 to 461 for the manufacture of a medicament for the treatment of Charcot-Marie-Tooth disease.
466. The composition for use, the method or the kit-of-parts for use according to any one of the preceding items, wherein the subject is a human being.
467. The composition for use, the method or the kit-of-parts for use according to any one of the preceding items, wherein the subject is suffering from symptoms of Charcot-Marie-Tooth disease.
468. The composition for use, the method or the kit-of-parts for use according to any one of the preceding items, wherein the subject has been diagnosed with Charcot-Marie-Tooth disease.
469. The composition for use, the method or the kit-of-parts for use according to any one of the preceding items, wherein the subject has been diagnosed with CMT1, such as CMT1A, CMT1B, CMT1C, CMT1D, CMT1E, CMT1F and/or CMT1G.
470. The composition for use, the method or the kit-of-parts for use according to any one of the preceding items, wherein the subject has been diagnosed with CMT2, such as CMT2A, CMT2B, CMT2C, CMT2D, CMT2E, CMT2F, CMT2G, CMT2H, CMT21, CMT2J, CMT2K, CMT2L, CMT2M, CMT2N, CMT20, CMT2P, CMT2Q, CMT2R, CMT2S, CMT2T, CMT2U, CMT2V, CMT2Z, CMT2X, CMT2Y, CMT2CC, CMT2DD and/or CMT2EE.
471. The composition for use, the method or the kit-of-parts for use according to any one of the preceding items, wherein the subject has been diagnosed with CMT3.
472. The composition for use, the method or the kit-of-parts for use according to any one of the preceding items, wherein the subject has been diagnosed with CMT4, such as CMT4A, CMT4B1, CMT4B2, CMT4B3, CMT4C, CMT4D,CMT4E, CMT4F, CMT4G, CMT4H and/or CMT4J.
473. The composition for use, the method or the kit-of-parts for use according to any one of the preceding items, wherein the subject has been diagnosed with CMT5.
474. The composition for use, the method or the kit-of-parts for use according to any one of the preceding items, wherein the subject has been diagnosed with CMT6.
475. The composition for use, the method or the kit-of-parts for use according to any one of the preceding items, wherein the subject has been diagnosed with CMTX1, CMTX2, CMTX3, CMTX4, CMTX5 and/or CMTX6.
476. The composition for use, the method or the kit-of-parts for use according to any one of the preceding items, wherein the subject has been diagnosed with CMTDI, such as CMTDIA, CMTDIB, CMTDIC, CMTDID, CMTDIE and/or CMTDIF.
477. The composition for use, the method or the kit-of-parts for use according to any one of the preceding items, wherein the subject has been diagnosed with CMTRI, such as CMTRIA and/or CMTRIB.
478. The composition for use, the method or the kit-of-parts for use according to any one of the preceding items, wherein the subject has been diagnosed with CMT1A, CMT1B, CMT2A and/or CMTX1.
479. The composition for use, the method or the kit-of-parts for use according to any one of the preceding items, wherein the subject has a level of serum uric acid below 6.5 mg/dL.

### EXAMPLES

### Example 1: CMT Model

CMT2D Gars^{P278KY} mice (Jackson laboratories, stock 17540) were used (Spauling et al. 2016). All handling and use of animals complied with Danish Animal Welfare regulations (licence 2017-15-0201-01223). Animals were fed ad libitum, kept at 12 h light/dark condition at 21° C. and observed daily. No animals were euthanized due to reaching humane endpoints. One male mouse showed exacerbated progression of muscular atrophy resulting in hunched posture and a broad wobbling gait.

Mice were removed from their housing on the day of experiment and sedated with isoflurane (2-3%) dissolved in oxygen (supplied in a mask placed around the mouth and nose (Microvent 1, Hallowell EMC)) and placed on a heated mat. Sedation was ensured by testing the reflexes of the front limbs by tweezing them with a set of tweezers. Temperature was monitored by rectal probe. When fully sedated the Achilles tendon was blotted, tied by cotton string to a force transducer (Grass instruments) and cut distal to the cotton string, to allow recording of force from muscle of the lower limb. To record compound muscle action potentials (CMAP), two small needle electrodes were placed subcutaneously, one over the belly of the triceps surae muscle, and one at the distal part close to the tendon. To allow stimulation of the sciatic nerve, two stimulation needle electrodes were inserted at the base of the tail, one on each side of the sciatic nerve. Optimal placement was tested by applying short pulses via the stimulation electrodes, and assessing the resulting force and CMAP. When a stable response was obtained, electrodes were secured with tape, and the animal was allowed to rest for 10 minutes.

The single fibre measurements of EPP was performed in animals aged 8 to 15 weeks, whilst the in situ stimulation protocol part was performed in animals 37 weeks of age, from the same litter.

### Intracellular Recordings of Single Fibre EPP and Action Potentials

Animals were sacrificed according to current animal legislation, and the soleus muscle was dissected with around 2 cm suppling motor nerve. The nerve-muscle preparations were mounted horizontally in an experimental chamber continuously perfused (25 mL/min) with Krebs-Ringer solution at 30° C. Prior to initiation of the experiment, the nerve-muscle preparations were allowed to rest in the Krebs-Ringer solution for at least 30 min. The motor innervation point and the endplates were located using a microscope and a glass pipette containing a chlorinated silver wire (suction-electrode) was placed close to the nerve. By decreasing the pressure inside the pipette relative to the outside, the nerve was sucked into the glass pipette and thus it could be stimulated electrically in isolation from the muscle fibres when desired. To obtain intracellular recordings of EPPs and action potentials, a glass microelectrode was inserted into fibres close to visible endplates. The same electrode was used several times when recording from different fibres during different stimulations. To elicit EPPs and action potentials for recordings by the inserted electrode, the motor nerve was stimulated via the suction-electrode. To allow for recording of EPPs without initiating action potentials in the muscle fibers, μ-Conotoxin GIIIB (12.5 μM in water) was added to a concentration of 1 μM to the Krebs-Ringer solution. μ-Conotoxin selectively blocks the voltage-dependent Na⁺ channels of skeletal muscle (NaV_{1,4}) without affecting the voltage-dependent Na⁺ channels of the motor-nerve. EPPs were recorded from several fibres before and after NMD670 (stock solution of 100 mM in DMSO) was added to the experimental chamber to a concentration of 10 μM. When action potentials were recorded μ-Conotoxin was not included by muscle contractions were prevented by including 50 to 100 μM Blebbistatin in Krebs-Ringer solution.

The recordings of EPP the EPP amplitudes were measured in muscle fibres from 3 WT mice and 3 CMT2D mice. The average EPP amplitude in CMT mice was lower than in WT mice at all tested stimulation frequencies, but only significantly lower from EPP number 3 to 18 in 12 and 30 Hz trains, and only from number 8 to 11 in the 80 Hz train (multiple T-test with correction, p-val<0.05). There was a clear difference in the rate of decline in EPP amplitudes with repetitive stimulation at all frequencies between fibres from WT and CMT mice, and the final EPP amplitude at the last stimulation was also lower in CMT mice at all frequencies, although this difference was not significant. After addition of 10 μM NMD670 to the CMT2D fibres a small non-significant increase in EPP amplitude was observed at all stimulation frequencies, with the largest increase observed in the 80 Hz stimulation trains (FIG. 1).

While the addition of NMD670 to isolated soleus muscle from CMT mice did not result in a significant increase in the EPP amplitude from single muscle fibres, the number of muscle fibres that were unable to sustain action potential generation for the entire stimulus train was reduced after addition of 10 μM NMD670. For 12 Hz stimulation 6/60 (10%) fibres were unable to sustain action potential generation, while after addition of NMD670, 3/47 (6,4%) fibres were unable. Likewise at 30 Hz, 9/60 (15%) were unable and after addition 3/47 (6.4%) were unable, and at 80 Hz 33/60 (55%) fibres were unable to sustain action potential generation before addition of NMD670 compared to 20/47 (42.5%) after addition. In addition, the percentage of action potentials that failed to be generated in each train was also reduced at all frequencies (FIGS. 2A, 2B and 2C).

### Stimulation Protocol In Situ

After optimal set-up and rest, the following stimulation paradigm was initiated: Every 30 seconds the sciatic nerve was stimulated at 8-12 Volt by pulses of 0.1 ms duration at 12 Hz with 10 pulses (12 Hz protocol). This was repeated 9 times after which a single stimulation at 120 Hz for 1 s was made. The 12 Hz protocol was then again repeated 9 times followed a stimulation at 30 Hz for 1 s. This cycle was repeated throughout the experiments providing recordings of force and CMAP at 120 Hz and 30 Hz every 10^{th} minute interspaced by 12 Hz protocols every 30 second. When 3 consecutive measurements of 120 Hz were similar, NMD670 was administered I.V. (8 mg/kg dissolved in phosphate buffered saline at 2 mg/mL), while the stimulation paradigm continued. Animals were monitored for 6 hours after injection of NMD670. Force and CMAP were measured and digitalized by an AD converter (CED, UK,) and analysed in “Signal 6” (Cambridge Electronic Designs, UK). When analysing force traces, the peak amplitude of each twitch peak at 12 and 30 Hz was measured, and the baseline subtracted. The force at 30 and 120 Hz was also quantified as a force-time integral, or area under the curve (AUC). The CMAP recordings were quantified as the peak-to-peak levels for each CMAP in every train. The peak heights or CMAP levels were then expressed as ratios relative to the first CMAP peak in the train e.g. forth peak relative to 1^{st} peak (T4/T1) and 10^{th} peak relative to 1^{st} peak (T10/T1).

The Gars^{P278KY} mice generated lower absolute force when compared to WT mice, but were also smaller due to their mixed genetic background, see Table 2. Peak twitch force per body weight (BW) from Gars^{P278KY} mice was significantly lower than that of WT mice. In addition, the Gars^{P278KY} mice displayed inability to maintain force with repeated stimulation, compared to WT mice (FIG. 3A). The ratio of the 4^{th} and 10^{th} peak force relative to the 1^{st} peak in a 12 Hz train of stimulation pulses is summarized in Table 2 for Gars^{P278KY} and WT mice. The WT mice never displayed a ratio lower than 94%, while the Gars^{P278KY} mice displayed a progressively reduced ratio with mean T4/T1 ratio of 88% and T10/T1 of 82%, indicating compromised neuromuscular transmission.

When stimulated for 1 s with a 120 Hz train (tetanic stimulation), WT mice produced more force and were able to sustain force throughout the stimulation train. In comparison, the Gars^{P278KY} mice were not able to sustain force, but instead presented an initial peak followed by decreased force trace, see FIG. 3B (right trace). The CMAP signals from the 120 Hz stimulation were significantly different between WT and Gars^{P278KY} mice, with those in the Gars^{P278KY} mice displaying prominent decrement and those from WT animals with slight elevation (FIG. 3B).

TABLE 2
Summary of Force and EMG values from 3 WT mice and 3 female and
1 male Gars^{P278KY} mice before and after addition of NMD 670

| | Wild Type | | Gars^{P278KY} | |
|---|---|---|---|---|
| Force | Before | After* | Before | After* |
| Twitch force (g) | 21.6 ± 1.3 | 22.2 ± 2.3 | 6.06 ± 0.8 | 7.5 ± 1.1 |
| Twitch force relative to BW | 1.08 ± 0.01 | 1.06 ± 0.02 | 0.42 ± 0.05 | 0.51 ± 0.05 |
| 12 Hz Ratios | | | | |
| T4/T1 | 96.6 ± 1.3 | 97.7 ± 06 | 88.2 ± 5.1 | 97.8 ± 5.0 |
| T10/T1 | 97.3 ± 0.6 | 99.2 ± 0.3 | 82.0 ± 6.0 | 93.8 ± 5.7 |
| 30 and 120 Hz | | | | |
| 120 Hz AUC (% of initial) | 100 | 100.7 ± 2.7 | 100 | 136.7 ± 29.9 |
| 30 Hz AUC (% of initial) | 100 | 102.9 ± 1.9 | 100 | 124.1 ± 3.7 |
| EMG (CMAP) | | | | |
| 120 Hz 5/1 | 110.2 ± 6.5 | 123.5 ± 20.6 | 83.8 ± 13.9 | 102.0 ± 4.3 |
| 30 Hz 5/1 | 105.8 ± 3.1 | 119.0 ± 11.6 | 82.4 ± 3.3 | 95.3 ± 2.4 |
| 12 Hz Ratios CMAP | | | | |
| T4/T1 | 102.2 ± 0.9 | 103.1 ± 0.2 | 75.5 ± 2.8 | 86.3 ± 2.44 |
| T10/T1 | 102.8 ± 0.1 | 102.8 ± 2.2 | 63.5 ± 4.91 | 76.1 ± 4.37 |

*All values were significantly different after i.v. administration of 8 mg/kg NMD670 in Gars^{P278KY} mice when subjected to a paired T-test.

In all Gars^{P278KY} mice an increase in force, CMAP and relative force peaks (T4 or T10/T1) was observed after administration of NMD670 (FIGS. 3C and 3D). The only value that displayed noticeable increase in the WT mice after injection of NMD670 was the ratio of the 5^{th} and 1^{st} CMAP peaks during the 30 and 80 Hz stimulations. The increase in these values in the WT groups was however not significantly different. In Gars^{P278KY} mice injection of NMD670 significantly increased all the values presented in Table 2. The effect of NMD670 was present at all measured parameters, with normalization of T4/T1 and T10/T1 ratios of twitch force, and a 25% increase in absolute twitch force. Force during 30 and 120 Hz stimulation increased 21 and 30% respectively but were not fully normalized (FIG. 3B).

In conclusion, the results demonstrate that administration of a CIC-1 inhibitor, specifically NMD670, reduces neuromuscular transmission failure and improves muscle force.

### Example 2: Tablet Formulation

The immediate release tablets were formulated using standard excipients as described in table 3.

A standard high shear granulation process was developed. Microcrystalline cellulose was used as filler and binder excipient and maltodextrin was also applied as binder.

The granulate contained 50-90% drug substance.

The granulate was dried and sieved and mixed with excipients to form a free-flowing blend. Silicified microcrystalline cellulose was added as a filler, Croscarmellose Sodium as a disintegrant and magnesium stearate as a lubricant. Silicified microcrystalline cellulose was found superior to microcrystalline cellulose as filler to achieve low weight variation. A single punch Diaf tablet press was used to manufacture the core tablets. The core tablets were film-coated with a standard white film-coating pre-mix (Opadry 03F180011 white) consisting of Hypromellose, polyethylene glycol 8000 and titanium dioxide. The term “Opadry White” refers to a composition coating prepared using Opadry® White obtained from Colorcon Pa, USA, in the form of the product sold as product code 03F180011 in 2022.

TABLE 3
Tablet Formulation

| | Quantity Per | Quantity Per | Quantity Per | |
|---|---|---|---|---|
| Substance | Tablet (mg) | Tablet (mg) | Tablet (mg) | Function |
| Product Strength | 50 mg | 100 mg | 300 mg | |

Active ingredient

| | | | | |
|---|---|---|---|---|
| (2S)-2-[4-bromo-2-(1,2- | 50.0 | 100.0 | 300.0 | Drug |
| oxazol-3- | | | | substance |
| yl)phenoxy]propanoic acid | | | | |

Excipients of tablet core

| | | | | |
|---|---|---|---|---|
| Silicified microcrystalline | 2.5-30 | 5-60 | 15-180 | Filler |
| cellulose | | | | |
| Microcrystalline cellulose | 1-30 | 2-60 | 6-180 | Binder |
| Maltodextrin | 0.5-7 | 1-14 | 3-42 | Binder |
| Magnesium Stearate | 0.12-1.5 | 0.25-3 | 0.75-9 | Lubricant |
| Croscarmellose Sodium | 0.12-2.5 | 0.25-5 | 0.75-15 | Disintegrant |

Excipient of the coating

| | | | | |
|---|---|---|---|---|
| Opadry 03F180011 white | 0.5-5 | 1-10 | 3-30 | Film-coating |
| | | | | pre-mix |
| Total | 94.5 | 189 | 567 | |

15-30 mg / 30-60 mg / 90-180 mg Purified water (Ph.Eur), respectively was used and evaporated during processing of the NMD670 50 mg tablet, NMD670 100 mg tablet and NMD670 300 mg tablet, respectively.

### Example 3: Tablet Dissolution

Tablet dissolution was determined as follows.

TABLE 4
Chromatographic conditions

| Apparatus | HPLC system with UV/DAD | | |
|---|---|---|---|
| | detector | | |
| Column | XBridge BEH phenyl 2.5 μm | | |
| | 3.0 × 150 mm | | |
| Column temperature | 40° C. | | |
| Wavelength | 220 nm UV (DAD) detector | | |
| Flow | 0.6 mL/minute | | |
| Injection volume | 3 μL | | |
| Auto sampler temperature | Ambient | | |
| Mobile phase A (MFA): | Milli Q water/Acetonitrile/TFA, | | |
| | 80/20/0.05% | | |
| Mobile phase B (MFB): | Milli Q water/Acetonitrile/TFA, | | |
| | 5/95/0.05% | | |
| Mobile phase | Gradient | | |
| Gradient | Time | % MFA | % MFB |
| | 0.00 | 90 | 10 |
| | 1.00 | 90 | 10 |
| | 11.00 | 10 | 90 |
| | 12.00 | 10 | 90 |
| | 12.10 | 90 | 10 |
| | 16.00 | 90 | 10 |

| | |
|---|---|
| Runtime | 16.00 min |

TABLE 5
Dissolution System

| Apparatus | USP2 (Paddles) |
|---|---|
| Medium | Dissolution Media pH 6.8 |
| Media Volume | 900 ml |
| Paddle Speed | 75 RPM |
| Temperature | 37° C. |
| Sample Extraction Timepoints | 0, 15, 30, 45, 60, 90, 120 minutes |
| Filter | 10 μm |
| Dissolution profile | No replacement of dissolution |
| | media after withdrawal of sample |
| Sampling Volume | 1.5 mL |
| Number of Vessels | 6 |

Dissolution Media pH 6.8 was prepared by dissolving 27.3g Na₂HPO₄·2H₂O and 4.9 g Citric Acid in 1 L Milli-Q water. The pH was measured and adjusted if necessary to pH 6.8.

900 mL of dissolution media pH 6.8 was accurately transferred into each dissolution vessel. The apparatus was assembled, and the dissolution media was heated to 37° C.±0.5° C. The T=0 point was extracted from each vessel prior to dropping the tablets into the dissolution vessels. One tablet was gently dropped into each dissolution vessel, and rotation of the paddle was immediately started. Samples were taken that the sample extraction timepoints and analysed using the HPLC method.

Not less than 80% of NMD670 is released from the tablets after a maximum of 60 minutes when performing release testing of the tablets.

### Example 4: Bioanalysis of Human Plasma Samples

### Sample Collection Method

Collect blood (3 mL recommended) onto wet ice (K₂EDTA tubes)
Immediately cool blood on crushed wet ice for maximum of 30 minutes
Harvest plasma as quickly as possible using refrigerated centrifuge by centrifuging at 2000 g for 10 minutes at 4° C.
Keep plasma on wet ice and immediately stabilise with 1:1 (v/v) addition of Water: Orthophosphoric Acid (100:2 v/v) prepared using≥85% orthophosphoric acid and deionised water (e.g. 750 μL plasma+750 μL stabiliser).
Mix sample thoroughly.
Keep the stabilised plasma on wet ice during subsequent steps.
Divide the stabilised plasma into 5×250 μL aliquots in plastic cryotubes.
Store aliquots at nominal −70° C./−80° C. promptly.

### Sample Preparation Procedure

1. Thaw frozen calibration standards, QC samples, blank matrix, and study samples on wet ice.
2. Vortex-mix all samples thoroughly.
3. On wet ice aliquot 150 μL of calibration standards, QC samples, study samples, and blanks into a 2 mL 96-well plate. For the reagent blank add the same volume of Water.
4. On wet ice add 25 μL of Methanol: Water (50:50) to wells containing blanks and the same volume of ISTD intermediate solution to all other wells.
5. On wet ice add 125 μL of Water to each well. Cap plate and vortex-mix (~5 minutes, 1000 rpm).
6. Centrifuge plate (~3000 g, 3 minutes, room temperature).
7. Condition SPE plate (Waters Oasis HLB μElution) with 250 μL Methanol.
8. Condition SPE plate with 250 μL of Water.
9. Load 250 μL of the sample onto the SPE plate (aspirate from the rim of the cone to avoid any debris collected at the bottom of the well). Pass through using minimum pressure.
10. Wash SPE plate with 250 μL of Water.
11. Wash SPE plate with 250 μL of Methanol: Water (5:95). Briefly increase pressure for one minute to ensure sorbent is partially dried.
12. Elute from SPE plate into a 1.2 mL 96-well plate using 30 μL of Methanol. Stand for one minute then pass through using absolute minimum pressure.
13. Elute again with another 30 μL of Methanol, collecting into the same plate. Briefly increase pressure for one minute to complete the elution.
14. Add 125 μL of Ammonium Formate 10 mM (aq): Formic Acid (100:0.3) to each well. Cap plate and vortex mix (~1 minute, ≥1200 rpm).
15. Store plate refrigerated prior to analysis.

TABLE 6
HPLC Conditions

| | |
|---|---|
| HPLC instrumentation | Waters, Acquity Classic, UPLC |
| | Auxiliary pump, Switching valve |
| Analytical column | Astec CHIROBIOTIC T 250 × 4.6 mm, |
| | 5 μm (Supelco catalogue number |
| | 12024AST) |
| In-line filter | KrudKatcher ULTRA HPLC In-Line Filter |
| | 2 μm, Phenomenex (p/n AF0-8497) |
| Column temperature setting | 25° C. |
| Autosampler temperature | 5° C. |
| Mobile Phase A | Ammonium Formate 10 mM (aq):Formic |
| | Acid (100:0.3) |
| Mobile Phase B | Methanol |
| Back pressure (typical) | 3000 psi |
| Injection volume | 6-15 μL |

TABLE 7
Gradient Conditions

| Time (minutes) | A (%) | B (%) | Flow rate (mL/min) |
|---|---|---|---|
| Initial | 67.5 | 32.5 | 1.05 |
| 8.00 | 27.5 | 72.5 | 1.05 |
| 8.05 | 1.0 | 99.0 | 1.05 |
| 8.30 | 1.0 | 99.0 | 1.05 |
| 9.50 | 1.0 | 99.0 | 2.00 |
| 10.50 | 1.0 | 99.0 | 2.00 |
| 10.55 | 67.5 | 32.5 | 2.00 |
| 12.50 | 67.5 | 32.5 | 1.05 |

TABLE 8
Mass Spectrometer Parameters

| Mass spectrometer | Sciex API 5000 | |
|---|---|---|
| Ionization | ESI- | |
| Resolution | Q1: Unit | |
| | Q3: Unit | |
| IonSpray voltage | −4500 V | |
| Temperature | 550° C. | |
| Collision gas (CAD) | Nitrogen | Setting: 9 |
| Curtain gas (CUR) | Nitrogen | Setting: 30 |
| Nebulizing gas (GS1) | Nitrogen | Setting: 55 |
| Auxiliary gas (GS2) | Nitrogen | Setting: 65 |

| | |
|---|---|
| Entrance potential (EP) | −10 V |
| Pause time | 5 ms |
| Acquisition time | Delay time: 210 seconds delay |
| | Acquisition time after delay: 4.80 minutes |
| | Total time: 8.30 minutes |
| Cycle time | 13.3 min (injection start to next injection start) |

TABLE 9
Compound Detection Parameters

| | | | | | | Approx- |
|---|---|---|---|---|---|---|
| | | | | | | imate |
| | | | | | | RT and |
| Compound | Transition | Dwell | DP | CE | CXP | window |
| Name | Monitored | Time (ms) | (V) | (V) | (V) | (min) |
| NMD670 | 310.0 → 237.9 | 60 | −80 | −19 | −12 | 5.80 min |
| ISTD1* | 315.0 → 242.9 | 60 | −80 | −19 | −12 | (4.06-7.54) |

*ITSD1 is NMD670-d₃.

### Example 5: Phase I Clinical Trial

Part A1 tested single doses of NMD670 in a double-blind, randomised, placebo-controlled, partial crossover and dose-escalating design in healthy male subjects (see the WHO International Clinicals Trials Registry Platform study reference NL8692). A total of nine dose levels were investigated in three cohorts of subjects. Each cohort consisted of nine subjects, each subject had three study sessions. Each subject received escalating doses of NMD670 on two occasions and placebo on one occasion, the order will be randomized in a cross-over fashion. Each dose level was randomized in a 6:3 ratio (active vs. placebo). See Table 10 for an overview of the randomization scheme.

TABLE 10
Randomisation Scheme

| Sequence | Period 1 | Period 2 | Period 3 |
|---|---|---|---|
| 1 | Placebo | Dose 2 | Dose 3 |
| 2 | Dose 1 | Placebo | Dose 3 |
| 3 | Dose 1 | Dose 2 | Placebo |

Dose escalation was stopped after an adverse event of myotonia of moderate intensity observed in one subject administered 1600 mg of NMD670 at dose level 7 (spontaneously and fully resolved within hours). Due to this temporary halt and partial unblinding of the study during dose level 7, a new randomization was necessary. After unblinding three subjects in dose level 7, the original randomization for dose 8 and 9 was changed in order to keep the study blinded. As subjects of cohort 3 only had 2 occasions left, the randomisation of the original design (3-way cross-over) was not possible without compromising the ratio of active and placebo treated subjects, and therefore the within-subject comparison which is important for the evaluation of PD markers. Therefore, for the remaining 2 doses, a full cross-over design investigating one previously tested dose level was used. The 9 subjects in cohort 3 were randomized to receive the study drug in level 8 and placebo in level 9, or vice versa. To determine the effect of food on the exposure of single oral doses of NMD670, dose level 5 was administered in both the fasted and fed condition. Subjects who received dose level 5 returned for a fourth visit in which they received dose level 5 (or matching placebo) in the fed condition, in the same randomization as the chosen dose level in the fasted condition.

Part A2 of the study investigated the safety, tolerability and pharmacokinetics of NMD670 in 8 healthy female subjects of non-childbearing potential, in a randomized, double-blind, placebo-controlled single dose administration of NMD670. Subjects received 800 mg NMD670. Subjects were randomized in a 6:2 ratio (active vs. placebo).

Main inclusion criteria healthy volunteers (Part A)
1. Signed informed consent prior to any study-mandated procedure
2. Part A1: Healthy male subjects, 18 to 45 years of age, inclusive at screening.
3. Part A2: Healthy female subjects of non-childbearing potential, 18-65 years of age, inclusive at screening.
5. Body mass index (BMI) between 18 and 30 kg/m2, inclusive at screening, and with a minimum weight of 50 kg.
6. All males must practice effective contraception during the study and be willing and able to continue contraception for at least 90 days after their last dose of study treatment.
7. Has the ability to communicate well with the Investigator in the Dutch language and willing to comply with the study restrictions.

### Main Exclusion Criteria Healthy Volunteers (Part A)

1. Evidence of any active or chronic disease or condition that could interfere with or for which the treatment of might interfere with, the conduct of the study, or that would pose an unacceptable risk to the subject in the opinion of the investigator (following a detailed medical history, physical examination, vital signs (systolic and diastolic blood pressure, pulse rate, body temperature) and 12-lead electrocardiogram (ECG). Minor deviations from the normal range may be accepted, if judged by the Investigator to have no clinical relevance.
2. Clinically significant abnormalities, as judged by the investigator, in laboratory test results (including hepatic and renal panels, complete blood count, chemistry panel and urinalysis). In the case of uncertain or questionable results, tests performed during screening may be repeated before randomization to confirm eligibility or judged to be clinically irrelevant for healthy subjects.
3. Positive Hepatitis B surface antigen (HBsAg), Hepatitis C antibody (HCV Ab), or human immunodeficiency virus antibody (HIV Ab) at screening.
4. Systolic blood pressure (SBP) greater than 140 or less than 90 mm Hg, and diastolic blood pressure (DBP) greater than 90 or less than 50 mm Hg at screening.
5. Abnormal findings in the resting ECG at screening defined as:
   a. QTcF>450 or <300 msec for men and QTcF>470 or <300 msec for women
   b. Notable resting bradycardia (HR<45 bpm) or tachycardia (HR>100 bpm)
   c. Personal or family history of congenital long QT syndrome or sudden death;
   d. ECG with QRS and/or T wave judged to be unfavourable for a consistently accurate QT measurement (e.g., neuromuscular artefact that cannot be readily eliminated, arrhythmias, indistinct QRS onset, low amplitude T wave, merged T- and U-waves, prominent U waves);
   e. Evidence of atrial fibrillation, atrial flutter, complete branch block, Wolf-Parkinson-White Syndrome, or cardiac pacemaker
6. Use of any medications (prescription or over-the-counter [OTC]), within 14 days of study drug administration, or less than 5 half-lives (whichever is longer). Exceptions are paracetamol (up to 4 g/day) and ibuprofen (up to 1g/day). Other exceptions will only be made if the rationale is clearly documented by the investigator.
7. Use of any vitamin, mineral, herbal, and dietary supplements within 7 days of study drug administration, or less than 5 half-lives (whichever is longer). Exceptions will only be made if the rationale is clearly documented by the investigator.
8. Participation in an investigational drug or device study (last dosing of previous study was within 90 days prior to first dosing of this study).
9. History of abuse of addictive substances (alcohol, illegal substances) or current use of more than 21 units alcohol per week, drug abuse, or regular user of sedatives, hypnotics, tranquillisers, or any other addictive agent.
10. Positive test for drugs of abuse at screening or pre-dose. Retesting is allowed at the discretion of the Investigator.
11. Alcohol will not be allowed from at least 24 hours before screening or pre-dose.
12. Smoker of more than 10 cigarettes per day prior to screening or who use tobacco products equivalent to more than 10 cigarettes per day and unable to abstain from smoking whilst in the unit.
13. Subjects will not be allowed to have excessive caffeine consumption, defined as >800 mg per day.
14. Any confirmed significant allergic reactions (urticaria or anaphylaxis) against any drug, or multiple drug allergies (non-active hay fever is acceptable).
15. Loss or donation of blood over 500 mL within three months (males) or four months (females) prior to screening or intention to donate blood or blood products during the study.
16. Any known factor, condition, or disease that might interfere with treatment compliance, study conduct or interpretation of the results such as drug or alcohol dependence or psychiatric disease.
17. History of trauma to the lower extremities or other conditions (most importantly neurological or muscle diseases) that, in the opinion of the investigator, could affect the electrophysiological measurements.
18. Excessive exercise within 7 days before study drug administration.
19. Clinically significant abnormalities in coagulation.
   Concomitant medications for Healthy volunteers (Part A)

No prescription medications and OTC medications were permitted within 14 days prior to study drug administrations, or less than 5 half-lives (whichever is longer), and during the course of the study.

No vitamin, mineral, herbal, and dietary supplements will be permitted within 7 days prior to study drug administrations, or less than 5 half-lives (whichever is longer), and during the course of the study.

Exceptions were paracetamol (up to 4 g/day) and ibuprofen (up to 1g/day). Other exceptions were only be made if the rationale was clearly documented by the investigator. No exceptions were made for drugs that could influence the MVRC outcomes, such as sodium channel blockers, dantrolene, or anti-epileptic drugs. Use of substrates of CYP2C9, and CYP2C19 were prohibited. For CYP2C19 substrates, a) substrates exhibiting ≥5-fold increase in substrate AUC with CYP2C19 inhibitors: s-mephenytoin, omeprazole; or b) substrates exhibiting 2-fold but <5-fold increase in substrate AUC: diazepam, lansoprazole, rabeprazole, voriconazole were prohibited. For CYP2C9 substrates, a) substrates exhibiting 5-fold increase in substrate AUC with CYP2C9 Inhibitors: celecoxib; or b) substrates exhibiting 2-fold but <5-fold increase in substrate AUC: glimepiride, phenytoin, tolbutamide, warfarin were prohibited.

Use of any vaccine (initial or follow-up): there was a minimum period of 1 week between vaccination and screening; and 1 week between vaccination and dosing; until end of study.

### Tolerability/Safety Endpoints

The following endpoints were determined at time points indicated in the Schedule of Assessments.
Serious adverse events (SAEs) and adverse events (AEs) were collected throughout the study at every study visit.
Concomitant medication
Clinical laboratory tests
   Haematology
   Chemistry
   Urinalysis
   Coagulation
Vital signs
   Pulse Rate (bpm)
   Systolic blood pressure (mmHg)
   Diastolic blood pressure (mmHg)
   Respiratory rate
ECG
   Heart Rate (HR) (bpm), PR, QRS, QT, QTcF
24-hour Holter recording
Hand grip dynamometry
   Grip release profile; time profile from 100% maximum voluntary contraction (MVC) to 5% of the 100% MVC

### Results of Phase 1 (Part A)

The demographic characteristics of the 35 subjects enrolled in Part A of the Phase 1 study are presented in Table 11.

TABLE 11
Demographic Characteristics of 35 Subjects

| | | |
|---|---|---|
| Age | Mean (SD) | 34.7 (14.0) |
| Weight (kg) | Mean (SD) | 76.2 (10.2) |
| Height (cm) | Mean (SD) | 178.5 (9.8) |
| BMI | Mean (SD) | 24.0 (3.1) |
| Sex | Female (n) | 8 |
| | Male (n) | 27 |
| Race | Asian (n) | 3 |
| | Black (n) | 1 |
| | Mixed (n) | 2 |
| | Other (n) | 1 |
| | White (n) | 28 |

(SD)—standard deviation; n—number of subjects

### Adverse Events

No serious or severe adverse effects were reported. There were no meaningful relationships between increases in NMD670 dose and the incidence of participants with AEs. A total of 70 AEs were reported, of which 47 (67%) were at least possibly drug related. There were no relationships between increases in dose and the incidence of these individual AEs following administration of a single dose of NMD670, except for transient myotonia which was reported at the highest dose levels tested (1200 mg and 1600 mg). Most AEs were mild, except for one AE of myotonia (1600 mg), and tooth extraction (unrelated, 50 mg), which were moderate in intensity. There were no subject discontinuations.

### Pharmacokinetic Results

The pharmacokinetic results from the volunteers is given in table 12 below.

TABLE 12
Pharmacokinetic Results

| | AUC_{0-infinity} (Mean ± | Cₘₐₓ (Mean ± SD, | T_{1/2} (mean ± SD, |
|---|---|---|---|
| Dose of NMD670 | SD, h · ng/mL) | ng/mL) | hr) |
| 50 mg | — | 1900 ± 580 | — |
| 100 mg | 18500 ± 1770 | 3980 ± 1190 | 5.07 ± 0.63 |
| 200 mg | 36100 ± 5030 | 7780 ± 1130 | 4.6 ± 0.67 |
| 400 mg | 87700 ± 21400 | 20100 ± 7340 | 4.68 ± 0.49 |
| 800 mg fasted | 191000 ± 31500 | 36700 ± 15400 | 5.39 ± 0.82 |
| (male) | | | |
| 800 mg fed (male) | 180000 ± 27900 | 37300 ± 7420 | 5.02 ± 1.04 |
| 800 mg (female) | 256000 ± 56200 | 44400 ± 7480 | 5.55 ± 0.55 |
| 1200 mg | 358000 ± 176000 | 55000 ± 21700 | 6.11 ± 1.28 |
| 1600 mg | 826000 | 123000 | 4.48 |

*AUC₀₋ₗₐₛₜ (Mean ± SD, h · ng/mL). “—” indicates the information is not available.

### Example 6: Observational Clinical Trial

### I. Introduction

An observational clinical trial was run to determine and characterize the presence of NMJ transmission abnormalities in patients with CMT1 or CMT2. The primary aim was to investigate NMJ transmission deficits in CMT types 1 and 2 patients compared with age-matched healthy controls.

### II.1. Study Overview and Timeline

The study protocol was reviewed and approved by The Ohio State University Wexner Medical Center Institutional Review Board and The Central Denmark region Committees on Health Research Ethics. All participants, both CMT patients and healthy controls, provided written informed consent before study participation. CMT participants underwent 4 clinic evaluations. After the baseline visit the between visit interval was 2 weeks +/−10 days (FIG. 4). Each visit involved clinical and electrophysiological testing. Healthy controls underwent electrophysiological testing at a single visit (Visit 1). (FIG. 4). The study was conducted in accordance with the Declaration of Helsinki and registered in the database Clinicaltrials.gov (NCT04980807)

### II.2. Participants

Study enrolment included participants with CMT types 1 and 2 and aged-matched healthy controls. The inclusion and exclusion criteria for CMT participants and healthy controls are listed in Tables 13 and 14 respectively. The inclusion and exclusion criteria were defined to ensure that the CMT diagnosis was certain, that participants could complete the physical tests, and that their condition was stable in order to eliminate factors that could potentially influence NMJ function.

TABLE 13
Inclusion and exclusion criteria for CMT participants
CMT Participants

| Inclusion criteria | Exclusion criteria |
|---|---|
| Age ≥18 years | Ulceration that would interfere with |
| | functional ability |
| Diagnosis of CMT Type 1 or 2 via: | Severe deformity or ankle contracture |
| genetic testing; or | that would sufficiently limit passive |
| a combination of clinical presentation | range of motion to affect assessment of |
| and electrodiagnostic testing | dorsiflexion strength |
| Physical ability (all must apply): | Significant change in physical activity or |
| ambulate 10 meters without bracing | exercise (e.g. significant in- or |
| bilateral ankle plantarflexion strength | decrease in intensity or frequency) |
| between 2− and 5 (inclusive) on the | within 8 weeks before Study Day 1 or |
| Medical Research Council (MRC) | inability to maintain the baseline level of |
| scale | physical activity throughout the study |
| bilateral ankle dorsiflexion strength | |
| between 2− and 4+ (inclusive) on the | |
| MRC scale | |
| Ability to adhere to study schedule and | Moderate to severe neuropathic or |
| procedures | inflammatory/musculoskeletal pain that |
| | would interfere with performance of the |
| | tests |
| Stable concomitant medications for 2 | A diagnosis of a primary NMJ disorder |
| months prior to enrolment | such as myasthenia gravis, Lambert |
| | Eaton Myasthenic Syndrome or |
| | congenital myasthenic disorder |
| Ability to provide written informed consent | Any disability or condition that would |
| | prevent completion of the study tasks |
| | Recent major surgery |

TABLE 14
Inclusion and exclusion criteria for healthy control participants
Healthy Control Participants

| Inclusion criteria | Exclusion criteria |
|---|---|
| Age ≥18 years | Pregnant |
| Healthy male or female | Requiring prescription medicines likely to |
| volunteers | interfere with electromyography (EMG) |
| | recordings |
| Ability to provide written | Presence of current or previous medical |
| informed consent | condition which might interfere with |
| | participation in the study |

### II.3. Study Outcomes

Outcome measures included NMJ electrophysiological testing, isometric dynamometry, Manual Muscle Testing, 10-meter walk/run time, Timed Up and Go, 6-minute walk test, Berg Balance Scale (total score), Six-Spot Step Test, 9-hole peg test, and CMT Examination Score (CMTES2). Adverse events (AEs) were collected throughout the study. Tolerability of outcome testing was evaluated in both healthy control participants (electrophysiological tests) and CMT participants (electrophysiological and clinical tests) at all study visits using on a scale from 0-9 with 0 indicating no discomfort and 9 indicating worst possible discomfort.

### II.3.A. NMJ Electrophysiology

Single fibre electromyography (SFEMG) was performed using a clinical electrodiagnostic system [Ohio State University: Cadwell Sierra Wave (Kennewick, WA, USA)] a concentric needle recording electrode (25 mm×30 gauge, Dantec) and 1-10 kHz for high- and low-pass filter settings to record and analysed up to 22 apparent single muscle fibre action potential pairs during voluntary contraction. A total of 50-100 consecutive discharges were analysed for each apparent single muscle fiber pair to determine parameters of jitter (variability of NMJ transmission) and blocking (i.e., Failure of NMJ transmission). Repetitive Nerve Stimulation (RNS) at 3 Hz stimulation rate was performed on trapezius and abductor pollicis brevis muscles. Compound muscle action potential (CMAP) decrement of amplitude and area of 1st to 5th and 1st to 10th stimuli was quantified.

### Manual Muscle Testing (MMT)

Manual muscle testing (MMT) was performed on 15 muscle groups, including shoulder abduction, elbow flexion/extension, wrist flexion/extension, long thumb flexors, hip flexors/extensors, hip abductors, knee flexors/extensors, ankle dorsi flexors/plantar flexors, and neck flexors/extensors. All but the neck muscle groups were tested bilaterally. Positioning and grading were standardized and applied as previously described (Personius, 1994; Kendall, 2005). The modified Medical Research Council (MRC) scale was translated into a 0-10 ordinal scale that was used for analysis (Table 15).

TABLE 15
Translation table for converting MRC score into ordinal scale score

| MRC Grade | Value for Analysis |
|---|---|
| 5 | 10 |
| 4+ | 9 |
| 4 | 8 |
| 4− | 7 |
| 3+ | 6 |
| 3 | 5 |
| 3− | 4 |
| 2+ | 3 |
| 2 | 2 |
| 2− | 1 |
| 1 | 0 |
| 0 | 0 |

### Isometric Dynamometry

Isometric dynamometry was performed using each site's respective available equipment. The Ohio State University utilized the Accurate Test of Limb Isometric Strength (ATLIS) chair, which is a fixed frame apparatus with an adjustable wireless loadcell. Ankle dorsiflexion was tested in an upright seated position; positioning was otherwise standardized and applied as previously described (Andres et al, 2012). Aarhus University Hospital utilized a Biodex Isokinetic Dynamometer, SYSTEM 3 PRO (Biodex Medical Systems, Inc.). Ankle dorsiflexion was tested on the non-dominant side. Positioning was standardized and applied as previously described (Harbo et al, 2012). A minimum of two trials were performed; additional trials were performed if there was greater than 15% variability. Since different methods were used, data collected for the two sites were not combined for analysis. The highest value for each muscle group was recorded.

### 11.3.B. Functional and Assessments

### Nine-Hole Peq Test (9HPT)

The nine-hole peg test (9HPT) is a timed test of finger dexterity and hand function. Participants place 9 pegs from a reservoir into holes aligned in a 3-by-3 grid and then move them back to the reservoir again one at a time. The 9HPT has been shown to be reliable and responsive in people with CMT (Svensson et al, 2006; Solari et al, 2008; Piscosquito et al, 2015). Set up and procedure were standardized as previously described (Mathiowetz et al, 1985). Two trials were performed with each hand; trials were averaged and used for analysis.

### Six-Spot Step Test (SSST)

The Six-spot Step Test (SSST) is a timed test of lower extremity function and balance. Participants walk in a standardized zig-zag pattern through a 1×5-meter course, sliding cylindrical blocks with their tested foot out of the course as they move through it (Nieuwenhuis et al, 2006). This test has been shown to be valid and reliable in neurodegenerative conditions and older adults (Sandroff et al, 2015; Brincks et al, 2019; Kondori et al, 2020); it has also shown responsiveness in polyneuropathy (Kreutzfeldt et al, 2017). Two trials were performed with each foot; trials were averaged and used for analysis. Participants were permitted to use their typical bracing and/or assistive devices, which was documented at each visit.

Ten-Meter Walk/Run Test (10mWRT)

The 10-meter Walk/Run Test (10mWRT) is a timed assessment meant to capture maximal performance over a short distance and does not suffer from the same ceiling effects as a test that permits only walking regardless of the person's ability (Krosschell et al, 2022). It has been shown to be reliable in CMT (Bray et al, 2020). Two trials were performed and averaged for analysis. Participants were permitted to use their typical bracing and/or assistive devices, which was documented at each visit.

### Timed Up and Go (TUG)

The Timed Up and Go (TUG) is a timed mobility assessment involving rising from a chair, walking 3 meters at a comfortable pace, and returning to sit in the chair (Podsiadlo et al, 1991). It has been validated neurologic, neuromuscular, and geriatric populations (Dunaway et al, 2014). Two trials were performed and averaged for use in data analysis. Participants were permitted to use their typical bracing and/or assistive devices, which was documented at each visit.

### Six-Minute Walk Test (6MWT)

The Six-minute Walk Test (6MWT) is a measure of endurance and can also provide an index of fatigability (Montes et al, 2010). Validity has been established in people with CMT (Mori et al, 2020). The test was administered according to the American Thoracic Society guidelines on a 30-meter course with standardized cues (ATS, 2002). Participants were permitted to use their typical bracing and/or assistive devices, which was documented at each visit. A 10-minute rest was required prior to the start of the test. Total distance as well as percent change in distance walked between the first and sixth minutes were used for analyses.

### Berg Balance Scale (BBS)

The Berg Balance Scale (BBS) is a 14-item assessment of balance during functional tasks. Each item is scored on a 0 to 4 scale where higher scores indicate better performance. The total score used for analysis is the sum of the individual scores for each item with a maximum score of 56. It has demonstrated validity in CMT (Bragadin et al, 2015). Participants were permitted to wear bracing but could not use mobility devices. Items were administered as previously described (Berg et al, 1989).

### CMT Examination Score (CMTES2)

The CMT Examination Score version 2 is a composite scoring system to assess sensory and motor impairment in subjects with CMT. It is a subscore of the CMT Neuropathy Score. Each item is scored on a 0-4 ordinal scale where higher scores indicate greater severity of impairment. It has demonstrated excellent inter- and intra-rate reliability in people with CMT (Murphy et al, 2011).

### II.4. Statistical Analysis

Statistical analyses were performed using SAS by an independent statistical consultancy group, Phastar. Statistical coding was performed prior to database lock and in accordance with the statistical analysis plan for the study. For the primary aim comparing NMJ transmission in CMT patients and controls, data that followed parametric (normal) distribution was analysed with unpaired t-tests. For data with unequal variances, Welch's test was used. For data that did not follow parametric distribution, a Mann Whitney test was applied. For the secondary aim of test-retest reliability interclass correlation coefficient (ICC) were calculated. For the exploratory analysis investigating associations between electrophysiological and clinical outcomes, Spearman correlation coefficients were calculated. No corrections for multiple testing were performed due to the exploratory nature of the study.

### III. Results

III.1. Study Participants, Demographics, Baseline Characteristics, and Adverse Events A total of 23 CMT patients and 10 healthy controls were screened for participation. A total of 21 CMT patients and 10 healthy controls were enrolled. Of those, 18 CMT patients and 10 healthy controls completed all visits while 2 CMT patients dropped out after visit 3 (1 patient was unable to make the final visit within the allowed time-period and another enrolled in a drug study), and 1 patient withdrew consent before first visit for personal reasons. For these 3 subjects, available data are included in the analyses. Demographic and baseline characteristics are presented in Table 16. No AEs were reported for the healthy controls. A total of 4 AEs were reported in CMT patients: 1 patient fell during 6MWT—graded mild, 1 patient fell during 10MWT—graded mild, 1 patient experienced knee pain following isometric dynamometry—graded moderate, and 1 patient had a syncope—graded mild.

TABLE 16
Demographics and Clinical Baseline Characteristics

| | HC | CMT | CMT1 | CMT2 |
|---|---|---|---|---|
| N | 10 | 21 | 16 | 5 |
| Age (Years) | 43.3 ± 12.68 | 46.4 ± 14.4 | 43 ± 11.7 | 56 ± 19.3 |
| Gender (M/F) | 4/6 | 5/16 | 3/13 | 2/3 |
| CMTES2 Total | NA | 10.1 ± 3.2 | 10.3 ± 2.7 | 9.4 ± 3.4 |
| Score | | N = 20 | N = 15 | N = 5 |
| 9 Hole Peg Test (s) | NA | 21.6 ± 4.7 | 21.9 ± 5.3 | 21.0 ± 2.2 |
| | | N = 20 | N = 15 | N = 5 |
| 6 Spot Step Test | NA | 9.9 ± 2.6 | 9.5 ± 2.7 | 10.9 ± 1.8 |
| (s) | | N = 20 | N = 15 | N = 5 |
| 10 Meter Walk/Run | NA | 6.1 ± 2.1 | 5.8 ± 1.8 | 6.9 ± 3.1 |
| Test (s) | | N = 20 | N = 15 | N = 5 |
| Timed Up and Go | NA | 7.9 ± 1.9 | 7.4 ± 1.6 | 9.4 ± 2.2 |
| (s) | | N = 20 | N = 15 | N = 5 |
| Berg Balance Scale | NA | 51.3 ± 5.0 | 52.1 ± 4.8 | 49.0 ± 5.6 |
| Total Score | | N = 20 | N = 15 | N = 5 |
| Manual Muscle | NA | 249.6 ± 17.3 | 251.2 ± 15.6 | 245.2 ± 23.0 |
| Testing Total Score | | N = 19 | N = 14 | N = 5 |
| Non-Dominant | NA | 12.0 ± 4.5 | 12.1 ± 4.8 | 11.3 |
| Ankle Dorsi Flexor | | N = 9 | N = 8 | N = 1 |
| Strength - Aarhus | | | | |
| (Nm) | | | | |
| Right Ankle Dorsi | NA | 95.9 ± 58.9 | 96.9 ± 65.5 | 94.6 ± 62.9 |
| Flexor Strength - | | N = 7 | N = 4 | N = 3 |
| Ohio (N) | | | | |
| 6 Minute Walk Test | NA | 455.3 ± 102.8 | 464.9 ± 93.7 | 426.7 ± 134.8 |
| Total Distance (m) | | N = 20 | N = 15 | N = 5 |
| 6 Minute Walk Test | NA | −6.6 ± 13.9 | −5.2 ± 14.7 | −10.8 ± 11.2 |
| 6th/1st minute (%) | | N = 20 | N = 15 | N = 5 |

Data are mean ± SD.

### III.2.CMT Patients Demonstrate Notable NMJ Transmission Deficits Compared with Healthy Controls

For the primary aim, comparing NMJ transmission on SFEMG and RNS between CMT patients and healthy controls, data from study visit 1 (baseline) was used. Median SFEMG jitter, a measure of NMJ transmission reliability, was increased by >90% in CMT patients compared with healthy controls [CMT: median jitter 56.3 μs (range: 35.3; 196.6) versus Healthy controls: median 29.4 μs (range: 18.6; 35.6), p<0.05](FIG. 5A). Similarly, SFEMG blocking, a measure of NMJ transmission failure, was increased in CMT patients [CMT: median blocking 13.39% (range: 0; 90.9) versus Healthy controls: median 0% (range: 0; 4.5), p<0.05]. In addition to comparing CMT patients to healthy controls, SFEMG jitter was also compared between CMT1 and CMT2. Jitter was increased in CMT2 versus CMT1 [CMT2: median jitter 100.2 μs (range: 62.2; 152.2) versus CMT1: median jitter 49.3 μs (range: 35.3; 196.6), p<0.05](FIG. 5C). Similarly, SFEMG blocking was increased in CMT2 versus CMT1 [CMT2: median blocking 75.0% (range: 12.5; 87.5) versus CMT1: median blocking 10.0% (range: 0.0; 90.9), p<0.05](FIG. 5D).

Compound muscle action potential (CMAP) amplitude was assessed following a single supramaximal stimulation and following a train of 10 stimulations at 3 Hz. CMAP amplitude following a single stimulation demonstrated ~40% reduction in CMT patients compared with healthy controls recording from the abductor pollicis brevis (APB) muscle and ~35% reduction in CMT patients compared with healthy controls recording from the trapezius muscle. In contrast to SFEMG, RNS at 3 Hz demonstrated no significantly increased decrement when comparing the 1st stimulation to both the 5th and 10th stimulation in a train of 10 stimulations and recording from both the APB and trapezius muscles. Neither CMAP amplitude following a single stimulation nor decrement following a train of stimuli showed differences between CMT1 and CMT2 for the APB or trapezius muscles.

### IV.3. Secondary Analysis—Test-Retest Reliability

Test-retest reliability was assessed for both electrophysiological and clinical outcomes by calculating interclass correlation coefficient (ICC) values based on data from study visit 1-4 from CMT patients. Test-retest reliability estimates between visits are summarised in Table 17.

TABLE 17
Test-Retest Reliability

| | | | | | |
|---|---|---|---|---|---|
| Outcome Measure | ICC | Visit 1 | Visit 2 | Visit 3 | Visit 4 |

Electrophysiological Outcomes

| | | | | | |
|---|---|---|---|---|---|
| Jitter (us) | 0.50 | 75.6 ± 47.7 | 76.2 ± 47.7 | 66.3 ± 33.4 | 71.2 ± 38.5 |
| Blocking (%) | 0.48 | 29.9 ± 33.2 | 32.0 ± 30.3 | 25.2 ± 26.9 | 23.8 ± 23.4 |
| CMAP Decrement of Area | 0.14 | −9.9 ± 8.7 | −10.1 ± 4.4 | −7.5 ± 4.2 | −7.4 ± 5.5 |
| Stim 1-10 APB (%) | | | | | |
| CMAP Decrement of Area | 0.01 | −7.8 ± 4.8 | −8.3 ± 4.7 | −9.0 ± 4.6 | −6.5 ± 3.6 |
| Stim 1-10 Trapezius (%) | | | | | |

Clinical Outcomes

| | | | | | |
|---|---|---|---|---|---|
| 10 Meter Walk/Run Test | 0.97 | 6.1 ± 2.1 | 5.9 ± 2.2 | 5.8 ± 2.3 | 5.5 ± 2.0 |
| (s) | | | | | |
| 9 Hole Peg Test (s) | 0.92 | 21.6 ± 4.7 | 21.3 ± 5.2 | 20.2 ± 5.0 | 20.0 ± 3.0 |
| Right Ankle Dorsi Flexor | 0.88 | 95.9 ± 58.8 | 83.7 ± 46.3 | 93.4 ± 48.6 | 100.8 ± 60.7 |
| Strength - Ohio (N) | | | | | |
| Non-Dominant Ankle Dorsi | 0.87 | 12.0 ± 4.5 | 13.5 ± 4.3 | 13.7 ± 4.1 | 13.8 ± 5.8 |
| Flexor Strength - Aarhus | | | | | |
| (Nm) | | | | | |
| 6 Minute Walk Test Total | 0.81 | 455.3 ± 102.9 | 474.4 ± 101.0 | 481.3 ± 103.9 | 490.4 ± 90.8 |
| Distance (m) | | | | | |
| 6 Spot Step Test (s) | 0.79 | 9.9 ± 2.6 | 9.1 ± 2.8 | 8.8 ± 2.6 | 8.3 ± 2.5 |
| Timed Up and Go (s) | 0.76 | 7.9 ± 1.9 | 7.6 ± 1.8 | 7.6 ± 1.9 | 7.3 ± 1.6 |
| Berg Balance Scale | 0.72 | 51.3 ± 5.1 | 53.2 ± 3.4 | 53.9 ± 3.3 | 54.0 ± 3.3 |
| Manual Muscle Testing | 0.69 | 249.6 ± 17.3 | 256.3 ± 12.4 | 256.5 ± 13.2 | 259.7 ± 10.2 |
| 6 Minute Walk Test 6th/1st | 0.52 | −6.6 ± 13.9 | −6.5 ± 12.5 | −3.0 ± 7.7 | −3.3 ± 6.0 |
| minute (%) | | | | | |

Data are mean ± SD and interclass correlation coefficient (ICC).

### IV.4. Secondary Analysis—Tolerability of Electrophysiological and Clinical Outcomes

Tolerability scores for the different procedures at each visit for CMT patients and healthy controls are summarised in Table 18. Tolerability was evaluated on a scale from 0-9 with 0 indicating no discomfort and 9 indicating worst possible discomfort.

TABLE 18
Tolerability of Electrophysiological and Clinical Outcomes

| | | | | | |
|---|---|---|---|---|---|
| | HC | CMT | CMT | CMT | CMT |
| Outcome Measure | Visit 1 | Visit 1 | Visit 2 | Visit 3 | Visit 4 |

Electrophysiological Outcomes

| | | | | | |
|---|---|---|---|---|---|
| SFEMG | 2 (1-6) | 3.5 (0-6) | 2.5 (0-9) | 3 (0-9) | 2.5 (0-8) |
| RNS - Trapezius | 2 (0-7) | 4.5 (0-8) | 4 (0-8) | 5 (0-9) | 4(0-9) |
| RNS - APB | 3 (0-6) | 4 (1-8) | 5 (1-9) | 3 (0-9) | 3 (0-8) |

Clinical Outcomes

| | | | | | |
|---|---|---|---|---|---|
| CMTES2 | NA | 0 (0-2) | NA | NA | NA |
| 9 Hole Peg Test | NA | 0 (0-4) | 0 (0-4) | 0 (0-4) | 0 (0-4) |
| 6 Spot Step Test | NA | 0 (0-4) | 0 (0-4) | 0 (0-4) | 0 (0-4) |
| 10 Meter Walk/Run | NA | 0.5 (0-4) | 1 (0-4) | 1 (0-4) | 0 (0-4) |
| Test | | | | | |
| Timed Up and Go | NA | 0 (0-4) | 0 (0-4) | 0 (0-4) | 0 (0-4) |
| Berg Balance Scale | NA | 1 (0-7) | 0 (0-5) | 0 (0-6) | 0.5 (0-3) |
| Manual Muscle Testing | NA | 0.5 (0-5) | 1 (0-3) | 0 (0-4) | 1 (0-4) |
| Isometric Dynamometry | NA | 1.5 (0-5) | 2 (0-5) | 2 (0-4) | 0.5 (0-4) |
| 6 Minute Walk Test | NA | 2 (0-5) | 1 (0-6) | 1.5 (0-5) | 1 (0-5) |

Data are medians with range

IV.5. NMJ Transmission Defects are Correlated with Measures of Strength and Function in CMT

Associations between electrophysiological and clinical endpoints are summarized in Table 19. Clinical parameters related to strength, balance and mobility were associated with jitter and blocking. 6 Spot Step Test, 10 Meter Walk/Run Test and Timed Up and Go were positively associated with jitter and blocking indicating that longer time to complete these tasks (worse performance) are associated with higher levels of jitter and blocking (worse NMJ transmission). Isometric ankle dorsi flexor strength, the total score of the Berg Balance Scale, and 6 Minute Walk Test were negatively associated with jitter and blocking indicating that lower performance in these tasks were associated with higher levels of jitter and blocking. CMTES2, Manual Muscle Testing and 9 Hole Peg Test were not associated with jitter and blocking. In general, and in contrast to SFEMG measurements, clinical endpoints were not associated with CMAP decrement assessed with RNS (data not shown).

TABLE 19
Association of clinical outcomes with jitter and blocking

| | Association with | Association with |
|---|---|---|
| Clinical Outcome Measure | Jitter | Blocking |
| Right Ankle Dorsi Flexor Strength - Ohio | −0.893 (p = 0.007) | −0.750 (p = 0.052) |
| Non-Dominant Ankle Dorsi Flexor Strength - Aarhus | −0.783 (p = 0.013) | −0.630 (p = 0.069) |
| 6 Spot Step Test | 0.674 (p = 0.001) | 0.533 (p = 0.015) |
| Berg Balance Scale Total Score | −0.608 (p = 0.004) | −0.548 (p = 0.012) |
| Timed Up and Go | 0.606 (p = 0.005) | 0.457 (p = 0.043) |
| 6 Minute Walk Test Total Distance | −0.598 (p = 0.005) | −0.484 (p = 0.030) |
| 10 Meter Walk/Run Test | 0.541 (p = 0.014) | 0.333 (p = 0.137) |
| 6 Minute Walk Test 6th / 1st minute | −0.474 (p = 0.035) | −0.616 (p = 0.004) |
| 9 Hole Peg Test | 0.284 (p = 0.225) | 0.016 (p = 0.947) |
| Manual Muscle Testing Total Score | −0.190 (p = 0.437) | −0.067 (p = 0.784) |
| CMTES2 Total Score | 0.081 (p = 0.733) | 0.018 (p = 0.940) |

Associations between SFEMG parameters and clinical outcomes are Spearman's correlations. Isometric dynamometry was performed by separate procedures by Aarhus and Ohio and hence these data were analysed by site.

### V. Discussion and Conclusions

As the final link between the nervous system and skeletal muscle, reliable NMJ transmission is critical for neural control of muscle contraction. Based on sparse animal studies and findings in other motor axonal and motor neuronal disorders, we hypothesized that patients with CMT would demonstrate features of NMJ transmission deficits. We further hypothesized that NMJ transmission deficits would be correlated with assessments of disease severity and function. Using SFEMG, we confirmed that CMT patients have notable NMJ transmission deficits compared with healthy age-matched controls. Furthermore, NMJ transmission deficits showed significant correlations with clinical outcome measures of strength, mobility, and balance.

This study further provides reliability and tolerability estimates for clinical and electrophysiological outcomes in CMT patients. In general, clinical outcome measures were well tolerated and showed moderate to excellent reliability. SFEMG showed better reliability and tolerability than RNS, but both tests were associated with stronger discomfort in a subset of patients.

Two standard clinical electrophysiological assessments of NMJ transmission were applied in the current study. Notably, SFEMG demonstrated clear evidence for NMJ transmission deficits. The fact that jitter and blocking showed significant correlations with clinical outcomes raises the possibility of targeting NMJ transmission dysfunction to improve symptoms in patients with CMT. In contrast to SFEMG parameters, CMAP amplitude decrement recorded from the APB and trapezius muscle following a train of stimuli at 10 Hz demonstrated no significant differences with age-matched healthy controls. This important finding supports the high sensitivity of SFEMG for detecting NMJ transmission defects which is aligned with prior studies in primary NMJ disorders such as myasthenia gravis (Witoonpanich et al, 2011). It is also worth noting that SFEMG was better tolerated, showed better reliability, and was more closely associated with clinical outcomes compared with RNS. The improved reliability of SFEMG as compared with RNS may at least partly explain why jitter and blocking were able to clearly distinguish patients from controls and CMAP decrement was not (i.e. the sample size was too low to detect a difference).

Another interesting result of our study includes the finding of more severe NMJ transmission deficits in CMT2 patients as compared to CMT1 patients. It is also worth noting that CMT2 patients were weaker and performed worse on outcomes related to gait and balance. This is consistent with previous studies demonstrating that at a given age gait patterns and muscle strength are more severely affected in CMT2 than CMT1 patients (Pogemiller et al, 2020). Interestingly, when comparing trapezius and APB CMAP amplitudes between CMT1 and CMT2, there were no significant differences in our study suggesting relatively similar burden of axonal loss between the cohorts. Together, these findings suggest that NMJ transmission deficits may disproportionately impact patients with CMT2 compared to CMT1 and raises the possibility that this contributes to decreased muscle strength and performance. Perhaps contrasting this possibility is the finding of very high levels of jitter and blocking in two patients in the CMT1 cohort. Even though demyelination is the primary pathology in CMT1 patients, axonal degeneration (primary pathology in CMT2 patients) is a common long-term consequence of demyelination (Scherer et al, 2011). Thus, it could be speculated that while these patients have a type 1 genotype their phenotype may resemble CMT2 patients. It is however also worth noting that the high levels of jitter and blocking seen in these two CMT1 patients at visit 1 dropped down to levels seen in other CMT1 patients at visit 2-4. Finally, it should be emphasized that due to limited sample size and age differences between CMT1 and CMT2 patients further work is needed to investigate genotype phenotype correlations between NMJ deficits, functional status and CMT type.

In conclusion, the current study demonstrates for the first time NMJ transmission deficits in patients with CMT types 1 and 2. This deficit seems to be more exacerbated in type 2 CMT patients. In general, clinical tests were well tolerated and showed moderate to excellent reliability. SFEMG was better tolerated and showed better reliability as compared to RNS. Clinical outcomes of strength mobility and balance were associated with jitter and blocking.

### Example 7: NMD670 in rat model of CMT1A

### Purpose

To evaluate the increase of neuromuscular transmission and muscle force generation in a mouse model of CMT1A following a single IP injection of NMD670.

Male and female mice (Jacson Laboratory Strain #:030052) approx. 6-7 months of age at time of study start were used. These genetically modified mice are used as a disease model to study Charcot Marie Tooth (CMT) disease type 1A (CMT1A).

TABLE 20
Groups and dose levels

| | Animal | | | Wash out | | |
|---|---|---|---|---|---|---|
| Group | numbers | Test 1 | Dose | for 48 h | Test 2 | Dose |
| 1 | 1-4 | Vehicle | 0 | | NMD670 | 10 mg/kg |
| | | (PBS) | | | | 1 mg/ml |
| 2 | 5-8 | NMD670 | 10 mg/kg | | Vehicle | 0 |
| | | | 1 mg/ml | | (PBS) | |

### Test Equipment Systems

EMG equipment: Cadwell Sierra Summit EMG machine Kennewick, WA, clinically approved for electrical stimulation and recording of EMG signals. Electrical stimulators and recording electrodes were made sterile before use.

Force equipment: Aurora 1300A in vivo force measure for mice.

Anaesthesia: Somnosuite® low-flow anaesthesia system (Kent Scientific, Torrington, CT) isoflurane inhalation at 2% isoflurane at 300 mL 02 flow.

### Dose and Administration

The sodium salt of NMD670 was dissolved in phosphate buffered saline (PBS) to a dose volume of 1 mg/mL. The entire amount of dose-solution was then transferred to a vial labelled A or B (for blinding purposes). The vehicle control solution was PBS and this was transferred to a vial labelled A or B (for blinding purposes). Compound was dosed IP at a volume of 10 mL/kg, from a 1 mg/mL dose solution.

### Identification

Animals were identified by ear-punches.

### Groups

Animals were randomized into group A or B based on: Bodyweight, EMG response at initial assessment, and force production at initial assessment.

Only animals that display clear phenotype of neuromuscular transmission defect (identified by decrement on EMG and/or reduced force production) were used. Animals that did not show EMG decrement or reduced force at the initial test during the study, were not included.

### Housing, Bedding and Environment

Animals were group housed 2 individuals per cage, at 20-23° C. at 55% humidity, on corncob bedding with at a 12:12 hour light/dark cycle.

### Water and Diet

Free access to water and chow (fed ad libitum). NIH diet 7913

### Enrichments Used in the Cage

All mouse cages received one 2 in square Nestlet™, purchased from AnCare. Cages also contained a single 4g Bed-r' Nest™ Crinkle paper puck, manufactured by The Andersons Plant Nutrient company. All enrichment was autoclaved prior to use. Other enrichment options included paper Bio-Huts™ from Bio-Serv, hard plastic mouse “Enrichment Lofts” From Allentown, wood gnawing blocks from Bio-Serv, and Twist-n'Rich™ paper twists from The Andersons Plant Nutrient company.

### Test Procedure and Method

### Force 1

Animal was sedated (isoflurane inhalation 2% in ambient air) and prepared for assessment of force production from the hind leg, via electrical stimulation of the sciatic nerve in a static position. Stimulation electrodes were inserted at the sciatic nerve, and test pulses of low current amplitude were used to adjust position of all electrodes until a stable reproducible signal was achieved twice. The force resulting from supramaximal electrical stimulation of the supplying motor nerve was recorded from a foot-pad moved by the foot/paw of the animal. See table Table 21 for detailed stimulation protocol. The baseline of tension in the hindleg/foot was adjusted throughout the experiment to ensure comparable results/avoid drift.

### EMG 1

Animal was transferred to recording station for EMG measures. Animal remained sedated throughout the procedure. Electrodes were inserted under the skin to electrically stimulate the Sciatic nerve, and recording ring electrodes were placed at the lower leg of the animal. Test pulses of low current amplitude were used to adjust position of all electrodes until a stable reproducible signal was achieved twice. Then a series of electrical stimulation was initiated with fixed frequencies and durations, see Table 22, for details. This recorded the Compound Muscle Action Potentials (CMAP). No oscillations that would indicate artifact/moving electrodes (such as smooth decrement if present) were noted confirming that electrodes were still in place at end.

NMD670 or vehicle was administered by IP injection at a volume of 10 mL/kg from a 1 mg/mL dose solution while sedated in the EMG measurement set up. 10 to 15 minutes was allowed to elapse before next step was initiated.

### EMG 2

The procedure from EMG 1 was repeated. Adjustment of electrodes was performed then CMAP was recorded.

### Force 2

The animal remained sedated and prepared for assessment of force production from the hind leg, via electrical stimulation of the sciatic nerve in a static position. Stimulation electrodes were inserted at the sciatic nerve, and test pulses of low current amplitude were used to adjust position of all electrodes until a stable reproducible signal was achieved twice. The force resulting from supramaximal electrical stimulation of the supplying motor nerve was recorded from a foot-pad moved by the foot/paw of the animal. See Table 21 for detailed stimulation protocol. The baseline of tension in the hindleg/foot was adjusted to similar levels as in “Force 1” if possible and then throughout the experiment to ensure comparable results/avoid drift.

Baseline torque, +−0.25 mNm (milliNewtonmeter)

### Return to Home Cage

The animal was released from the set up and returned to home cage where it was allowed to recover until fully mobile (under observation).

TABLE 21
test of in vivo force
Mice were sedated and mounted to record in vivo force.
A protocol of a range of frequencies (1, 15, 30, 60, 120 and
200 Hz) for 1 second duration, at 0.2 ms pulse-duration.
The optimal stimulation voltage/current was identified for each
animal individually by adjusting the stimulation and the tension
of the muscle/tendon on the foot-plate. When optimised, the range
of stimulation frequencies was tested, as follows:

| Stimulation Frequency | Pause until next stimulation: |
|---|---|
| 1 Hz, | wait 5 seconds |
| 15 Hz, | wait 10 seconds |
| 30 Hz | wait 10 seconds |
| 60 Hz | wait 60 seconds |
| 120 Hz | wait 120 seconds |
| 200 | END |

TABLE 21
test of EMG - CMAP
A protocol of a range of frequencies (10 to 50 Hz) for
10 pulses, various duration, at 0.2 ms pulse-duration
was used. The optimal stimulation voltage/current was
identified for each animal individually by adjusting the
stimulation and the position of the recording electrodes.
All stimulations were performed once per test.

| Stimulation Frequency | Pause between stimulation |
|---|---|
| 10 Hz, | wait 10 seconds |
| 20 Hz, | wait 10 seconds |
| 30 Hz | wait 10 seconds |
| 40 Hz | wait 10 seconds |
| 50 Hz | wait 10 seconds |

### Results and Discussion

Each mouse was tested prior to intervention with test article, and then tested after IP injection of 10 mg/kg NMD670 or vehicle (PBS). A washout period was then allowed (48 hours), and the mouse was subjected to the same test, but receiving the opposite treatment to that on the first day. All mice thus received both vehicle and NMD670, in a randomized order (FIG. 6).

Primary endpoints were CMAP from stimulated EMG (RNS), and force from sciatic nerve stimulation. 11 animals were screened in the study, and 8 were included into the study.

CMAP decrement was not significantly different between the two groups prior to intervention, nor after treatment (FIG. 7). Both groups displayed a tendency to reduced decrement after intervention, with animals receiving vehicle recovering 8.6% point at 40 Hz, and animals receiving NMD670 recovering 15.2% point at 40 Hz.

Peak muscle force increased in animals receiving NMD670, but not in animals receiving vehicle (FIG. 8). At 60 Hz, force increased 1.13 mNm in animals receiving NMD670, while it declined 1,49 mNm in animals receiving vehicle. Similarly force increased 1.67 mNm in animals receiving NMD670, while it declined 1.36 mNm in animals receiving vehicle at 120 Hz. This difference was statistically significantly different at all tested stimulation frequencies, except at 30 Hz (table 22).

TABLE 22
Difference in peak stimulated force, from before to after treatment

| Stimulation | Change in Torque (mN*m) | | Significance |
|---|---|---|---|
| Frequency (Hz) | NMD670 | Vehicle (PBS) | (two-way RM ANOVA) |
| 1 | 0.95 | −0.37 | 0.048 |
| 15 | 0.59 | −0.46 | 0.0003 |
| 30 | 0.49 | −0.95 | 0.082 |
| 60 | 1.13 | −1.49 | 0.0016 |
| 120 | 1.67 | −1.36 | 0.0029 |
| 200 | 1.90 | −1.42 | 0.0018 |

The ability to sustain force during stimulated contraction was also markedly improved in animals receiving NMD670 compared to Vehicle (FIG. 9). The relative force from the initial part of the stimulation to the end of stimulation improved at all tested frequencies in animals receiving NMD670 but declined in all animals receiving vehicle. The ability to sustain force at 200 Hz improved 18.3% point in animals receiving NMD670, while it decreased 24% in animals receiving vehicle. This difference was statistically significantly different at 120 and 200 Hz stimulation frequencies (table 23).

TABLE 23
Difference in fade, from before to after treatment

| Stimulation | Change in Fade (%) | | Significance |
|---|---|---|---|
| Frequency (Hz) | NMD670 | Vehicle (PBS) | (two-way RM ANOVA) |
| 60 | 2.2 | −1.5 | 0.91 |
| 120 | 8.1 | −14.1 | 0.0031 |
| 200 | 18.3 | −24.1 | <0.0001 |

### CONCLUSION

10 mg/kg NMD670 administered intra peritoneally was able to significantly increase stimulated force in an animal model of Charcot-Marie-Tooth disease type 1A, when compared to vehicle treatment.

### Example 8: Phase IIA Clinical Trial

A phase 2a, randomised, double-blind, placebo-controlled study to evaluate the efficacy, safety, and tolerability of NMD670 over 21 days in ambulatory adult patients with Type 1 and Type 2 Charcot-Marie-Tooth disease.

Overall Design Summary:

This is a Phase 2, multicentre, randomised, double-blind, placebo-controlled study.

The study will enroll ambulatory male and female participants diagnosed with CMT types 1 and 2 and enriched to reach approximately 30% participants with CMT type 2. Randomization will be stratified by CMT type, sex and age (≥45 years), which will allow for sensitivity analysis. After screening, eligible participants will be randomised to receive placebo or NMD670 400 mg bis in die/twice a day (BID) for 21 days. Both the participant and Investigator will be blinded to treatment assignment and will not know which treatment is given during the treatment period.

### BRIEF SUMMARY

The study will evaluate the efficacy, safety, and tolerability of oral administration of NMD670. The intended population is ambulatory male and female participants aged 18 to 70 years with a genetically confirmed diagnosis of CMT type 1 or type 2. Randomisation will be stratified by CMT type, sex and age (≥45 years). Patients are eligible if the following criteria are met; 1) complete 6MWT within 1 standard deviation of the provided CMT norm, and 2) have a bilateral ankle dorsiflexion strength of 4 (inclusive) or less on manual muscle testing. To participate in this study, participants will be required to have a body mass index between 18 and 35 kg/m² and a minimum weight of 40 kg. Key exclusion criteria include use of daily assistive devices such as ankle-foot orthosis, a severe deformity or ankle contracture that would limit range of motion, clinical diagnosis of gout or serum uric acid >upper limit of normal, clinically significant electrocardiogram abnormalities, pregnancy, and breastfeeding.

Number of Participants:

Assuming equal allocation of participants (i.e., in a 1:1 ratio), a 1-sided alpha of 10%, a standard deviation of 60 metres (Thomas et al, 2022) and 80% power, it is estimated that a total of 40 participants per group is needed to demonstrate a difference of 28.6 meters or more in the 6MWT between NMD670 and placebo using a 2-sample t-test.

Clinical efficacy will be investigated using an MMRM analysis, which will have higher power than the 2-sample t-test used in this sample size calculation. Approximately 110 participants will be screened (assuming a screen failure rate of 25% based on historical data (Attarian et al, 2021)) to achieve 80 randomised participants (40 per group). The study will be enriched to reach approximately 30% participants with CMT type 2.

Study Treatment Periods and Duration:

The study duration will be up to 8 weeks for each participant, including the following:
Screening (up to 4 weeks): Visit 1
Baseline (day ~1): Visit 2
1-week treatment visit (day 7): Visit 3
2-week treatment visit (day 14): Visit 4
End-of-treatment (EOT) visit (day 21): Visit 5
Follow-up (day 28): Visit 6

Eligible participants will be randomised in a 1:1 ratio to receive one of the following treatments for 21 days as shown in FIG. 10.
Placebo BID
NMD670 400 mg BID

The Schedule of Activities is provided in FIGS. 7A, 7B and 7C.

### Data Monitoring/Other Committee: Yes

An independent Data Safety Monitoring Board will be implemented for this study to ensure the safety of participants and the continued validity and scientific merit of the study. A blinded review of safety data will be conducted after approximately 25% of the planned population has completed the EOT Visit (or more frequently if needed to review emergent safety concerns). An unblinded review of safety and efficacy data will be performed via interim analysis conducted when approximately 50% of the planned population has completed the EOT visit.

### Objectives, Endpoints, and Estimands

| | |
|---|---|
| Objectives | Endpoints |

Primary

| | |
|---|---|
| Objectives | Endpoints |
| To assess the clinical | Change from baseline to day 21 in the total distance |
| efficacy of NMD670 as | walked during the 6MWT for NMD670 vs placebo. |
| rated by clinicians. | Change from baseline to day 21 in the time to |
| | complete the 10MW/RT for NMD670 vs placebo. |
| | Change from baseline to day 21 in the total time taken |
| | to perform the TUG for NMD670 vs placebo. |

Secondary

| | |
|---|---|
| To assess the clinical | Change from baseline to day 21 in CMT-FOM total |
| efficacy of NMD670 as | score and individual items for NMD670 vs placebo. |
| rated by clinicians and | Change from baseline to day 21 in fatigue index |
| participants. | calculated from the 6MWT for NMD670 vs placebo. |
| | Change from baseline to day 21 in ONLS total score |
| | and individual items for NMD670 vs placebo. |
| | Change from baseline to day 21 in CMT-HI total score |
| | and individual domains for NMD670 vs placebo. |
| | Change from baseline to day 21 in SF-36 total score |
| | and individual domains for NMD670 vs placebo. |
| | Change from baseline to day 21 in jitter and blocking |
| | for NMD670 vs placebo. |
| To assess responder rates | Proportion of participants with clinically meaningful |
| on clinical efficacy. | change from baseline in CMT-FOM total score and |
| | individual items for NMD670 vs placebo. |
| | Proportion of participants with clinically meaningful |
| | change from baseline in ONLS total score for NMD670 |
| | vs placebo. |
| | Proportion of participants with clinically meaningful |
| | change from baseline in CMT-HI total score and |
| | individual domains for NMD670 vs placebo. |
| | Proportion of participants with clinically meaningful |
| | change from baseline in SF-36 total score and |
| | individual domains for NMD670 vs placebo. |
| To assess the safety and | AEs, physical examination results, ophthalmological |
| tolerability of NMD670 over | examination results, clinical laboratory parameters, |
| 21 days compared with | vital signs, ECG results, and suicidal ideation and |
| placebo. | behaviour. |

Exploratory

| | |
|---|---|
| To explore the drug | Plasma levels of NMD670. |
| exposure of NMD670. | |
| To assess the clinical | Change from baseline to day 21 in CMT-FOM total |
| efficacy of NMD670 in | score and individual items for NMD670 vs placebo. |
| subgroups of interest (e.g., | Change from baseline to day 21 in fatigue index |
| CMT type 1 and CMT type | calculated from the 6MWT for NMD670 vs placebo. |
| 2 participants as rated by | Change from baseline to day 21 in ONLS total score |
| clinicians and participants). | and individual items for NMD670 vs placebo. |
| | Change from baseline to day 21 in 10MW/RT for |
| | NMD670 vs placebo. |
| | Change from baseline to day 21 in CMT-HI total score |
| | and individual domains for NMD670 vs placebo. |
| | Change from baseline to day 21 in SF-36 total score |
| | and individual domains for NMD670 vs placebo. |
| | Change from baseline to day 21 in jitter and blocking |
| | for NMD670 vs placebo. |
| To assess the clinical | Change from baseline to day 7 and day 21 in CMT- |
| efficacy of NMD670 at | FOM, ONLS, CMT-HI, and SF-36 total score and |
| various time points as rated | individual items as well as 10MW/RT for NMD670 vs |
| by clinicians and | placebo. |
| participants. | Change from baseline to day 14 in SF-36 total score |
| | and individual items. |
| To assess the biomarker | Skeletal muscle troponin I, type II plasma levels. |
| Skeletal muscle troponin I, | |
| type II, a potential marker | |
| of skeletal muscle fibre | |
| integrity. | |

Abbreviations: 6MWT=6-minute walk test; 10MW/RT=10-metre walk/run test;
AE=adverse event; CMT=Charcot-Marie-Tooth; CMT-FOM=Charcot-Marie-Tooth Functional Outcome Measure; CMT-HI=Charcot-Marie-Tooth Health Index;
ECG=electrocardiogram; ONLS=Overall Neuropathy Limitations Scale; SF-36=Short Form 36; TUG=Timed Up and Go test.

Primary Estimand:

The primary clinical question of interest for the objective of clinical efficacy is:
Is there a change from baseline to day 21 in 6MWT distance in ambulatory adult participants with CMT type 1 and type 2 treated with NMD670 vs placebo?
For the primary objective of the trial, it is also clinically important to evaluate if there is a change from baseline to day 21 in 10MW/RT time and TUG time in ambulatory adult participants with CMT type 1 and type 2 treated with NMD670 vs placebo.

Clinical efficacy will be investigated using a mixed model repeated measures analysis (MMRM).

Specifically, the estimand has the following attributes:
Population:
   The target population of interest is male and female participants aged 18 to 70 years (inclusive) with a diagnosis of CMT disease type 1 or type 2. These are all defined by the inclusion and exclusion criteria.
Patient-Level Endpoint:
   The change from baseline to day 21 in 6MWT distance will be derived for each participant. The baseline value will be derived from the assessment closest to (but before) the first dose.
Intercurrent Events:
   Anticipated intercurrent events include treatment discontinuation and treatment noncompliance. The treatment policy strategy will be used with respect to these types of events; thus, all available data will be used (with imputation if data is missing).
Population-Level Summary:
   The difference in least-squares mean taken from the model contrasts will be used to compare the treatment arms.
   The primary efficacy endpoint will be analysed in all randomly assigned participants by means of the treatment assigned for analysis (full analysis set [FAS]).

For the other endpoints evaluating the primary clinical question of interest, namely 10MW/RT time and TUG time, the estimand will be defined with the same attributes with the exception of the Patient-Level Endpoint, for which the corresponding assessment (10MW/RT time or TUG time, respectively) will be used.

Study Design—Overall Design
This is a Phase 2a, multicentre, placebo-controlled, randomised study of NMD670 (FIG. 10).
The study will enrol ambulatory male and female participants diagnosed with CMT types 1 and 2 and enriched to reach approximately 30% participants with CMT type 2.
Randomisation will be stratified by CMT type, sex and age (>45 years), which will allow for sensitivity analysis.
After screening, eligible participants will be randomised to receive placebo or NMD670 400 mg BID for 21 days.
Both the participant and Investigator will be blinded to treatment assignment and will not know which treatment is given during the treatment period.

The study duration will be up to 8 weeks for each participant, including the following:
Screening (up to 4 weeks): Visit 1
Baseline (day ~1): Visit 2
1-week treatment visit (day 7): Visit 3
2-week treatment visit (day 14): Visit 4
End-of-treatment (EOT) visit (day 21): Visit 5
Follow-up (day 28): visit 6

Eligible participants will be randomised in a 1:1 ratio to receive one of the following treatments for 21 days (FIG. 10).
Placebo BID
NMD670 400 mg BID

The study diagram is displayed in (FIG. 10), and the schedule of activities (SoA) is provided in (FIGS. 7A, 7B and 7C). Participants will attend a baseline Visit on the day before starting treatment and be dispensed with study treatment to be taken at home with a dosing diary. On the morning after the baseline Visit, they will start taking their treatment at home. They will attend a clinic visit on day 7 and day 14 and return to the site on the last day of treatment (day 21). During the visits to the clinic on day 7 and day 21, participants will take their morning dose at home before coming to the site and the second dose will be administered at the site during the visit. Three blood samples will be collected for PK analysis: on day 7 (Visit 3), 1 sample at any time during the visit; on day 21 (EOT), 1 sample prior to the second dose (trough) and 1 sample 2 to 4 hours after the second dose. After the EOT, participants will return to the site for a follow-up visit 7 days after the last dose of study treatment.

A Data Safety Monitoring Board (DSMB) will be implemented for the study as detailed later. Blinded review of safety data will be conducted after approximately 25% of the planned population has completed the EOT Visit (or more frequently if needed to review emergent safety concerns). An unblinded review of safety and efficacy data will be performed via interim analysis conducted when approximately 50% of the planned population has completed the EOT visit.

### Scientific Rationale for Primary and Secondary Efficacy Assessment

The following hierarchical endpoints, based on 3 clinical outcomes, are proposed as primary outcomes for this study: 1) the change from baseline to day 21 in the total distance walked during the 6MWT for NMD670 vs placebo; 2) the change from baseline to day 21 in the time to complete the 10MW/RT for NMD670 vs placebo; and 3) the change from baseline to day 21 in the time taken to perform the TUG for NMD670 vs placebo.

The use of these 3 endpoints combines various components of the CMT syndrome, such as muscle weakness, gait and balance impairment and fatigability, all of which are considered among the most debilitating symptoms in patients with CMT (White et al, 2019). Thus, these outcomes are positioned to capture clinically meaningful changes. The 6MWT measures the distance that a patient can quickly walk in 6 minutes on a flat surface and has recently been validated in patients with CMT (Mori et al, 2019). The 10MW/RT is a functional test that measures how fast a patient can walk/run 10 metres and is a commonly used outcome measure in clinical trials in CMT (Attarian et al, 2021; Watson, 2002; Pareyson et al, 2011). The TUG is a functional test in which patients are asked to get up from a seated position, walk 3 metres and return to their chair and sit down. The time to complete the task is recorded. It is commonly used to evaluate balance and mobility activity in patients with CMT (de França Costa et al, 2018). These 3 clinical outcomes have been shown to correlate with each other and with measures of neuromuscular transmission dysfunction (Gronnebok et al, 2023; Mori et al, 2019). Based on the mechanism of action for NMD670, it is therefore expected that the 3 outcomes will be responsive to change with NMD670.

Secondary efficacy outcome measures include the change from baseline to day 21 in the CMT Functional Outcome Measure (CMT-FOM), the fatigue index calculated from the 6MWT, the Overall Neuropathy Limitations Scale (ONLS), CMT Health Index (CMT-HI) and Short Form 36 (SF-36) for NMD670 vs placebo. The CMT-FOM is a validated clinician-rated outcome measure that measures physical abilities across different domains in adults with CMT, including muscle strength, endurance, mobility and balance (Eichinger et al, 2018), areas that have been identified as having an impact on quality of life in individuals with CMT (Johnson et al, 2013). The CMT-FOM is modelled on the CMT Pediatric Scale and has been designed to meet FDA guidance of a valid, responsive, and meaningful measure of function for application in trials in CMT (Eichinger et al, 2018). An updated Rasch-validated version of the CMT-FOM was recently published and will be used for the study (Mandarakas et al, 2024). In an observational study sponsored by NMD Pharma, it was demonstrated that deficits on several of the individual items of the CMT-FOM (including measures of muscle strength, balance, mobility and endurance) correlate with the level of transmission dysfunction measured by sfEMG in patients with CMT (Gronnebok et al, 2023). The ONLS is a clinician-rated outcome measure that evaluates disability in patients with neuropathy on a 12-point scale (Graham et al, 2006). The CMT-HI is a patient-reported outcome measure to evaluate disease burden in patients with CMT (Johnson et al, 2018). SF-36 is a patient-reported outcome measure to assess quality of life (Vinci et al, 2005). Finally, at selected sites where sfEMG is conducted routinely, the PD effect on neuromuscular transmission will also be assessed by sfEMG (Sanders et al, 2019). Thus, the study will provide information for selection of appropriate outcome measures for subsequent pivotal studies of NMD670 in CMT.

### Justification for Dose

NMD670 will be administered at 400 mg BID to give a total daily dose of 800 mg. The dose level has been selected to explore safety and efficacy of BID administered NMD670 in patients with CMT based on the dose levels tested in the Phase 1 FIH study and the nonclinical data.

The nonclinical data from the 4-week toxicity study support administration of NMD670 to humans up to 400 mg BID.

NMD670 has been tested in a Phase 1, FIH, single ascending and multiple ascending dose study in healthy volunteers. The dose of 400 mg BID was the maximum repeated dose administered to healthy volunteers, and this dose was well tolerated. In addition, the Phase 1 FIH study also tested single doses of 400 mg and 1200 mg in 12 MG patients.

The majority of AEs in the Phase 1 FIH study were mild in intensity, and no AEs were severe. No serious adverse reactions were reported in the Phase 1 study. Myotonia of moderate severity was reported in 1 healthy subject receiving a single dose of 1600 mg. This adverse reaction resolved spontaneously, and no further subjects were dosed at 1600 mg. At 1200 mg once a day, the presence of myotonic symptoms was observed in 2 healthy subjects, was mild in intensity, and resolved spontaneously within a few hours. Myotonia is considered to be exaggerated on-target CIC-1 pharmacology. Myotonia was observed in healthy subjects and was not observed in patients with MG. Overall, NMD670 was safe and well tolerated and the incidence of patients with TEAEs during the study were similar between NMD670 and placebo treatments. Administration of a dosage of 400 mg BID is considered safe.

Furthermore, in the FIH study in MG patients, a single dose of 400 mg led to statistically significant and clinically relevant improvements of symptoms. The dosage of 400 mg BID will be administered to test the pharmacological activity in CMT. The doses will be administered approximately 6 hours apart and are expected to provide a potentially therapeutic effect for the entire waking day.

### Justification for Study Duration

ICH GLP nonclinical repeat-dose toxicology studies in 2 species (rats and minipigs) have been conducted for 28 days with recovery periods. The maximum permitted duration of dosing in humans is 28 days. The duration in this trial is 21 days and is less than the permitted dosing supported by the nonclinical repeat-dose toxicology studies.

### Justification for Placebo

There are no approved medical therapies for CMT. Therefore, the safety and efficacy of NMD670 will be tested against placebo rather than against an already-approved therapy. Comparison with placebo is essential to show that NMD670 is effective while minimising the total number of participants needed to be exposed to the experimental drug. Placebo use is short-term (21 days), and participants will continue to receive their standard of care during the study period including physical or occupational therapy or receive medication to manage pain. The participants will be well monitored, and there are clear guidelines for withdrawing participants if needed. At a study level, the DSMB will provide monitoring of the study and the safety of participants. Importantly, the informed consent form (ICF) also clearly explains the use of placebo and what it is. Participation in this Phase 2 study does not exclude participants from participating in a potential future pivotal trial where they might have the opportunity to receive active treatment. For these reasons, CMT participants would not be further disadvantaged if enrolled in this placebo-controlled trial and short-term use of placebo in this clinical trial is considered justified.

### End-of-Study Definition

The end of the study is defined as the date of the last visit of the last participant in the study globally.

A participant is considered to have completed the study if the participant has completed all periods of the study including the follow-up (Visit 6).

### Study Population

Prospective approval of protocol deviations from recruitment and enrolment criteria, also known as protocol waivers or exemptions, is not permitted.

The study population will consist of participants with a diagnosis of CMT type 1 and type 2. The study will be enriched to reach approximately 30% participants with CMT2 to allow sensitivity analysis. Participants must be able to provide written consent and meet all the inclusion criteria and none of the exclusion criteria. Participants >70 years of age are not eligible to participate to avoid exposing patients of advanced age to an experimental drug substance, as they are known to have larger physiological variations on PK and PD variables and higher rate of comorbidities and polypharmacy, all of which could confound the interpretation of the study results. Participants who use daily assistive devices such as ankle-foot orthosis (AFOs) are not eligible for participation in this Phase 2 study in order to maintain a homogeneous population for efficacy evaluation. As described in the literature, AFOs can affect walking and, consequently, the primary efficacy outcome of this study (Ramdharry et al, 2012). It is important to note that future studies are anticipated to include these patient populations.

### Inclusion Criteria

Participants are eligible to be included in the study only if all of the following criteria apply:

### Age

1. Male or female participants must be 18 to 70 years inclusive at the time of signing the ICF.

Type of Participant and Disease Characteristics
2. Diagnosis of CMT type 1 or 2 confirmed by genetic testing.
3. The following physical capabilities must be met without assistive devices such as AFOs (orthopaedic inserts are allowed) at screening (both must apply):
   a. 6MWT total distance within 1 standard deviation of the mean adjusted by age and sex from the provided CMT norm.
   b. Bilateral ankle dorsiflexion strength of 4 (inclusive) or less on the manual muscle testing scale.
4. Participants already receiving physical or occupational therapy or following a prescribed training regimen for more than 30 days prior to screening should continue their current treatment regimen throughout the study. Participants are not allowed to start physical or occupational therapy or a prescribed training regimen within 30 days of screening or during the study.
5. Participants taking medication for muscle cramps (e.g., magnesium) and/or pain (e.g., nonsteroidal anti-inflammatory drug [NSAID], neuropathic pain medication, or opiates) should be on a stable dose for at least 30 days prior to screening and should maintain a stable dose for the duration of the study.

### Weight

6. Body mass index between 18 and 35 kg/m² inclusive at screening and a minimum weight of 40 kg.

### Contraceptive/Barrier Requirements

7. Female participants who are women of childbearing potential (WOCBP) and male participants with partners who are WOCBP must agree to use a highly effective contraception method during the study. Contraceptive use by men and women must be consistent with local regulations regarding the methods of contraception for those participating in clinical studies (refer to Appendix 4 for additional information).

### Informed Consent

8. Participant is capable of giving signed informed consent.

### Exclusion Criteria

Participants are excluded from the study if any of the following criteria apply:

### Medical Conditions

1. Participants with other significant disease that may interfere with the interpretation of study data (e.g., other neuromuscular diseases) and/or ability to complete the tests, in the opinion of the Investigator.
2. Use of daily assistive devices such as AFOs (orthopaedic inserts are allowed).
3. Severe deformity or ankle contracture that in the opinion of the Investigator would limit passive range of motion to interfere with performance of the tests.
4. Ankle surgery or other limb-surgery procedures related to CMT within 9 months of screening.
5. Moderate-to-severe neuropathic or inflammatory/musculoskeletal pain that in the opinion of the Investigator would interfere with performance of the tests.
6. Participants with a clinical diagnosis of gout or with serum uric acid greater than the upper limit of normal (ULN) at screening.
7. Study participants with a centrally read 12-lead ECG with findings considered to be clinically significant upon medical review. The clinical significance of the findings needs to be assessed by the Investigator to determine eligibility. Participants with any of the following abnormalities in QT interval corrected for heart rate using Fridericia's correction (QTcF) at screening:
   a. QTcF>450 msec for male participants without bundle branch block.
   b. QTcF>470 msec for female participants without bundle branch block.
   c. QTcF>480 msec in participants with bundle branch block.
8. Participants with any of the following:
   a. Abnormal liver function test result defined as total bilirubin>1.5×ULN (participants with Gilbert's syndrome can be included with total bilirubin>1.5×ULN if direct bilirubin is:1.5×ULN and 535% of total bilirubin).
   b. Abnormal liver transaminase levels at baseline and confirmed current or chronic history of liver disease including (but not limited to) hepatitis virus infections, drug- or alcohol-related liver disease, non-alcoholic steatohepatitis, autoimmune hepatitis, hemochromatosis, Wilson's disease, α-1 antitrypsin deficiency, primary biliary cholangitis, primary sclerosing cholangitis, or any other liver disease considered clinically significant by the Investigator.
   c. Known hepatic or biliary abnormalities (with the exception of Gilbert's syndrome or asymptomatic gallstones).
   d. Any renal impairment or kidney abnormalities at screening.
9. Participants with laboratory test result abnormalities at screening considered clinically significant by the Investigator.
10. Participants with breast cancer, lymphoma, leukaemia, or any malignancy within the past 5 years. An exception of this 5-year requirement is basal cell or squamous epithelial carcinomas of the skin that have been resected with no evidence of metastatic disease.
11. Participants with an ongoing significant psychiatric disorder (e.g., unstable and newly diagnosed major depressive disorder or unstable and newly diagnosed anxiety disorders).
12. Participants with alcohol abuse as judged by the Investigator or with positive drug screen results (cocaine, heroin, opiates, or marijuana) at screening.
   a. If positive for marijuana, criteria for drug abuse (as determined by the Investigator) is also needed to meet this exclusion criterion.
   b. If positive for opiates, the exclusion criterion is not met if opiates have been medically prescribed for the treatment of pain in CMT.
13. Participants with a positive HIV antibody test result.
14. Participants with positive serology test results for hepatitis B (unless due to vaccination, resolved natural infection, or passive immunisation, as confirmed with the Medical Monitor).
15. Participants positive for hepatitis C antibody.
16. Participants with a clinically significant history of allergic conditions (including drug allergies and anaphylactic reactions) or hypersensitivity to any component of the IMP.
17. Participant with myotonic disorders or those on drugs that induce or mask myotonia.
18. Participants unable to undergo the ophthalmologic evaluation at screening.

### Prior/Concomitant Therapy

19. Participants who require prohibited medication within 30 days of screening (or 5 half-lives of the medication, whichever is shorter) and/or are likely to require them during the study; prohibited medications include drugs showing relevant effects on NMJ transmission and drugs where the effect might be affected by potential metabolic drug-drug interactions with NMD670. Other current and recent (within 1 month prior to screening) treatments will be allowed if judged by the Investigator to be of no relevance for the study.

### Prior/Concurrent Clinical Study Experience

20. Participants who have received treatment with another IMP within 30 days (or 5 half-lives of the medication, whichever is longer) prior to day 1.

### Other Exclusion Criteria

21. Participants with history of poor compliance with relevant therapy in the opinion of the Investigator.
22. Female participants who plan to become pregnant during the study or are currently pregnant or breastfeeding.

### Lifestyle Considerations

1. Participants should take their treatment with a regular glass of water (approximately a quarter litre or 8 oz).
2. During the treatment period, participants should be careful to drink sufficiently and remain properly hydrated. On average it might mean 2 litres of water a day for an adult person.
3. Participants should avoid changing their daily activities during the study.
4. Participants should avoid out-of-the-ordinary, strenuous, or excessive physical activity (e.g., heavy lifting or fitness training) for 7 days prior to each site visit, while maintaining usual ambulatory activities. Participants will be asked to maintain minimal physical activities on the day preceding and the morning of each site visit. See also Inclusion Criterion 4.
5. Alcohol abuse as judged by the Investigator is not allowed for the duration of the study, and participants should abstain from alcohol for 24 hours prior to each site visit.
6. During the overall duration of the clinical trial, prescription and use of assistive devices such as AFOs are not allowed.

### Screen Failures

A screen failure occurs when a participant who has consented to participate in the clinical study is not subsequently assigned to study IMP. A minimal set of screen failure information is required to ensure transparent reporting of screen failure participants to meet the Consolidated Standards of Reporting Trials publishing requirements and to respond to queries from regulatory authorities. Minimal information includes demography, reason for screen failure (e.g., eligibility requirements failed), and documentation of any medical occurrences that qualify as SAEs.

Retesting of eligibility assessments during the screening period may be permitted if, in the opinion of the Investigator, the original laboratory result was abnormal due to a transient condition or potential error.

Individuals who do not meet the criteria for participation in this study (screen failures) may be rescreened at the discretion of the Investigator. The individual is eligible for enrolment if the results of the new screening visit meet eligibility criteria. Rescreened participants should sign a new ICF and be assigned a new participant number for every screening/rescreening event.

### Screening and Enrolment Log and Participant Identification Numbers

Details of the participant's enrolment will be recorded in the electronic data capture system and interactive response technology (IRT) system.

Upon enrolment, each participant will be assigned a unique participant identification number by the IRT system. Participant numbers will not be re-used for different participants.

### Study Management of IMP and Concomitant Therapy

TABLE 24
Study IMP Administered

| IMP | NMD670 | Placebo |
|---|---|---|
| Dosage | Film-coated tablet | Film-coated tablet |
| Formulation | | |
| Unit Dose | 200-mg tablets | Matching tablets with |
| Strength(s) | | no active treatment |
| Dosage | 400 mg BID | Placebo BID |
| Level(s) | 2 × 200-mg tablets | 2 matching placebo |
| | of NMD670 BID | tablets BID |
| Route of | Oral | Oral |
| Administration | | |
| Use | Experimental | Placebo-comparator |
| IMP or NIMP | IMP | IMP |

| | |
|---|---|
| Sourcing | Provided centrally by the Sponsor |
| Packaging and | The IMPs (NMD670 and placebo) will be packaged |
| Labelling | and labelled as required per country requirements. |

Abbreviations: BID = bis in die/twice a day; IMP = investigational medicinal product; NIMP = non-investigational medicinal product.

### Administration of Study IMP

Placebo or NMD670 tablets should be swallowed as whole tablets together with a regular glass of water (approximately a quarter litre or 8 oz).

One day before the treatment period, participants will attend a baseline visit (Visit 2) at the study site and be dispensed with tablets in bottles to be taken at home. The next day, participants will take their first dose of study treatment in the morning and their second approximately 6 hours later in the afternoon (within 5 to 8 hours after the morning dose). Participants will follow this morning and afternoon dosing regimen at home for the duration of the study with the exception of the day of the 7-day treatment Visit and EOT Visit, where the second dose will be administered in the clinic. Participants will take 2 tablets each in the morning and in the afternoon. Doses should be taken at approximately the same time every day. Participants who miss a morning and/or an afternoon dose should not attempt to add or recover the missed dose, neither at a later time nor the next day.

On day 7 (Visit 3) and on the EOT day 21 (Visit 5), participants will attend a visit to the study site. They will be asked to take the morning dose at home, and the second dose will be dispensed in the clinic and administered at approximately noon. If participants forget to take the morning dose at home before the visit, it will be administered at the site.

During Visit 2 (baseline), participants will be dispensed a sufficient number of tablets covering the first treatment week. At Visit 3 (day 7) participants should be asked to bring remaining tablets and bottles to the site. Participants will then be dispensed a sufficient number of tablets to cover the remaining treatment period. At Visit 5 (EOT), participants should be asked to bring remaining unused tablets and bottles to the site.

### Preparation, Handling, Storage, and Accountability of IMP

The Investigator or designee must maintain a log to confirm appropriate conditions (e.g., temperature) have been maintained during transit for all study IMP received and any discrepancies are reported and resolved before use of the study IMP.

All supplies of study IMP must be stored in accordance with the manufacturer's instructions between 15 and 25° C. The study IMP will be stored in a securely locked area, accessible to authorised persons only, until needed for dosing.

Only participants enrolled in the study may receive study IMP, and only authorised site staff may supply, prepare, or administer study IMP. All study IMP must be stored in a secure, environmentally monitored (manual or automated) area in accordance with the labelled storage conditions with access limited to the Investigator and authorised site staff.

The Investigator or authorised site staff is responsible for study IMP accountability, reconciliation, and record maintenance (e.g., receipt, reconciliation, and final disposition records).

Further guidance and information regarding handling, storage, accountability, and administration of study IMP as well as the final disposition of unused study IMP are provided in the Pharmacy Manual.

### Assignment to Study IMP

All participants will be centrally randomised using an IRT. Each participant will be assigned a unique number that encodes the participant's assignment to 1 of the 2 treatment arms of the study according to the randomisation schedule. Details of the procedure are described in an IRT Manual.

Study IMP will be administered/dispensed at the study visits as summarised in the SoA (FIGS. 7A, 7B and 7C).

Returned study IMP should not be re-dispensed to the participants.

### Blinding/Masking

This is a double-blind study in which participants, Investigators, site staff, Sponsor, monitors, and members of the Data Safety Monitoring Board are blinded to study IMP assignment. The IRT will be programmed with blind-breaking instructions. In case of an emergency, the Investigator has the sole responsibility for determining if unblinding of a participant's study IMP assignment is warranted. Participant safety must always be the first consideration in making such a determination. If the Investigator decides that unblinding is warranted, the Investigator may, at the Investigator's discretion, contact the Sponsor to discuss the situation prior to unblinding a participant's study IMP assignment unless this could delay emergency treatment for the participant. It is not mandatory for the Investigator to contact the Sponsor prior to emergency unblinding. If a participant's study IMP assignment is unblinded, the Sponsor must be notified within 24 hours of this occurrence. The date and reason for the unblinding must be recorded.

The Sponsor (or designee) may unblind the IMP assignment for any participant with an SAE. If the SAE requires that an expedited regulatory report be sent to 1 or more regulatory agencies, a copy of the report identifying the participant's IMP assignment may be sent to Investigators in accordance with local regulations and/or Sponsor policy.

For the planned interim analysis, the DSMB will review unblinded data from the study efficacy and safety outcomes (as detailed in the Data Safety Monitoring Board section and the statistical analysis plan [SAP]).

### Study IMP Compliance

When participants self-administer study IMP at home, they will be instructed to record the time of each morning and afternoon dosing in a diary every day and return unused tablets to the study site. Compliance with study IMP will be assessed by the Investigator and/or delegated site staff when participants return to the site on day 7 and day 21, by reviewing the diary entries and counting returned tablets. Compliance will be documented in the source documents and relevant form. Deviations from the prescribed dosage regimen should be recorded.

When participants are dosed at the site, they will receive study IMP directly from the Investigator or designee, under medical supervision. The date and time of each dose administered at the study site will be recorded in the source documents.

A record of the quantity of tablets dispensed to and administered by each participant must be maintained and reconciled with study IMP and compliance records. IMP start and stop dates, including dates for IMP delays, will also be recorded.

### Dose Modification

No dose modifications are planned during the study.

### Continued Access to Study IMP After the End of the Study

Study IMP will be administered for 21 days. After completing the study treatment period, participants will stop receiving study IMP. The maximum permitted duration of dosing is 28 days based on toxicology data.

### Treatment of Overdose

For this study, any dose of NMD670 greater than 400 mg BID (i.e., 800 mg per day) will be considered an overdose.

In the event of an overdose, the Investigator should:
Evaluate if an urgent medical consultation is needed.
Evaluate if an unscheduled study visit is needed.
In all cases, closely monitor the participant for any AEs/SAEs and laboratory abnormalities as medically appropriate until at least the next scheduled follow-up visit.
Document the quantity of the tablets taken as well as the duration of the overdose.
Evaluate the participant to determine, in consultation with the Medical Monitor, if possible, whether study IMP should be interrupted.

### Prior and Concomitant Therapy

Any medication or vaccine (including over-the-counter or prescription medicines, recreational drugs, vitamins, and/or herbal supplements) that the participant is receiving at the time of enrolment or receives during the study must be recorded along with:
Reason for use.
Dates of administration including start and end dates.
Dosage information including dose and frequency.

The Medical Monitor should be contacted if there are any questions regarding concomitant or prior therapy.

### Prior and Concomitant Medications Review

The Investigator or qualified designee will review prior medication use and record prior medications taken by the participant within 30 days prior to screening as well as all relevant CMT therapies since diagnosis.

The Investigator or qualified designee will record medication, if any, taken by the participant during the study through the last visit. Concomitant medications will be recorded until the follow-up visit (Visit 6) or longer if related to an SAE.

Participants taking medication for muscle cramps (e.g., magnesium) and/or pain (e.g., NSAID, neuropathic pain medication, or opiates) should be on a stable dose for at least 30 days prior to screening and should maintain a stable dose for the duration of the study.

Participants using corticosteroids (for any reason) should maintain a stable dose for at least 2 months before the first dose of study IMP and should be expected to require no dose changes until the end of the study.

The following medications are prohibited during the study:
Anticholinergic drugs (e.g., atropine, benztropine, orphenadrine, and scopolamine).
Drugs that may interfering with NMJ transmission.
Muscle relaxants (e.g., carisoprodol, cyclobenzaprine, diazepam, metaxalone) or benzodiazepines.
   NOTE: Short-acting benzodiazepines (estazolam [Prosom®], flurazepam [Dalmane®], temazepam [Restoril®], triazolam [Halcion®], and midazolam [Versed®]) used for nighttime treatment of insomnia are permitted if on a stable dose for 2 months prior to screening and expected to remain stable for the duration of the study.
Drugs lowering serum uric acid:
   Xanthine oxidase inhibitors (e.g., allopurinol, febuxostat, and topiroxostat).
   Uricosuric agents (e.g., benzbromarone, probenecid, and arhalofenate).
Potent or moderately potent inhibitors of OATP1B1, breast cancer resistance protein (BCRP), OAT1, OAT3, multidrug and toxic extrusion protein 2-K, and OAT2 (e.g., probenecid, diclofenac, and pyrimethamine).
Sensitive or moderately sensitive substrates of OAT3, BCRP, and URAT1 (e.g., topotecan, rosuvastatin, oseltamivir, and olmesartan).

COVID-19 vaccines should be avoided from 1 week prior to screening to 1 week after the last dose of study IMP.

Other current and recent (within 1 month prior to screening) treatments will be allowed if judged by the Investigator to be of no relevance for the study.

### Discontinuation of Study IMP and Participant Discontinuation (Including Stopping Rules)

Discontinuation of specific sites or of the study as a whole are handled as part of the appendix on study oversight (Appendix 1 and section on Study and Site Start and Closure).

Participants who are discontinued prematurely before completing Visit 5 (EOT) may be replaced.

### Discontinuation of Study IMP

Participants may be discontinued from study IMP in the following situations:
Participant decision. The participant is at any time free to discontinue treatment, without prejudice to further treatment.
An AE that, in the opinion of the Investigator and/or Sponsor, warrants discontinuation from further dosing (e.g., in the event of mild or moderate myotonia, the Investigator might consult with the Sponsor to evaluate permanent treatment discontinuation).
Safety reasons as judged by the Investigator and/or Sponsor where continued treatment may put the participant at undue risk.

Participants will be discontinued from study IMP in the following situations:
The participant experiences an event of severe myotonia (i.e., severe stiffness exists, which impairs every duty and activity).
The participant becomes pregnant during the course of the study; see the section on Pregnancy.
The participant experiences an increase in Lens Opacity Classification System III (LOCS III) 1.0 (in any single category: nuclear, cortical, or posterior subcapsular) that is associated with a decrease in Early Treatment Diabetic Retinopathy Study (ETDRS) of ≥2 lines.
The participant has any of the following liver chemistry findings:
   Alanine aminotransferase (ALT) or aspartate aminotransferase (AST) >8×ULN.
   ALT or AST >5×ULN for more than 2 weeks.
   AST or ALT>3×ULN and total bilirubin>2×ULN
   AST or ALT>3×ULN and international normalised ratio >1.5.
   AST or ALT>3×ULN with the appearance of fatigue, nausea, vomiting, right upper quadrant pain or tenderness, fever, rash, and/or eosinophilia (>5%).
The participant has any of the following QTcF findings:
   Participant without bundle branch block:
      QTcF>500 msec or change from baseline of QTcF>60 msec.
   Participant with bundle branch block:
      QTcF>500 msec if baseline value was <450 msec.
      QTcF ≥530 msec if baseline value was between 450 and 480 msec (inclusive).

If study IMP is permanently discontinued, the participant should, if at all possible, remain in the study to be evaluated for follow-up assessments for as many visits as possible until the end-of-study (EOS) Visit (included). The timing of the visits and the assessments that should be conducted at the different visits are listed in the SoA (FIGS. 7A, 7B and 7C). PK sampling is not required at the EOT Visit if IMP is permanently discontinued.

Participants who meet study IMP discontinuation or stopping criteria will be followed up for improvement and for determination of whether dosing will be allowed to restart based on the following criteria for restarting study IMP; treatment can restart once the event that prompted the discontinuation from study IMP has been resolved. The restart needs to be evaluated on a case-by-case basis by the Investigator and the Medical Monitor. All of this needs to be registered in the electronic case report form (eCRF). Should abnormalities recur after restarting study IMP, the study IMP should be permanently discontinued.

Participant Discontinuation/Withdrawal from the Study

A participant may withdraw from the study at any time at the participant's own request or for any reason (or without providing any reason). A participant may be withdrawn at any time at the discretion of the Investigator for safety or compliance reasons. The participant will be permanently discontinued from the study IMP and the study at that time.

If the participant withdraws consent for disclosure of future information, the Sponsor may retain and continue to use any data collected before such a withdrawal of consent.

If a participant withdraws from the study, the participant may request destruction of any samples taken and not tested, and the Investigator must document this in the site study records.

At the time of discontinuation from the study, an EOT Visit should be conducted immediately, and if possible, an EOS Visit should be conducted 1 week later as shown in the SoA (FIGS. 7A, 7B and 7C). Refer to the SoA for data to be collected at the time of study discontinuation and follow-up and for any further evaluations that need to be completed.

Participation may be terminated prior to completing the study and the reason recorded as follows:
AE
Protocol violation
Loss to follow-up
Participant withdrawal of consent
Other

### Loss to Follow-Up

A participant will be considered lost to follow-up if the participant repeatedly fails to return for scheduled visits and is unable to be contacted by the study site.

The following actions must be taken if a participant fails to return to the study site for a required study visit:
The site must attempt to contact the participant and reschedule the missed visit as soon as possible (and within the visit window, where one is defined), counsel the participant on the importance of maintaining the assigned visit schedule and ascertain whether the participant wishes to and/or should continue in the study.
In cases in which the participant is deemed lost to follow-up, the Investigator or designee must make every effort to regain contact with the participant. Contact attempts should be documented in the participant's medical record/source documentation.

### Study Assessments and Procedures

Study procedures are summarised in the SoA (FIGS. 7A, 7B and 7C). If possible, assessments should be conducted at the same time at the different visits.

On day 7 (V3) and day 21 (EOT), participants will take their morning dose at home and the second dose will be administered at the site at approximately noon. If possible, the 6MWT should be performed 2 hours after the afternoon dose followed by 10MW/RT, TUG, CMT-FOM (remaining items; do not repeat 6MWT and TUG items of the CMT-FOM), ONLS and sfEMG (in that order). If possible, the CMT-HI and the SF-36 should be completed by the participants prior to the clinician-rated efficacy assessments.

On day 7, 1 PK sample is taken at any time during the visit. On day 21, 1 sample will be taken close to trough (pre-second daily dose) for NMD670, and 1 sample will be taken around maximal concentration (Cₘₐₓ) (2 to 4 hours post-second daily dose) (see the section on Pharmacokinetics for details).

Protocol waivers or exemptions are not allowed.

Adherence to the study design requirements, including those specified in the SoA, is required for study conduct.

All screening evaluations must be completed and reviewed to confirm that potential participants meet all eligibility criteria. The Investigator will maintain a screening log to record details of all participants screened and to confirm eligibility or record reasons for screening failure, as applicable.

Procedures conducted as part of the participant's routine clinical management (e.g., blood count) and obtained before signing the ICF may be used for screening or baseline purposes provided the procedures met the protocol-specified criteria and were performed within the timeframe defined in the SoA.

In the event of a significant study-continuity issue (e.g., caused by a pandemic), alternate strategies for participant visits, assessments, medication distribution and monitoring may be implemented by the Sponsor or the Investigator, as per local health authority/ethics requirements.

Safety/laboratory/analyte results that could unblind the study will not be reported to investigative sites or other blinded personnel until the study has been unblinded.

Over the duration of the study, the expected maximum amount of blood collected from each participant, including any extra assessments that may be required, will be much lower than 250 mL over an 8-week period.

Repeat or unscheduled samples may be taken for safety reasons or for technical issues with the samples.

### Participant Identification Card

All participants will be given a participant identification card identifying them as participants in a research study. The card will contain study site contact information (including direct telephone numbers) to be used in the event of an emergency. The Investigator or qualified designee will provide the participant with a participant identification card immediately after the participant provides written informed consent.

### Calibration of Equipment

The Investigator (or qualified designee) is responsible for ensuring that any device or instrument used for a clinical evaluation/test during the study that provides information about eligibility criteria and/or safety or efficacy parameters is suitably calibrated and/or maintained to ensure that the data obtained are reliable and/or reproducible. Documentation of equipment calibration must be adequately maintained at the study site according to the site's procedures.

### Administrative and Baseline Procedures

### Medical History

A medical history will be obtained by the Investigator or qualified designee. The medical history will collect all active conditions and any condition diagnosed that the Investigator considers to be clinically relevant. Details regarding the disease for which the participant has enrolled in this study will be recorded separately and not listed as medical history.

### Substance Use/Drug Screen

The use of drugs (of potential abuse) can influence the measurements of and the ability to perform the functional outcome measures of the study. Therefore, a drug test, including cocaine, heroin, opiates, and marijuana will be performed at screening. Participants with positive drug screen results (cocaine, heroin, opiates or marijuana) will not be allowed in the study. If positive for marijuana, criteria for drug abuse (as determined by the Investigator) is also needed to meet this exclusion criterion. If positive for opiates, the exclusion criterion is not met if opiates have been medically prescribed for the treatment of pain in CMT. Participants taking opiates to manage pain will need to be on a stable dose for 30 days prior to screening and maintain a stable dose for the duration of the study.

### Efficacy Assessments

The efficacy assessments planned at each visit are provided in the SoA (FIGS. 7A, 7B and 7C). To the extent possible, the efficacy assessments should be performed in the order outlined in the SoA. On day 7 and day 21, the primary efficacy assessments should be performed approximately 2 hours after administering the afternoon dose.

Clinical assessments will be performed by a trained evaluator at the site. For each test, each participant should, to the extent possible, be assessed by the same evaluator for all sessions. The use of assistive devices such as AFOs are not permitted during the study.

Before and after each clinical assessment, the assessors should ensure that participants are sufficiently rested, including at least 10 minutes of rest after each clinical assessment.

If study IMP is permanently discontinued, the participant should, if at all possible, remain in the study to be evaluated for follow-up assessments including efficacy assessments for as many visits as possible until the EOT Visit (included) as detailed in the section on Discontinuation of Study IMP.

### Primary Efficacy Assessment

The following hierarchical endpoints are proposed as primary efficacy assessments for this study:
1. The change from baseline to day 21 in the total distance walked during the 6MWT for NMD670 vs placebo. The 6MWT measures the distance that a patient can walk in 6 minutes on a flat surface and has recently been validated in patients with CMT (Mori et al, 2019).
2. The change from baseline to day 21 in the time to complete the 10MW/RT in seconds for NMD670 vs placebo. The 10MW/RT is a functional test that measures how fast a patient can safely walk 10 metres and has frequently been used in clinical trials in CMT (Attarian et al, 2021; Pareyson et al, 2011).
3. The change from baseline to day 21 in the total time taken to perform the TUG for NMD670 vs placebo. The TUG is a functional test in which patients are asked to get up from a seated position, walk 3 metres and return to their chair and sit down. The time to complete the task is recorded. It is commonly used to evaluate balance and mobility activity in patients with CMT (de França Costa et al, 2018).

Further details regarding the methodology will be provided in the study-specific training material.

Calculation of derived variables will be defined in the SAP.

### Other Clinician-Rated Efficacy Outcomes

### CMT Functional Outcome Measure

The CMT-FOM is a validated clinician-rated outcome measure that measures physical abilities across different domains in adults with CMT including muscle strength, endurance, mobility and balance (Eichinger et al, 2018). The CMT-FOM has been designed to meet FDA guidance of a valid, responsive, and meaningful measure of function for application in trials in CMT. An updated Rasch-validated version of the CMT-FOM was recently published and will be used for the study (Mandarakas et al, 2024).

Further details regarding the methodology will be provided in the study-specific training material.

Calculation of derived variables will be defined in the SAP.

### Overall Neuropathy Limitation Scale

The ONLS is a composite scale that evaluates disability in patients with neuropathy on a 12-point scale (Graham et al, 2006). ONLS includes both an arm and a leg scale. It is a modified version of the Overall Disability Sum Score. The ONLS has previously been used as a primary outcome measure in Phase 3 studies in CMT to establish clinical efficacy (Attarian et al, 2021).

Further details regarding the methodology will be provided in the study-specific training material.

Calculation of derived variables will be defined in the SAP.

### 6-Minute Walk Test Fatigue Index

The fatigue index will be included as secondary variable and derived from the 6MWT as the percentage reduction in distance walked in the sixth minute compared to the first minute.

### Patient-Reported Outcomes

Participants will be asked to complete 3 questionnaires: the CMT-HI, SF-36, and Myotonia Behaviour Scale (MBS).

### CMT Health Index

The CMT-HI is a validated patient-reported outcome measure to assess disease burden in patients with CMT (Johnson et al, 2018).

Calculation of derived variables will be defined in the SAP.

### Short Form 36

The SF-36 is a validated generic questionnaire for the assessment of quality of life. SF-36 has previously been used to demonstrate quality of life impairments in patients with CMT (Vinci et al, 2005).

Calculation of derived variables will be defined in the SAP.

### Myotonia Behaviour Scale

The MBS is a patient-reported outcome that evaluates the subjective nuisance from myotonia on a scale from 0 to 5.

The MBS will be assessed at all clinic visits and at home every day during the treatment period.

### Safety Assessments

The safety assessments planned at each visit are provided in the SoA (FIGS. 7A, 7B and 7C).

### Height and Weight

Height and weight will be measured and recorded.

### Physical Examinations

A complete physical examination will be conducted at the screening Visit and follow-up Visit (EOS) and will include assessments of the neurological, cardiovascular, respiratory, gastrointestinal, musculoskeletal and skin systems.
At all other in-clinic visits, assessments of the neurological and musculoskeletal systems must be performed.

The neurological and musculoskeletal examinations to be performed shall include assessment of mental status, cranial nerves, motor examination, sensory examination, coordination, reflexes, and gait, as well as the forced handgrip test and forced eyelid closure test. In the handgrip test, the participants are instructed to squeeze both of their hands shut for 10 seconds then open quickly. Similarly, in the eyelid closure test, the participants are instructed to squeeze their eyes closed for 10 seconds then open quickly. The tests are documented as either normal (normal hand and eyelid opening) or abnormal (delayed hand and eyelid opening).

Investigators should pay special attention to clinical signs related to previous serious illnesses.

### Ophthalmological Examinations

Ophthalmologic assessments will be performed at screening (to determine eligibility and as the baseline observation) and during follow-up as specified in (FIGS. 7A, 7B and 7C). The examination will be performed by a licensed ophthalmologist or optometrist (according to local regulations) and includes the following:
Visual acuity examination on both eyes using the ETDRS, without pupil dilation (Solomon et al, 2019).
Slit lamp examination on both eyes using the LOCS III grading scale, with pupil dilation (Chylack et al, 1993).

### Vital Signs

Vital signs will be measured in a supine position after 5 minutes of rest and will include tympanic temperature, systolic and diastolic blood pressure, and pulse and respiratory rate.

Three readings of blood pressure and pulse will be taken at least 1 minute apart. The mean of these 3 consecutive replicates will be used as the value for the visit.

### Electrocardiograms

Triplicate 12-lead ECGs will be obtained after 5 minutes of rest as outlined in the SoA (FIGS. 7A, 7B and 7C) using an ECG machine that is centrally provided and that automatically calculates the heart rate and measures PR, QRS, QT, RR, and QTcF intervals.

For triplicate ECG recordings, 3 individual ECG tracings should be obtained as closely as possible in succession, no more than 2 minutes apart. The average of the triplicate measures should be used for assessing ECG parameters.

For participants showing an increase of QTcF by ≥60 msec (regardless of the baseline value) or abnormalities in QTcF (as defined in Exclusion Criterion 7), persisting for ≥5 minutes, further evaluation is recommended, including repeated ECGs. A cardiologist may be consulted. Refer to section Discontinuation of Study IMP for QTcF discontinuation criteria.

The central ECG review vendor, as the responsible company for the centralised ECG assessments, will provide the study sites with standardised ECG equipment and supplies, specific training and written instructions. Following acquisition of a quality ECG tracing, the Investigator or designee will electronically transfer the data to the central ECG review vendor.

Investigators will assess the eligibility of the participant according to the ECG report of Visit 1 from the central reader. Any abnormal finding in the ECG tracing will also be evaluated by the Investigator or by a hospital cardiologist during the visit and will be specifically documented and registered in the case report form (CRF). Throughout the study, clinically relevant new findings or worsening of pre-existing findings in the ECGs (parameters or abnormal findings in the tracing) must be considered AEs and recorded in the AE CRF.

### Clinical Safety Laboratory Tests

Refer to Appendix 2 for the list of clinical laboratory tests to be performed and the SoA (FIGS. 7A, 7B and 7C) for the timing and frequency.

The Investigator must review the laboratory results, document this review, and record any clinically significant changes occurring during the study as AEs. The laboratory results must be retained with source documents.

Abnormal laboratory findings associated with the underlying disease are not considered clinically significant unless judged by the Investigator to be more severe than expected for the participant's condition.

All laboratory tests with values considered clinically significant during participation in the study until the follow-up visit (Visit 6) should be repeated until the values return to normal or baseline or are no longer considered clinically significant by the Investigator or Medical Monitor.

If clinically significant values do not return to normal/baseline within a period of time judged reasonable by the Investigator, the aetiology should be identified, where possible, and the Sponsor notified.

All protocol-required laboratory tests, as defined in Appendix 2, must be conducted in accordance with the Laboratory Manual and the SoA (FIGS. 7A, 7B and 7C).

If laboratory values from laboratory tests not specified in the protocol and performed at the institution's local laboratory result in the need for a change in participant management or are considered clinically significant by the Investigator (e.g., are considered to be SAEs or AEs or require dose modification), then the results must be recorded.

In the Phase 1 clinical study, a dose-dependent decrease in serum uric acid was observed, which was not considered to be clinically significant. Due to the dose-dependency of the decrease in serum uric acid, reporting of the serum uric acid values during trial conduct would be unblinding. Therefore, all serum uric acid values will be reported at the end of the trial (except at screening where data is needed to determine eligibility). Since the decrease in serum uric acid is generally considered non-clinically significant, it is not reported as an AE during the study. In order to ensure participant safety throughout the trial, the Medical Monitor will review uric acid values in a blinded fashion.

### Pregnancy Testing

Refer to Inclusion Criterion 7 and Exclusion Criterion 22 for pregnancy entry criteria.
Serum or urine pregnancy testing should be conducted as specified in the SoA (FIGS. 7A, 7B and 7C).
Additional serum or urine pregnancy tests may be performed, as determined necessary by the Investigator or required by local regulation, to establish the absence of pregnancy at any time during participation in the study.

### Suicidal Ideation and Behaviour Risk Monitoring

SIB assessments should be conducted whether or not a particular product is known or suspected to be associated with treatment-emergent SIB (Guidance for Industry Suicidal Ideation and Behavior: Prospective Assessment of Occurrence in Clinical Trials. 2012). Participants being treated with NMD670 will be monitored appropriately and observed closely for SIB or any other unusual changes in behaviour, especially at the beginning and end of the course of intervention. Potential suicidality will be evaluated by trained study personnel using the C-SSRS at all visits as specified in the SoA (FIGS. 7A, 7B and 7C).

All factors contributing to SIB should be evaluated, and consideration should be given to discontinuation of the study IMP. In general, an answer of “yes” to questions 4 and/or 5 of the C-SSRS should trigger the risk assessment.

### Adverse Events, Serious Adverse Events, and Other Safety Reporting

The definitions of AEs and SAEs can be found in Appendix 3.

The Investigator and any qualified designees are responsible for detecting, documenting, and reporting events that meet the definition of an AE or SAE and remain responsible for following up on all AEs (see Discontinuation of Study IMP and Participant Discontinuation for stopping rules). This includes events reported by the participant (or, when appropriate, by a caregiver, surrogate, or the participant's legally authorised representative).

The method of recording, evaluating, and assessing causality of AEs and SAEs and the procedures for completing and transmitting SAE reports are provided in Appendix 3.

### Time Period and Frequency for Collecting AE and SAE Information

All AEs and SAEs observed by the Investigator or participant must be reported by the Investigator and evaluated. All events meeting the definition of an AE must be collected and reported. This includes events from the signing of the ICF until the follow-up visit, as specified in the SoA (FIGS. 7A, 7B and 7C).

All SAEs will be recorded and reported to the Sponsor or designee within 24 hours of awareness, as indicated in Appendix 3. The Investigator will submit any updated SAE data to the Sponsor within 24 hours of it being available.

Investigators are not obliged to actively seek information on AEs or SAEs after the conclusion of study participation. However, if the Investigator learns of any SAE, including a death, at any time after a participant has been discontinued from the study, and the Investigator considers the event reasonably related to the study IMP or study participation, the Investigator must promptly notify the Sponsor.

### Method of Detecting Adverse Events and Serious Adverse Events

Care will be taken not to introduce bias when detecting AEs and/or SAEs. Open-ended and non-leading verbal questioning of the participant is the preferred method to inquire about AE occurrence.

### Follow-Up of Adverse Events and Serious Adverse Events

After the initial AE/SAE report, the Investigator is required to proactively follow up with each participant at subsequent visits/contacts. All SAEs will be followed until resolution or stabilisation, until the event is otherwise explained, or until the participant is lost to follow-up (as defined in section on Loss to Follow-Up). Further information on follow-up procedures is given in Appendix 3.

### Regulatory Reporting Requirements for Serious Adverse Events

Prompt notification (within 24 hours; see Appendix 3) by the Investigator to the Sponsor (or designee) of an SAE is essential so that legal obligations and ethical responsibilities towards the safety of participants and the safety of a study IMP under clinical investigation are met.

The Sponsor has a legal responsibility to notify both the local regulatory authority and other regulatory agencies about the safety of a study IMP under clinical investigation. The Sponsor will comply with country-specific regulatory requirements relating to safety reporting to the regulatory authority, institutional review board (IRB)/independent ethics committee (IEC), and Investigators.

An investigator who receives an Investigator Safety Report describing an SAE or other specific safety information (e.g., summary or listing of SAEs) from the Sponsor will review and then file it along with the Investigator's Brochure and will notify the IRB/IEC, if appropriate, according to local requirements.

All SAEs that occur during the study, and all SAEs occurring up to the follow-up Visit 7 days after receiving the last dose of study IMP, whether considered to be associated with the study IMP or not, must be reported within 24 hours to the Sponsor (or designee), as detailed in section Reporting of Adverse Events/Serious Adverse Events.

Investigator safety reports must be prepared for suspected unexpected serious adverse reactions according to local regulatory requirements and Sponsor policy and forwarded to Investigators as necessary.

Regarding SAEs after the conclusion of study participation, refer to section Time Period and Frequency for Collecting AE and SAE Information.

### Pregnancy

Details of all pregnancies in female participants and partners of male participants will be collected from the start of study IMP until the follow-up visit (Visit 6).

If a pregnancy is reported, the Investigator will record pregnancy information on the appropriate form and submit it to the Sponsor (or designee) within 24 hours of learning of the pregnancy in the female participant or partner of a male participant (after obtaining the necessary signed informed consent from the partner).

While pregnancy itself is not considered an AE or SAE, any pregnancy complication or elective termination of a pregnancy for medical reasons will be reported as an AE or SAE.

Abnormal pregnancy outcomes (e.g., maternal serious complications, therapeutic abortion, spontaneous abortion, foetal death, stillbirth, congenital anomalies, and ectopic pregnancy) are considered SAEs and will be reported as such.

The participant/pregnant partner will be followed to determine the outcome of the pregnancy. The Investigator will collect follow-up information on the participant/pregnant partner and the neonate, and the information will be forwarded to the Sponsor.

Any post-study pregnancy-related SAE considered reasonably related to the study IMP by the Investigator will be reported to the Sponsor as described in section Regulatory Reporting Requirements for Serious Adverse Events. While the Investigator is not obligated to actively seek this information in former study participants/pregnant partners, he or she may learn of an SAE through spontaneous reporting.

Any female participant who becomes pregnant while participating in the study will discontinue study IMP.

### Adverse Events of Special Interest

The following AEs are considered AEs of special interest and should be reported promptly to the Sponsor:
Severe myotonia. Spontaneous recovery is the most likely outcome. However, study treatment will be discontinued (see section Discontinuation of Study IMP). This AE of special interest is distinct from non-severe muscle tone abnormalities that may be identified during physical examination, evaluation of muscle strength by dynamometry, and other clinical assessments, which should be reported as AEs if considered clinically relevant.
Urate crystallisation.
Lens opacity worsening. An increase in LOCS III 1.0 (in any single category: nuclear, cortical, or posterior subcapsular) that is associated with a decrease in ETDRS of ≥2 lines.

Any AE of special interest meeting seriousness criteria should also be reported to the Sponsor (or designee), as detailed in section Reporting of Adverse Events/Serious Adverse Events.

The full list of Medical Dictionary for Regulatory Activities preferred terms corresponding to myotonia events will be defined in the SAP.

### Pharmacokinetics

A population PK (PopPK) approach will be used for the study. The PopPK model is based on FIH data in healthy individuals and MG patients.

The following blood samples for PopPK analysis of NMD670 will be collected in all participants:
On day 7, 1 PK sample is taken at any time during the clinic visit.
On day 21 (EOT), 1 sample will be taken close to trough (pre-second daily dose) for NMD670, and 1 sample will be taken around Cₘₐₓ (2 to 4 hours post-second daily dose) as specified in the SoA (FIGS. 7A, 7B and 7C).

For each sample, approximately 3 mL will be collected for measurement of plasma concentrations of NMD670 and its metabolite (NMD1190). The actual date and time of the PK sample and study IMP doses taken on the day of the samples will be recorded in the eCRF. This approach has been well documented to estimate exposures in a new population with good precision from 2 concentrations, in conjunction with a PopPK approach (Sathe et al, 2021).

Instructions for the collection and handling of biological samples will be provided by the Sponsor. Blood sample collection, processing and shipping details will be outlined in a separate Laboratory Manual. In brief, blood will be processed and plasma analysed using validated assays. IMP concentration information that would unblind the study will not be reported to investigative sites or blinded personnel until the study has been unblinded.

### Pharmacodynamics

### Single Fibre Electromyography

Assessment of jitter and blocking by sfEMG will be included as secondary endpoints to evaluate the PD effects of NMD670 on neuromuscular transmission. sfEMG will be performed in the tibialis anterior muscle and possibly in additional muscle groups (e.g., in the upper limb), as scheduled in the SoA (FIGS. 7A, 7B and 7C) (Sanders et al, 2019). Only selected sites where sfEMG is conducted routinely will perform this procedure, and the procedure will be optional for the individual participants at any sites that agree to perform this assessment.

The sfEMG electrode is a needle electrode with a small recording surface that allows for the recording of 1 or more single fibre action potentials from a given motor unit. Time-locked action potentials from at least 2 muscle fibres triggered by voluntary contraction of the tested muscle are recorded. Neuromuscular jitter represents the variation in time intervals between pairs of action potentials. Blocking represents the percentage of pairs in which the action potential is not observed for 1 of the fibres. Recordings will be performed using standardised equipment.

### Genetics

The genetic confirmation of CMT diagnosis will be available from medical history and not routinely performed in this study. Exceptions to this criterion may be allowed on a case-by-case basis if agreed in advance by the Sponsor (e.g., known tested genetic mutation but with unavailable records).

### Biomarkers

The exploratory biomarker Skeletal muscle troponin I, type II, a potential marker of skeletal muscle fibre integrity, will be collected and measured during the study.

Recently, the Critical Path Predictive Safety Testing Consortium evaluated the diagnostic performance of a muscle injury biomarker panel (MIP) compared to creatine kinase (CK) and AST and their correlations with histology scores across 34 different rat studies. The MIP is comprised of Skeletal muscle troponin I, type II, myosin light chain 3, fatty acid binding protein 3, and creatine kinase muscle type. The MIP panel was shown to be more sensitive than the CK and AST activity assays (Burch et al, 2015). Furthermore, in an in vivo rat study performed by NMD Pharma Skeletal muscle troponin I, type II was confirmed as the most adequate and sensitive biomarker of skeletal muscle fibre integrity of those investigated. Based on these data, Skeletal muscle troponin I, type II plasma measurements will be added as an exploratory biomarker to this Phase 2 clinical study. Samples will be taken at all in-clinic visits.

### Immunogenicity Assessments

Immunogenicity is not assessed during the study.

### Storage and Future Use of Biological Samples

NMD Pharma will be responsible for ensuring all biological sample collection and storage is secure and with adequate measures to protect confidentiality, per all applicable local regulations.

All biological samples will be destroyed after analyses; no future use of biological samples is planned for this study.

### Medical Resource Utilisation and Health Economics

Medical resource utilisation and health economics parameters are not evaluated in this study.

### Statistical Considerations

### Statistical Hypotheses

The primary clinical question of interest for the objective of clinical efficacy is:
Is there a change from baseline to day 21 in 6MWT distance in ambulatory adult participants with CMT type 1 and type 2 treated with NMD670 vs placebo?
For the primary objective of the trial, it is also clinically important to evaluate if there is a change from baseline to day 21 in 10MW/RT time and TUG time in ambulatory adult participants with CMT type 1 and type 2 treated with NMD670 vs placebo.

The null hypothesis is that there is no difference between placebo and NMD670. The alternative hypothesis is that there is a difference between placebo and NMD670.

### Multiplicity Adjustment

Consistent with the early phase nature of this study, there will be no adjustment of alpha when testing multiple endpoints and the nominal p-values will be reported.

TABLE 25
Participant Analysis Sets

| Participant | |
|---|---|
| Analysis Set | Description |
| FAS | All randomised participants who received at |
| | least 1 dose of study IMP. |
| EAS | Participants in the FAS without major protocol |
| | violations that may affect the efficacy analysis. |
| PK analysis set | All randomised participants with at least |
| | 1 quantifiable PK concentration for NMD670 or |
| | its metabolite NMD1190. |

Abbreviations: EAS = efficacy analysis set; FAS = full analysis set; IMP = investigational medicinal product; PK = pharmacokinetic.

The FAS will be used to analyse endpoints related to the efficacy and safety. The efficacy analysis set will be used for sensitivity analysis of endpoints and assessments related to efficacy. The PK data will be summarised using the PK analysis set.

For the efficacy analyses, participants will be included in the analyses according to the planned study IMP, whereas for safety analyses, participants will be included in the analyses according to the study IMP they actually received in the treatment period.

Additional analysis sets may be defined to support specific analyses and will be documented in the SAP prior to unblinding.

### Statistical Analyses

### General Considerations

Listings will be generated for all endpoints.

Summary tables and analysis for safety, efficacy, PK, and PD data will be presented by treatment. The main comparison of interest will always be NMD670 vs placebo.

Unless stated otherwise, mean, standard deviation, median, first and third quartiles, minimum and maximum will be presented when summarising continuous variables.

Counts and frequencies will be used for categorical variables.

For safety, efficacy, and PD measurements, baseline will be the last pre-dose value.

Statistical hypotheses will be tested at a 1-sided significance level of 10%. All CIs will have an 80% level of confidence unless stated otherwise.

An overview of study disposition will be provided, including participants randomised and treated with NMD670 and placebo at each site. Reasons for withdrawals will be summarised by primary reason.

Randomisation will be stratified by CMT type, sex and age (>45 years), which will allow for sensitivity analysis.

### Primary Endpoints Analysis

### Definition of Estimand

The primary clinical question of interest for the objective of clinical efficacy is:
Is there a change from baseline to day 21 in 6MWT distance in ambulatory adult participants with CMT type 1 and type 2 treated with NMD670 vs placebo?
For the primary objective of the trial, it is also clinically important to evaluate if there is a change from baseline to day 21 in 10MW/RT time and TUG time in ambulatory adult participants with CMT type 1 and type 2 treated with NMD670 vs placebo.

Clinical efficacy will be investigated using an MMRM.

Specifically, the estimand has the following attributes:
Population:
   The target population of interest is male and female participants aged 18 to 70 years (inclusive) with a diagnosis of CMT disease type 1 or type 2. These are all defined by the inclusion and exclusion criteria.
Patient-Level Endpoint:
   The change from baseline to day 21 in 6MWT distance will be derived for each participant. The baseline value will be derived from the assessment closest to (but before) the first dose.
Intercurrent Events:
   Anticipated intercurrent events include treatment discontinuation and treatment noncompliance. The treatment policy strategy will be used with respect to these types of events; thus, all available data will be used (with imputation if data is missing).
Population-Level Summary:
   The difference in least-squares mean taken from the model contrasts will be used to compare the treatment arms.
   The primary efficacy endpoint will be analysed in all randomly assigned participants by means of the treatment assigned for analysis (FAS).

For the other endpoints evaluating the primary clinical question of interest, namely 10MW/RT time and TUG time, the estimand will be defined with the same attributes with the exception of the Patient-Level Endpoint, for which the corresponding assessment (10MW/RT time or TUG time, respectively) will be used.

### Analytical Method

A comparison will be made between placebo and NMD670 using the change from baseline to day 21 6MWT distance, 10MW/RT time and TUG time using MMRM. The MMRM model will include terms for the stratification factors, namely, CMT type, sex and age (as a continuous covariate). Any other covariates to be included in the model will be described in the SAP. The ordered hierarchy of these endpoints is 6MWT, 10MW/RT time then TUG time, for which nominal p-values will be taken from the respective MMRM analysis.

The SAP will describe supplementary estimands and sensitivity analyses that address both study-related and non-study-related missing data. Missing data for the primary endpoint will be imputed using a multiple imputation approach (the details of which will be included in the SAP, which will be finalised before unblinding).

### Secondary Endpoints Analysis

Comparison of placebo and NMD670 will be performed for all secondary endpoints. For endpoints involving change from baseline, similar methods will be used as for the primary endpoint, i.e., MMRM. For responder endpoints, differences between groups will be compared using chi-squared tests. Logistic regression will also be used to explore the influence of covariates on responder endpoints with further details in the SAP.

### Safety Analyses

Safety outcomes will be analysed in all randomly assigned participants who received at least 1 dose of treatment according to treatment actually received (FAS). Safety endpoints will be evaluated using descriptive statistics.

Further details will be provided in the SAP, which will be finalised prior to unblinding.

### Other Analyses

PK parameters and/or plasma concentrations will be listed.

Other exploratory endpoints will be summarised descriptively.

Exploratory analyses will be detailed in the SAP.

### Interim Analyses

One interim analysis is planned when approximately half of the planned number of participants have reached day 21. Multiplicity adjustment for alpha will be applied for performing the interim analysis using the O'Brien-Fleming type boundary of a Lan-Demets alpha spending function (Lan et al, 1983). Further details of the interim analysis will be included in the DSMB Charter. This unblinded interim analysis (see section Data Safety Monitoring Board) will allow the DSMB to recommend terminating the study for futility reasons or overwhelming success, to continue the study as planned, or to continue the study with some modifications, including re-estimations of the study sample size. It is foreseeable that other adaptations of the study would be possible based on the interim analysis unblinded results.

### Sample Size Determination

Assuming equal allocation of participants (i.e., in a 1:1 ratio), a 1-sided alpha of 10%, a standard deviation of 60 metres (Thomas et al, 2022) and 80% power, it is estimated that a total of 40 participants per group is needed to demonstrate a difference of 28.6 meters or more in the 6MWT between NMD670 and placebo using a 2-sample t-test. Clinical efficacy will be investigated using an MMRM analysis, which will have higher power than the 2-sample t-test used in this sample size calculation. Approximately 110 participants will be screened (assuming a screen failure rate of 25% based on historical data (Attarian et al, 2021)) to achieve 80 randomised participants (40 per group). The study will be enriched to reach approximately 30% participants with CMT type 2.

### Supporting Documentation and Operational Considerations

### Appendix 1: Regulatory, Ethical, and Study Oversight Considerations Regulatory and Ethical Considerations

This study will be conducted in accordance with the protocol and with:
Consensus ethical principles derived from international guidelines including the Declaration of Helsinki and Council for International Organizations of Medical Sciences international ethical guidelines.
Applicable International Council for Harmonisation of Technical Requirements of Pharmaceuticals for Human Use (ICH) Good Clinical Practice (GCP) guidelines.
Applicable laws and regulations including European Union (EU) and United States (US) laws and regulations.

The protocol, protocol amendments, informed consent form (ICF), Investigator's Brochure, and other relevant documents (e.g., advertisements) must be submitted to an institutional review board (IRB)/independent ethics committee (IEC) by the Investigator and reviewed and approved by the IRB/IEC before the study is initiated.

Any amendments to the protocol will require IEC/IRB approval before implementation of changes made to the study design, except for changes necessary to eliminate an immediate hazard to study participants.

Protocols and any substantial amendments to the protocol will require health authority approval prior to initiation, except for changes necessary to eliminate an immediate hazard to study participants.

The Investigator will be responsible for the following, as applicable:
Providing written summaries of the status of the study to the IRB/IEC annually or more frequently in accordance with the requirements, policies, and
procedures established by the IRB/EC.
Notifying the IRB/IEC of serious adverse events or other significant safety findings as required by IRB/IEC procedures.
Overall conduct of the study at the site and adherence to requirements of 21 Code of Federal Regulations (CFR), ICH GCP guidelines, the IRB/IEC, European regulation 536/2014 for clinical studies, European Medical Device Regulation 2017/745 and all other applicable local regulations.

### Financial Disclosure

Investigators and sub-Investigators will provide the Sponsor with sufficient, accurate financial information in accordance with local regulations to allow the Sponsor to submit complete and accurate financial certification or disclosure statements to the appropriate regulatory authorities. Investigators are responsible for providing information on financial interests before study participation, during the course of the study and for 1 year after completion of the study.

### Informed Consent Process

The Investigator or the Investigator's representative will explain the nature of the study, including the risks and benefits, to the potential participant and answer all questions regarding the study.

Potential participants must be informed that their participation is voluntary. The potential participant will be required to sign a statement of informed consent that meets the requirements of 21 CFR 50; local regulations; ICH guidelines; privacy and data protection requirements, where applicable; and the IRB/IEC or study site.

The medical record must include a statement that written informed consent was obtained before the participant was enrolled in the study and the date the written consent was obtained. The authorised person obtaining the informed consent must also sign the ICF.

Participants must be re-consented to the most current version of the ICF(s) during their participation in the study.

A copy of the ICF(s) must be provided to the participant.

Participants who are rescreened are required to sign a new ICF.

If a protocol amendment is required, the ICF may need to be revised to reflect the changes to the protocol. If the ICF is revised, it must be reviewed and approved by the appropriate IEC/IRB and signed by all participants subsequently enrolled in the study as well as those currently enrolled in the study.

### Recruitment Strategy

Participants will be recruited from the clinics of the Investigators and are likely to be known by the Investigators.

Potential participants may be also contacted and informed about the study via patient advocacy groups or via referrals from other physicians or advertisement.

This is a multicentre study, and there will be competitive recruitment.

### Data Protection

Participants will be assigned a unique identifier by the Sponsor. Any participant records or datasets that are transferred to the Sponsor will contain the identifier only; participant names or any information that would make the participant identifiable will not be transferred. The information that is collected by the Sponsor includes sex, age, race, ethnicity, height and weight, medical and surgical history, prior and concomitant medications, current medical condition, and results of the study assessments and procedures as described in FIGS. 7A, 7B and 7C.

The participant must be informed that their personal study-related data will be used by the Sponsor in accordance with local data protection law. The level of disclosure must also be explained to the participant, who will be required to give consent for their data to be used as described in the ICF. All study-related personal data will be stored by the Sponsor in accordance with applicable data privacy regulations for a minimum of 25 years. All bodily material will be destroyed after the analysis has been done and the study is complete.

The participant must be informed that their medical records may be examined by Clinical Quality Assurance auditors or other authorised personnel appointed by the Sponsor, by appropriate IRB/IEC members, or by inspectors from regulatory authorities.

The contract between Sponsor and study sites specifies responsibilities of the parties related to data protection, including handling of data security breaches and respective communication and cooperation of the parties.

Information technology systems used to collect, process, and store study-related data are secured by technical and organisational security measures designed to protect such data.

All clinical trial information shall be recorded, processed, handled, and stored by the Sponsor, Sponsor's representatives or Investigator, as applicable, in such a way that it can be accurately reported, interpreted and verified while the confidentiality of records and the personal data of the participants remain protected in accordance with the applicable law on personal data protection. Organisational and technical arrangements will be implemented to avoid unauthorised access, disclosure, dissemination, and alteration or loss of information and personal data processed.

### Data Safety Monitoring Board

An independent DSMB will be implemented for this study to ensure the safety of participants and the continued validity and scientific merit of the study.

A blinded review of safety data will be conducted after approximately 25% of the planned population has completed the EOT Visit (or more frequently if needed to review emergent safety concerns). An unblinded review of safety and efficacy data will be performed via interim analysis conducted when approximately 50% of the planned population has completed the EOT visit as detailed in the statistical analysis plan.

After each of these data reviews, the DSMB will provide a recommendation to stop the study, continue with modifications, or continue as planned.

Working procedures (including potential unblinding, if necessary) and composition of the DSMB will be described in the DSMB Charter.

### Dissemination of Clinical Study Data

A summary of the results of the clinical study together with a summary that is understandable to a layperson will be provided after the global end (or early termination) of the study in all countries concerned to ensure full availability of all clinical data under this protocol, within 12 months.

A description of this clinical trial will be available on http://www.clinicaltrials.gov, as will the summary of the study results when they are available. The clinical trial and/or summary of study results may also be available on other websites according to the regulations of the countries in which the study is conducted.

Within the EU and the US, the Sponsor will submit a Development Safety Update Report on an annual basis for the study investigational medicinal product (IMP) in this clinical study.

### Data Quality Assurance

All participant data relating to the study will be recorded on printed case report forms (CRFs) or electronic CRFs (eCRFs) unless transmitted to the Sponsor or designee electronically (e.g., laboratory data). The Investigator is responsible for verifying that data entries are accurate and correct by physically or electronically signing the CRF.

Guidance on completion of CRFs will be provided in the CRF completion guideline.

The Investigator must permit study-related monitoring, audits, IRB/IEC review, and regulatory agency inspections and provide direct access to source documents.

Monitoring details describing strategies, including definition of study critical data items and processes (e.g., risk-based initiatives in operations and quality such as risk management and mitigation strategies and analytical risk-based monitoring), methods, responsibilities and requirements, including handling of noncompliance issues and monitoring techniques (central, remote, or on-site monitoring) are provided in the Monitoring Plan.

The Sponsor or designee is responsible for the data management of this study including quality checking of the data.

The Sponsor assumes accountability for actions delegated to other individuals (e.g., contract research organisations).

Records and documents, including signed ICFs, pertaining to the conduct of this study must be retained by the Investigator for 25 years after study completion unless local regulations or institutional policies require a longer retention period. No records may be destroyed during the retention period without the written approval of the Sponsor. No records may be transferred to another location or party without written notification to the Sponsor.

All data generated by the site personnel will be captured electronically at each study site using eCRFs. Data from external sources (such as laboratory data) will be imported into the database. Once the eCRF clinical data have been submitted to the central server at the independent data centre, corrections to the data fields will be captured in an audit trail. The reason for change and the name of the person who performed the change, together with the time and date, will be logged to provide an audit trail.

If additional corrections are needed, the responsible monitor or data manager will raise a query in the electronic data capture (EDC) application. The appropriate staff at the study site will answer queries sent to the Investigator. The name of the staff member responding to the query and the time and date stamp will be captured to provide an audit trail. Once all source data verification is complete and all queries are closed, the monitor will freeze the eCRF page.

The specific procedures to be used for data entry and query resolution using the EDC system/eCRF will be provided to study sites in a training manual. In addition, site personnel will receive training on the EDC system/eCRF.

### Source Documents

Source documents provide evidence for the existence of the participant and substantiate the integrity of the data collected. Source documents are filed at the Investigator's site.

Data reported in the eCRF that are transcribed from source documents must be consistent with the source documents, or the discrepancies must be explained. The Investigator may need to request previous medical records or transfer records, depending on the study. Also, current medical records must be available.

The Investigator must maintain accurate documentation (source data) that supports the information entered in the eCRF.

The Sponsor or designee will perform monitoring to confirm that data entered into the eCRF by authorised site personnel are accurate, complete, and verifiable from source documents; that the safety and rights of participants are being protected; and that the study is being conducted in accordance with the currently approved protocol and any other study agreements, ICH GCP, and all applicable regulatory requirements.

### Study and Site Start and Closure

### First Act of Recruitment

The study start date is the date on which the clinical study will be open for recruitment of participants.

The first act of recruitment is the opening of the first site and will be the study start date.

### Study/Site Termination

The Sponsor or designee reserves the right to close a study site or terminate the study at any time for any reason at the sole discretion of the Sponsor. Study sites will be closed upon study completion. A study site is considered closed when all required documents and study supplies have been collected and a study site closure visit has been performed.

The Investigator may initiate study site closure at any time, provided there is reasonable cause and sufficient notice is given in advance of the intended termination.

Reasons for the early closure of a study site by the Sponsor or Investigator may include but are not limited to:

For study termination:
Discontinuation of further study IMP development.
Sponsor's decision that continuation of the study is unjustifiable for medical or ethical reasons.
Poor enrolment of participants making completion of the study within an acceptable time frame unlikely.

For site termination:
Failure of the Investigator to comply with the protocol, the requirements of the IRB/IEC or local health authorities, the Sponsor's procedures, or GCP guidelines.
Inadequate or no recruitment (evaluated after a reasonable amount of time) of participants by the Investigator.
Total number of participants included earlier than expected.

If the study is prematurely terminated or suspended, the Sponsor shall promptly inform the Investigators, the IECs/IRBs, the regulatory authorities, and any contract research organisations used in the study of the reason for termination or suspension, as specified by the applicable regulatory requirements. The Investigator shall promptly inform the participants and should assure appropriate participant therapy and/or follow-up.

### Publication Policy

The results of this study may be published or presented at scientific meetings.

The Sponsor will comply with the requirements for publication of the study results, regardless of the results. In accordance with standard editorial and ethical practice, the Sponsor will generally support publication of multicentre studies only in their entirety and not as individual site data. In this case, a coordinating Investigator will be designated by mutual agreement.

If additional publications or presentations at scientific meetings are planned by the Investigator, she/he agrees to submit all manuscripts or abstracts to the Sponsor before submission. This allows the Sponsor to protect proprietary information and provide comments.

Authorship will be determined by mutual agreement and in line with International Committee of Medical Journal Editors authorship requirements.

### Protocol Approval and Amendment

Before the start of the study, the study protocol and/or other relevant documents will be approved by the IEC/IRB/Competent Authorities, in accordance with local legal requirements. The Sponsor must ensure that all ethical and legal requirements have been met before the first participant is enrolled in the study.

This protocol is to be followed exactly. To alter the protocol, amendments must be written, receive approval from the appropriate personnel, and receive IRB/IEC/Competent Authority approval prior to implementation (if appropriate). Following approval, the protocol amendment(s) will be submitted to the Investigational New Drug under which the study is being conducted.

Administrative changes (not affecting the participant benefit/risk ratio) may be made without the need for a formal amendment. All amendments will be distributed to all protocol recipients, with appropriate instructions.

### Liability and Insurance

The Sponsor will take out reasonable third-party liability insurance cover in accordance with all legal requirements. The civil liability of the Investigator; the persons instructed by him or her; and the hospital, practice, or institute in which they are employed and the liability of the Sponsor with respect to financial loss due to personal injury and other damage that may arise as a result of carrying out this study are governed by the applicable law.

The Sponsor will arrange for study participants to be insured against financial loss due to personal injury caused by the pharmaceutical products being tested or by medical steps taken in the course of the study.

### Access to Source Data

During the study, a monitor will make site visits to review protocol compliance, compare eCRF entries and individual participant's medical records, assess drug accountability, and ensure that the study is being conducted according to pertinent regulatory requirements. The eCRF entries will be verified with source documentation. The review of medical records will be performed in a manner to ensure that participant confidentiality is maintained.

Checking the eCRF entries for completeness and clarity and cross-checking with source documents will be required to monitor the progress of the study. Moreover, regulatory authorities of certain countries, IRBs, IECs, and/or the Sponsor's Clinical Quality Assurance Group may wish to carry out such source data checks and/or on-site audit inspections. Direct access to source data will be required for these inspections and audits; they will be carried out giving due consideration to data protection and medical confidentiality. The Investigator assures the Sponsor, if involved in monitoring/data management, of the necessary support at all times.

### Appendix 2: Laboratory Tests

The tests detailed in Table 26 will be performed by the central laboratory (exception is the local urine pregnancy test as outlined in footnote 3 of Table 26) at time points detailed in the schedule of activities.

Blood tests for clinical chemistry (glucose) will be drawn after 4-hour fasting.

Local laboratory results are only required in the event that the central laboratory results are not available in time for either confirmation of eligibility, study investigational medicinal product (IMP) administration and/or response evaluation. If a local sample is required, it is important that the sample for central analysis is obtained at the same time. Additionally, if the local laboratory results are used to make either a study IMP decision or response evaluation, the results must be recorded.

Protocol-specific requirements for inclusion or exclusion of participants are detailed in the Study Population section of the protocol.

Additional tests may be performed at any time during the study as determined necessary by the Investigator or required by local regulations.

Investigators must document their review of each laboratory safety report.

TABLE 26
Protocol-Required Laboratory Tests

| Laboratory Tests | Parameters | |
|---|---|---|
| Haematology | Platelet count | WBC count with differential: |
| | RBC count | Neutrophils |
| | Haemoglobin | Lymphocytes |
| | Haematocrit | Monocytes |
| | RBC indices: | Eosinophils |
| | MCV | Basophils |
| | MCH | |
| | MCHC | |
| Haemostasis | INR | aPTT |
| | Prothrombin time | Fibrinogen |
| Clinical | BUN | AST |
| chemistry¹ | Potassium | Total and direct bilirubin |
| | Creatinine | ALT |
| | Sodium | GGT |
| | Glucose (after at least 4-hour | Total protein |
| | fasting) | Albumin |
| | Calcium | Triglycerides |
| | Inorganic phosphate | LDH |
| | Creatine kinase | Alkaline phosphatase² |
| | Chloride | Uric acid |
| | Bicarbonate | |
| Routine | Specific gravity | |
| urinalysis | Uric acid, creatinine, and uric-acid-to-creatinine ratio | |
| | pH, glucose, protein, blood, ketones, bilirubin, urobilinogen, nitrite, and | |
| | leukocyte esterase by dipstick | |
| | Microscopic examination | |
| Pregnancy | Highly sensitive serum or urine hCG pregnancy test (as needed for | |
| testing | women of childbearing potential) at time points detailed in the SoA | |
| | (FIGS. 7A, 7B and 7C)³. | |
| Other | Follicle-stimulating hormone and oestradiol (as needed in women of | |
| screening | non-childbearing potential only) | |
| tests | Urine drug screen (to include cocaine, heroin, opiates, and marijuana)⁴ | |
| | Serology (HIV antibody, HBsAg, and hepatitis C antibody) | |
| Exploratory | Skeletal muscle troponin I, type II | |
| biomarkers | | |
| Pharmacokinetic | NMD670 and its metabolite (NMD1190) | |
| analysis | | |

¹Details of liver chemistry follow-up are given in section Discontinuation of Study IMP
²If alkaline phosphatase is elevated, consider fractionating.
³After screening, local urine testing will be standard for the protocol unless serum testing is required by local regulations or the IRB/IEC or in order to confirm a positive or inconclusive urine test result.
⁴Refer to Exclusion Criterion 12 and Inclusion Criterion 5 for details.
Abbreviations: ALT = alanine aminotransferase; aPTT = activated partial thromboplastin time; AST = aspartate aminotransferase; BUN = blood urea nitrogen; GGT = gamma-glutamyl transferase; HbsAg = hepatitis B surface antigen; hCG = human chorionic gonadotropin; HIV = human immunodeficiency virus; IEC = independent ethics committee; INR = international normalised ratio; IRB = institutional review board; LDH = lactate dehydrogenase; MCH = mean corpuscular haemoglobin; MCHC = mean corpuscular haemoglobin concentration; MCV = mean corpuscular volume; RBC = red blood cell; SoA = schedule of activities; WBC = white blood cell.

### Appendix 3: Adverse Events and Serious Adverse Events: Definitions and Procedures for Recording, Evaluating, Follow-Up, and Reporting

### Definition of Adverse Event

### Adverse Event Definition

An adverse event (AE) is any untoward medical occurrence in a patient or clinical study participant, whether or not considered associated with the study investigational medicinal product (IMP).

AEs detected after administration of the first IMP dose and until the end-of-study Visit are defined as treatment-emergent AEs (TEAEs), whether or not considered related to the study IMP.

NOTE: An AE/TEAE can therefore be any unfavourable and unintended sign (including an abnormal laboratory finding), symptom, or disease (new or exacerbated) temporally not associated or associated with the use of a study IMP.

### Events Meeting the AE Definition

Any abnormal laboratory test results (haematology, clinical chemistry, or urinalysis) or other safety assessment results (e.g., electrocardiogram results, radiological scan results, or vital signs measurements), including those that worsen from screening, considered clinically significant in the medical and scientific judgement of the Investigator (e.g., not related to progression of the underlying disease or more severe than expected for the participant's condition).
Exacerbation of a chronic or intermittent pre-existing condition including either an increase in frequency and/or intensity of the condition.
New condition detected or diagnosed after signing the informed consent form (ICF) even though it may have been present before the start of the study.
Signs, symptoms, or the clinical sequelae of a suspected drug-drug interaction.
Signs, symptoms, or the clinical sequelae of a suspected overdose of either study IMP or a concomitant medication. Intentional overdose taken with possible suicidal/self-harming intent. Such overdoses should be reported regardless of sequelae.
Lack of efficacy or failure of expected pharmacological action per se will not be reported as AEs or serious AEs (SAEs). Such instances will be captured in the efficacy assessments. However, the signs, symptoms, and/or clinical sequelae resulting from lack of efficacy will be reported as AEs or SAEs if they fulfil the definition of an AE or SAE.

### Events NOT Meeting the AE Definition

Any abnormal laboratory findings or other abnormal safety assessment results that are associated with the underlying disease, unless judged by the Investigator to be more severe than expected for the participant's condition.
The disease/disorder being studied or expected progression, signs, or symptoms of the disease/disorder being studied, unless more severe than expected for the participant's condition.
Medical or surgical procedure (e.g., endoscopy or appendectomy): the condition that leads to the procedure is the AE.
Situations in which an untoward medical occurrence did not occur (social and/or convenience admission to a hospital).
Anticipated day-to-day fluctuations of pre-existing diseases or conditions present or detected at the start of the study that do not worsen.

### Definition of SAE

An SAE is an AE that:

a. Results in Death

It should be noted that death per se is not an AE, rather an outcome. The Investigator should always try to report an AE that was considered an immediate cause of death.

b. Is Life-Threatening

The term life-threatening in the definition of serious refers to an event in which the participant was at risk of death at the time of the event. It does not refer to an event that hypothetically might have caused death if it were more severe.

c. Requires Inpatient Hospitalisation or Prolongation of Existing Hospitalisation

In general, hospitalisation signifies that the participant has been admitted (usually involving at least an overnight stay) to the hospital or emergency ward for observation and/or intervention that would not have been appropriate in the physician's office or an outpatient setting. Complications that occur during hospitalisation are AEs. If a complication prolongs hospitalisation or fulfils any other seriousness criteria, the event is serious. When in doubt as to whether hospitalisation occurred or was necessary, the AE should be considered serious.

Hospitalisation for elective intervention of a pre-existing condition that did not worsen from baseline is not considered an AE.

d. Results in Persistent or Significant Disability/Incapacity

The term disability means a substantial disruption of a person's ability to conduct normal life functions.

This definition is not intended to include experiences of relatively minor medical significance such as uncomplicated headache, nausea, vomiting, diarrhoea, influenza, and accidental trauma (e.g., sprained ankle) that may interfere with or prevent everyday life functions but do not constitute a substantial disruption.

e. Is a Congenital Anomaly/Birth Defect
f. Other Situations

Medical or scientific judgement should be exercised by the Investigator in deciding whether SAE reporting is appropriate in other situations such as significant medical events that may jeopardise the participant or may require medical or surgical intervention to prevent one of the other outcomes listed in the above definition. These events should usually be considered serious.

Examples of such events are intensive treatment in an emergency room or at home for allergic bronchospasm, blood dyscrasias or convulsions that do not result in hospitalisation, or development of drug dependency or drug abuse.

### Recording and Follow-Up of Adverse Events and Serious Adverse Events

### AE and SAE Recording

AEs should be collected and recorded for each participant from the date the ICF is signed until the end of their participation in the study, i.e., when the participant has discontinued or completed the study.

When an AE/SAE occurs, it is the responsibility of the Investigator to review all documentation (e.g., hospital progress notes and laboratory and diagnostics reports) related to the event.

The Investigator will then record all relevant AE/SAE information.

AEs may be volunteered spontaneously by the participant or discovered by the study staff during physical examinations or by asking an open, non-leading question such as “How have you been feeling since you were last asked?” All AEs and any required remedial action will be recorded. The nature of the AE, date (and time, if known) of the AE onset, date (and time, if known) of the AE outcome to date, and severity of and action taken for the AE will be documented together with the Investigator's assessment of the seriousness of the AE and causal relationship to study IMP and/or study procedure.

All AEs should be recorded individually in the participant's own words (verbatim) unless, in the opinion of the Investigator, the AEs constitute components of a recognised condition, disease or syndrome. In the latter case, the condition, disease, or syndrome should be named rather than each individual symptom. The AEs will subsequently be coded using the current version of Medical Dictionary for Regulatory Activities.

It is not acceptable for the Investigator to send photocopies of the participant's medical records to the Sponsor (or designee) in lieu of completion of the required form.

There may be instances when copies of medical records for certain cases are requested by the Sponsor (or designee). In this case, all participant identifiers, with the exception of the participant number, will be blinded on the copies of the medical records before submission to the Sponsor (or designee).

The Investigator will attempt to establish a diagnosis of the event based on signs, symptoms, and/or other clinical information. In such cases, the diagnosis (not the individual signs/symptoms) will be documented as the AE/SAE.

### Assessment of Intensity

The Investigator will make an assessment of intensity for each AE and SAE reported during the study and assign a grade according to National Cancer Institute (NCI) Common Terminology Criteria for Adverse Events (CTCAE). The CTCAE displays Grades 1 through 5 with unique clinical descriptions of severity for each AE based on this general guideline:
Grade 1: Mild; asymptomatic or mild symptoms; clinical or diagnostic observations only; intervention not indicated.
Grade 2: Moderate; minimal, local or non-invasive intervention indicated; limiting age-appropriate instrumental activities of daily living (e.g., preparing meals, shopping for groceries or clothes, using the telephone, or managing money).
Grade 3: Severe or medically significant but not immediately life-threatening; hospitalisation or prolongation of hospitalisation indicated; disabling; limiting self-care activities of daily living (e.g., bathing, dressing and undressing, feeding self, using the toilet, and taking medications; not bedridden).
Grade 4: Life-threatening consequences; urgent intervention indicated.
Grade 5: Death related to the AE (not appropriate for some AEs).
Not all grades are appropriate for all AEs. Therefore, some AEs are listed with fewer than 5 options for grade selection.

An event is defined as “serious” when it meets at least 1 of the predefined outcomes as described in the definition of an SAE, independently of NCI-CTCAE grading.

When changes in the intensity of an AE occur more frequently than once a day, the maximum intensity for the event should be noted for that day. Any change in severity of signs and symptoms over a number of days will be captured by recording a new AE, with the amended severity grade and the date (and time, if known) of the change.

### Assessment of Causality

The Investigator is obligated to assess the relationship between study IMP and each occurrence of each AE/SAE. The Investigator will use clinical judgement to determine the relationship.

A reasonable possibility of a relationship conveys that there are facts, evidence, and/or arguments to suggest a causal relationship, rather than a situation where a relationship cannot be ruled out.

Alternative causes, such as underlying diseases, concomitant therapy, and other risk factors, as well as the temporal relationship of the event to study IMP administration, will be considered and investigated.

The Investigator will also consult the Investigator's Brochure in their assessment.

For each AE/SAE, the Investigator must document in the medical notes that he/she has reviewed the AE/SAE and has provided an assessment of causality.

There may be situations in which an SAE has occurred and the Investigator has minimal information to include in the initial report in the electronic data collection tool. However, it is very important that the Investigator always make an assessment of causality for every event before the initial transmission of the SAE data to the electronic data collection tool.

The Investigator may change their opinion of causality in light of follow-up information and send an SAE follow-up report with the updated causality assessment.

The following “binary” decision choice will be used by the Investigator to describe the initial causality assessment:
Related: Reasonable possibility of relatedness
Not related: No reasonable possibility of relatedness

The causality assessment is one of the criteria used when determining regulatory reporting requirements.

### Follow-Up of AEs and SAEs

The Investigator is obligated to perform or arrange for the conduct of supplemental measurements and/or evaluations as medically indicated or as requested by the Sponsor (or designee) to elucidate the nature and/or causality of the AE or SAE as fully as possible. This may include additional laboratory tests or investigations, histopathological examinations, or consultation with other health care professionals.

If a participant dies during participation in the study or during a recognised follow-up period, the Investigator will provide the Sponsor (or designee) with a copy of any post-mortem findings including histopathology.

New or updated information will be recorded in the originally completed form.

The Investigator will submit any updated SAE data to the Sponsor within 24 hours of receipt of the information.

### Reporting of Adverse Events/Serious Adverse Events

In agreement with the provisions of this protocol, the Investigator accepts all legal responsibilities for immediate reporting of SAEs to the Medical Monitor and the Sponsor.

All SAEs must be immediately reported (i.e., within 24 hours of awareness) to the Sponsor. SAEs are reported by entering the SAE data into the study-specific EDC system. Entering the SAE data into the EDC system will automatically notify the Sponsor of the SAE. In the event that the EDC system is inaccessible, an SAE may be reported by:
Email to the SAE email address: safetyreporting@syneoshealth.com
Fax using a paper SAE report form: +1-877-464-7787

If an SAE is initially reported by email or fax, the SAE must also be entered in the EDC system as soon as the EDC system is accessible.

At a minimum, the following information should be provided at the time of the initial report:
Participant number
Event term
At least 1 criterion classifying the event as serious
Name and title of the reporting individual
Causal relationship to the investigational product

The Investigator will assess whether the event is causally related to investigational product. The Sponsor will also assess whether the event is causally related to investigational product, in addition to assessing the overall safety profile of the investigational product. The Sponsor will notify the appropriate regulatory agencies as well as all participating Investigators (where required) of Expedited Safety Reports that occur during the study within the time frames required by each regulatory agency.

Following the initial report, any additional information obtained by the Investigator about the SAE must be reported promptly to the Sponsor in the same manner as described above for the initial SAE report. Any supporting source documentation should be faxed to +1-877-464-7787 or emailed to safetyreporting@syneoshealth.com as soon as possible. Redacted medical record source documentation will be requested for all SAEs.

Where applicable, the Investigator is responsible for submitting information on Expedited Safety Reports received from the Sponsor to her/his local institutional review board (IRB)/independent ethics committee (IEC) as directed by the local-country requirements. Documentation of the applicable submissions to IECs/IRBs must be retained in the appropriate study file(s). As instructed by the Sponsor, Expedited Safety Reports should be retained in the appropriate investigative site study files or with the Investigator's Brochure.

### Reporting of Suspected Unexpected Serious Adverse Reactions by the Sponsor to the Agency

1. The Sponsor's safety representative shall report electronically and without delay to the EudraVigilance database, to the Food and Drug Administration per 21 Code of Federal Regulations 312.32, and to countries outside of these territories all domestic and foreign suspected unexpected serious adverse reactions (SUSARs) as follows:
(a) all SUSARs to IMP(s) occurring in that clinical trial, irrespective of whether the SUSAR has occurred at a clinical trial site in the European Union, United States or a third country outside of these territories.
(b) all SUSARs to IMP(s), occurring in any of the participants of the clinical trial, that are identified by or come to the attention of the Sponsor after the end of the clinical trial.
2. The period for the reporting of SUSARs by the Sponsor to the Agency shall take into account the seriousness of the reaction and shall be as follows:
(a) in the case of fatal or life-threatening SUSARs, as soon as possible and in any event not later than 7 days after the Sponsor became aware of the reaction;
(b) in the case of non-fatal or non-life-threatening SUSARs, not later than 15 days after the Sponsor became aware of the reaction;
(c) in the case of a SUSAR that was initially considered non-fatal or non-life-threatening but that turns out to be fatal or life-threatening, as soon as possible and in any event not later than 7 days after the Sponsor became aware of the reaction being fatal or life-threatening.

The Sponsor shall submit a Development Safety Update Report and a 6-monthly line listing (where required) which include an overview of all SUSARs for the study IMP that have occurred. to all relevant Health Authorities and IECs/IRBs,

### Appendix 4: Contraceptive and Barrier Guidance

### Definitions

### Women of Childbearing Potential

Women in the following categories are considered women of childbearing potential (fertile):
Following menarche
From the time of menarche until becoming postmenopausal unless permanently sterile (see below)
A postmenopausal state is defined as no menses for 12 months without an alternative medical cause.
   A high follicle-stimulating hormone (FSH) level in the postmenopausal range may be used to confirm a postmenopausal state in women not using hormonal contraception or hormone replacement therapy (HRT). However, in the absence of 12 months of amenorrhea, confirmation with more than 1 FSH measurement is required.
   Females on HRT and whose menopausal status is in doubt will be required to use one of the non-oestrogen hormonal highly effective contraception methods if they wish to continue their HRT during the study. Otherwise, they must discontinue HRT to allow confirmation of postmenopausal status before study enrolment.
Permanent sterilisation methods (for the purpose of this study) include:
   Documented hysterectomy
   Documented bilateral salpingectomy
   Documented bilateral oophorectomy
   For individuals with permanent infertility due to an alternate medical cause other than the above (e.g., Mullerian agenesis, androgen insensitivity, or gonadal dysgenesis), Investigator discretion should be applied to determining study entry.

Note: Documentation can come from the site personnel's review of the participant's medical records, medical examination, or medical history interview.
If fertility is unclear (e.g., amenorrhea in adolescents or athletes) and a menstrual cycle cannot be confirmed before the first dose of study investigational medicinal product, additional evaluation should be considered.

Contraception Guidance

Contraceptives^{a} Allowed During the Study
Highly Effective Methods^{b} That Have Low User Dependency
Implantable progestogen-only hormone contraception associated with inhibition of
ovulation^{c}
Intrauterine device
Intrauterine hormone-releasing system^{c}
Bilateral tubal occlusion
Azoospermic partner (vasectomised or due to a medical cause)
Azoospermia is a highly effective contraceptive method provided that the partner is the
sole sexual partner of the woman of childbearing potential and the absence of sperm has
been confirmed. Documentation of azoospermia is required; if not, an additional highly
effective method of contraception should be used.
Highly Effective Methods^{b} That are User Dependent
Combined (oestrogen- and progestogen-containing) hormonal contraception associated
with inhibition of ovulation^{c}
Oral
Intravaginal
Transdermal
Injectable
Progestogen-only hormone contraception associated with inhibition of ovulation^{c}
Oral
Injectable
Sexual abstinence
Sexual abstinence is considered a highly effective method only if defined as refraining
from heterosexual intercourse during the entire period of risk associated with the study
IMP. The reliability of sexual abstinence needs to be evaluated in relation to the duration
of the study and the preferred and usual lifestyle of the participant.
^{a}Contraceptive use by men or women should be consistent with local regulations regarding the use of contraceptive methods for those participating in clinical studies.
^{b}Failure rate of <1% per year when used consistently and correctly. Typical use failure rates differ from those when used consistently and correctly.
^{c}Male condoms must be used in addition to hormonal contraception. If locally required, in accordance with Clinical Trial Facilitation Group guidelines, acceptable contraceptive methods are limited to those that inhibit ovulation as the primary mode of action.
NOTES:
Periodic abstinence (calendar, symptothermal, and postovulation methods), withdrawal (coitus interruptus), spermicides only, and lactational amenorrhea method are not acceptable methods of contraception. Male condoms and female condoms should not be used together (due to risk of failure from friction).

### REFERENCES

Allard et al, 2014. Allard L, Rode G, Jacquin-Courtois S, Pouget M C, Rippert P, Hamroun D, Poirot I, Berard C, Vuillerot C; le groupe d'etude CMT MFM. The motor function measure to study limitation of activity in children and adults with Charcot-Marie-Tooth disease. Ann Phys Rehabil Med. 2014 December; 57(9-10):587-99. The entire content of the foregoing document is incorporated by reference herein.
Andres et al, 2012. Andres P L, Skerry L M, Munsat T L, Thornell B J, Szymonifka J, Schoenfeld D A, Cudkowicz M E. Validation of a new strength measurement device for amyotrophic lateral sclerosis clinical trials. Muscle Nerve. 2012 January; 45(1):81-5. The entire content of the foregoing document is incorporated by reference herein.
ATS 2002. ATS Committee on Proficiency Standards for Clinical Pulmonary Function Laboratories. ATS statement: guidelines for the six-minute walk test. Am J Respir Crit Care Med. 2002; 166(1):111-7. Erratum in: Am J Respir Crit Care Med. 2016; 193(10):1185. The entire content of the foregoing document is incorporated by reference herein.
Attarian et al, 2021 Attarian S, Young P, Brannagan T H, Adams D, Van Damme P, Thomas F P, Casanovas C, Tard C, Walter M C, Pereon Y, et al. A double-blind, placebo-controlled, randomized trial of PXT3003 for the treatment of Charcot-Marie-Tooth type 1A. Orphanet J Rare Dis. 2021; 16:433. The entire content of the foregoing document is incorporated by reference herein.
Berg et al, 1989. Berg K, Wood-Dauphine S, Williams J I, Gayton D. Measuring balance in the elderly: preliminary development of an instrument, Physiotherapy Canada. 1989; 41:304-311. The entire content of the foregoing document is incorporated by reference herein.
Boentert et al, 2010. Boentert M, Dziewas R, Heidbreder A, Happe S, Kleffner I, Evers S, Young P. Fatigue, reduced sleep quality and restless legs syndrome in Charcot-Marie-Tooth disease: a web-based survey. J Neurol. 2010 April; 257(4):646-52. The entire content of the foregoing document is incorporated by reference herein.
Bragadin et al, 2015. Bragadin M M, Francini L, Bellone E, Grandis M, Reni L, Canneva S, Gemelli C, Ursino G, Maggi G, Mori L, et al. Tinetti and Berg balance scales correlate with disability in hereditary peripheral neuropathies: a preliminary study. Eur J Phys Rehabil Med. 2015; 51:423-427. The entire content of the foregoing document is incorporated by reference herein.
Bray et al, 2020. Bray P, Cornett KMD, Estilow T, Pareyson D, Zuccarino R, Skorupinska M, Pipis M, Sowden J E, Scherer S, Reilly M M, et al. Reliability of the Charcot-Marie-Tooth functional outcome measure. J Peripher Nerv Syst. 2020; 25:288-291. The entire content of the foregoing document is incorporated by reference herein.
Brincks et al, 2019. Brincks J, Callesen J, Dalgas U, Johnsen E. Test-retest reliability and limits of agreement of the Six-Spot Step Test in people with Parkinson's disease. Clin Rehabil. 2019; 33:285-292. The entire content of the foregoing document is incorporated by reference herein.
Burch et al, 2015 Burch P M, Pogoryelova O, Goldstein R, Bennett D, Guglieri M, Straub V, Bushby K, Lochmuller H, Morris C. Muscle-Derived Proteins as Serum Biomarkers for Monitoring Disease Progression in Three Forms of Muscular Dystrophy. J Neuromuscul Dis. 2015; 2:241-255. The entire content of the foregoing document is incorporated by reference herein.
Chylack et al, 1993 Chylack L T, Jr., Wolfe J K, Singer D M, Leske M C, Bullimore M A, Bailey I L, Friend J, McCarthy D, Wu S Y. The Lens Opacities Classification System III. The Longitudinal Study of Cataract Study Group. Arch Ophthalmol. 1993; 111:831-836. The entire content of the foregoing document is incorporated by reference herein.
de França Costa et al, 2018 de França Costa IMP, Nunes P S, de Aquino Neves E L, Lima Santos Barreto L C, Garcez C A, Souza C C, Pereira Oliveira P M, Sales Ferreira L A, Brandão Lima V N, de Souza AraOjo A A. Evaluation of muscle strength, balance and functionality of individuals with type 2 Charcot-Marie-Tooth Disease. Gait Posture. 2018; 62:463-467. The entire content of the foregoing document is incorporated by reference herein.
Dunaway et al, 2014. Dunaway S, Montes J, Garber C E, Carr B, Kramer S S, Kamil-Rosenberg S, Strauss N, Sproule D, De Vivo D C. Performance of the timed “up & go” test in spinal muscular atrophy. Muscle Nerve. 2014; 50:273-277. The entire content of the foregoing document is incorporated by reference herein.
Eichinger et al, 2018. Eichinger K, Burns J, Cornett K, Bacon C, Shepherd M L, Mountain J, Sowden J, Shy R, Shy M E, Herrmann D N. The Charcot-Marie-Tooth Functional Outcome Measure (CMT-FOM). Neurology. 2018 Oct. 9; 91(15):e1381-e1384. The entire content of the foregoing document is incorporated by reference herein.
Febrer et al, 2010. Febrer A, Rodriguez N, Alias L, Tizzano E. Measurement of muscle strength with a handheld dynamometer in patients with chronic spinal muscular atrophy. J Rehabil Med. 2010 March; 42(3):228-31. The entire content of the foregoing document is incorporated by reference herein.
Fridman et al, 2015. Fridman V, Bundy B, Reilly M M, Pareyson D, Bacon C, Burns J, Day J, Feely S, Finkel R S, Grider T, Kirk C A, Herrmann D N, Laurá M, Li J, Lloyd T, Sumner C J, Muntoni F, Piscosquito G, Ramchandren S, Shy R, Siskind C E, Yum S W, Moroni I, Pagliano E, Zuchner S, Scherer S S, Shy M E; Inherited Neuropathies Consortium. CMT subtypes and disease burden in patients enrolled in the Inherited Neuropathies Consortium natural history study: a cross-sectional analysis. J Neurol Neurosurg Psychiatry. 2015 August; 86(8):873-8. The entire content of the foregoing document is incorporated by reference herein.
Graham et al, 2006. Graham R C, Hughes R A. A modified peripheral neuropathy scale: the Overall Neuropathy Limitations Scale. J Neurol Neurosurg Psychiatry. 2006 August; 77(8):973-6. The entire content of the foregoing document is incorporated by reference herein.
Grønnebak et al, 2023 Gronnebok T, Haahr-Lillevang H, Kelly K, Bartlett A, Tankisi H, Bold J, Hutchison J, Quiroz J, Pedersen T, Andersen H, et al. Neuromuscular Junction Transmission Dysfunction in Patients with Charcot-Marie-Tooth Disease—The ESTABLISH Study In: Peripheral Nerve Society Annual Meeting 2023.
Copenhagen; 2023. The entire content of the foregoing document is incorporated by reference herein.
Harbo et al, 2012. Harbo T, Brincks J, Andersen H. Maximal isokinetic and isometric muscle strength of major muscle groups related to age, body mass, height, and sex in 178 healthy subjects. Eur J Appl Physiol. 2012 January; 112(1):267-75. The entire content of the foregoing document is incorporated by reference herein.
Johnson et al, 2013 Johnson N E, Heatwole C R, Ferguson M, Sowden J E, Jeanat S, Herrmann D N. Patient identification of the symptomatic impact of charcot-marie-tooth disease type 1A. J Clin Neuromuscul Dis. 2013; 15:19-23. The entire content of the foregoing document is incorporated by reference herein.
Johnson et al, 2018 Johnson N E, Heatwole C, Creigh P, McDermott M P, Dilek N, Hung M, Bounsanga J, Tang W, Shy M E, Herrmann D N. The Charcot-Marie-Tooth Health Index: Evaluation of a Patient-Reported Outcome. Ann Neurol. 2018; 84:225-233. The entire content of the foregoing document is incorporated by reference herein.
Kendall et al, 2005. Kendall F P, Rodgers M, Romani W, Provance P G et al. Muscle testing and function with posture and pain. 5th ed. Philadelphia: Lippincott Williams & Wilkins; 2005. The entire content of the foregoing document is incorporated by reference herein.
Kondori et al, 2020. Kondori R E, Cetin S Y, Erel S. The Validity and Reliability of the Six-Spot Step Test (SSST) in Older Adults. Topics in Geriatric Rehabilitation. 2020; 36. The entire content of the foregoing document is incorporated by reference herein.
Krajewski et al, 2000. Krajewski K M, Lewis R A, Fuerst D R, Turansky C, Hinderer S R, Garbern J, et al. (July 2000). Neurological dysfunction and axonal degeneration in Charcot-Marie-Tooth disease type 1A. Brain. 123 (7): 1516-1527. The entire content of the foregoing document is incorporated by reference herein.
Krosschell et al, 2022. Krosschell K J, Townsend E L, Kiefer M, Simeone S D, Zumpf K, Welty L, Swoboda K J. Natural history of 10-meter walk/run test performance in spinal muscular atrophy: A longitudinal analysis. Neuromuscul Disord. 2022 February; 32(2):125-134. The entire content of the foregoing document is incorporated by reference herein.
Kreutzfeldt et al, 2017. Kreutzfeldt M, Jensen H B, Ravnborg M, Markvardsen L H, Andersen H, Sindrup S H. The six-spot-step test—a new method for monitoring walking ability in patients with chronic inflammatory polyneuropathy. J Peripher Nerv Syst. 2017; 22:131-138. The entire content of the foregoing document is incorporated by reference herein.
Krupp et al, 1989. Krupp L B, LaRocca N G, Muir-Nash J, Steinberg A D. The fatigue severity scale. Application to patients with multiple sclerosis and systemic lupus erythematosus. Arch Neurol. 1989 October; 46(10):1121-3. The entire content of the foregoing document is incorporated by reference herein.
Lan et al, 1983 Lan KKG, DeMets D L. Discrete Sequential Boundaries for Clinical Trials. Biometrika. 1983; 70:659-663. The entire content of the foregoing document is incorporated by reference herein.
Mandarakas et al, 2024 Mandarakas M R, Eichinger K J, Bray P, Cornett KMD, Shy M E, Reilly M M, Ramdharry G M, Scherer S S, Pareyson D, Estilow T, et al. Multicenter Validation of the Charcot-Marie-Tooth Functional Outcome Measure. Neurology. 2024; 102:e207963. The entire content of the foregoing document is incorporated by reference herein.
Mathiowetz et al, 1985. Mathiowetz V, Weber K, Kashman N, Volland G. Adult Norms for the Nine Hole Peg Test of Finger Dexterity. The Occupational Therapy Journal of Research. 1985; 5(1):24-38. The entire content of the foregoing document is incorporated by reference herein.
Merlini et al, 2002. Merlini L, Mazzone E S, Solari A, Morandi L. Reliability of hand-held dynamometry in spinal muscular atrophy. Muscle Nerve. 2002 July; 26(1):64-70. The entire content of the foregoing document is incorporated by reference herein.
Montes et al, 2010. Montes J, McDermott M P, Martens W B, Dunaway S, Glanzman A M, Riley S, Quigley J, Montgomery M J, Sproule D, Tawil R, et al. Six-Minute Walk Test demonstrates motor fatigue in spinal muscular atrophy. Neurology. 2010; 74:833-838. The entire content of the foregoing document is incorporated by reference herein.
Mori et al, 2019 Mori L, Prada V, Signori A, Pareyson D, Piscosquito G, Padua L, Pazzaglia C, Fabrizi G M, Smania N, Picelli A, et al. Outcome measures in the clinical evaluation of ambulatory Charcot-Marie-Tooth 1A subjects. Eur J Phys Rehabil Med. 2019; 55:47-55. The entire content of the foregoing document is incorporated by reference herein.
Mori et al, 2020. Mori L, Signori A, Prada V, Pareyson D, Piscosquito G, Padua L, Pazzaglia C, Fabrizi G M, Picelli A, Schenone A. Treadmill training in patients affected by Charcot-Marie-Tooth neuropathy: results of a multicenter, prospective, randomized, single-blind, controlled study. Eur J Neurol. 2020; 27:280-287. The entire content of the foregoing document is incorporated by reference herein.
Murphy et al, 2011. Murphy S M, Herrmann D N, McDermott M P, Scherer S S, Shy M E, Reilly M M, Pareyson D. Reliability of the CMT neuropathy score (second version) in Charcot-Marie-Tooth disease. J Peripher Nerv Syst. 2011 September; 16(3):191-8. The entire content of the foregoing document is incorporated by reference herein.
Nieuwenhuis et al, 2006. Nieuwenhuis M M, Van Tongeren H, Sorensen P S, Ravnborg M. The six spot step test: a new measurement for walking ability in multiple sclerosis. Mult Scler. 2006 August; 12(4):495-500. The entire content of the foregoing document is incorporated by reference herein.
Niu et al, 2017. Niu H X, Wang R H, Xu H L, Song B, Yang J, Shi C H, Li Y S, Zhang B Q, Wang S P, Yong Q, Wang Y Y, Xu Y M. Nine-hole Peg Test and Ten-meter Walk Test for Evaluating Functional Loss in Chinese Charcot-Marie-Tooth Disease. Chin Med J (Engl). 2017 Aug. 5; 130(15):1773-1778. The entire content of the foregoing document is incorporated by reference herein.
Pareyson et al, 2011 Pareyson D, Reilly M M, Schenone A, Fabrizi G M, Cavallaro T, Santoro L, Vita G, Quattrone A, Padua L, Gemignani F, et al. Ascorbic acid in Charcot-Marie-Tooth disease type 1A (CMT-TRIAAL and CMT-TRAUK): a double-blind randomised trial. Lancet Neurol. 2011; 10:320-328. The entire content of the foregoing document is incorporated by reference herein.
Pedersen et al, 2021. Pedersen T H, Macdonald W A, Broch-Lips M, Halldorsdottir O, Baekgaard Nielsen O. Chloride channel inhibition improves neuromuscular function under conditions mimicking neuromuscular disorders. Acta Physiol (Oxf). 2021 October; 233(2):e13690. The entire content of the foregoing document is incorporated by reference herein.
Personius et al, 1994. Personius K E, Pandya S, King W M, Tawil R, McDermott M P. Facioscapulohumeral dystrophy natural history study: standardization of testing procedures and reliability of measurements. The FSH D Y Group. Phys Ther. 1994 March; 74(3):253-63. The entire content of the foregoing document is incorporated by reference herein.
Piscosquito et al, 2015. Piscosquito G, Reilly M M, Schenone A, Fabrizi G M, Cavallaro T, Santoro L, Manganelli F, Vita G, Quattrone A, Padua L, et al. Responsiveness of clinical outcome measures in Charcot-Marie-Tooth disease. European Journal of Neurology. 2015; 22:1556-1563. The entire content of the foregoing document is incorporated by reference herein.
Podsiadlo et al, 1991. Podsiadlo D, Richardson S. The timed “Up & Go”: a test of basic functional mobility for frail elderly persons. J Am Geriatr Soc. 1991; 39:142-148. The entire content of the foregoing document is incorporated by reference herein.
Pogemiller et al, 2020. Pogemiller K, Garibay E, Pierz K, Acsadi G, Ounpuu S. Comparison of gait patterns and functional measures between Charcot-Marie-Tooth disease type I and II in children to young adults. Gait Posture. 2020; 77:236-242. The entire content of the foregoing document is incorporated by reference herein.
Ramdharry et al, 2012 Ramdharry G M, Pollard A J, Marsden J F, Reilly M M. Comparing gait performance of people with Charcot-Marie-Tooth disease who do and do not wear ankle foot orthoses. Physiother Res Int. 2012; 17:191-199. The entire content of the foregoing document is incorporated by reference herein.
Sanders et al, 2019. Sanders D B, Arimura K, Cui L, Erta M, Farrugia M E, Gilchrist J, Kouyoumdjian J A, Padua L, Pitt M, Stalberg E. Guidelines for single fiber EMG. Clin Neurophysiol. 2019 August; 130(8):1417-1439. The entire content of the foregoing document is incorporated by reference herein.
Sandroff et al, 2015. Sandroff B M, Motl R W, Sosnoff J J, Pula J H. Further validation of the Six-Spot Step Test as a measure of ambulation in multiple sclerosis. Gait Posture. 2015; 41:222-227. The entire content of the foregoing document is incorporated by reference herein.
Saporta et al, 2011. Saporta A S, Sottile S L, Miller L J, Feely S M, Siskind C E, Shy M E. Charcot-Marie-Tooth disease subtypes and genetic testing strategies. Ann Neurol. 2011 January; 69(1):22-33. The entire content of the foregoing document is incorporated by reference herein.
Sathe et al, 2021 Sathe A G, Brundage R C, Ivaturi V, Cloyd J C, Chamberlain J M, Elm J J, Silbergleit R, Kapur J, Coles L D. A pharmacokinetic simulation study to assess the performance of a sparse blood sampling approach to quantify early drug exposure. Clinical and Translational Science. 2021; 14:1444-1451. The entire content of the foregoing document is incorporated by reference herein.
Scherer, 2011. Scherer S S. The debut of a rational treatment for an inherited neuropathy?J Clin Invest. 2011; 121:4624-4627. The entire content of the foregoing document is incorporated by reference herein.
Sleigh et al, 2014. Sleigh J N, Grice S J, Burgess R W, Talbot K, Cader M Z. Neuromuscular junction maturation defects precede impaired lower motor neuron connectivity in Charcot-Marie-Tooth type 2D mice. Hum Mol Genet. 2014 May 15; 23(10):2639-50. The entire content of the foregoing document is incorporated by reference herein.
Solari et al, 2008. Solari A, LaurA M, Salsano E, Radice D, Pareyson D. Reliability of clinical outcome measures in Charcot-Marie-Tooth disease. Neuromuscular Disorders. 2008; 18:19-26. The entire content of the foregoing document is incorporated by reference herein.
Solomon et al, 2019 Solomon S D, Goldberg M F. ETDRS Grading of Diabetic Retinopathy: Still the Gold Standard?Ophthalmic Res. 2019; 62:190-195. The entire content of the foregoing document is incorporated by reference herein.
Spaulding et al, 2016. Spaulding E L, Sleigh J N, Morelli K H, Pinter M J, Burgess R W, Seburn K L. Synaptic Deficits at Neuromuscular Junctions in Two Mouse Models of Charcot-Marie-Tooth Type 2d. J Neurosci. 2016 Mar. 16; 36(11):3254-67. The entire content of the foregoing document is incorporated by reference herein.
Svensson et al, 2006. Svensson E, Hager-Ross C. Hand function in Charcot Marie Tooth: test retest reliability of some measurements. Clin Rehabil. 2006 October; 20(10):896-908. The entire content of the foregoing document is incorporated by reference herein.
Thomas et al, 2022 Thomas F P, Brannagan T H, 3rd, Butterfield R J, Desai U, Habib A A, Herrmann D N, Eichinger K J, Johnson N E, Karam C, Pestronk A, et al. Randomized Phase 2 Study of ACE-083 in Patients With Charcot-Marie-Tooth Disease. Neurology. 2022; 98:e2356-e2367. The entire content of the foregoing document is incorporated by reference herein.
Vincent et al, 2007. Vincent K A, Carr A J, Walburn J, Scott D L, Rose M R. Construction and validation of a quality of life questionnaire for neuromuscular disease (INQoL). Neurology. 2007 Mar. 27; 68(13):1051-7. The entire content of the foregoing document is incorporated by reference herein.
Vinci et al, 2005. Vinci P, Serrao M, Millul A, Deidda A, De Santis F, Capici S, Martini D, Pierelli F, Santilli V. Quality of life in patients with Charcot-Marie-Tooth disease. Neurology. 2005 Sep. 27; 65(6):922-4. The entire content of the foregoing document is incorporated by reference herein.
White et al, 2019 E. White A M, C. Hollett, J. Aldrich, E. Lugo, A. Gray, A. H. Phillips, B. Hernandez, L. Grajkowska. Voice of the Patient Report for Charcot-Marie-Tooth Disease and Inherited Neuropathies. In; 2019. The entire content of the foregoing document is incorporated by reference herein.
Witoonpanich et al, 2011. Witoonpanich R, Dejthevaporn C, Sriphrapradang A, Pulkes T. Electrophysiological and immunological study in myasthenia gravis: diagnostic sensitivity and correlation. Clin Neurophysiol. 2011; 122:1873-1877. The entire content of the foregoing document is incorporated by reference herein.

1. A method for treating Charcot-Marie-Tooth disease in a subject comprising administering a therapeutic dose of a composition comprising (2S)-2-[4-bromo-2-(1,2-oxazol-3-yl)phenoxy]propanoic acid, a pharmaceutically acceptable salt thereof, a hydrate thereof, a polymorph thereof, a tautomer thereof, or a solvate thereof, wherein the therapeutic dose ranges from 100 to 1500 mg.

2. The method according to claim 1, wherein the therapeutic dose ranges from 200 to 600 mg.

3. The method according to claim 1, wherein the therapeutic dose is administered one, two or three times daily.

4. The method according to claim 1, wherein the composition is administered orally.

5. The method according to claim 1, wherein the composition is a solid dosage form.

6. The method according to claim 5, wherein the composition comprises silicified microcrystalline cellulose.

7. The method according to claim 1, wherein the subject is suffering from CMT1, CMT2, CMT3 or CMT4.

8. The method according to claim 1, wherein the composition is administered orally using a solid dosage form and provides a plasma concentration-time profile of (2S)-2-[4-bromo-2-(1,2-oxazol-3-yl)phenoxy]propanoic acid, the pharmaceutically acceptable salt thereof, the hydrate thereof, the polymorph thereof, the tautomer thereof, or the solvate thereof, that achieves a mean Cₘₐₓ of about 12,760 to about 27,440 ng/mL after administration with a single dose of 400 mg of (2S)-2-[4-bromo-2-(1,2-oxazol-3-yl)phenoxy]propanoic acid, the pharmaceutically acceptable salt thereof, the hydrate thereof, the polymorph thereof, the tautomer thereof, or the solvate thereof.

9. The method according to claim 1, wherein the subject experiences an increase in a total distance walked when determined using a 6-minute walk test after treatment with (2S)-2-[4-bromo-2-(1,2-oxazol-3-yl)phenoxy]propanoic acid, the pharmaceutically acceptable salt thereof, the hydrate thereof, the polymorph thereof, the tautomer thereof, or the solvate thereof.

10. A kit-of-parts comprising:
(2S)-2-[4-bromo-2-(1,2-oxazol-3-yl)phenoxy]propanoic acid, a pharmaceutically acceptable salt thereof, a hydrate thereof, a polymorph thereof, a tautomer thereof, or a solvate thereof, and
a peripheral myelin protein 22 (PMP22) down regulator.

11. The kit-of-parts for use according to claim 10, wherein the kit-of-parts comprises 100 to 1500 mg of (2S)-2-[4-bromo-2-(1,2-oxazol-3-yl)phenoxy]propanoic acid, the pharmaceutically acceptable salt thereof, the hydrate thereof, the polymorph thereof, the tautomer thereof, or the solvate thereof.

12. (canceled)

13. The kit-of-parts according to claim 10, wherein the kit-of-parts contains a therapeutic dose of 100 to 1500 mg of (2S)-2-[4-bromo-2-(1,2-oxazol-3-yl)phenoxy]propanoic acid.

14. The kit-of-parts according to claim 10, wherein (2S)-2-[4-bromo-2-(1,2-oxazol-3-yl)phenoxy]propanoic acid, the pharmaceutically acceptable salt thereof, the hydrate thereof, the polymorph thereof, the tautomer thereof, or the solvate thereof is present in the kit-of-parts at a therapeutic dose ranging from 200 to 600 mg.

15. A composition comprising:
a therapeutic dose of (2S)-2-[4-bromo-2-(1,2-oxazol-3-yl)phenoxy]propanoic acid, a pharmaceutically acceptable salt thereof, a hydrate thereof, a polymorph thereof, a tautomer thereof, or a solvate thereof, wherein the therapeutic dose ranges from 100 to 1500 mg.

16. The composition of claim 15, wherein the therapeutic dose ranges from 200 to 600 mg.

17. The composition of claim 15, wherein the composition is a solid dosage form.

18. The composition of claim 17, wherein the composition comprises silicified microcrystalline cellulose.

19. The composition of claim 15, comprising a peripheral myelin protein 22 (PMP22) down regulator.
